(19) United States
(12) Patent Application Publication
(10) Pub. No.: US 2010/0298314 A1
(43) Pub. Date: Nov. 25, 2010

(54) NOVEL JNK INHIBITORS

(75) Inventors: Panduranga Adulla P. Reddy, Walpole, MA (US); M. Arshad Siddiqui, Newton, MA (US); Praveen K. Tadikonda, Norwood, MA (US); Umar Faruk Mansoor, Framingham, MA (US); Gerald W. Shipps, JR., Stoneham, MA (US); David B. Belanger, Cambridge, MA (US); Lianyun Zhao, Burlington, MA (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/519,733

(22) PCT Filed: Dec. 17, 2007
(86) PCT No.: PCT/US07/25780

§ 371 Date: Feb. 24, 2010

Related U.S. Application Data
(60) Provisional application No. 60/875,989, filed on Dec. 20, 2006.

(51) Int. Cl. A61K 31/5377; C07D 471/04; A61K 31/437; A61K 31/496; A61K 31/497; A61K 31/519; A61K 31/4985; A61P 29/00; A61P 19/02; A61P 11/06; A61P 17/06; A61P 3/10; A61P 9/00; A61P 37/00

(52) U.S. Cl. 514/233.2; 546/121; 544/127; 544/362; 544/357; 544/121; 544/324; 546/114; 544/405; 544/131; 544/122; 544/281; 544/350; 514/300; 514/253.04; 514/252.11; 514/301; 514/255.05; 514/259.3; 514/249

(57) ABSTRACT

Disclosed are substituted imidazo[1,2-a]pyridines, imidazo[1,2-a]pyrazines, imidazo[1,2-c]pyrimidines and imidazo[1,2-d]triazines compounds of the formula: (1.0) Also disclosed are methods for treating JNK1 and ERK mediated diseases using the compounds of formula 1.0.

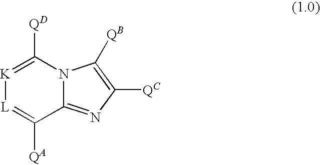

### REFERENCE TO RELATED APPLICATION

This application claims the benefit of U.S. Provisional Application No. 60/875,989 filed Dec. 20, 2007, the disclosure of which is incorporated herein by reference thereto.

### FIELD OF THE INVENTION

The present invention relates to novel substituted imidazo[1,2-a]pyridines, imidazo[1,2-a]pyrazines, imidazo[1,2-c]pyrimidines and imidazo[1,2-d]triazines, pharmaceutical compositions comprising said compounds, and methods for treating diseases or conditions, such as, for example, inflammation, autoimmune diseases, rheumatoid arthritis (RA), psoriasis, metabolic diseases, cardiovascular disease, and neurodegenerative diseases, by administering at least one of said compounds. The novel compounds of this invention are inhibitors of Kinases, and are therefore inhibitors of MAP kinases, and in turn are therefore inhibitors of JNK, ERK1 and ERK2. Thus, for example, the novel compounds of this invention inhibit c-Jun-N-terminal kinase, and therefore the novel compounds of this invention are used to treat or inhibit diseases mediated by c-Jun-N-terminal kinase.

### BACKGROUND OF THE INVENTION

Protein Kinases are divided into two families (1) tyrosine kinase family and (2) serine and threonine kinase family depending on their phosphorylation site (tyrosine, or serine and threonine. Protein kinse activity controls a wide variety of cell life such as growth, differentiation and proliferation. Some of the examples for tyrosine kinase are ALK4, Azl, Brk, EphB4, Fer, Fgr, JAK family (JAK1 and JAK2), Ret, TrkA, Tec family BTK, IKK, ITK and examples for serine and threonine kinase are Ark5, Msk1, Nek2, Pim (Pim1 and Pim2), PLK, Rockl and II, SGK1,2 3, MEK, Erk, Chk, Aurrora and C-met kinases.

C-Jun-N-terminal kinases (i.e., JNKs), which belong to the mitogen activated protein kinase family, are triggered in response to cytokines, mitogens, osmotic stress and ultraviolet readiation. JNKs are divided into three (JNK1, JNK2 and JNK3) major isoforms depending on their gene sequence. Further, these JNKs are divided into 10 splicing isoforms in cells (Gupta, S., T. Barret, A. J., Whitmarsh, J. Cavanagh, H. K. Sluss, B. Derijard, and R. J. Davis 1996, EMBO J. 15, 2760-2770). JNK1 and JNK2 are ubiquitously expressed (Mohit, A. A., Martin, J. H., Miller, C. A Neuron 14, 67-70, 1995), where as JNK3 is expressed in brain and to a lesser extent in the heart and testes.

JNKs are activated by dual phosphorylation of Thr 183 and Tyr 185 by MKK4 and MKK7 kinases (Lin A., Minden A., Martinetto H., Claret F.-Z., Lange-Carter C., Mercurio F., Johnson G. L., and Karin M. Science 268: 286-289, 1995). MKK4 preferentially phosphorylates JNK on tyrosine whereas MKK7 phosphorylates JNK on threonine. Activated c-Jun-N-terminal kinase in turn activates by phosphorylating various transcription factors such as c-Jun, AP1, ATF2, IRS1, NFAT4 and Bcl-2, etc. (Karin M and Hunter T. Curr. Biol. 5, 747-757, 1995 and Shaulian, E., and Karin, M., Nat. Cell Biol. 4, E131-136, 2002). Either JNK1 or JNK2 knockout studies in mice revealed a deficiency in T-helper cells (Dong, C.; Yang, D. D.; Wysk, M.; Whitmarsh, A. J.; Davis, R. J.; Flavell, R. A., Science 1998, 282, 2092-2095; Yang, D. D.; Conze, D.; Whitmarsh, A. J.; Barrett, T.; Davis, R. J.; Rincon, M.; Flavell, R. A. Immunity 1998, 9, 575-585.; Sabapathy, K.; Hu, Y.; Kallunki, T.; Schreiber, M.; David, J. P.; Jochum, W.; Wagner, E. F.; Karin, M., Curr. Biol. 1999, 9, 116-125), whereas double knockouts are embryonic lethal (Tournier, C.; Hess, P.; Yang, D. D.; Xu, J.; Turner, T. K.; Nimnual, A.; Bar-Sagi, D.; Jones, S. N.; Flavell, R. A.; Davis, R. J., Science 2000, 288, 870-874). The JNK3 knockout mouse exhibit resistance to kainic acid induced apoptosis in the hippocampus and to subsequent seizures (Yang, D. D.; Kuan, C. Y.; Whitmarsh, A. J.; Rincon, M.; Zheng, T. S.; Davis, R. J.; Rakic, P.; Flavell, R. A., Nature 1997, 389, 865-870).

Those skilled in the art know that the JNK pathway is activated in several diseases, such as, for example, inflammatory, neurodegenerative and metabolic diseases. Those skilled in the art also know that JNK activation is required for the transformation induced by RAS, an oncogene activated in many human cancers.

In view of the interest in treating diseases mediated by c-Jun-N-terminal kinase, compounds that inhibit c-Jun-N-terminal kinase would be a welcome contribution to the art. This invention provides that contribution.

The processes involved in tumor growth, progression, and metastasis are mediated by signaling pathways that are activated in cancer cells. The ERK pathway plays a central role in regulating mammalian cell growth by relaying extracellular signals from ligand-bound cell surface tyrosine kinase receptors such as erbB family, PDGF, FGF, and VEGF receptor tyrosine kinase. Activation of the ERK pathway is via a cascade of phosphorylation events that begins with activation of Ras. Activation of Ras leads to the recruitment and activation of Raf, a serine-threonine kinase.

Activated Raf then phosphorylates and activates MEK1/2, which then phosphorylates and activates ERK1/2. When activated, ERK1/2 phosphorylates several downstream targets involved in a multitude of cellular events including cytoskeletal changes and transcriptional activation. The ERK/MAPK pathway is one of the most important for cell proliferation, and it is believed that the ERK/MAPK pathway is frequently activated in many tumors. Ras genes, which are upstream of ERK1/2, are mutated in several cancers including colorectal, melanoma, breast and pancreatic tumors. The high Ras activity is accompanied by elevated ERK activity in many human tumors. In addition, mutations of BRAF, a serine-threonine kinase of the Raf family, are associated with increased kinase activity. Mutations in BRAF have been identified in melanomas (60%), thyroid cancers (greater than 40%) and colorectal cancers. These observations indicate that the ERK1/2 signalling pathway is an attractive pathway for anticancer therapies in a broad spectrum of human tumours.

Therefore, a welcome contribution to the art would be small-molecules (i.e., compounds) that inhibit ERK activity (i.e., ERK1 and ERK2 activity), which small-molecules would be useful for treating a broad spectrum of cancers, such as, for example, melanoma, pancreatic cancer, thyroid cancer, colorectal cancer, lung cancer, breast cancer, and ovarian cancer. Such a contribution is provided by this invention.

### SUMMARY OF THE INVENTION

The present invention provides novel compounds useful for treating or preventing diseases (or conditions) associated with the Kinase pathway. Thus, the present invention provides novel compounds useful for treating or preventing diseases (or conditions) associated with MAP Kinases, such as, for example, JNK1, ERK1 and ERK2.

Thus, for example, the present invention provides a method of treating or preventing conditions associated with JNK activation or JNK pathway using novel compounds of formula 1.0.

This invention provides novel compounds that are inhibitors of Kinase, and therefore MAP Kinases, such as, for example, inhibitors of JNK (e.g., JNK1). The novel compounds of this invention have the formula:

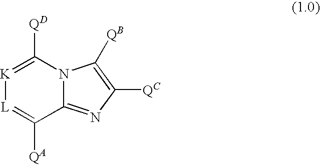

or the pharmaceutically acceptable salts, esters and solvates thereof.

This invention also provides Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794.

This invention also provides compounds of formula 1.0 (e.g., Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794.) in purified and isolated form.

This invention also provides compounds of formula 1.0 (e.g., Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794) in purified form.

This invention also provides compounds of formula 1.0 (e.g., Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794) in isolated form.

This invention also provides pharmaceutically acceptable salts of the compounds of formula 1.0 (e.g., Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794).

This invention also provides pharmaceutically acceptable esters of the compounds of formula 1.0 (e.g., Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794).

This invention also provides solvates of the compounds of formula 1.0 (e.g., Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794).

This invention also provides a pharmaceutical composition comprising at least one (e.g., 1, 2 or 3, or 1 or 2, or 1, and usually 1) compound of formula 1.0, and a pharmaceutically acceptable carrier.

This invention also provides a pharmaceutical composition comprising a compound of formula 1.0, and a pharmaceutically acceptable carrier.

This invention also provides a method of inhibiting JNK (e.g., JNK1) in a patient in need of such treatment, said method comprising administering to said patient an effective amount of at least one (e.g., 1, 2 or 3, or 1 or 2, or 1, and usually 1) compound of formula 1.0.

This invention also provides a method of inhibiting JNK (e.g., JNK1) in a patient in need of such treatment, said method comprising administering to said patient an effective amount of a compound of formula 1.0.

This invention also provides a method of treating a JNK (e.g., JNK1) mediated disease in a patient in need of such treatment, said treatment comprising administering to said patient an effective amount of at least one (e.g., 1, 2 or 3, or 1 or 2, or 1, and usually 1) compound of formula 1.0.

This invention also provides a method of treating a JNK (e.g., JNK1) mediated disease in a patient in need of such treatment, said treatment comprising administering to said patient an effective amount of a compound of formula 1.0.

This invention also provides any one of the above methods for treating a JNK mediated disease wherein said JNK mediated disease is selected from the group consisting of: inflammation, autoimmune disorders (such as, for example, rheumatoid arthritis, multiple sclerosis, asthma, inflammatory bowel disease, psoriasis, pancreatitis, septic shock, transplant rejection and bronchitis), metabolic diseases (such as, for example, diabetes, insulin resistance, and obesity), neurological diseases (such as, for example, Alzeimer's, epilepsy, parkinson's disease, spinal card injury, memory and attention disorders), pain and related syndromes, cancer (such as, for example, breast, colorectal, pancreatic, ovarian, prostate and small cell lung cancer), cardiovascular diseases (such as, for example, hypertrophy and other types of left ventricular remodeling, ischemia/reperfusion injury, angiogenesis and atherogenesis), hepatic ischemia, reperfusion injury, lung fibrosism and liver fibrosis.

This invention also provides any one of the above methods for treating a JNK mediated disease wherein inflammation is treated.

This invention also provides any one of the above methods for treating a JNK mediated disease wherein rheumatoid arthritis is treated.

This invention also provides any one of the above methods for treating a JNK mediated disease wherein asthma is treated.

This invention also provides any one of the above methods for treating a JNK mediated disease wherein multiple sclerosis is treated.

This invention also provides any one of the above methods for treating a JNK mediated disease wherein inflammatory bowel disease is treated.

This invention also provides any one of the above methods for treating a JNK mediated disease wherein psorisis is treated.

This invention also provides any one of the above methods for treating a JNK mediated disease wherein diabetes is treated.

This invention also provides any one of the above methods for treating a JNK mediated disease wherein autoimmune disorders are treated.

This invention also provides any one of the above methods for treating a JNK mediated disease wherein metabolic diseases are treated.

This invention also provides any one of the above methods for treating a JNK mediated disease wherein neurological diseases are treated.

This invention also provides any one of the above methods for treating a JNK mediated disease wherein pain is treated.

This invention also provides any one of the above methods for treating a JNK mediated disease wherein cancer is treated.

This invention also provides any one of the above methods for treating a JNK mediated disease wherein cardiovascular diseases are treated.

This invention is provides any one of the above methods for treating a JNK mediated disease wherein the compound of formula 1 is administered in combination with at least one other active ingredient know in the art for the treatment of said disease. For example, in the treatment of cancer, the compound of formula 1 is administered in combination with at least one (e.g., 1, 2 or 3, or 1 or 2, or 1) chemotherapeutic agent. Administration “in combination with” means the drugs are administered during the same treatment protocol, for example, administration sequentially or consecutively during the treatment protocol. Examples of a chemotherapeutic agents include, for example, antimetabolites, such as, for example, taxol.

This invention also provides any one of the above methods wherein said treatment comprises administering to said patient an effective amount of a pharmaceutical composition comprising at least one (e.g., 1, 2 or 3, or 1 or 2, or 1, and usually 1) compound of formula 1.0 and a pharmaceutically acceptable carrier.

This invention also provides any one of the above methods wherein said treatment comprises administering to said patient an effective amount of a pharmaceutical composition comprising a compound of formula 1.0 and a pharmaceutically acceptable carrier.

This invention also provides a method of inhibiting ERK (i.e., inhibiting the activity of ERK) in a patient in need of such treatment comprising administering to said patient an effective amount of at least one (e.g., 1, 2 or 3, 1 or 2, and usually 1) compound of formula 1.0.

This invention also provides a method of inhibiting ERK1 (i.e., inhibiting the activity of ERK1) in a patient in need of such treatment comprising administering to said patient an effective amount of at least one (e.g., 1, 2 or 3, 1 or 2, and usually 1) compound of formula 1.0.

This invention also provides a method of inhibiting ERK2 (i.e., inhibiting the activity of ERK2) in a patient in need of such treatment comprising administering to said patient an effective amount of at least one (e.g., 1, 2 or 3, 1 or 2, and usually 1) compound of formula 1.0.

This invention also provides a method of inhibiting ERK1 and ERK2 (i.e., inhibiting the activity of ERK1 and ERK2) in a patient in need of such treatment comprising administering to said patient an effective amount of at least one (e.g., 1, 2 or 3, 1 or 2, and usually 1) compound of formula 1.0.

This invention also provides a method for treating cancer in a patient in need of such treatment, said method comprising administering to said patient an effective amount of at least one (e.g., 1, 2 or 3, 1 or 2, and usually 1) compound of formula 1.0.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method of treating cancer in a patient in need of such treatment, said method comprising administering to said patient an effective amount of at least one (e.g., 1, 2 or 3, 1 or 2, and usually 1) compound of formula 1.0 in combination with at least one (e.g., 1, 2 or 3, 1 or 2, and usually 1) signal transduction inhibitor.

This invention also provides a method of treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 in combination with at least one (e.g., 1, 2 or 3, 1 or 2, and usually 1) signal transduction inhibitor.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma, in a patient in need of such treatment, said method comprising administering to said patient an effective amount of at least one (e.g., 1, 2 or 3, 1 or 2, and usually 1) compound of formula 1.0.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating cancer in a patient in need of such treatment, said method comprising administering to said patient an effective amount of at least one (e.g., 1, 2 or 3, 1 or 2, and usually 1) compound of formula 1.0, wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent wherein said cancer is selected from the group consisting of: melanoma, pancreatic cancer, thyroid cancer, colorectal cancer, lung cancer, breast cancer, and ovarian cancer.

This invention also provides a method for treating melanoma in a patient in need of such treatment, said method comprising administering to said patient an effective amount of at least one (e.g., 1, 2 or 3, 1 or 2, and usually 1) compound of formula 1.0.

This invention also provides a method for treating melanoma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating melanoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating melanoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating pancreatic cancer in a patient in need of such treatment, said method comprising administering to said patient an effective amount of at least one (e.g., 1, 2 or 3, 1 or 2, and usually 1) compound of formula 1.0.

This invention also provides a method for treating pancreatic cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating pancreatic cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating pancreatic cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating thyroid cancer in a patient in need of such treatment, said method comprising administering to said patient an effective amount of at least one (e.g., 1, 2 or 3, 1 or 2, and usually 1) compound of formula 1.0.

This invention also provides a method for treating thyroid cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating thyroid cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating thyroid cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating colorectal cancer in a patient in need of such treatment, said method comprising administering to said patient an effective amount of at least one (e.g., 1, 2 or 3, 1 or 2, and usually 1) compound of formula 1.0.

This invention also provides a method for treating colorectal cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating colorectal cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating colorectal cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating lung cancer in a patient in need of such treatment, said method comprising administering to said patient an effective amount of at least one (e.g., 1, 2 or 3, 1 or 2, and usually 1) compound of formula 1.0.

This invention also provides a method for treating lung cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating lung cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating lung cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating breast cancer in a patient in need of such treatment, said method comprising administering to said patient an effective amount of at least one (e.g., 1, 2 or 3, 1 or 2, and usually 1) compound of formula 1.0.

This invention also provides a method for treating breast cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating breast cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating breast cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating ovarian cancer in a patient in need of such treatment, said method comprising administering to said patient an effective amount of at least one (e.g., 1, 2 or 3, 1 or 2, and usually 1) compound of formula 1.0.

This invention also provides a method for treating ovarian cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating ovarian cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating ovarian cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides methods of treating breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents).

This invention also provides methods of treating breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents).

This invention also provides methods of treating breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

This invention also provides methods of treating breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

The methods of treating breast cancer described herein include the treatment of hormone-dependent metastatic and advanced breast cancer, adjuvant therapy for hormone-dependent primary and early breast cancer, the treatment of ductal carcinoma in situ, and the treatment of inflammatory breast cancer in situ.

The methods of treating hormone-dependent breast cancer can also be used to prevent breast cancer in patients having a high risk of developing breast cancer.

Thus, this invention also provides methods of preventing breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents).

This invention also provides methods of preventing breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents).

This invention also provides methods of preventing breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

This invention also provides methods of preventing breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) a in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating brain cancer (e.g., glioma, such as glioma blastoma multiforme) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0, in combination with an effective amount of a chemotherapeutic agent wherein said chemotherapeutic agent is temozolomide.

This invention also provides a method for treating brain cancer (e.g., glioma, such as glioma blastoma multiforme) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of a chemotherapeutic agent, wherein said chemotherapeutic agent is temozolomide.

This invention also provides a method for treating prostate cancer in a patient in need of such treatment, said method comprising administering to said patient an effective amount of at least one (e.g., 1, 2 or 3, 1 or 2, and usually 1) compound of formula 1.0.

This invention also provides a method for treating prostate cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating prostate cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating prostate cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myelodysplastic syndrome in a patient in need of such treatment, said method comprising administering to said patient an effective amount of at least one (e.g., 1, 2 or 3, 1 or 2, and usually 1) compound of formula 1.0.

This invention also provides a method for treating myelodysplastic syndrome in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myelodysplastic syndrome in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating myelodysplastic syndrome in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating acute myelogenous leukemia (AML) in a patient in need of such treatment, said method comprising administering to said patient an effective amount of at least one (e.g., 1, 2 or 3, 1 or 2, and usually 1) compound of formula 1.0.

This invention also provides a method for treating acute myelogenous leukemia (AML) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating acute myelogenous leukemia (AML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating acute myelogenous leukemia (AML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelomonocytic leukemia (CMML) in a patient in need of such treatment, said method comprising administering to said patient an effective amount of at least one (e.g., 1, 2 or 3, 1 or 2, and usually 1) compound of formula 1.0.

This invention also provides a method for treating chronic myelomonocytic leukemia (CMML) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelomonocytic leukemia (CMML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating chronic myelomonocytic leukemia (CMML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelogenous leukemia (chronic myeloid leukemia, CML) in a patient in need of such treatment, said method comprising administering to said patient an effective amount of at least one (e.g., 1, 2 or 3, 1 or 2, and usually 1) compound of formula 1.0.

This invention also provides a method for treating chronic myelogenous leukemia (chronic myeloid leukemia, CML) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelogenous leukemia (chronic myeloid leukemia, CML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating chronic myelogenous leukemia (chronic myeloid leukemia, CML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating bladder cancer in a patient in need of such treatment, said method comprising administering to said patient an effective amount of at least one (e.g., 1, 2 or 3, 1 or 2, and usually 1) compound of formula 1.0.

This invention also provides a method for treating bladder cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating bladder cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating bladder cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating non-Hodgkin's lymphoma in a patient in need of such treatment, said method comprising administering to said patient an effective amount of at least one (e.g., 1, 2 or 3, 1 or 2, and usually 1) compound of formula 1.0.

This invention also provides a method for treating non-Hodgkin's lymphoma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating non-Hodgkin's lymphoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating non-Hodgkin's lymphoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating multiple myeloma in a patient in need of such treatment, said method comprising administering to said patient an effective amount of at least one (e.g., 1, 2 or 3, 1 or 2, and usually 1) compound of formula 1.0.

This invention also provides a method for treating multiple myeloma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating multiple myeloma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating multiple myeloma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

In the methods of this invention the compounds of this invention can be administered concurrently or sequentially (i.e., consecutively) with the chemotherapeutic agents or the signal transduction inhibitor.

The methods of treating cancers described herein can optionally include the administration of an effective amount of radiation (i.e., the methods of treating cancers described herein optionally include the administration of radiation therapy).

### DETAILED DESCRIPTION OF THE INVENTION

As used herein, unless indicated otherwise, the abbreviations below have the meanings indicated.
ACN Acetonitrile
AcOH Acetic acid
DCC Dicyclohexylcarbodiimide
DCU Dicyclohexylurea
DCM Dichloromethane
DIAD Diisopropylazodicarboxylate
DIEA Diisopropylethylamine
DMAP 4-Dimethylaminopyridine
DME Dimethoxyethane
DMF Dimethylformamide
DMFDMA N,N-Dimethylformamide dimethylacetal
DMSO Dimethyl sulfoxide
EtOAc Ethyl acetate
EtOH Ethanol
HATU N,N,N′,N′-Tetramethyl-O-(7-Azabenzotriazol-1-yl)Uronium hexafluorophosphate
Hex hexanes
HPLC High pressure liquid chromatography
LCMS Liquid chromatography mass spectrometry
mCPBA meta-Chloroperoxybenzoic acid
MeOH Methanol
NaH Sodium hydride
NMR Nuclear magnetic resonance
PFP Pentafluorophenol
PMB p-methoxybenzyl
Pyr Pyridine
RT Room temperature
TFA Trifluoroacetic acid
THF Tetrahydrofuran
TLC Thin layer chromatography
TMS Trimethylsilyl

As herein, the following terms, unless indicated otherwise, have thefollowing meanings indicated:

“Patient” includes both human and animals (and preferably a human being).

“Mammal” means humans and other mammalian animals.

“One or more” includes, for example, 1, 2 or 3, or 1 or 2, or 1.

“At least one” includes, for example, 1, 2 or 3, or 1 or 2, or 1.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Z₁Z₂N—, Z₁Z₂N-alkyl-, Z₁Z₂NC(O)—, Z₁Z₂NSO₂— and —SO₂NZ₁Z₂, wherein Z₁ and Z₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

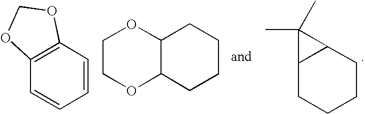

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” (e.g., “heterocycloalkyl”) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a ring system (as described above) that is substituted with a single moiety (e.g., ═O) which simultaneously replaces two available hydrogens on the same carbon atom on a ring system. An example of such a heterocyclyl is pyrrolidone:

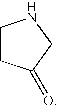

“Heterocyclylalkyl” (e.g., “heterocycloalkylalkyl”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a ring systemt (as described above) that is substituted with a single moiety (e.g., ═O) which simultaneously replaces two available hydrogens on the same carbon atom on a ring system. An example of such a heterocyclenyl is pyrrolidinone:

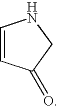

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

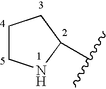

there is no —OH attached directly to carbons marked 2 and 5.

It should also be noted that tautomeric forms such as, for example, the moieties:

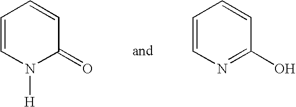

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R³, etc.) occurs more than one time in any constituent or in Formula 1.0, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

“Prodrug” represents compounds that are rapidly transformed, for example, by hydrolysis in blood, in vivo to the parent compound, i.e., to the compounds of formula 1.0 or to a salt and/or to a solvate thereof; A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. The scope of this invention includes Prodrugs of the novel compounds of this invention.

For example, if a compound of Formula 1.0 or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyl-oxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxy-carbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkyl-carbamoyl-(C₁-C₂)alkyl and piperidino-, pyrrolidino- or morpholino(C₂-C₃)alkyl, and the like.

Similarly, if a compound of Formula 1.0 contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)-ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula 1.0 incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

This invention is also provides compounds of formula 1.0 in pure or isolated form.

This invention also includes pharmaceutically esters of the compounds of formula 1.0.

This invention also includes pharmaceutically acceptable solvates of the compounds of formula 1.0.

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of formula 1.0 can form salts which are also within the scope of this invention. Reference to a compound of formula 1.0 herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula 1.0 contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formula 1.0 may be formed, for example, by reacting a compound of formula 1.0 with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of formula 1.0, and salts, solvates, esters and prodrugs thereof, may exist in their tautomeric form (for example, as an amide or imino ether). All such tautomeric forms are contemplated herein as part of the present invention.

The compounds of formula 1.0 may contain asymmetric or chiral centers, and, therefore, exist in different stereoisomeric forms. It is intended that all stereoisomeric forms of the compounds of formula 1.0 as well as mixtures thereof, including racemic mixtures, form part of the present invention. In addition, the present invention embraces all geometric and positional isomers. For example, if a compound of formula 1.0 incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of formula 1.0 may be atropisomers (e.g., substituted biaryls) and are considered as part of this invention. Enantiomers can also be separated by use of chiral HPLC column.

It is also possible that the compounds of formula 1.0 may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula 1.0 can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of formula 1.0, and of the salts, solvates, esters and prodrugs of the compounds of formula 1.0, are intended to be included in the present invention.

The compounds according to the invention have pharmacological properties; in particular, the compounds of formula 1.0 are inhibitors of JNK (e.g., JNK1, 2 or 3).

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

“Anti-cancer agent”, “chemotherapeutic agent”, and “antineoplastic agent” have the same meaning, and these terms represent the drugs (medicaments) used to treat cancer.

“Antineoplastic agent” represents a chemotherapeutic agent effective against cancer.

“Compound”, with reference to the antineoplastic agents, includes the agents that are antibodies.

“Concurrently” represents (1) simultaneously in time (e.g., at the same time); or (2) at different times during the course of a common treatment schedule;

“Consecutively” means one following the other;

“Different”, as used in the phrase “different antineoplastic agents”, means that the agents are not the same compound or structure. Preferably, “different” as used in the phrase “different antineoplastic agents” means not from the same class of antineoplastic agents. For example, one antineoplastic agent is a taxane, and another antineoplastic agent is a platinum coordinator compound.

“Effective amount” or “therapeutically effective amount” is meant to describe an amount of compound or a composition of the present invention effective in inhibiting or treating the diseases described herein, e.g., cancer, or effective in inhibiting JNK (e.g., JNK1). That is, an effective amount is that amount that produces the desired therapeutic, ameliorative, inhibitory or preventative effect. For example, the amount of the compound or composition that results in: (a) the reduction, alleviation or disappearance of one or more symptoms caused by the disease (e.g., the cancer), (b) the reduction of tumor size, (c) the elimination of the tumor, and/or (d) long-term disease stabilization (growth arrest) of the tumor.

“Sequentially” means (1) administration of one component of the method ((a) compound of the invention, or (b) chemotherapeutic agent and/or radiation therapy) followed by administration of the other component or components. After adminsitration of one component, the next component can be administered substantially immediately after the first component, or the next component can be administered after an effective time period after the first component. The effective time period is the amount of time given for realization of maximum benefit from the administration of the first component; and

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Lines drawn into the ring systems indicate that the indicated bond may be attached to any of the substitutable ring carbon atoms of any ring when more than one ring is present.

It should also be noted that any carbon or heteroatom with unsatisfied valences in the text, schemes, examples, structural formulae, and any Tables herein is assumed to have the hydrogen atom or atoms to satisfy the valences.

This invention provides novel compounds that are JNK (e.g., JNK1) inhibitors. The novel compounds of this invention have the formula:

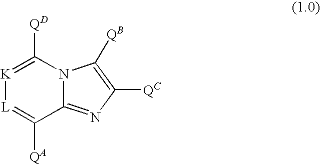

or the pharmaceutically acceptable salts, esters, and solvates thereof, wherein:

K is selected from the group consisting of: CH, N, —C(alkyl)- (e.g., —C(CH₃)—), —C(aryl)- (e.g., —C(phenyl)-), —C(halo)- (e.g., —C(F)—, or —C(Cl)— or —C(Br)—), and —C(R^{C})— wherein R^{C} is selected from the group consisting of:

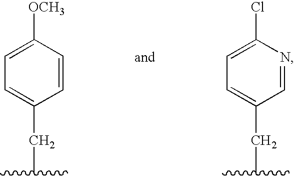

(and preferably K is CH);

L is CH or N (and preferably CH);

Q^{A} is selected from the group consisting of:
(A) —C(O)NR¹R²;
(B) —N(R¹⁴)₂ (e.g., —NH₂);
(C) unsubstituted heteroaryl (such as, for example, imidazolyl, pyrazolyl, oxadiazolyl, pyrimidinyl, pyridazinyl, and benzo fused heteroaryls (i.e., a heteroaryl fused to a benzene ring such that the heteroaryl ring and the benzene ring have two adjacent carbons in common, such as, for example, benzoimidazolyl and quinolinyl);
(D) substituted heteroaryl (such as, for example, substituted imidazolyl, substituted pyrazolyl, substituted oxadiazolyl, substituted pyrimidinyl, substituted pyridazinyl, and substituted benzo fused heteroaryls (i.e., a heteroaryl fused to a benzene ring such that the heteroaryl ring and the benzene ring have two adjacent carbons in common, such as, for example, substituted benzoimidazolyl and substituted quinolinyl), and wherein said substituted heteroaryl is substituted with one or more (e.g., 1 to 3) substituents selected from the group consisting of: (1) halo (e.g., Cl, F, Br, and I), (2) heteroaryl (e.g., pyridyl and pyrazinyl), benzo fused heteroaryl (e.g., benzoimidazolyl), (3) heterocycloalkyl (e.g., morpholinyl and pyrrolidinyl), (4) benzodioxolyl, (5) aryl (e.g., phenyl), (6) substituted aryl (e.g., substituted phenyl) wherein the substituent is —S(O)₂alkyl (e.g., —S(O)₂CH₃), (7) alkyl (e.g., methyl), (8) —CF₃, and wherein examples of said substituted heteroaryl moiety (D) include, but are not limited to:

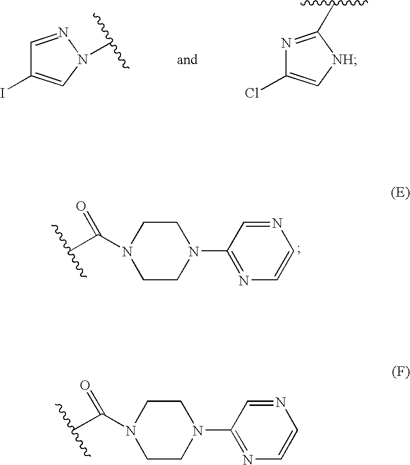

substituted with one or more (e.g., 1 to 3) substituents selected from the group consisting of:
   (1) -(alkylene)₁₋₆-heterocycloalkyl (e.g., -(alkylene)₁₋₂-heterocycloalkyl), such as, for example, —(CH₂)₂morpholinyl and —CH₂piperidinyl,
   (2) aryl (e.g., phenyl),
   (3) substituted aryl (e.g., substituted phenyl, such as, for example, chlorophenyl, fluorophenyl and cyanophenyl),
   (4) —C(O)R¹¹,
   (5) —C(O)-aryl (e.g. —C(O)phenyl), and
   (6) -(alkylene)₁₋₆-N(R¹²)₂ (e.g., -(alkylene)₁₋₃-N(R¹²)₂), such as, for example, —(CH₂)₃N(R¹²)₂, and
   wherein said substituted aryl moiety (3) (e.g., substituted phenyl) is substituted with one or more (e.g., 1 to 3) substitutents independently selected from the group consisting of: halo (e.g., Cl and F), and —CN;

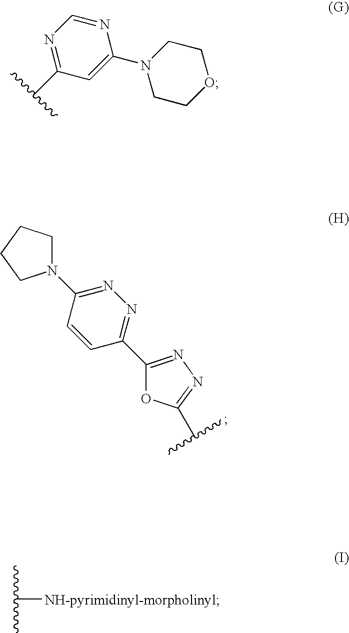

(J) H;
(K) —C(O)-heterocycloalkyl-heteroaryl (e.g., —C(O)-piperazinyl-piperidyl);
(L) —C(O)-piperazinyl-(alkylene)₁₋₆-substituted aryl wherein the substituents are independently selected from halo (e.g., Cl, F, Br);
(M) —C(O)-heterocycloalkyl-(alkylene)₁₋₆-heterocycloalkyl (e.g., —C(O)-piperazinyl-(alkylene)₁₋₆-heterocycloalkyl);
(N) —C(O)-piperazinyl-(alkylene)₁₋₆-heteroaryl;
(O) alkyl (e.g., C₁₋₆alkyl);
(P) —C(O)-heterocycloalkyl wherein said heterocycloalkyl is substituted with -(alkylene)₁₋₆-N(R¹²)₂ wherein each R¹² is independently selected;
(Q) —C(O)-heterocycloalkyl-(alkylene)₁₋₆-(alkyl (e.g., C₁₋₆alkyl) substituted heterocycloalkyl) (e.g., —C(O)-piperazinyl-CH₂—N-methylpiperidinyl);
(R) -(alkylene)₁₋₆-benzo[1,3]dioxolyl;
(S) -(alkylene)₁₋₆-N(R¹)(R²) wherein R¹ and R² are as defined above,
(T) —NH-heteroaryl-heteroaryl (e.g.,

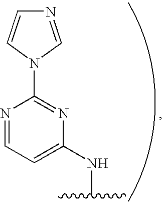

(U) —NH-(fused heteroarylheteroaryl), such as, for example,

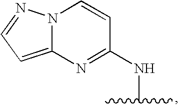

(V) —NH-(substituted heteroaryl), such as, for example:
   —NH-heteroaryl-heterocycloalkyl, such as, for example,

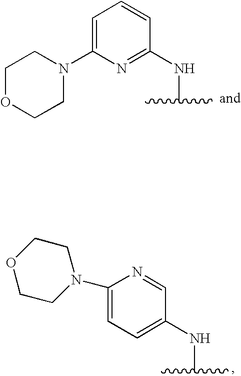

and
—NH-heteroaryl-heteroaryl, such as, for example,

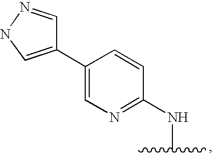

(W) —NH-heteroaryl-NH-heterocycloalkyl, such as, for example,

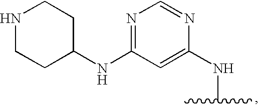

(X) biaryl (i.e., -aryl-aryl),
(Y) biheteroaryl (-heteroaryl-heteroaryl),
(Z) substituted biaryl (i.e., substituted aryl-aryl), and
(AA) substituted biheteroaryl (i.e., -substitued heteroaryl-heteroaryl), such as, for example, -heteroaryl-heteroaryl-heterocycloalkyl, such as,

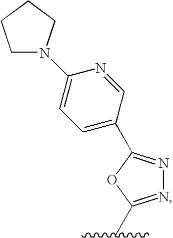

Q^{B} is selected from the group consisting of:
(A) —C(O)NR¹⁵R¹⁶;
(B) —C(O)—R²¹, and
   wherein examples of said —C(O)—R²¹ moiety include, but are not limited to:

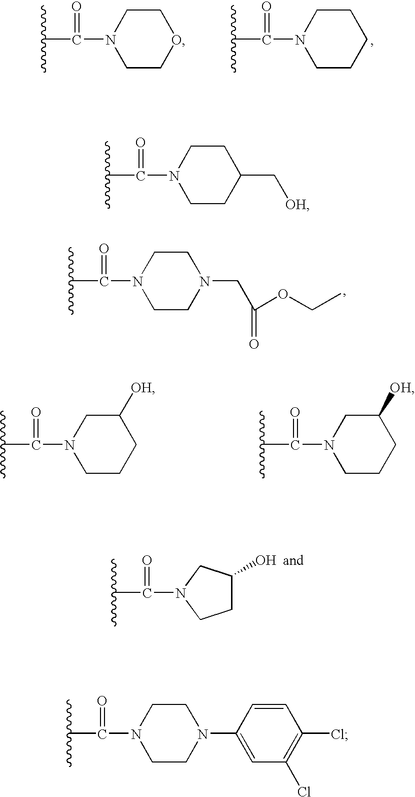

(C) H;
(D) —N(R¹²)₂, wherein each R¹² isindependently selected, and wherein one example of said (D) moiety is —NH₂;
(E) —CH₂OH;
(F) —CH₂OCH₃;
(G) —CH₂SCH₃,
(H) —CH₂N(R^{B}) wherein each R^{B} is independently selected from the group consisting of: H, alkyl, cycloalkyl, heterocycloalkyl, heteroaryl (e.g., pyrazolyl, thiazolyl, and imidazolyl), and aryl (e.g., phenyl);
(I) —N(R¹²)₂ wherein each R¹² is independently selected, examples or said —N(R¹²)₂ moiety include, for example, —NH₂, and —NHalkyl;
(J) —NH—C(O)-alkyl (e.g., —NH—C(O)—CH₃ and —NH—C(O)—(CH₂)₂CH(CH₃)₂);
(K) —NH—C(O)-(hydroxyl substituted alkyl);
(L) —NH—S(O)₂-alkyl (e.g., —NH—S(O)₂—CH₃);
(M) —NH—C(O)—C(═CH₂)CH₂(CH₃)₂;
(N) —NH—C(O)—C(O)—CH₂(CH₃)₂;
(O) alkyl (e.g., ethyl); and
(P) aryl (e.g., phenyl);

Q^{C} is selected from the group consisting of:
(A) heteroaryl (e.g., thienyl and pyridyl);
(B) heterocycloalkyl (e.g., pyrrolidinyl);
(C) H;
(D) alkyl (e.g., C₁ to C₆ alkyl, such as, for example, C₁ to C₄ alkyl) such as, for example, methyl, ethyl, and t-butyl;
(E) —C(O)N(R¹²)₂, such as, for example, —C(O)NHCH₃;
(F) cycloalkyl (e.g., C₃₋₇ cycloalkyl);
(G) halo (e.g., Cl, Br, and I);
(H) —CN;
(I) —CF₃;
(J) —CH₂CF₃;
(K) —SR^{A} wherein R^{A} is selected from the group consisting of: alkyl, cycloalkyl, heterocycloalkyl, heteroaryl (e.g., pyrazolyl, thiazolyl, and imidazolyl), and aryl (e.g., phenyl);
(L) —N(R^{B})₂ wherein each R^{B} is independently selected from the group consisting of: H, alkyl, cycloalkyl, heterocycloalkyl, heteroaryl (e.g., pyrazolyl, thiazolyl, and imidazolyl), and aryl (e.g., phenyl);
(M) —OR^{A} wherein R^{A} is as defined above;
(N) —C(O)R^{A} wherein R^{A} is as defined above;
(O) aryl (e.g., phenyl);
(P) arylalkyl-;
(Q) heteroarylalkyl-;
(R) substituted aryl (e.g., substituted phenyl), such as for example, halo substituted aryl (such has halo substituted phenyl) wherein each halo is independently selected (examples of said halo are Cl, Br, F) and wherein there are 1 to 3 substituents on said substituted aryl;
(S) substituted heteroaryl;
(T) substituted heteroarylalkyl;
(U) substituted aralkyl;

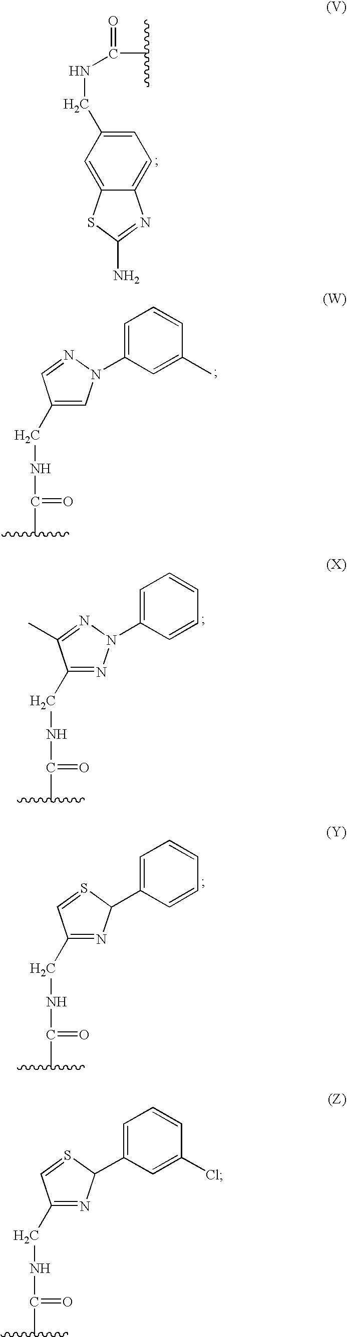

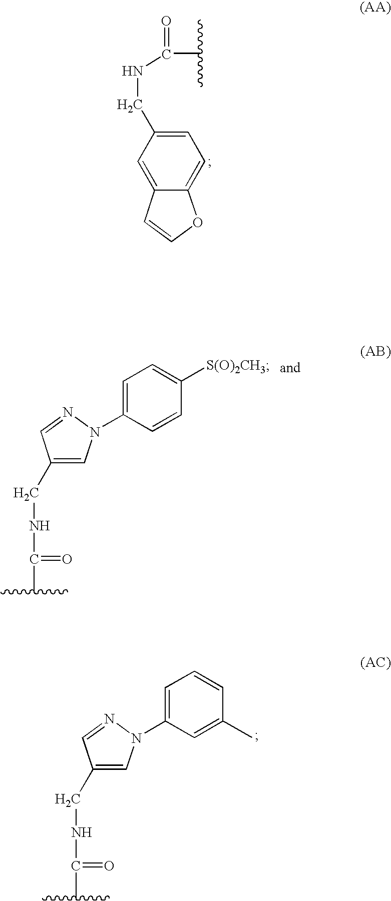

Q^{D} is selected from the group consisting of: H and alkyl (e.g., methyl);

R¹ and R² are each independently selected from the group consisting of:
(1) H;
(2) unsubstituted -(alkylene)₁₋₆-benzoheteroaryl (e.g., unsubstituted —CH₂-benzoheteroaryl), wherein examples of said benzoheteroaryl moiety include, but are not limited to, benzothiazolyl, indazolyl, benzothienyl, quinolinyl and benzoimidazolyl, and wherein examples also include, but are not limited to:

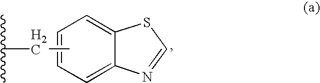

for example,

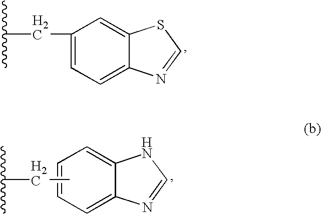

for example,

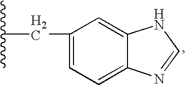

and

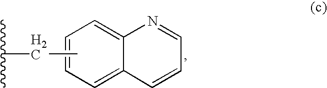

for example,

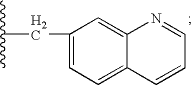

(3) substituted -(alkylene)₁₋₆-benzoheteroaryl, wherein examples of said benzoheteroaryl moiety include, but are not limited to, benzothiazolyl, indazolyl, benzothienyl, quinolinyl and benzoimidazolyl, and wherein:
   (a) either the alkylene or benzoheteroaryl moieties are substituted, or both the alkylene and benzoheteroaryl moieties are substituted,
   (b) when the alkylene moiety is substitued the substitutents (e.g., 1 to 3 substituents) are independently selected from the group consisting of: alkyl (e.g., C₁ to C₆ alkyl), cycloalkyl (e.g., C₃ to C₆ cycloalkyl), —C(O)OH, —C(O)Oalkyl (e.g., —C(O)O(C₁ to C₆ alkyl)), and wherein the substituted alkylene moieties comprise R or S stereochemical centers,
   (c) when the benzoheteroaryl moiety is substituted the substituents (one or more, e.g., 1 or 2 substituents) are independently selected from the group consisting of: (1) —NH₂, (2) —NH(alkyl) (e.g., —NH(C₁-C₆alkyl), such as, for example, —NHCH₃), (3) —NHC(O)(alkyl) (e.g., —NHC(O)(C₁-C₆alkyl), such as, for example, —NHC(O)CH₃), (4) alkyl (e.g., C₁ to C₆ alkyl, such as, for example, methyl and isopropyl), (5) —S(alkyl) (e.g., —S(C₁-C₆ alkyl), such as, for example, —SCH₃), and (6) heteroaryl (e.g., pyridyl, such as, for example, m-pyridyl),
   (d) wherein examples of said substituted -(alkylene)₁₋₆-benzoheteroaryl include, but are not limited to:

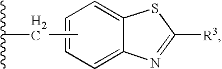

for example,

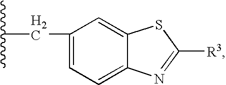

wherein R³ is selected from the group consisting of: (1) —NH₂, (2) —NH(alkyl) (e.g., —NH(C₁-C₆alkyl), such as, for example, —NHCH₃), (3) —NHC(O)(alkyl) (e.g., —NHC(O)(C₁-C₆alkyl), such as, for example, —NHC(O)CH₃), (4) alkyl (e.g., C₁ to C₆ alkyl, such as, for example, methyl and isopropyl (5) —S(alkyl) (e.g., —S(C₁-C₆ alkyl), such as, for example, —SCH₃), and (6) heteroaryl (e.g., pyridyl, such as, for example, m-pyridyl); and wherein R³ is preferably —NH₂; and

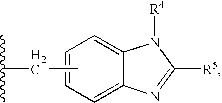

for example,

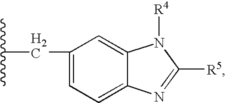

wherein R⁴ and R⁵ are each independently selected from the group consisting of: H and alkyl (e.g., C₁ to C₆ alkyl, such as, for example, methyl and isopropyl) provided that at least one of R⁴ or R⁵ is other than H; and in one example R⁴ is H and R⁵ is alkyl; in another example R⁴ is H and R⁵ is methyl; in another example R⁴ is H and R⁵ is isopropyl; in another example R⁴ is alkyl and R⁵ is H; in another example R⁴ is methyl and R⁵ is H; in another example R⁴ is alkyl and R⁵ is alkyl; and in another example R⁴ is methyl and R⁵ is methyl;
(4) unsubstituted -(alkylene)₁₋₆-heteroaryl (e.g., unsubstituted -(alkylene)₁₋₂-heteroaryl), wherein examples of said heteroaryl moiety include, but are not limited to: imidazolyl, pyridyl (e.g., o-pyridyl, m-pyridyl, and p-pyridyl), thiophenyl (i.e., thienyl), pyrimidinyl, and pyrazinyl, one example of said unsubstituted -(alkylene)₁₋₆-heteroaryl is:

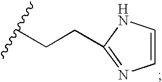

(5) substituted -(alkylene)₁₋₆-heteroaryl (e.g., substituted -(alkylene)₁₋₂-heteroaryl) substituted with one or more (e.g. 1 to 3) substitutents independently selected from the group consisting of: halo (e.g., Cl, F, and Br), —C(O)N(R⁶)₂, and —NHS(O)₂R⁷, wherein each R⁶ is independently selected from the group consisting of H and alkyl (e.g., C₁ to C₆ alkyl), and wherein R⁷ is alkyl (e.g., C₁ to C₆ alkyl), and wherein examples of the substituted heteroaryl moiety include, but are not limited to: substituted imidazolyl, substituted pyridyl (e.g., substituted o-pyridyl, m-pyridyl, and p-pyridyl), substituted thiophenyl (i.e., substituted thienyl), substituted pyrimidinyl, and substituted pyrazinyl;
(6) unsubstituted -benzoheteroaryl, wherein examples of said benzoheteroaryl moiety include, but are not limited to, benzothiazolyl, indazolyl, benzothienyl, quinolinyl and benzoimidazolyl, and wherein in one example said unsubstituted -benzoheteroaryl moiety is:

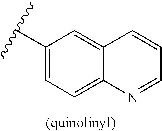

(7) substituted -benzoheteroaryl, wherein examples of said substituted benzoheteroaryl moiety include, but are not limited to, substituted benzothiazolyl, substituted indazolyl, substituted benzothienyl, substituted quinolinyl and substitued benzoimidazolyl, and wherein said substituted benzoheteroaryl is substituted with one or more (e.g., 1 to 3) substitutents independently selected from the group consisting of: heteroaryl (e.g., pyridyl, imidazolyl, and pyrazolyl), heterocycloalkyl (e.g., morpholinyl and piperidyl), and —S(alkyl) (e.g., —S(C₁ to C₆ alkyl) such as, for example, —SCH₃);
(8) heteroaryl (e.g., pyrimidinyl, pyridyl, and pyrazolo[1,5-a]pyrimidinyl);
(9) substituted heteroaryl substituted with one or more substitutents (e.g., 1 to 3 substituents) independently selected from the group consisting of: heteroaryl (e.g., pyridyl, imidazolyl, and pyrazolyl), heterocycloalkyl (e.g., morpholinyl and piperidyl), and —S(alkyl) (e.g., —S(C₁ to C₆ alkyl) such as, for example, —SCH₃), and wherein examples of the heteroaryl moiety of said substituted heteroaryl include but are not limited to pyrimidinyl, pyridyl, and pyrazolo[1,5-a]pyrimidinyl;
(10) aryl (e.g., phenyl);
(11) substituted aryl (e.g., substituted phenyl) substituted with one or more (e.g., 1 to 3) substitutents independently selected from the group consisting of: heteroaryl (e.g., pyridyl, imidazolyl, and pyrazolyl), heterocycloalkyl (e.g., morpholinyl and piperidyl), and —S(alkyl) (e.g., —S(C₁ to C₆ alkyl) such as, for example, —SCH₃);
(12)

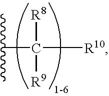

and wherein an example of said moiety (12) is:

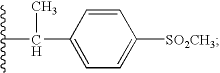

(13) unsubstituted -(alkylene)₁₋₆-heterocycloalkyl (e.g., unsubstituted -(alkylene)₁₋₂-heterocycloalkyl), wherein examples of said heterocycloalkyl include, but are not limited to: piperidinyl (e.g. p-piperidinyl, i.e., the N of the piperidinyl is para to the carbon bonded to the rest of the molecule) and pyrrolidinyl, and in one example said heterocycloalkyl moiety is piperidinyl;
(14) substituted -(alkylene)₁₋₆-heterocycloalkyl (e.g., substituted -(alkylene)₁₋₂-heterocycloalkyl), wherein examples of said heterocycloalkyl include, but are not limited to: piperidinyl (e.g. p-piperidinyl, i.e., the N of the piperidinyl is para to the carbon bonded to the rest of the molecule) and pyrrolidinyl, and in one example said heterocycloalkyl moiety is piperidinyl, wherein said substituted moiety (14) is substituted with one or more substituents (e.g., 1 to 3) selected from the group consisting of —SO₂R¹³, and wherein R¹³ is selected from the group consisting of:
   (a) alkyl (e.g., C₁ to C₈ alkyl, and in one example, methyl),
   (b) aryl (e.g., phenyl),
   (c) substituted aryl (e.g., substitued phenyl, such as, for example, chlorophenyl, fluorophenyl, and cyanophenyl),
   (d) heteroaryl (e.g., pyrazinyl and pyridyl),
   (e) substituted heteroaryl (e.g., substituted pyrazinyl and substituted pyridyl),
   (f) -(alkylene)₁₋₆heterocylcoalkyl (e.g., -(alkylene)₁₋₂heterocycloalkyl), such as, for example, —(CH₂)₂-morpholinyl and —CH₂-piperidinyl,
   (g) -(alkylene)₁₋₆-heteroaryl (e.g., -(alkylene)₁₋₂heteroaryl), such as, for example, —CH₂-pyridyl,
   (h) —C(O)R¹¹ (wherein R¹¹ is as previously defined),
   (i) —C(O)aryl (e.g., —C(O)phenyl), and
   (j) -(alkylene)₁₋₆N(R¹²)₂ (e.g., -(alkylene)₁₋₃N(R¹²)₂), such as, for example, —(CH₂)₃N(R¹²)₂, and
   (k) wherein said substituted groups (c) and (e) of said moiety (14) are independently substituted with one or more (e.g., 1 to 3) substitutents independently selected from the group consisting of: (i) halo (e.g., Cl, F, Br, and I), (ii) —OH, (iii) —OR¹¹, (iv) —CF₃, (v) —S(O)₂R¹¹ (e.g., —S(O)₂CH₃), and (vi) —S(O)₂N(R¹²)₂, and
   (l) wherein an example of said moiety (14) is:

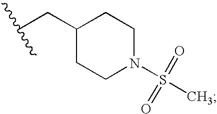

(15) -(alkylene)₁₋₆-bicyclic bridged cycloalkyl (e.g., -(alkylene)₁₋₆-adamantyl);
(16) -(alkylene)₁₋₆-bicyclic bridged heterocycloalkyl;
(17) -(alkylene)₁₋₆-bicyclic bridged spirocycloalkyl;
(18) -(alkylene)₁₋₆-bicyclic bridged spiroheterocycloalkyl;
(19) -(alkylene)₁₋₆-(substituted heteroaryl) wherein the substituents on said heteroaryl are independently selected from the group consisting of: —C(O)N(R¹²)₂ wherein each R¹² is independently selected, —NHS(O)₂-alkyl (e.g., —NHS(O)₂—(C₁₋₆alkyl), such as, for example, —NHS(O)₂—CH₃), and -(alkylene)₁₋₆-NHS(O)₂-alkyl (e.g., -(alkylene)₁₋₆-NHS(O)₂—(C₁₋₆alkyl), such as, for example, -(alkylene)₁₋₆-NHS(O)₂—CH₃),
(20) -cycloalkyl-benzodioxolyl (e.g.,

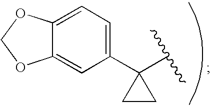

(21) -cycloalkyl-(substituted aryl) wherein the substituents are independently selected from the group consisting of methylene dioxy and —S(O)₂CH₃ (examples of said -cycloalkyl-(substituted aryl) include but are not limited to:

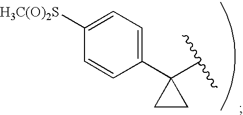

(22) alkyl (e.g., (C₁₋₆ alkyl, such as for example, methyl)
(23) cycloalkyl;
(24) alkyl;
(25) hydroxyl substituted alkyl;

R⁸ and R⁹ are each independently selected from the group consisting of: H, alkyl (e.g., C₁ to C₆ alkyl, such as, for example, methyl), cycloalkyl (e.g., C₃ to C₆ cycloalkyl), C(O)OH, —C(O)OR¹¹, substituted alkyl (e.g., substituted C₁ to C₆ alkyl) and substituted cycloalkyl (e.g., C₃ to C₆ cycloalkyl);

R¹⁰ is selected from the group consisting of:
(a) aryl (e.g., phenyl),
(b) substituted aryl (e.g., substituted phenyl),
(c) heteroaryl (e.g., pyrazinyl, pyridyl (such as, for example, o-pyridyl, m-pyridyl and p-pyridyl), thiophenyl (i.e., thienyl), pyrazolyl (e.g., 3-pyrazolyl and 4-pyrazolyl), thiazolyl, oxazolyl, and pyrimidinyl),
(d) substituted heteroaryl (e.g., substitued pyrazinyl, substituted pyridyl (such as, for example, substituted o-pyridyl, substituted m-pyridyl and substituted p-pyridyl), substituted thiophenyl (i.e., substituted thienyl), substituted pyrazolyl (e.g., substituted 3-pyrazolyl and substituted 4-pyrazolyl), substituted thiazolyl, substituted oxazolyl, and substituted pyrimidinyl),
(e) benzoheteroaryl,
(f) heterocycloalkyl,
(g) substituted heterocycloalkyl,
(h) -piperidinyl-S(O)₂-(alkyl substituted heteroaryl),
(i) -piperidinyl-S(O)₂-aryl-heteroaryl),
(j) -piperidinyl-C(O)-pyridyl,
(k) -piperidinyl-C(O)-alkyl,
(l) -piperidinyl-(substituted aryl) wherein said substituents are independently selected from the groups consisting of: halo (e.g., F) and CN,
(m) -piperidinyl-pyridyl (such as, for example,

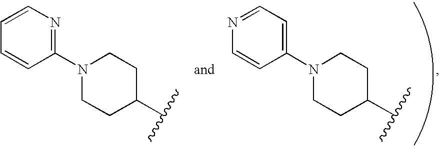

(n) benzodioxolyl (i.e.,

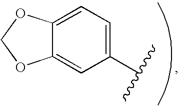

(o) -heteroaryl-NH-cycloalkylalkyl (e.g., -pyridyl-NH-cycloalkylalkyl), and
(p) -heteroaryl-NH-cycloalkyl (e.g., e.g., -pyridyl-NH-cycloalkyl), and
(wherein examples of said R¹⁰ groups (g)-(j) include but are not limited to:

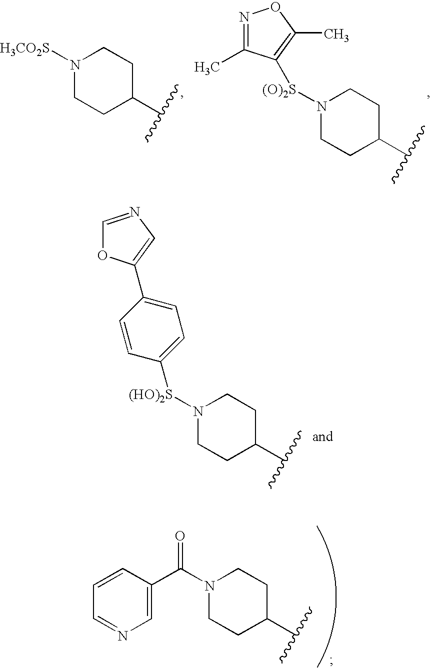

wherein said substituted R⁸, R⁹ and R¹⁰ groups are substituted with one or more (e.g., 1 to 3) substitutents independently selected from the group consisting of:
(a) halo (e.g., Cl, F, Br, and I),
(b) —OH,
(c) —OR¹¹,
(d) —CF₃,
(e) heterocycloalkyl (e.g., pyrrolidinyl, piperazinyl, morpholinyl, and piperidinyl),
(f) substituted heterocycloalkyl (e.g., substituted pyrrolidinyl (e.g., pyrrolidinonyl, i.e., pyrrolidinyl substituted with ═O), substituted piperazinyl, substituted morpholinyl, and substituted piperidinyl),
(g) heteroaryl (e.g., pyrazolyl and thiazolyl),
(h) substituted heteroaryl (e.g., substituted pyrazolyl and substituted thiazolyl),
(i) aryl (e.g., phenyl),
(j) substituted aryl (e.g., substituted phenyl),
(k) —C(O)OR¹¹,
(l) —N(R¹²)₂ (e.g., —NHR¹²),
(m) alkyl (e.g., C₁ to C₆ alkyl),
(n) cycloalkyl (e.g., C₃ to C₆ alkyl),
(o) —SO₂R¹¹,
(p) —N(alkyl)-cycloalkyl,
(q) —C(O)OH,
(r) benzoheteroaryl (e.g., benzoimidazolyl), and
(s) substituted benzoheteroaryl (e.g., substituted benzoimidazolyl), such as for example substituted benzoheteroaryl substituted with 1 to 2 alkyl groups (e.g., methyl), such as for example, alkyl (e.g., methyl) substituted benzoimidazolyl,
and wherein said substituted groups (f), (h), and (j) are independently substituted with one or more substitutents (e.g., 1 to 3 substituents) independently selected from the group consisting of:
   (i) halo (e.g., Cl, F, Br, and I),
   (ii) —OH,
   (iii) —OR¹¹,
   (iv) —CF₃,
   (v) —S(O)₂R¹¹ (e.g., —S(O)₂CH₃),
   (vi) —S(O)₂N(R¹²)₂,
   (vii) ═O,
   (viii) substituted benzoheteroaryl (e.g., substituted benzoimidazolyl) substituted with 1 to 3 groups independently selected from the group consisting of: C₁ to C₆ alkyl, cycloalkyl, —NH₂, —NH(C₁ to C₆ alkyl), and —N(C₁ to C₆ alkyl)₂ wherein each alkyl is independently selected,
   (ix) alkyl (e.g., C₁₋₆alkyl, such as, for example, methyl),
   (x) CN,
   (xi) cycloalkyl, and
   (xii) —C(O)-morpholinyl,
   (xiii) amino,
   (xiv) alkylamino (e.g., —NHCH₃), and
   (xv) and dialkylamino;

R¹¹ is alkyl (e.g., C₁ to C₆ alkyl);

each R¹² is independently selected from the group consisting of H, alkyl (e.g., C₁ to C₆ alkyl), and hydroxyl substituted alkyl,

wherein an example of said moiety (12) is:

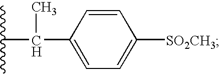

each R¹⁴ is independently selected from the group consisting of: H, —C(O)—(CH₂)₁₋₂-aryl (e.g., —C(O)—(CH₂)₁₋₂-phenyl, such as, for example, —C(O)—CH₂-phenyl), substituted aryl (e.g., substituted phenyl), and benzodioxyl, and wherein said substituted aryl (e.g., substituted phenyl) is substituted with one or more (e.g., 1 to 3) substituents independently selected from the group consisting of: halo (e.g., Cl, F, and Br), —OH, —OR¹¹ (wherein R¹¹ is as previously described), —CN, —CF₃, alkyl (e.g., C₁ to C₆ alkyl), —NH₂ and —NO₂;

R¹⁵ and R¹⁶ are each independently selected from the group consisting of:
(1) hydroxyl substituted alkyl, such as hydroxyl substituted C₁ to C₈ (preferably C₁ to C₆) alkyl, such as, for example, —CH(CH₂OH)CH₂CH(CH₃)₂, —CH₂OH, —(CH₂)₂OH, —CH(CH₂OH)CH₂CH₃, —CH(CH₂OH)C(CH₃)₃, —CH(CH₃)CH₂OH, and —CH(CH₂OH)₂, and when the carbon atom bound to the N has a chiral center then the S-isomer of said chiral center is preferred,
(2) alkyl (e.g., C₁ to C₆ alkyl) such as, for example, i-propyl, methyl, ethyl, —CH₂CH(CH₃)₂, and —(CH₂)₂CH(CH₃)₂,
(3) —SO₂R¹¹, e.g., —SO₂CH₃,
(4) unsubstituted -(alkylene)₁₋₆-R¹⁷ (e.g., unsubstituted -(alkylene)₁₋₂-R¹⁷) wherein R¹⁷ is selected from the group consisting of: (a) heterocycloalkyl (e.g., tetrahydrofuran, piperidinyl, pyrrolidinyl, piperazinyl, and morpholinyl), (b) heteroaryl (e.g., pyridyl), and (c) cycloalkyl (e.g., C₃ to C₆ cycloalkyl), and wherein in one example said alkylene-R¹⁷ moiety is:

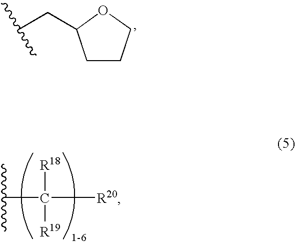

and wherein examples of said moiety (5) include, but are not limited to:

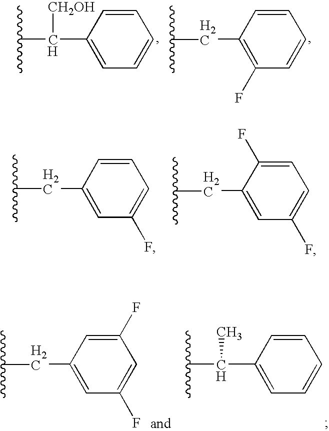

(6) —C(O)-alkyl (e.g., —C(O)(C₁ to C₆)alkyl) such as —C(O)CH₃,
(7) substituted alkyl wherein said substituents are selected from the group consisting of —OR¹¹, such as, for example, —(CHR¹²)₁₋₆—OR¹¹ (wherein R¹² is as previously defined), and also, for example, —(CHR¹²)₁₋₃—OR¹¹, wherein examples of said substituted alkyl moiety (7) include, but are not limited to: —CH(CH₃)CH₂OCH₃, and —(CH₂)₃OCH₃,
(8) saturated bicyclic rings, such as, for example,

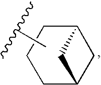

(9) hydroxyl substituted -(alkylene)₁₋₆-cycloalkyl, such as, for example, (e.g., substituted -(alkylene)₁₋₆-C₃-C₆ cycloalkyl, such as, for example, substituted -(alkylene)₁₋₂-C₃-C₆ cycloalkyl), such as for example,

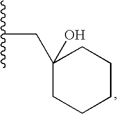

(10) H,
(11) heterocycloalkyl substituted with heterocycloalkyl,
(12) cycloalkyl (e.g., C₃₋₈ cycloalkyl, such as, for example, cyclohexyl), and
(13) cycloalkyl (e.g., C₃₋₈ cycloalkyl, such as, for example, cyclohexyl) substituted with 1 to 2 —OH groups,
(14) -(alkylene)₁₋₆-aryl (e.g., -(alkylene)₁₋₆-phenyl),
(15) -(alkylene)₁₋₆-aryl (e.g., -(alkylene)₁₋₆-phenyl) substituted with 1 to 2 substituents independently selected from the group consisting —OH and alkylamino (e.g., —NHCH₃),
(16) -(alkylene)₁₋₆-heteroaryl substituted with 1 to 2 substituents independently selected from the group consisting —OH and alkylamino (e.g., —NHCH₃);
(17) heterocycloalkyl,
(18) substituted heterocycloalkyl, such as heterocycloalkyl substituted with alkyl, such as heterocycloalkyl substituted with methyl,
(19) -(alkylene)₁₋₆-heterocycloalkyl wherein said alkylene moiety is substituted with hydroxyl,
(20) -(alkylene)₁₋₆-C(O)OH,
(21) fused hydroxyl substituted benzocycloalkyl (e.g.,

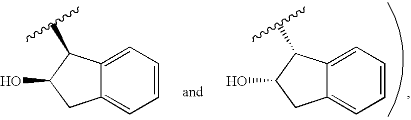

(22) fused hydroxyl substituted arylheteroaryl (e.g., fused hydroxyl substituted benzoheteroaryl),
(23) hydroxyl-(alkylene)₁₋₆-cycloalkyl (e.g.,

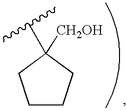

(24) hydroxyl-(alkylene)₁₋₆-bridged cycloalkyl (e.g.,

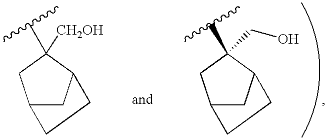

(25) hydroxylalkylene)₁₋₆-spirocycloalkyl,
(26) hydroxyl-(alkylene)₁₋₆-bridged heterocycloalkyl,
(27) hydroxyl-(alkylene)₁₋₆-spiroheterocycloalkyl, and
(28) heterocycloalkyl;

each R¹⁸ and each R¹⁹ is independently selected from the group consisting of: H, alkyl (e.g., C₁ to C₆ alkyl, such as, for example, methyl), and hydroxyalkyl- (e.g., —CH₂OH), and when the carbon atom to which R¹⁸, R¹⁹, and R²⁰ are bound is a chiral center then the S-isomer of said chiral center is preferred;

R²⁰ is selected from the group consisting of:
(a) aryl (e.g., phenyl),
(b) substituted aryl (e.g., substituted phenyl),
(c) heteroaryl (e.g., pyridyl),
(d) benzo fused heteroaryl (e.g., indolyl),
(e) -(alkylene)₁₋₆-heteroaryl (e.g., -(alkylene)₁₋₂-heteroaryl), such as, for example, —CH₂imidazolyl,
(f) -(alkylene)₁₋₆aryl,
(g) -(alkylene)₁₋₆aryl substituted with —OH,
(h) benzoheteroaryl-(alkylene)₁₋₆-,
(i) cycloalkylalkyl,
(j) cycloalkyl (e.g., hexyl),
(k) heterocycloalkyl,
(l) -(alkylene)₁₋₆aryl substituted with halo (e.g., Cl, F, and Br) such as p-chlorobenzyl,
(m) -(alkylene)₁₋₆-S-alkyl (e.g., —(CH₂)₂—S—CH₃),
(n) -(alkylene)₁₋₆-O-alkyl,
(o) -(alkylene)₁₋₆-N-alkyl,
(p) -(alkylene)₁₋₆-cycloalkyl,
and wherein said substituted aryl (e.g., substituted phenyl) is substituted with one or more substituents (e.g., 1 to 3) independently selected from the group consisting of: halo (e.g., Cl, F, and Br), —OH, —OR¹¹, —CN, —CF₃, alkyl (e.g., C₁ to C₆ alkyl), —NH₂ and —NO₂;

R²¹ is selected from the group consisting of:
(1) heterocycloalkyl (e.g., morpholinyl, piperidinyl, piperazinyl, and pyrrolidinyl),
(2) benzo fused cycloalkyl (i.e., a benzene ring fused to a cycloalkyl ring wherein there are two adjacent carbon atoms common to the benzene ring and the cycloalkyl ring), such as, for example, indanyl,
(3) cycloalkyl (e.g., C₃ to C₆ cycloalkyl), such as, for example, cyclopentyl,
(4) multicyclic cycloalkyl ring, such as, for example, adamantly, and
(5) substituted heterocycloalkyl (e.g., substituted morpholinyl, substituted piperidinyl, substituted piperazinyl, and substituted pyrrolidinyl) substituted with one or more (e.g., 1 to 3) substituents independently selected from the group consisting of: (a) hydroxyl substituted alkyl (e.g., —CH₂OH), (b) —OH, (c)-(alkylene)₁₋₆C(O)O-(alkyl)₁₋₆ (such as, for example, —CH₂C(O)OCH₂CH₃), (d) aryl (e.g., phenyl), and (e) substituted aryl (e.g., substituted phenyl) wherein said substituted aryl (e.g., said substituted phenyl) is substituted with one or more (e.g., 1-3) substitutents independently selected from the group consisting of: halo (e.g., F, Cl, and Br), and
(6) heterocycloalkyl substituted with 1 to 3 substituents selected from the group consisting of: amino, alkylamino, dialkylamino, and —C(O)alkyl,
(7) heterocycloalkyl (e.g., a 4 to 7 membered heterocycloalkyl ring, examples include but are not limited to piperazinyl, piperidinyl, and pyrrolidinyl),
(8) hydroxy substituted heterocycloalkyl (e.g., a 4 to 7 membered hydroxyl substituted heterocycloalkyl ring, examples include but are not limited to hydroxyl substituted piperazinyl, hydroxyl substituted piperidinyl, and hydroxyl substituted pyrrolidinyl), and
(9) —OH.

In one embodiment of the invention K is CH.

In one embodiment of the invention K is N.

In one embodiment of the invention K is —C(alkyl)- (e.g., —C(CH₃)—).

In one embodiment of the invention K is —C(aryl)- (e.g., —C(phenyl)-).

In one embodiment of the invention K is —C(halo)- (e.g., —C(F)—, or —C(Cl)— or —C(Br)—).

In one embodiment of the invention K is —C(R^{C})— wherein R^{C} is selected from the group consistina of:

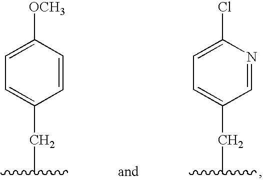

Examples of R¹ and R² groups include, but are not limited to:

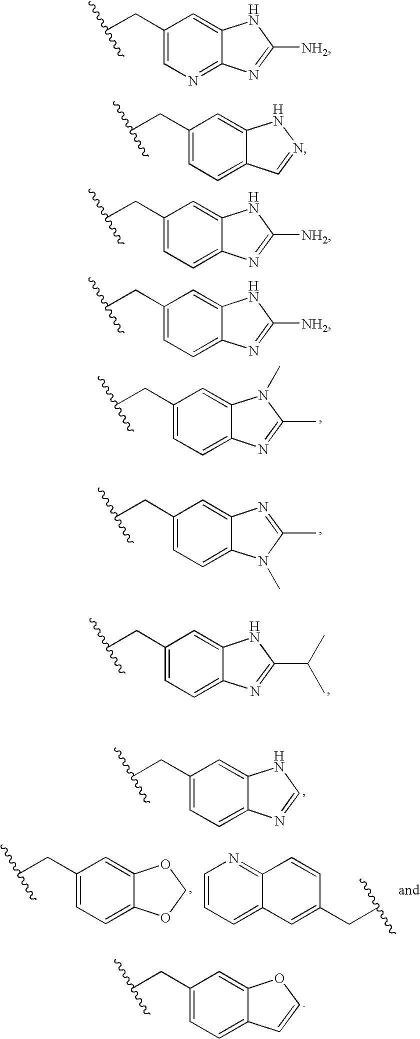

Examples of Q^{A} groups include, but are not limited to:

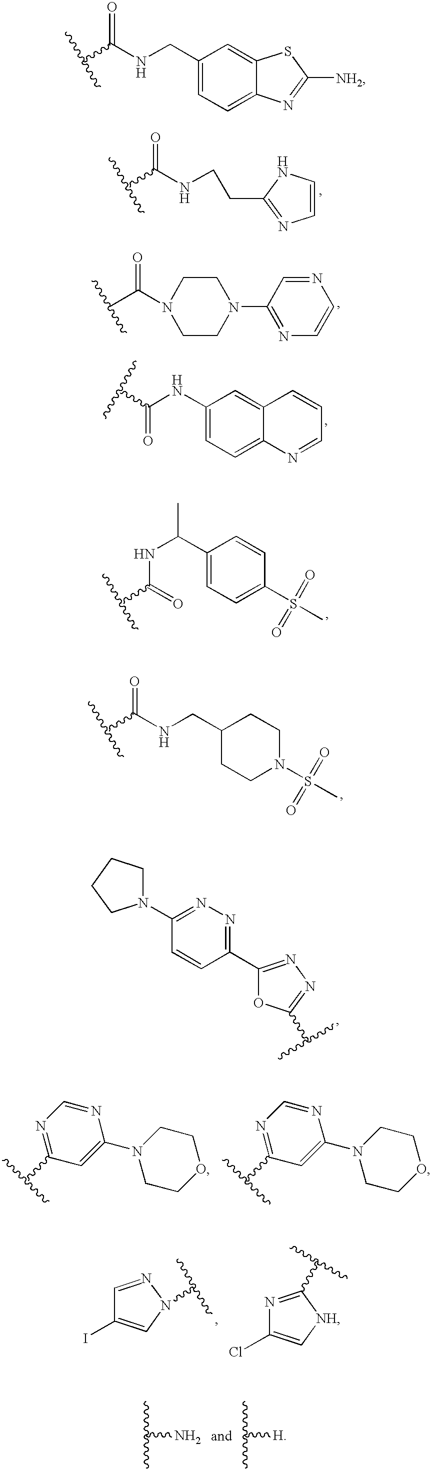

In one embodiment of this invention O^{A} is:

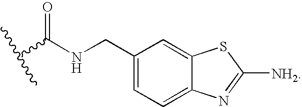

In another embodiment of this invention Q^{A} is:

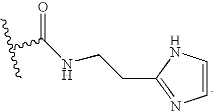

In another embodiment of this invention Q^{A} is:

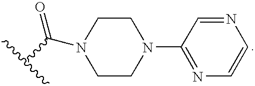

In another embodiment of this invention Q^{A} is:

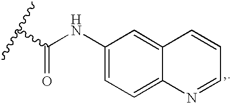

In another embodiment of this invention Q^{A} is:

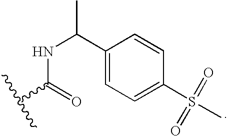

In another embodiment of this invention Q^{A} is:

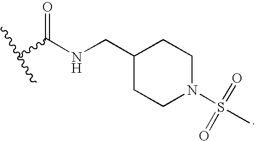

In another embodiment of this invention Q^{A} is:

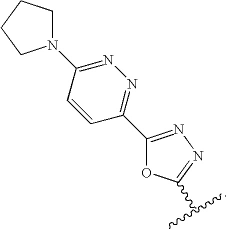

In another embodiment of this invention Q^{A} is:

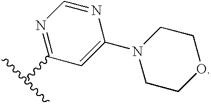

In another embodiment of this invention Q^{A} is:

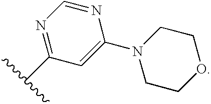

In another embodiment of this invention Q^{A} is:

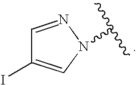

In another embodiment of this invention Q^{A} is:

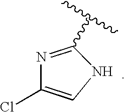

In another embodiment of this invention Q^{A} is —NH₂.

In another embodiment of this invention Q^{A} is H.

Examples of Q^{B} include, but are not limited to:

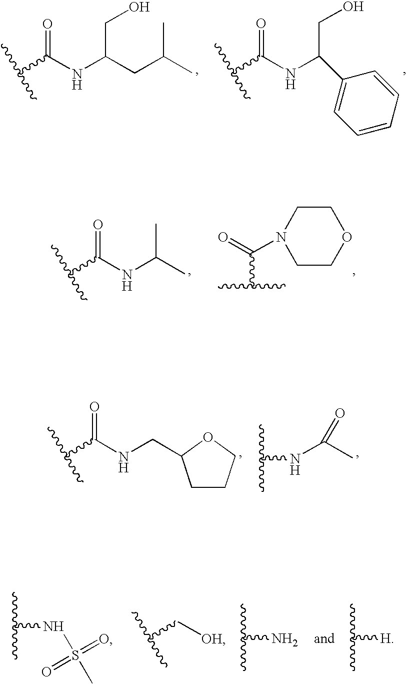

In one embodiment of this invention Q^{B} is:

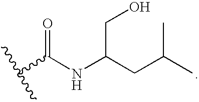

In another embodiment of this invention Q^{B} is:

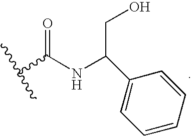

In another embodiment of this invention Q^{B} is:

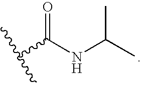

In another embodiment of this invention Q^{B} is

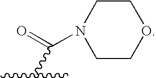

In another embodiment of this invention Q^{B} is:

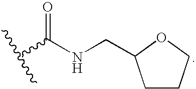

In another embodiment of this invention Q^{B} is:

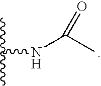

In another embodiment of this invention Q^{B} is:

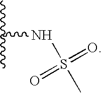

In another embodiment of this invention Q^{B} is:

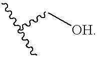

In another embodiment of this invention Q^{B} is —NH₂.

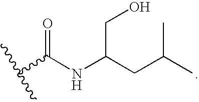

In another embodiment of this invention Q^{B} is H.

Examples of Q^{B} also include, but are not limited to:

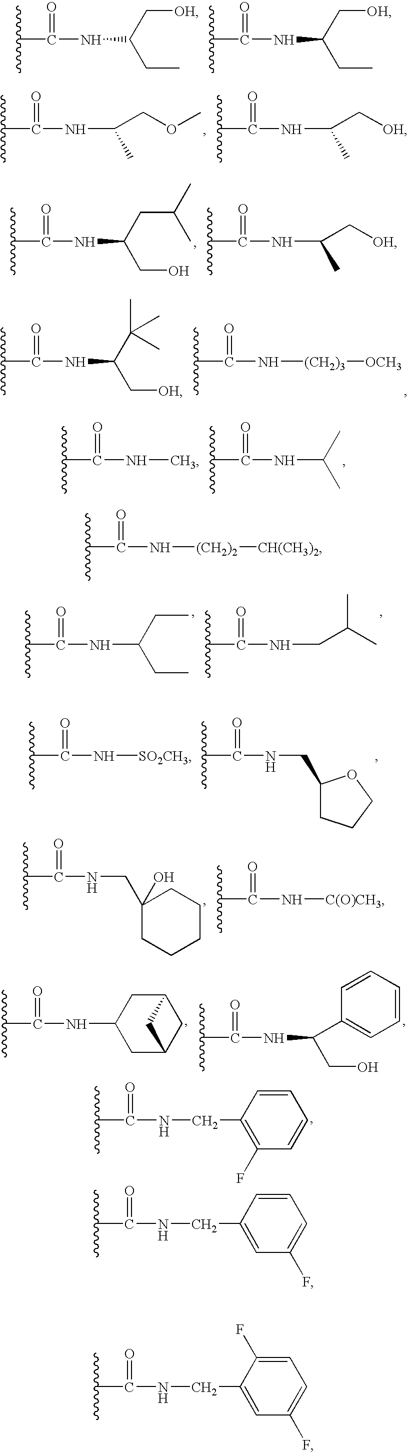

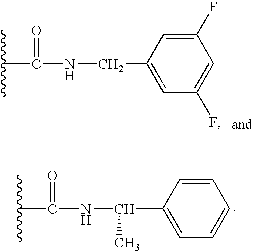

Examples of Q^{C} include, but are not limited to:

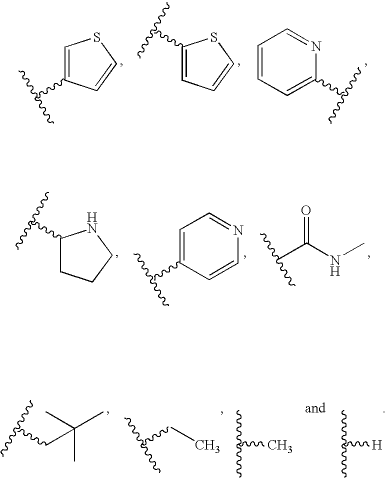

In one embodiment of this invention Q^{C} is:

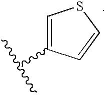

In another embodiment of this invention Q^{C} is:

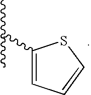

In another embodiment of this invention Q^{C} is:

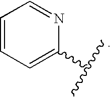

In another embodiment of this invention Q^{C} is:

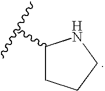

In another embodiment of this invention Q^{C} is:

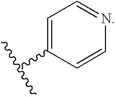

In another embodiment of this invention Q^{C} is:

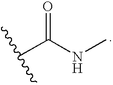

In another embodiment of this invention Q^{C} is:

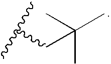

In another embodiment of this invention Q^{C} is:

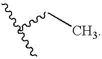

In another embodiment of this invention Q^{C} is —CH₃.

In another embodiment of this invention Q^{C} is H.

The compounds of the invention can be made according to the processes described below. The compounds of this invention are also exemplified in the examples below, which examples should not be construed as limiting the scope of the disclosure. Alternative mechanistic pathways and analogous structures within the scope of the invention may be apparent to those skilled in the art.

In the tables below EMW stands for Exact Molecular Weght. The LC-MS data for the EMW was obtained using an Agilent 1100 Series LC/MSD (quadrupole, API-ES (Atmospheric Pressure Interface Electrospray)) with a capillary voltage set to 3500 V and running in positive mode.

In the tables below, the retention time is for the purification via reverse phase chromatography which was accomplished using a C18 reverse phase column with a gradient of 0.1% trifluoroacetic acid in water to 95:5 acetonitrile:water at a flow rate of 20 mL/min. Samples were collected using a UV (Gilson, 254 nm) or mass spectra (Agilent 1100 Series LC/MSD model SL) signal.

### Example 1A

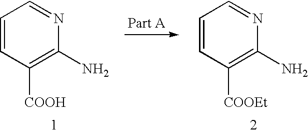

To a solution of 2-aminonicotinic acid (1) (5 g, 36 mmol) in ethanol (100 mL) was added concentrated sulfuric acid (10 mL). The reaction mixture was heated at reflux for 16 hours, and then cooled to room temperature. LC-MS analysis of the reaction indicated that the reaction was complete. The volatiles were removed in vacuo, water was added and the crude basified to pH 8.0 with 1N NaOH. The product was extracted into ethyl acetate (×2), dried over magnesium sulfate and concentrated to afford compound 2 (6.0 g, 100% yield) as a white crystalline solid. HPLC-MS t_{R}=0.41 min (UV_{254 nm}); mass calculated for formula C₈H₁₀N₂O₂ 166.1, observed LCMS m/z 167.1 (M+H).

### Example 1B

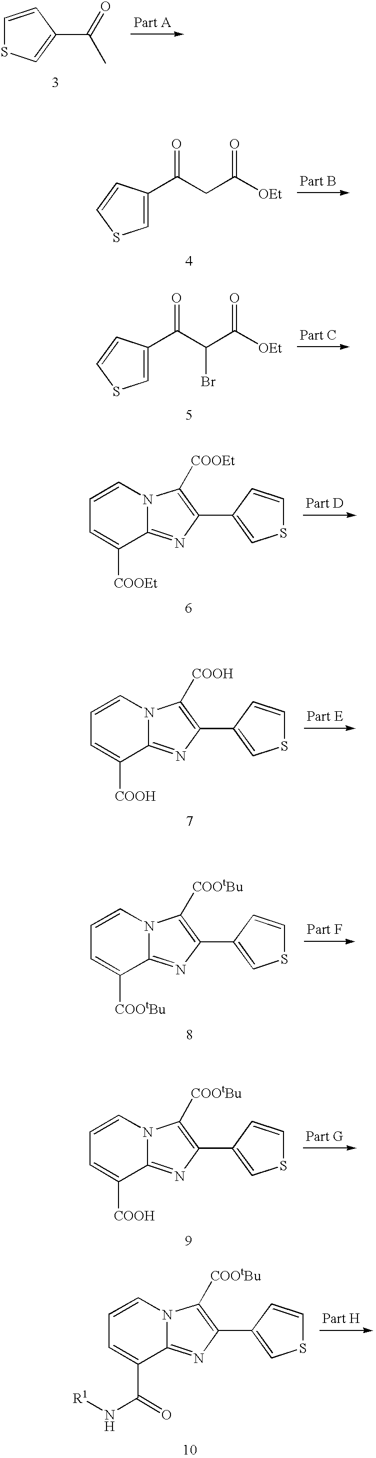

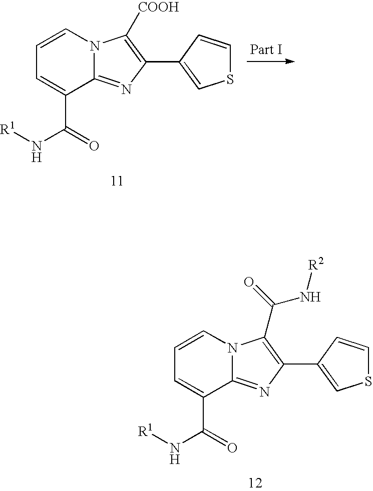

(wherein R¹ is remaining moiety of the Q^{A} group, and R² is the remaining moiety of the Q^{B} group in formula 1.0)

### Part A:

To a mixture containing sodium hydride (18.6 g, 465 mmol) (60% dispersion in mineral oil, washed with hexane to remove mineral oil) and diethyl carbonate (36 mL, 296 mmol) in toluene (200 mL) at reflux, was added 3-acetylthiophene (3) (18.7 g, 148 mmol) in toluene (60 mL) via dropwise addition using an addition funnel. After the addition was complete, the mixture was refluxed for an additional 30 minutes. The reaction mixture was then cooled to room temperature and placed in an ice bath, quenched with acetic acid (42 mL), water, and extracted with toluene. The combined toluene extracts were washed with water (×4), and brine, dried over magnesium sulfate and concentrated to give a brown oil which was subjected to vacuum distillation. The fraction boiling at approximately 140° C. afforded compound 4 (13.8 g, 47% yield).

### Part B:

Bromine (2.7 mL, 53 mmol) in chloroform (40 mL) was added dropwise via an addition funnel to a stirred solution of compound 4 (10.5 g, 53 mmol) in chloroform (60 mL) at 0° C. (ice-bath). After the addition was complete the solution was stirred at room temperature for 20 minutes, during which time the course of the reaction was monitored by Thin Layer Chromatography (dichloromethane as solvent). Bromine (0.3 mL) was added to ensure complete conversion of starting material. The reaction mixture was then washed with saturated NaHCO₃ solution, water, and brine, dried over magnesium sulfate and concentrated to afford compound 5 (14.4 g , 97% yield) as a yellow oil.

### Part C:

A mixture of compound 5 (31.6 g, 114 mmol) and compound 2 (18.9 g, 114 mmol) in ethanol (400 mL) was heated at reflux for 60 hours. After cooling to room temperature, some of the ethanol was removed under reduced pressure, and upon addition of ether a solid formed which was collected by filtration, and confirmed by 1H NMR to be the hydrobromide salt of compound 2 (12 g). The ether filtrate was concentrated to afford a residue which when dissolved in 10% HCl solution, separated out unreacted compound 5 as an oil. The oil was removed, and the acidic aqueous solution was neutralized with saturated NaHCO₃ to pH 7.0, and then extracted with dichloromethane (×2). The organics were concentrated to afford compound 6 (20 g, 51%) as a white solid.

### Part D:

A mixture of compound 6 (20 g, 58 mmol) and LiOH (1 M, 180 mL, 180 mmol) in THF (250 mL) was stirred at room temperature for 16 hours. The volatiles were removed in vacuo, water was added and the aqueous acidified to pH 2.0 with 1N HCl. The resulting precipitate was collected by filtration, washed with water, and dried to afford compound 7 (9.7 g, 58% yield).

### Part E:

A mixture of compound 7 (1.05 g, 3.6 mmol) and 2-tert-butyl-1,3-diisopropylisourea (6 g, 29.2 mmol) in dichloromethane (60 mL) was heated at reflux for 6 hours and then cooled to room temperature. LC-MS analysis of the reaction indicated that the reaction was complete. The resulting precipitate was removed by filtration and washed through with dichloromethane. The filtrate was concentrated, and purified by flash column chromatography (SiO₂, dichloromethane/ethyl acetate—100:1) to afford compound 8 as a white foam (1.22 g, 88% yield). HPLC-MS t_{R}=2.42 min (UV_{254 nm}); mass calculated for formula C₂₁H₂₄N₂O₄S 400.1, observed LCMS m/z 401.2 (M+H).

### Part F:

A mixture of compound 8 (1.22 g, 3.05 mmol) and LiOH (1M, 3.05 mL, 3.05 mmol) in THF (20 mL) and water (10 mL) was stirred at room temperature for 16 hours. LC-MS analysis of the reaction indicated that the reaction was complete. The volatiles were removed in vacuo, water was added and the aqueous acidified to pH 2.0 with 1N HCl. The product was extracted with ethyl acetate (×2), dried over magnesium sulfate and concentrated to afford compound 9 (0.85 g, 81% yield). HPLC-MS t_{R}=1.47 min (UV_{254 nm}); mass calculated for formula C17H16N2O4S 344.1, observed LCMS m/z 345.1 (M+H).

### Part G:

To a mixture of compound 9 (50 mg, 0.145 mmol) and O-(7-Azabenzotriazol-1-yl)-N,N,N′,N′-tetramethyluronium hexafluorophosphate (HATU) (66 mg, 0.174 mmol) in DMF (2 mL) was added amine building block (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and washed successively with saturated NaHCO₃(×1), water (×1), brine (×1), dried over magnesium sulfate and concentrated. Purification by flash column chromatography (SiO₂, ethyl acetate) afforded compound 10 as a white solid (50-90% yield).

### Part H:

To a solution of compound 10 (0.1 mmol) in dioxane (1 mL) was added 4 N HCl in dioxane (2 mL) and water (0.2 mL). The reaction mixture was stirred at room temperature for 3 hours. LC-MS analysis of the reaction indicated that the reaction was complete. The volatiles were removed in vacuo, acetonitrile was added, concentrated and dried to afford compound 11 (100% yield).

### Part I:

To a mixture of compound 11 (0.1 mmol) and HATU (46 mg, 0.12 mmol) in DMF (2 mL) was added amine building block (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and washed successively with saturated NaHCO₃ (×1), water (×1), brine (×1), dried over magnesium sulfate and concentrated. Purification by Prep-LC and conversion to a hydrochlorde salt afforded compounds as white solids.

Using procedures similar to those in Example 1B, the compounds in Table 1 were synthesized.

TABLE 1

| | | | MS | Ret. |
|---|---|---|---|---|
| Compd | | | m/z | Time |
| No. | Structure | EMW | (M⁺+ H) | (min) |
| 13 | 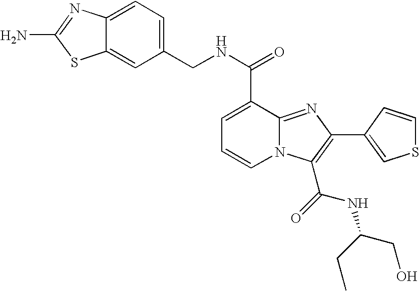 | 520.1 | 521.1 | 3.28 |
| 14 | 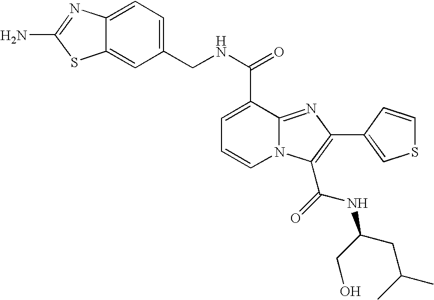 | 548.2 | 549.2 | 3.70 |
| 15 | 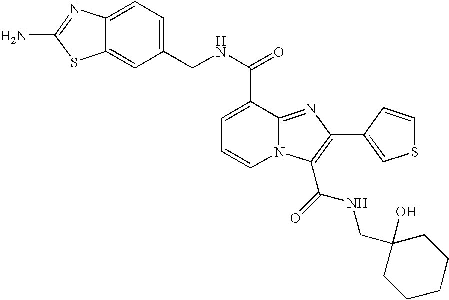 | 560.2 | 561.2 | 3.76 |
| 16 | 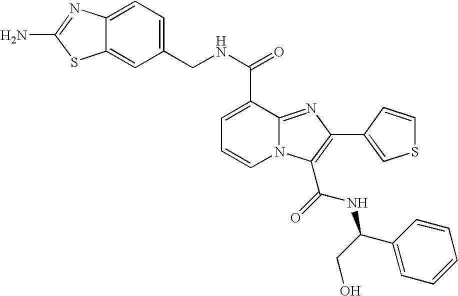 | 568.1 | 569.1 | 3.60 |
| 17 | 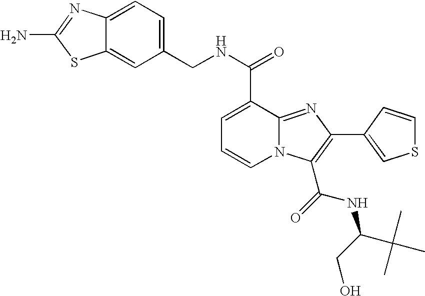 | 548.2 | 549.1 | 3.78 |
| 18 | 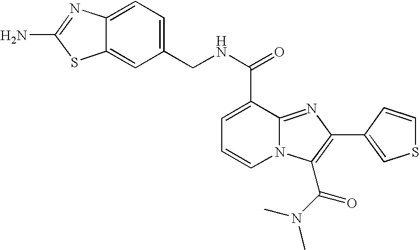 | 476.11 | 477.1 | 3.137 |
| 19 | 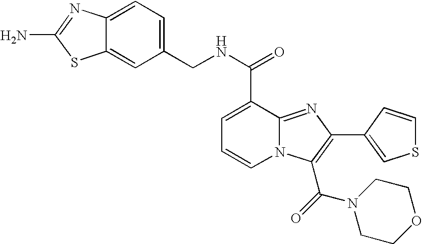 | 518.12 | 519.1 | 3.150 |
| 20 | 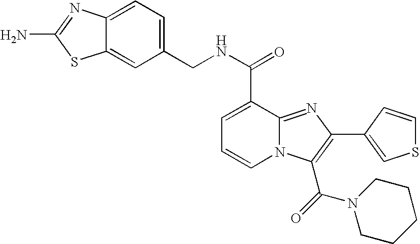 | 516.14 | 517.2 | 3.748 |
| 21 | 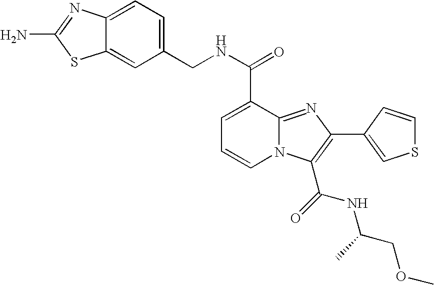 | 520.14 | 521.0 | 3.555 |
| 22 | 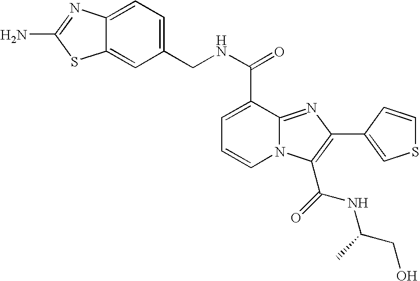 | 506.12 | 507.1 | 2.939 |
| 23 | 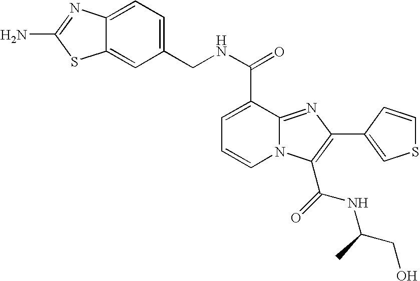 | 506.12 | 507.1 | 2.933 |
| 24 | 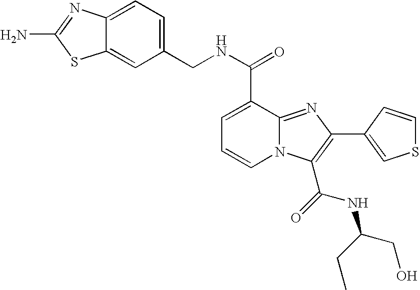 | 520.14 | 521.1 | 3.166 |
| 25 | 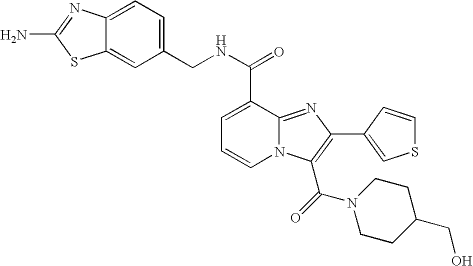 | 546.15 | 547.2 | 2.994 |
| 26 | 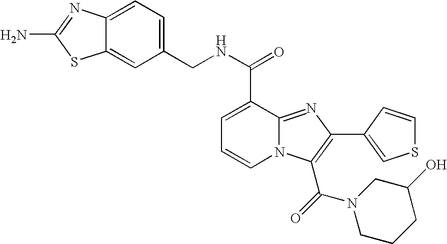 | 532.14 | 533.0 | 3.031 |
| 27 | 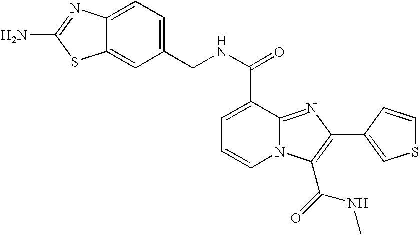 | 462.09 | 463.0 | 3.008 |
| 28 | 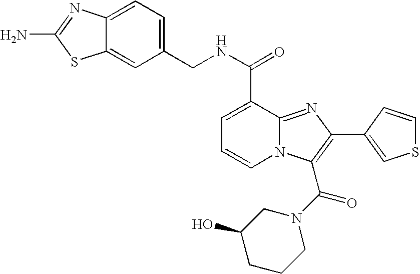 | 532.14 | 533.1 | 3.020 |
| 29 | 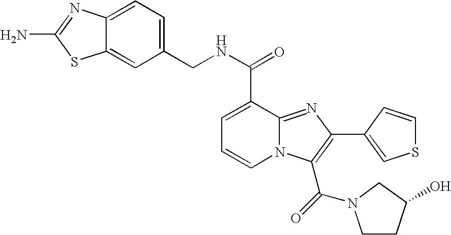 | 518.12 | 519.1 | 2.800 |
| 30 | 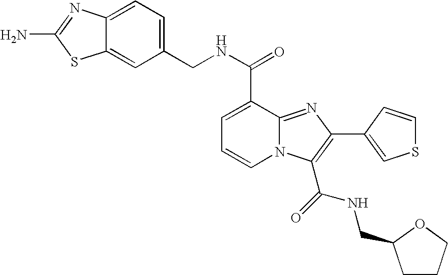 | 532.14 | 533.0 | 3.497 |
| 31 | 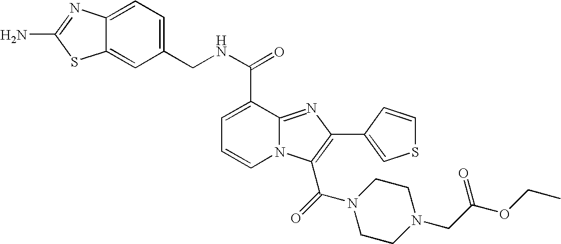 | 603.17 | 604.2 | 2.776 |
| 32 | 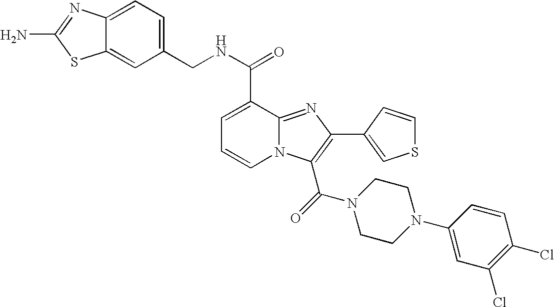 | 661.09 | 662.0 | 4.867 |
| 33 | 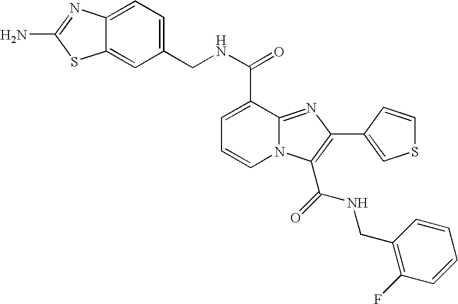 | 556.12 | 557.0 | 4.050 |
| 34 | 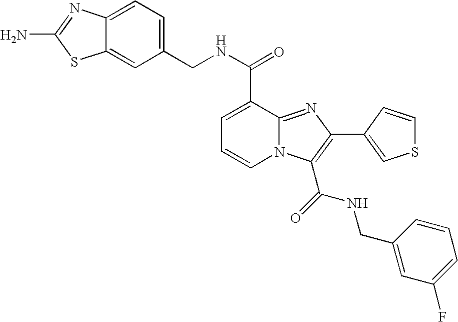 | 556.12 | 557.0 | 4.071 |
| 35 | 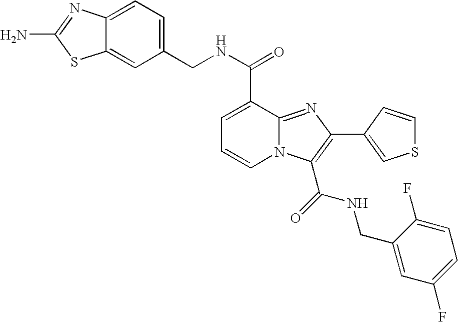 | 574.11 | 575.0 | 4.101 |
| 36 | 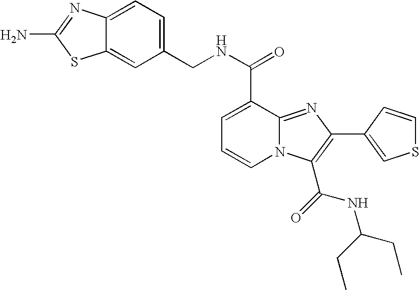 | 518.16 | 519.1 | 4.141 |
| 37 | 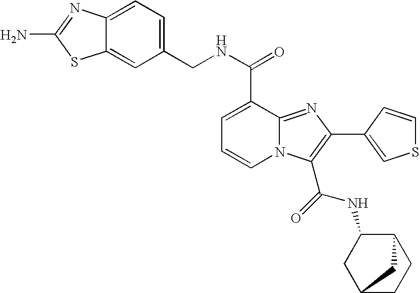 | 542.16 | 543.1 | 4.350 |
| 38 | 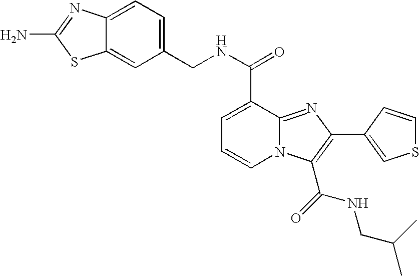 | 504.14 | 505.1 | 3.931 |
| 39 | 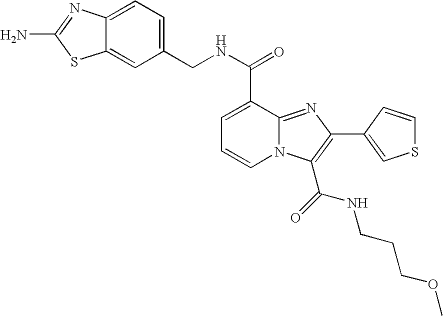 | 520.14 | 521.0 | 3.377 |
| 40 | 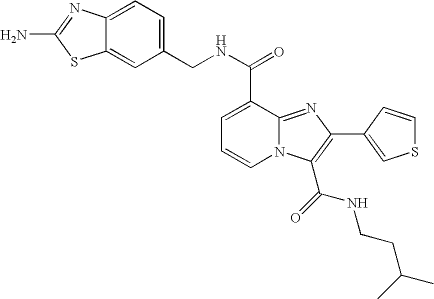 | 518.16 | 519.1 | 4.232 |
| 41 | 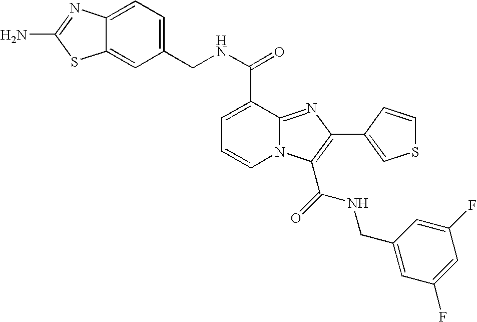 | 574.11 | 575.0 | 4.191 |
| 42 | 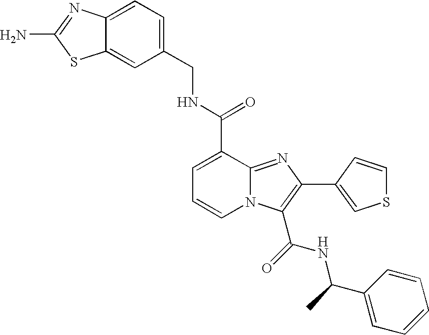 | 552.14 | 553.1 | 4.156 |
| 43 | 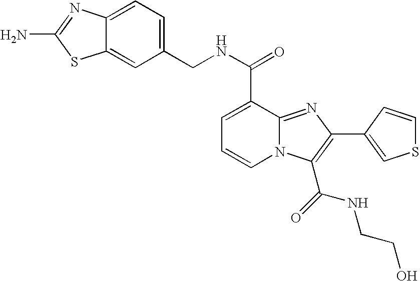 | 492.10 | 493.0 | 2.735 |
| 44 | 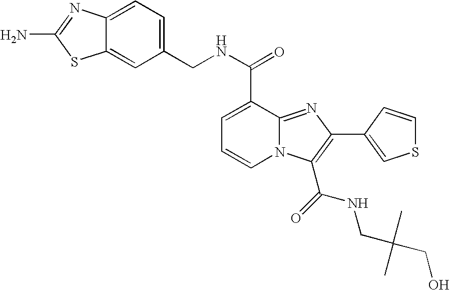 | 534.15 | 535.1 | 3.333 |
| 45 | 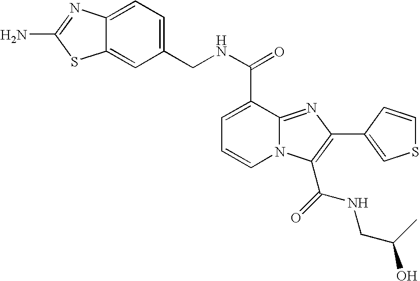 | 506.12 | 507.1 | 2.926 |
| 46 | 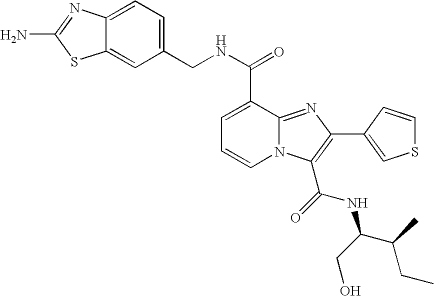 | 548.17 | 549.1 | 3.713 |
| 47 | 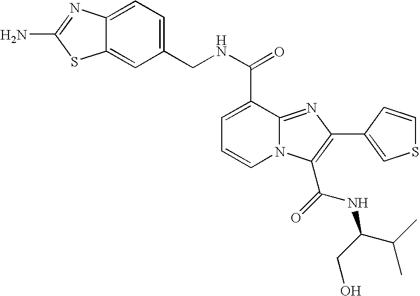 | 534.15 | 535.2 | 3.436 |
| 48 | 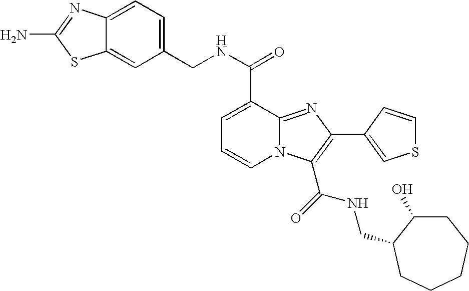 | 574.18 | 575.0 | 3.902 |
| 49 | 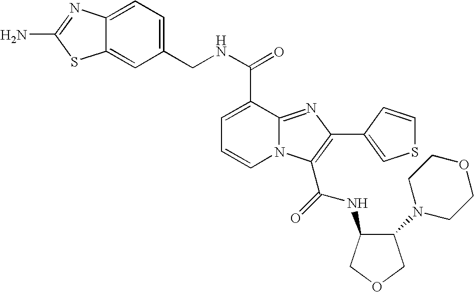 | 603.17 | 604.2 | 2.453 |
| 50 | 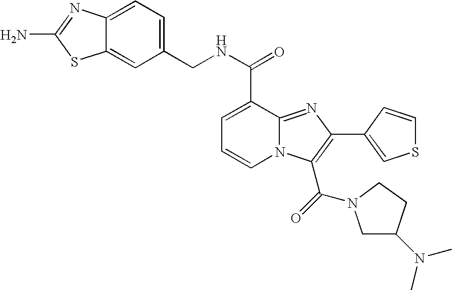 | 545.17 | 546.1 | 2.371 |
| 51 | 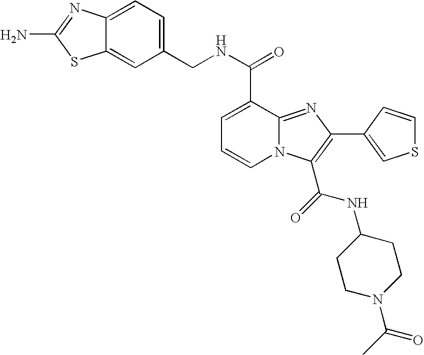 | 573.16 | 574.1 | 3.092 |
| 52 | 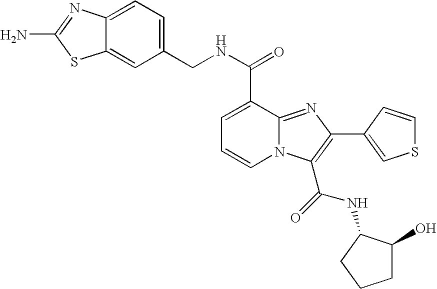 | 532.14 | 533.0 | 3.211 |
| 53 | 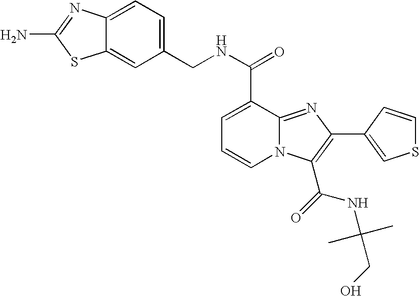 | 520.14 | 521.0 | 3.306 |
| 54 | 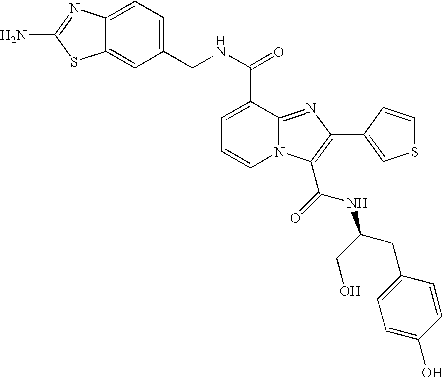 | 598.15 | 599.0 | 3.045 |
| 55 | 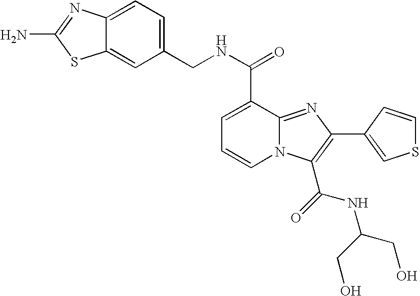 | 522.11 | 523.0 | 2.586 |
| 56 | 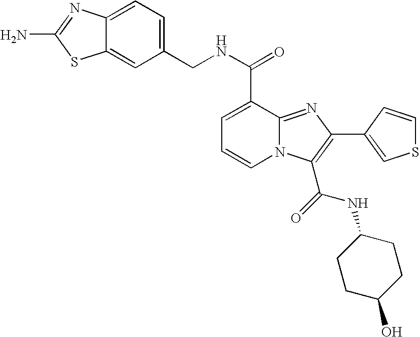 | 546.15 | 547.0 | 3.105 |
| 57 | 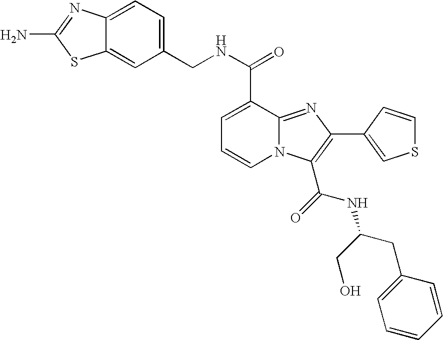 | 582.15 | 583.0 | 3.714 |
| 58 | 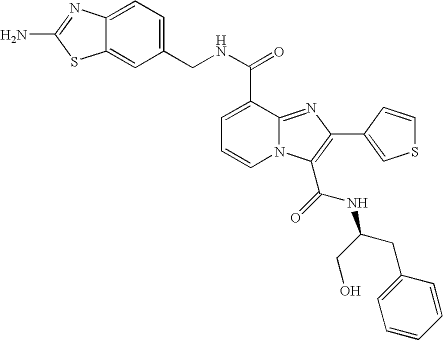 | 582.15 | 583.0 | 3.609 |
| 59 | 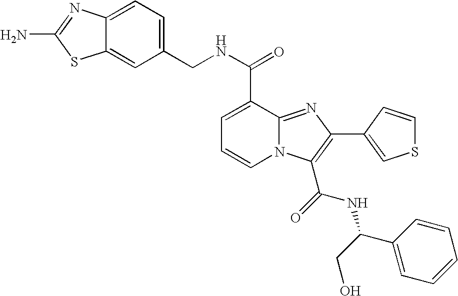 | 568.14 | 569.0 | 3.568 |
| 60 | 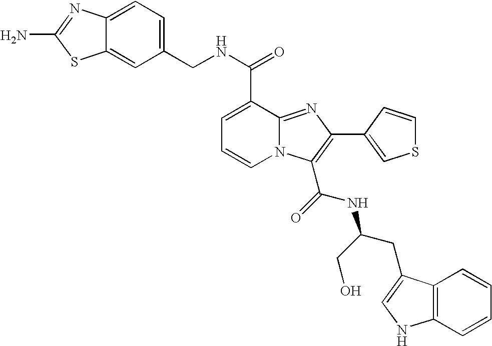 | 621.16 | 622.0 | 3.548 |
| 61 | 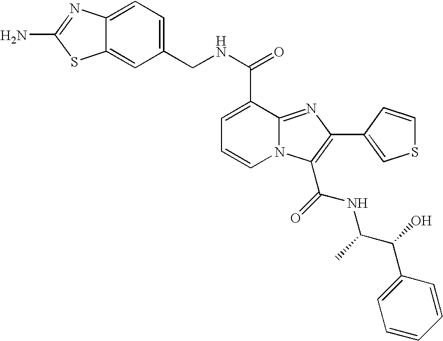 | 582.15 | 583.0 | 3.767 |
| 62 | 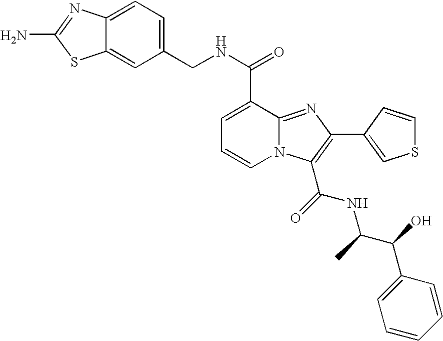 | 582.15 | 583.1 | 3.775 |
| 63 | 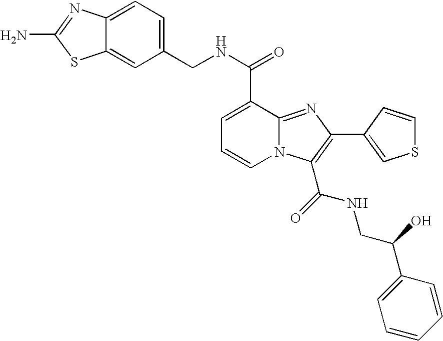 | 568.14 | 569.0 | 3.582 |
| 64 | 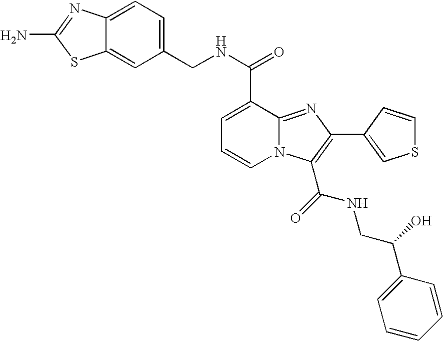 | 568.14 | 569.0 | 3.576 |
| 65 | 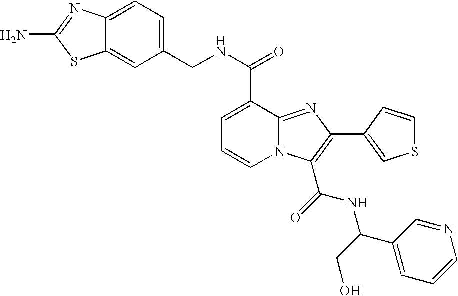 | 569.13 | 570.1 | 2.461 |
| 66 | 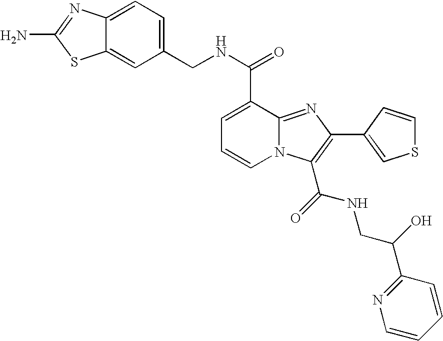 | 569.13 | 570.1 | 2.578 |
| 67 | 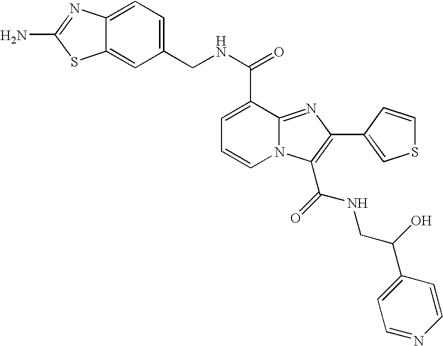 | 569.13 | 570.1 | 2.480 |
| 68 | 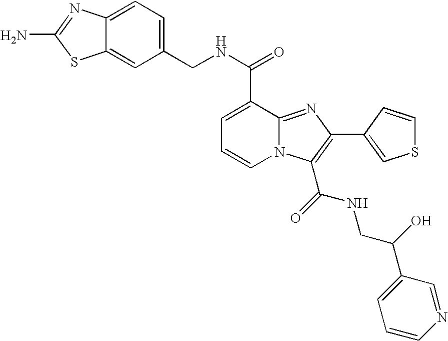 | 569.13 | 570.1 | 2.477 |
| 69 | 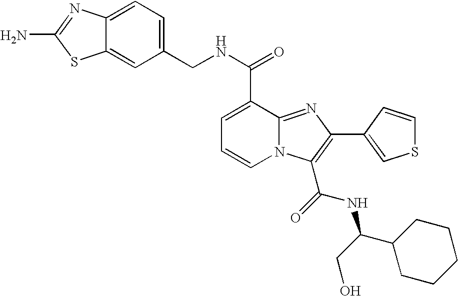 | 574.18 | 575.34 | 4.11 |
| 70 | 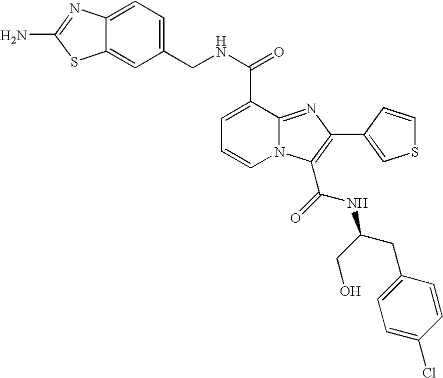 | 616.11 | 617.27 | 4.12 |
| 71 | 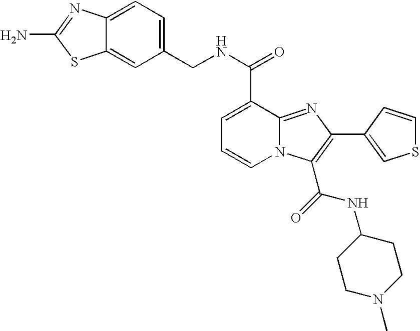 | 545.17 | 546.1 | 2.453 |
| 72 | 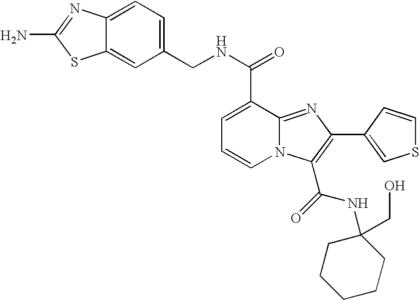 | 562.15 | 563.0 | 2.976 |
| 73 | 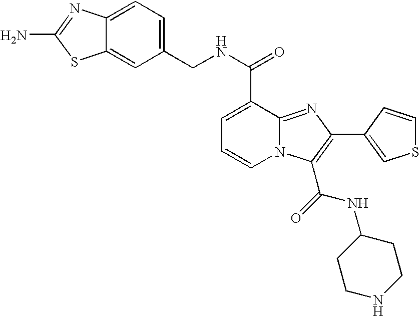 | 531.15 | 532.0 | 2.421 |
| 74 | 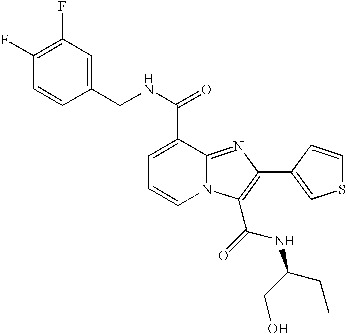 | 484.14 | 485.1 | 4.768 |
| 75 | 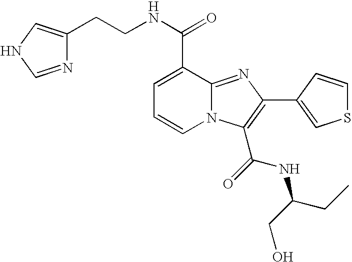 | 452.16 | 453.1 | 2.625 |
| 76 | 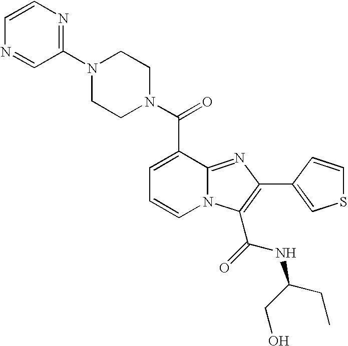 | 505.19 | 506.2 | 2.983 |
| 77 | 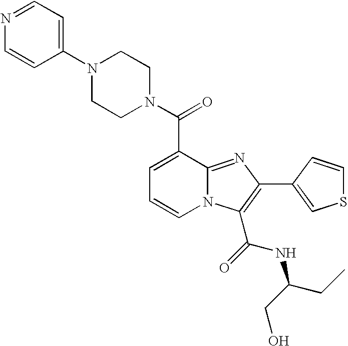 | 504.19 | 505.1 | 2.359 |
| 78 | 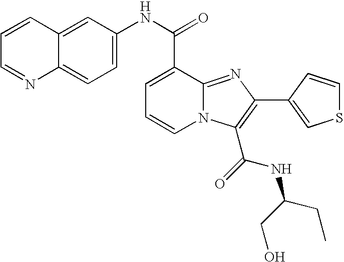 | 485.15 | 486.1 | 3.511 |
| 79 | 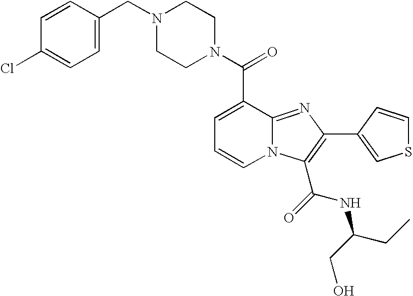 | 551.18 | 552.1 | 3.232 |
| 80 | 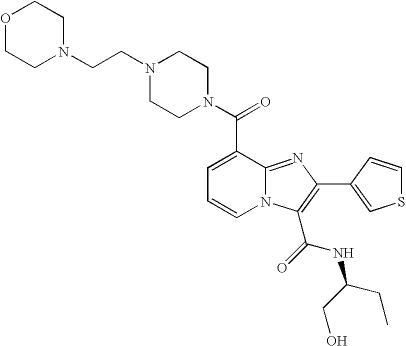 | 540.25 | 541.3 | 2.154 |
| 81 | 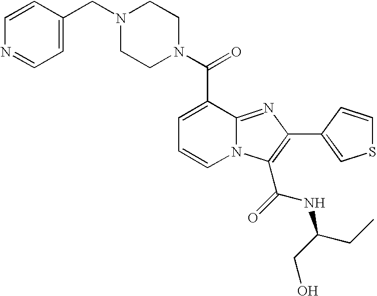 | 518.21 | 519.2 | 2.203 |
| 82 | 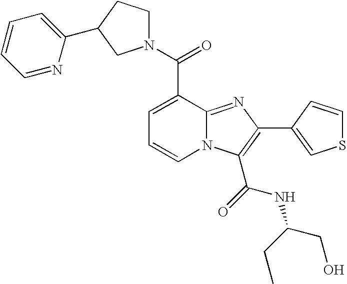 | 489.18 | 490.1 | 2.418 |
| 83 | 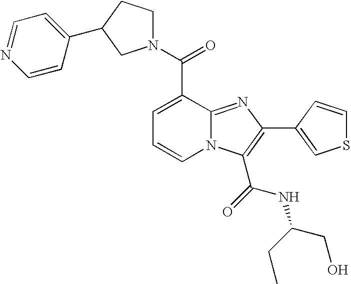 | 489.18 | 490.1 | 2.299 |
| 84 | 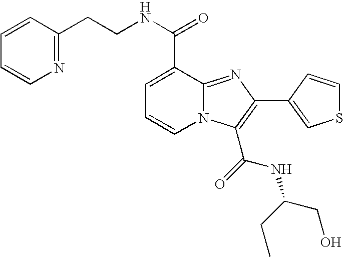 | 463.17 | 464.1 | 2.795 |
| 85 | 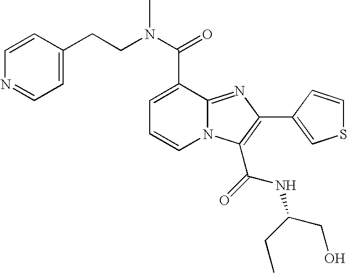 | 477.18 | 478.1 | 2.329 |
| 86 | 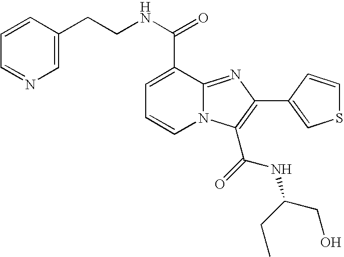 | 463.17 | 464.1 | 2.790 |
| 87 | 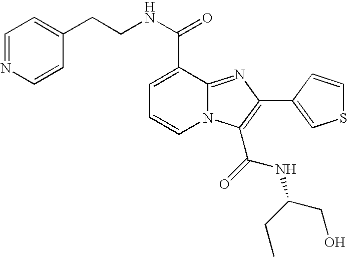 | 463.17 | 464.1 | 2.768 |
| 88 | 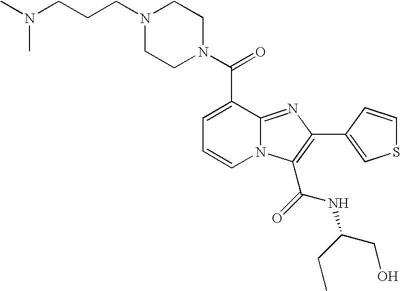 | 512.26 | 513.3 | 2.946 |
| 89 | 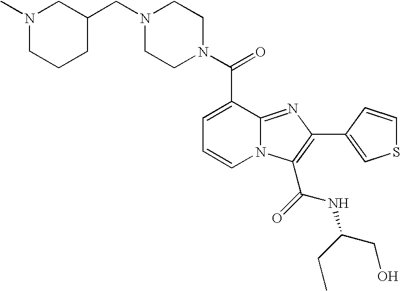 | 538.27 | 539.2 | 2.191 |
| 90 | 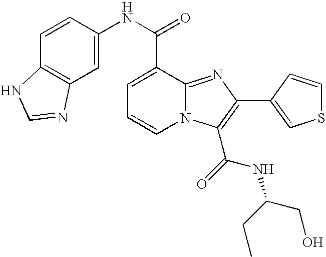 | 474.15 | 475.1 | 3.195 |
| 91 | 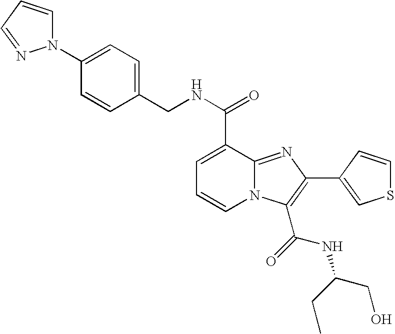 | 514.18 | 515.1 | 4.345 |
| 92 | 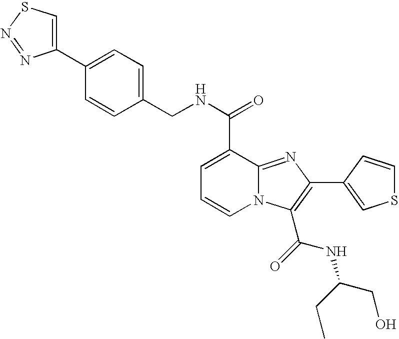 | 532.14 | 533.0 | 4.424 |
| 93 | 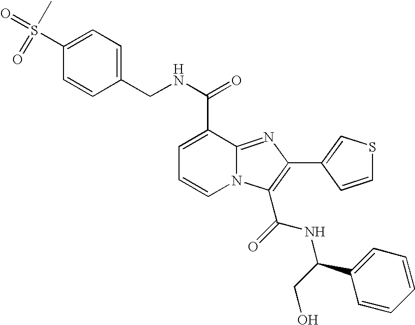 | 574.13 | 575.23 | 4.45 |
| 94 | 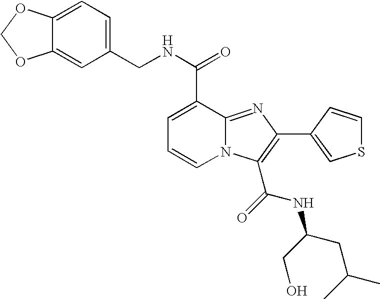 | 520.18 | 521.1 | 4.759 |

### Example 1C

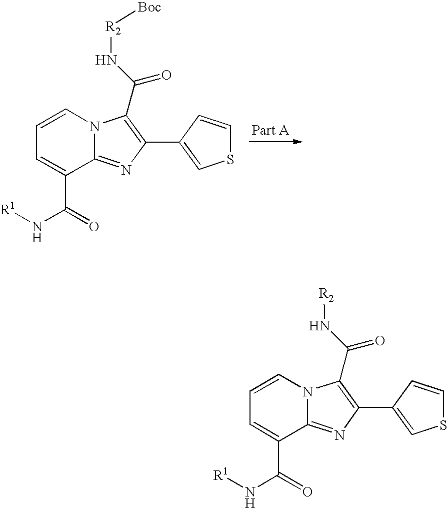

(wherein R¹ is remaining moiety of the Q^{A} group, and R² is the remaining moiety of the Q^{B} group in formula 1.0)

### Part A:

The crude compounds which were synthesized using methods described in Example 1B, were dissolved in dioxane (1 mL), and a solution of 4 N HCl in dioxane (2 mL) and water (0.2 mL) was added 0° C. The reaction mixture was stirred at room temerature for 3 hours. LC-MS analysis of the reaction indicated that the hydrolysis was complete. The volatiles were removed in Vacuo, acetonitrile was added, concentrated and dried to afford the desired compounds. Purification by Prep. LC and conversion to the hydrochloride salt afforded compounds as white solids. The compounds prepared are in Table 2.

TABLE 2

| Compd | | | MS m/z | Ret. Time |
|---|---|---|---|---|
| No. | Structure | EMW | (M⁺ + H) | (min) |
| 97 | 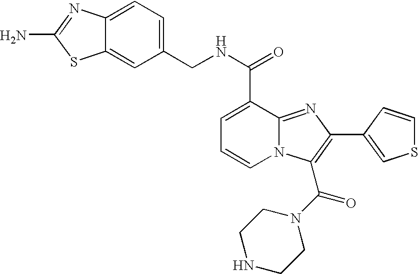 | 517.14 | 518.0 | 2.406 |
| 98 | 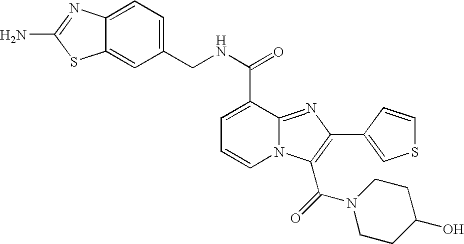 | 532.14 | 533.0 | 2.871 |
| 99 | 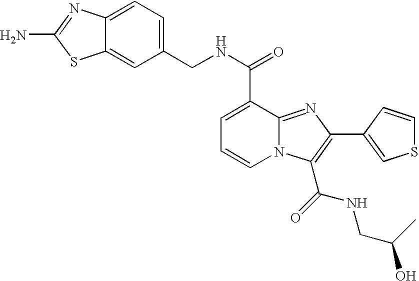 | 506.12 | 507.1 | 2.940 |
| 100 | 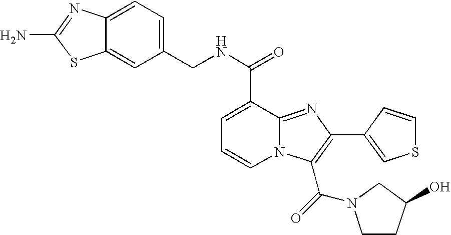 | 518.12 | 519.1 | 2.799 |
| 101 | 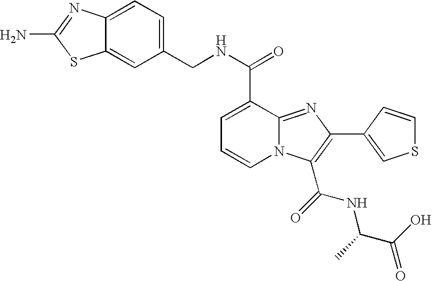 | 520.10 | 521.0 | 3.002 |

### Example 1D

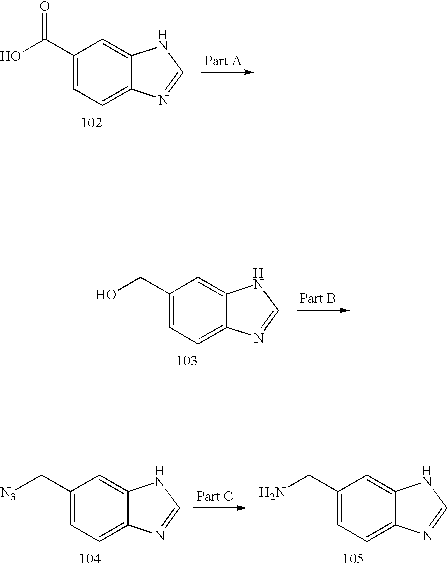

### Part A

Benzimidazole-5-carboxylic acid 102 (1 g, 6.17 mmol) in THF (100 mL) was added 1 N. LAH soln. (13 mL) at 0° C. After the complete addition of LAH soln., reaction mixture was warmed to room temperature and then refluxed for 3 hours. The solution was cooled to 0° C. and then excess of LAH is quenched with satd. soln. of Na₂SO₄. Filtered and solid was washed with ethyl acetate. The solution was concentrated to obtain compound 103.

### Part B

To a solution of 5-(hydroxymethyl)-benzimidazole 103 (0.74 g., 5 mmol) in THF was added DPPA (5.5 mmol) followed by DBU (1.2 mmol). The resulting solution was heated to reflux for 5 hours, cooled to room temperature, and concentrated. The residue was dissolved in ethyl acetate and washed with sodium NaHCO₃ solution, brine and dried over anhydrous sodium sulfate. Crude product 104 was purified on silica gel chromatography using Methanol-Chloroform solvents. HPLC-MS t_{R}=0.855 min (UV_{254 nm}), mass calculated for formula C₈H₇N₅ 173.07, observed LCMS m/z 174.1 (M+H).

### Part D

To a stirred solution of 5-(azidomethyl)benzimidazole 104 (0.519 g., 3 mmol) in THF (10 mL), was added Ph₃P (6 mmol) followed by water 0.20 mL and the reaction mixture was stirred overnight at room temperature. The reaction mixture was concentrated. The residue was dissolved in ethyl acetate and dry HCl gas was bubbled through the solution. The precipitate was filtered to obtain compound 105. HPLC-MS t_{R}=0.2 min (UV_{254 nm}); mass calculated for formula C₈H₉N₃ 147.08, observed LCMS m/z 148.1 (M+H).

The building blocks in Table 3 are synthesized using above procedures.

TABLE 3

| Com- | | | |
|---|---|---|---|
| pound | | MW | M⁺+ H |
| # | Structure | calculated | observed |
| 106 | 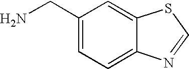 | 164.04 | 165.1 |
| 107 | 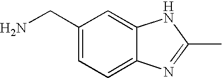 | 161.10 | 162.1 |
| 108 | 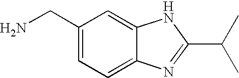 | 189.13 | 190.1 |
| 109 | 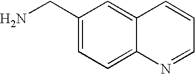 | 158.08 | 159.1 |
| 110 | 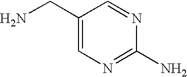 | 124.07 | 125.1 |

The compounds in Table 4 are synthesized using the building blocks from Table 3 and methods similar to those described in Example 1B. Compounds are purified on prep. LC either after the reaction part H or part I in Example 1B and converted to their hydrochloride salts.

TABLE 4

| | | | | Ret. |
|---|---|---|---|---|
| Compd | | | MS m/z | Time |
| # | Structure | EMW | (M⁺ + H) | (min) |
| 111 | 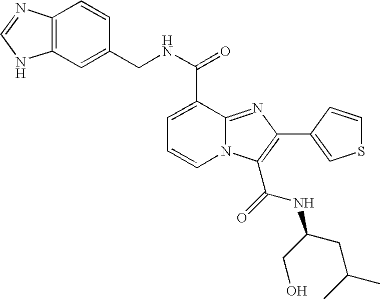 | 516.19 | 517.1 | 3.478 |
| 112 | 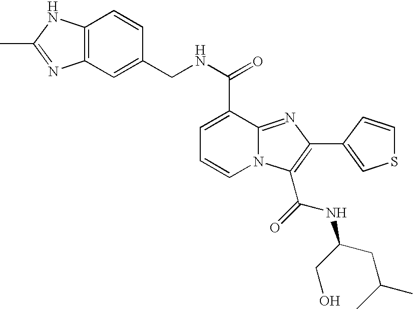 | 530.21 | 531.38 | 3.75 |
| 113 | 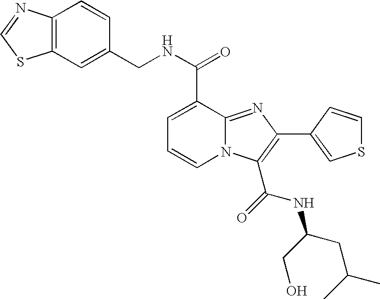 | 533.16 | 534.1 | 4.614 |
| 114 | 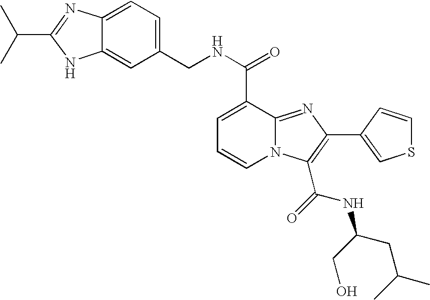 | 558.24 | 559.32 | 3.89 |
| 115 | 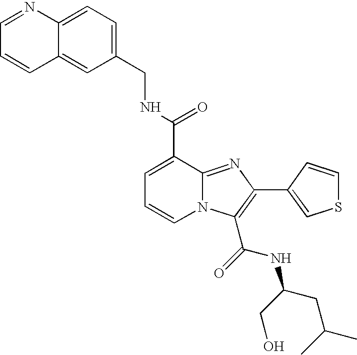 | 527.20 | 528.29 | 3.61 |
| 116 | 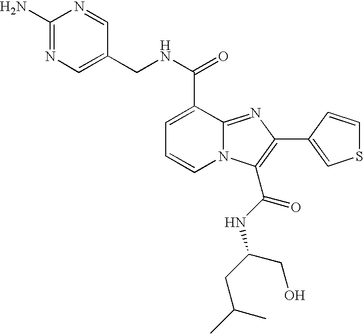 | 493.19 | 494.36 | 3.48 |
| 117 | 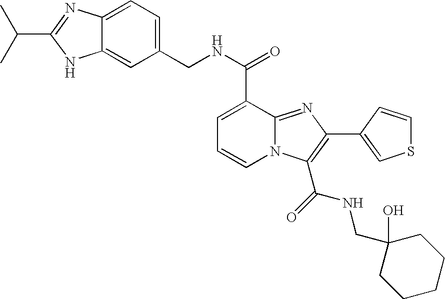 | 570.24 | 571.33 | 3.94 |
| 118 | 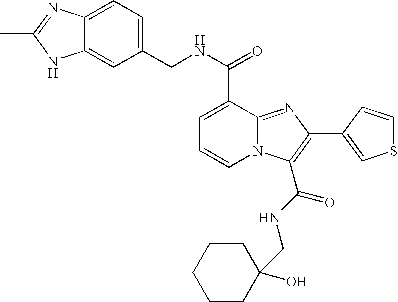 | 542.21 | 543.27 | 3.69 |
| 119 | 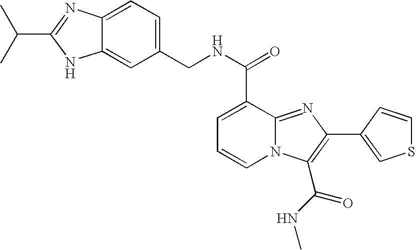 | 472.17 | 473.21 | 3.14 |
| 120 | 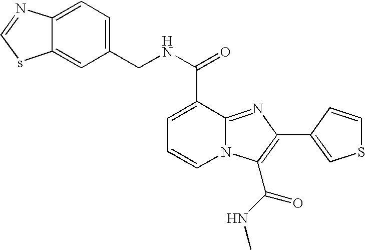 | 447.08 | 448.16 | 4.22 |
| 121 | 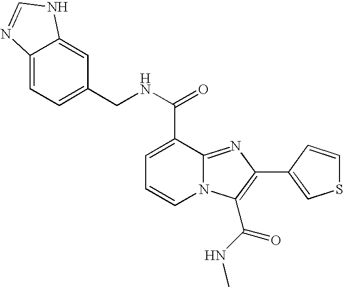 | 430.12 | 431.21 | 2.8 |
| 122 | 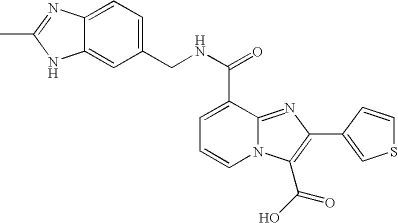 | 431.11 | 432.18 | 2.92 |
| 123 | 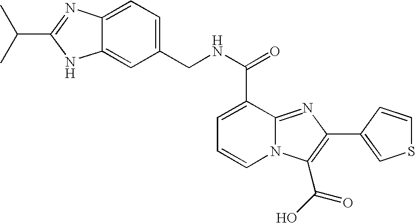 | 459.14 | 460.2 | 3.17 |
| 124 | 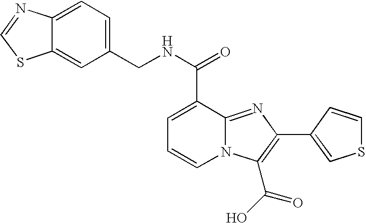 | 434.05 | 435.11 | 4.28 |
| 125 | 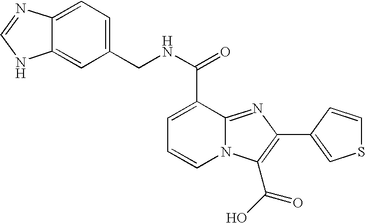 | 417.09 | 418.17 | 2.83 |

### Example 1E

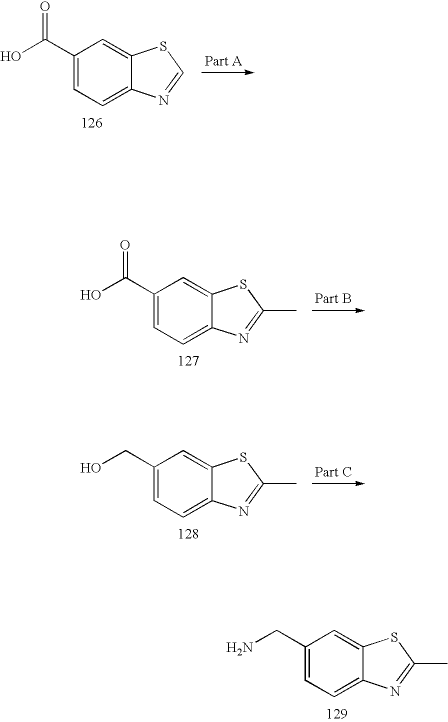

### Part A

Benzothiazole-6- carboxylic acid 126 (1.79 g., 10 mmol) was suspended in THF (200 mL) and cooled to −78° C. n BuLi (2.5 N soln. in Hexane, 10 mL) was added and the reaction mixture was stirred for an hour followed by the addition of MeI (1.2 equiv. 1.7 g.) in 10 mL of THF. The reaction mixture was warmed to room temperature and the stirring was continued overnight. Reaction was cooled to 0° C. and then quenced with brine solution and extracted with ethyl acetate. The organic layer was dried over anhydrous Na₂SO₄ and concentrated to yield compound 127. HPLC-MS t_{R}=1.123 min (UV_{254 nm}); mass calculated for formula C₉H₇NO₂S 193.02, observed LCMS m/z 193.9 (M+H).

### Part B

2-Methyl benzothiazole-6-carboxylic acid 127 was converted to its alcohol 128 using the procedure described in Example 1D. HPLC-MS t_{R}=0.955 min (UV_{254 nm}); mass calculated for formula C₉H₉NOS179.04, observed LCMS m/z 180.0 (M+H).

### Part C

(2-Methyl-benzothiaole-6-yl)-methanol 128 was converted to (2-Methyl-benzothiaole-6-yl)-methylamine 129 using procedures described in Example 1D, Part C and Part D HPLC-MS t_{R}=0.295 min (UV_{254 nm}); mass calculated for formula C₉H₁₀N₂S 178.06, observed LCMS m/z 179.1 (M+H).

The compounds in Table 5 are made using compound 129 and core 8 according to the methods described in Example 1B.

TABLE 5

| Compd | | | MS m/z | Ret. Time |
|---|---|---|---|---|
| # | Structure | EMW | (M⁺ + H) | (min) |
| 130 | 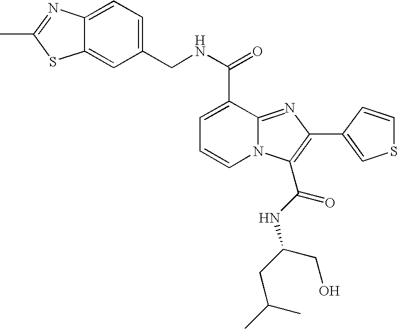 | 547.17 | 548.25 | 5.16 |
| 131 | 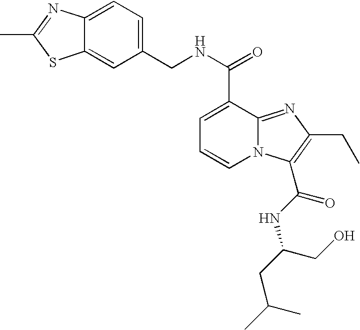 | 493.22 | 494.28 | 4.33 |

### Example 1F

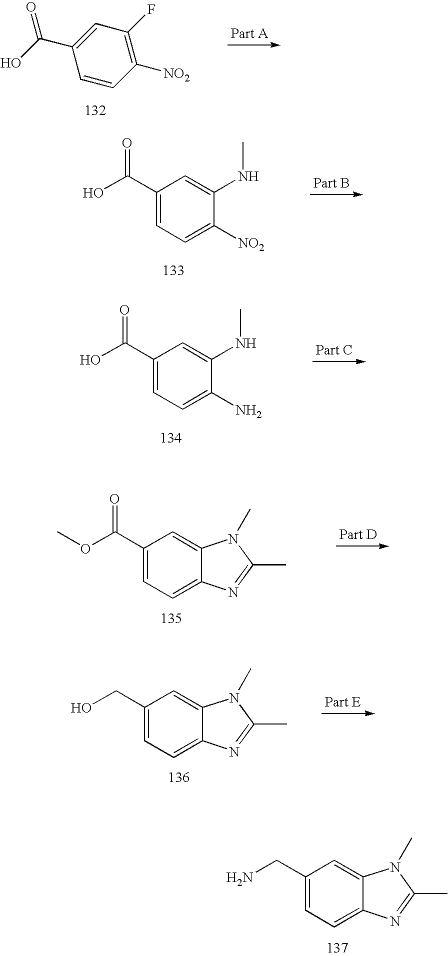

### Part A

To the solution of 3-fluoro-4-nitrobenzoic acid 132 (1 g, 5.40 mmol) was suspended in Ethanol (20 mL) and methylamine (40 wt % in water, 10 mL) was added and refluxed overnight. Reaction mixture was cooled to room tepecrature and concentrated to obtain compound 133. HPLC-MS t_{R}=1.088 min (UV_{254 nm}); mass calculated for formula C₈H₈N₂O₄ 196.05, observed LCMS m/z 197.1 (M+H).

### Part B

3-methylamino-4-nitro benzoic acid 133 (1 g, 5.10 mmol) was suspended in Ethanol (20 mL) and catalytic amount of 5% Pd on carbon was added. The reaction flask was sealed with septum, evacuated by applying vacuum and hydrogen balloon was inserted and stirred overnight. The solution was filtered through celite pad and concentrated to yield compound 134. HPLC-MS t_{R}=0.229 min (UV_{254 nm}); mass calculated for formula C₈H₁₀N₂O₂ 166.07, observed LCMS m/z 167.1 (M+H).

### Part C

4-Amino-3-methylamino benzoic acid 134 was taken in 20 mL of acetic acid and refluxed for overnight. The reaction mixture was cooled and concentrated. The residue was taken in methanol and acetonitrile mixture (1:1) and added (Trimethylsilyl) diazomethane (2 M soln. in hexanes, 10 mmol) at 0° C. The solution was stirred for 1 hr and concentrated. The crude product was purified on silica column using Methanol/Ethylacetate solvent system. HPLC-MS t_{R}=0.797 min (UV₂₅₄ nm); mass calculated for formula C₁₁H₁₂N₂O₂ 204.09, observed LCMS m/z 205.1 (M+H).

### Part D

To a suspension of 2,3-Dimethyl-benzimidazole-5-carboxylic acid methyl ester 135 (0.5 g., 2.5 mmol) in 50 mL of DCM was added 3 equivalents of 1 M solution of DIBAL-H at −78° C. and the mixture was stirred for 4 hrs. The reaction mixture was warmed to room temperature. The reaction was cooled to 0° C. quenched by the sequential addition of 1 M sodium hydroxide and 30% Rochelle salt (10 mL). The mixture was filtered, and the residue was washed with DCM. The filtrate was concentrated to obtain compound 136. HPLC-MS mass calculated for formula C₈H₈N₂O 148.06, observed LCMS m/z 149.1 (M+H).

### Part E

2,3-Dimethyl-3H-benzimidazol-5-yl)-methanol has been converted to the compound 137 using the procedures illustrated in Example 1D. Part B and Part C. HPLC-MS t_{R}=0.210 min (UV_{254 nm}); mass calculated for formula C10H13N3 175.11, observed LCMS m/z 176.2 (M+H).

### Example 1G

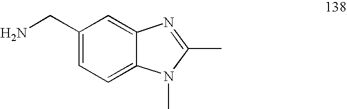

(1,2-Dimethyl-1H-benzimidazol-5-yl)-methylamine has been synthesized starting from 4-Fluoro 3 nitro benzoic acid using procedures described in Example 1F

HPLC-MS t_{R}=0.177 min (UV_{254 nm}); mass calculated for formula C₁₀H₁₃N₃ 175.11, observed LCMS m/z 176.2 (M+H).

The compounds in Table 6 are made using compounds 137 and 138 and the methods described in Example 1B.

TABLE 6

| Compd | | | MS m/z | Ret. Time |
|---|---|---|---|---|
| # | Structure | EMW | (M⁺+ H) | (min) |
| 139 | 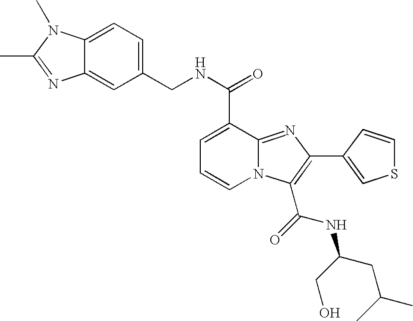 | 544.23 | 545.31 | 3.72 |
| 140 | 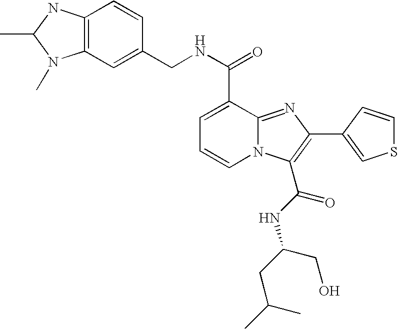 | 544.23 | 545.23 | 3.41 |

### Example 1H

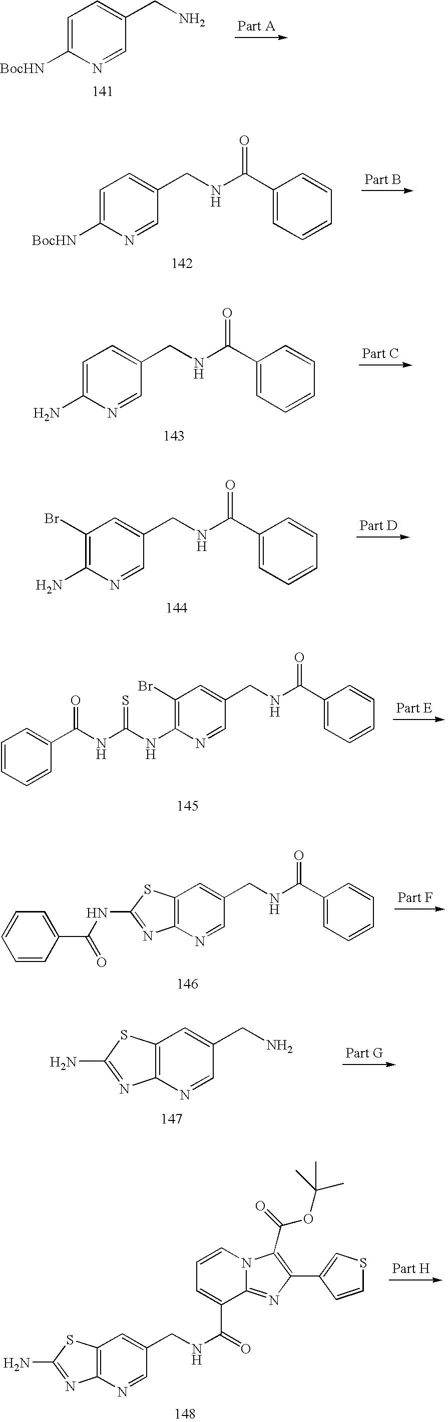

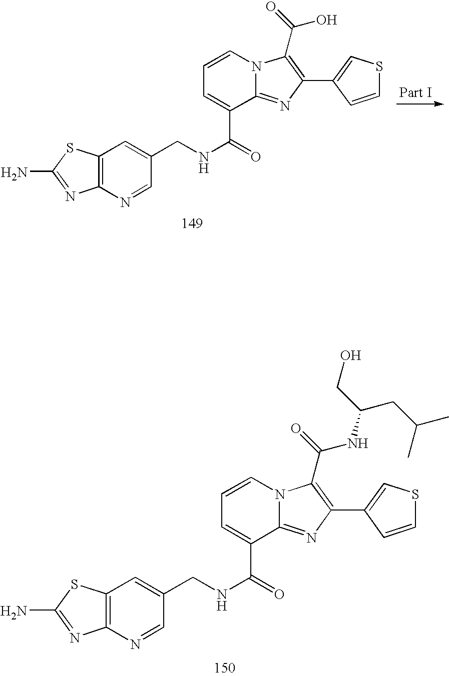

### Part A

The compound 141 (1.0 g, 4.5 mmol) was dissolved in DCM (20 mL) and TEA (1.36 mL, 10 mmol) was added. The mixture was cooled to 0° C. with ice-water bath and benzoyl chloride (0.675 g, 4.8 mmol) was added. The resulting mixture was allowed to warm to room temperature and stirred for 3 hours. The mixture was diluted with EtOAc (200 mL) and washed with H₂O, NaHCO₃, and brine and dried over Na₂SO₄. After concentration, the crude residue was purified with short column (silica gel, hexane/EtOAc=70/30) gave the product 142 (1.31 g). HPLC-MS t_{R}=1.48 min (UV_{254 nm}); mass calculated for formula C₁₈H₂₁N₃O₃ 327.2, observed LCMS m/z 328.1 (M+H).

### Part B

The compound 142 (1.0 g, 3.0 mmol) was dissolved in MeOH (3 mL) and HCl (6N, 5 mL) was added. The mixture was stirred at room temperature for 1 hour and concentrated. The aqueous was treated with NaHCO₃ (sat. aq., 30 mL) and extracted with EtOAc. The organics were dried over Na₂SO₄ and concentrated to give the crude product 143. It was used in the next step without further purification. HPLC-MS t_{R}=0.61 min (UV_{254 nm}); mass calculated for formula C₁₃H₁₃N₃O 227.1, observed LCMS m/z 228.1 (M+H).

### Part C

The 2-aminopyridine compound 143 (1.14 g, 5 mmol) was dissolved in HOAc (20 mL) and bromine (0.260 mL, 5.0 mmol) was added at room temperature. The mixture was stirred for 1 hour and concentrated. The resulting residue was diluted with Na₂CO₃ (aq.) and extracted with EtOAc. After concentration, the product was purified with column (silica gel, hexane/EtOAc=40/60) gave the pure product 144 (1.28 g) as white solid. HPLC-MS t_{R}=0.91 min (UV_{254 nm}); mass calculated for formula C₁₃H₁₂BrN₃O 305.0, observed LCMS m/z 306.0 (M+H).

### Part D

A mixture of ammonium thiocyanate (0.35 g, 4.3 mmol) and acetone (1.5 mL) was warmed until a clear solution was obtained. Benzoyl chloride (0.53 mL, 4.3 mmol) was then slowly dropped in and the resulting suspension refluxed 5 min. The 2-amino-3-bromopyridine 144 (1.28 g, 4.3 mmol) in acetone (1.5 mL) was added and the reaction mixture was refluxed for 1 hour. After cooling to room temperature, the solution was poured into water and the solid was collected by filtration, washed with water, ethyl ether and dried under vacuum. Gave the product 145 (1.15 g) as white solid. HPLC-MS t_{R}=1.32 min (UV_{254 nm}); mass calculated for formula C₂₁H₁₇BrN₄O₂S 468.0, observed LCMS m/z 469.0 (M+H).

### Part E

The compound 145 (1.15 g, 2.5 mmol) was dissolved in NMP (10 mL) and NaOMe (0.810 g, 15 mmol) was added. The mixture was heated up to 120° C. under Ar for 4 hours. After cooling down to room temperature, the mixture was diluted with EtOAc and washed with NH₄Cl (aq.) and brine. After drying over Na₂SO₄, the organics were concentrated and the residue was purified by column (silica gel, hexane/EtOAc=20/80) gave the compound 146 (0.710 g) as yellowish solid. HPLC-MS t_{R}=1.53 min (UV_{254 nm}); mass calculated for formula C₂₁H₁₆N₄O₂S 388.1, observed LCMS m/z 389.0 (M+H).

### Part F

Compound 146 (710 mg, 1.8 mmol) was treated with HCl (6N, 5 mL) and heated up to refluxed overnight. After cooling to room temperature, the aqueous was extracted with ethyl ether. The aqueous was concentrated and dried with lyophlization gave the product 147 which was used in the next step directly without further purification. HPLC-MS t_{R}=0.18 min (UV_{254 nm}); mass calculated for formula C₇H₈N₄S 180.0, observed LCMS m/z 181.1 (M+H).

### Part G

Compound 148 was prepared using the peptide coupling conditions described in Example 1B Part F. HPLC-MS t_{R}=1.70 min (UV_{254 nm}); mass calculated for formula C₂₄H₂₂N₆O₃S₂ 506.1, observed LCMS m/z 507.1 (M+H).

### Part H

Compound 149 was prepared using the hydrolysis conditions described in Example 1B Part H. HPLC-MS t_{R}=1.06 min (UV_{254 nm}); mass calculated for formula C₂₀H₁₄N₆O₃S₂ 450.0, observed LCMS m/z 451.0 (M+H).

### Part I

Compound 150 was prepared using the peptide coupling conditions described in Example 1B Part I. HPLC-MS t_{R}=1.35 min (UV_{254 nm}); mass calculated for formula C₂₆H₂₇N₇O₃S₂ 549.1, observed LCMS m/z 550.0 (M+H).

| Compd | | | MS m/z | Ret. Time |
|---|---|---|---|---|
| # | Structure | EMW | (M⁺ + H) | (min) |
| 150 | 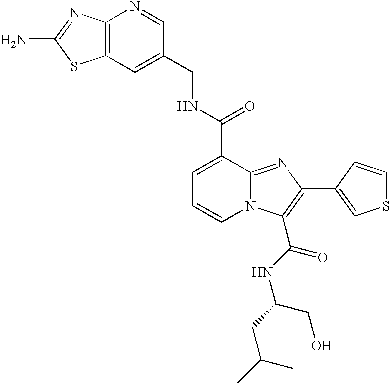 | 549.1 | 550.0 | 1.35 |

### Example 2A

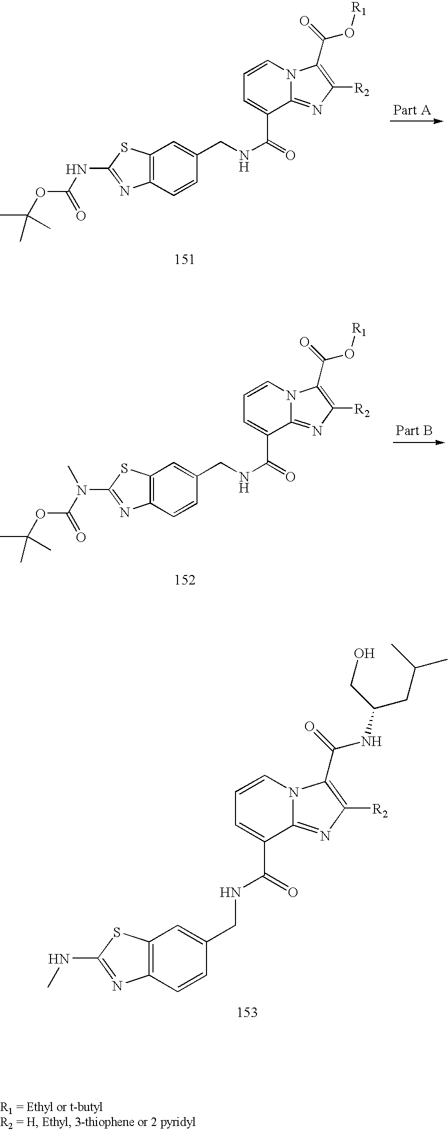

### Part A

Compounds of structure 151 was synthesized using methods described in Example 1B (Part F and G). To a stirred solution of compound 151 (0.064 mmol) in anhydrous THF (1 mL) was added the Methanol (1 equivalent), triphenylphosphine (1.5 equivalents) and DIAD (1.5 equivalents) at room temperature. The reaction mixture was continued to stirr at room temperature for 5 hours at which time LC-MS analysis indicated the reaction was complete. The reaction mixture was concentrated and purified using column chromatography.

### Part B

The final compounds in Table 7 are synthesized using the methods described in Example 1B.

TABLE 7

| | | | | Ret. |
|---|---|---|---|---|
| Compd | | | MS m/z | Time |
| # | Structure | EMW | (M⁺+ H) | (min) |
| 154 | 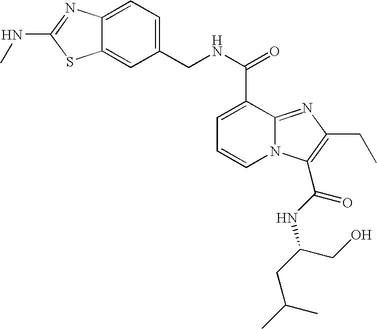 | 508.23 | 509.30 | 3.24 |
| 155 | 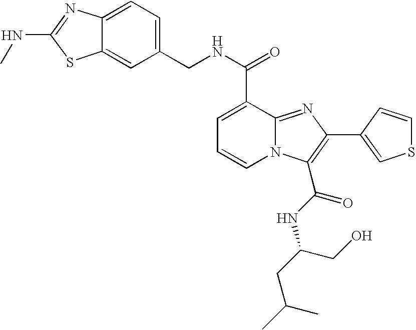 | 562.18 | 563.23 | 3.89 |
| 156 | 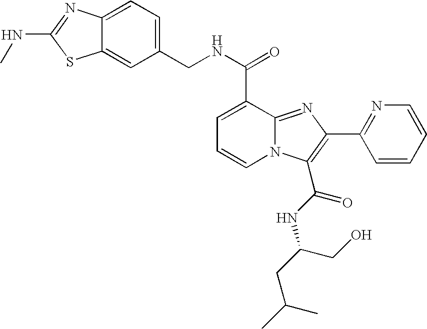 | 557.2 | 558.1 | 4.05 |
| 157 | 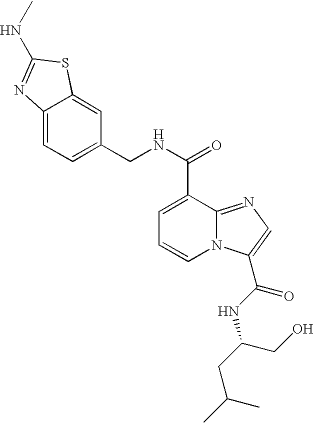 | 480.19 | 481.1 | 3.147 |
| 158 | 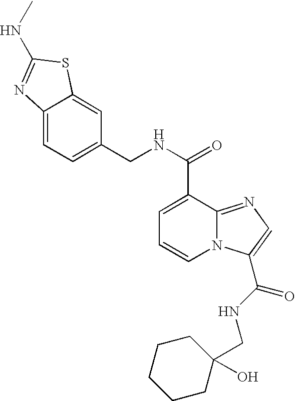 | 492.19 | 493.1 | 3.102 |

### Example 2B

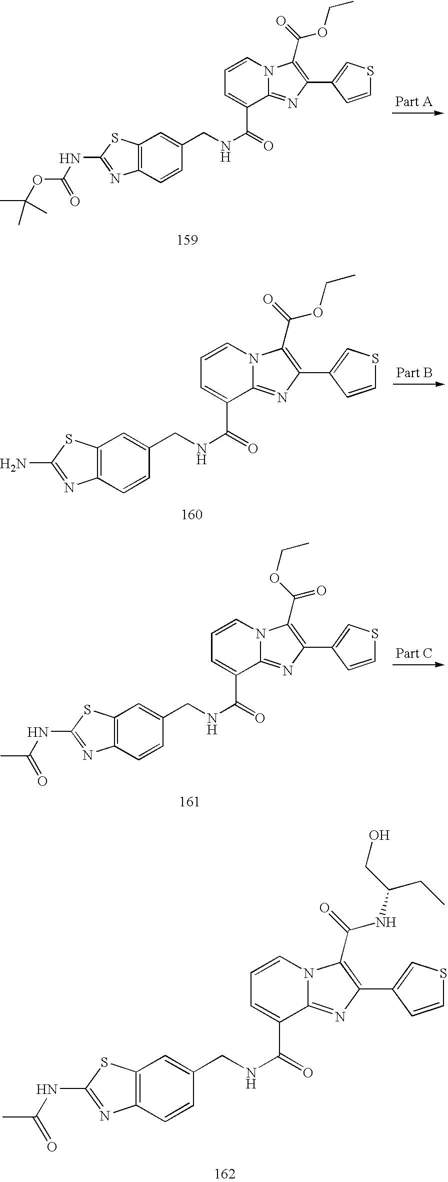

### Part A:

The Boc protecting group in compound 159 was deprotected using conditions described in Example 1B (part H).

### Part B:

To the stirred solution (0.1 mmol) in DCM (5 mL), DIEA (100 mL, 0.6 mmol) was added followed by the addition of acetyl chloride (0.15 mmol). The mixture was stirred at room temperature overnight. The reaction mixture was diluted with EtOAc and the oraganic layer was washed with NaHCO₃ soln. Water, brine and dried over anhydrous Na₂SO₄. The solvent was removed under vacuum and the resulting residue was used for the next reaction with out any further purification. HPLC-MS t_{R}=1.929 min (UV_{254 nm}); mass calculated for formula C₂₅H₂₁N₅O₄S₂ 519.10, observed LCMS m/z 520.0 (M+H).

### Part C:

The compound 161 was converted to the final product using methods described in Example 1B (Part F and Part I)

| | | | | Ret. |
|---|---|---|---|---|
| Compd | | | MS m/z | Time |
| # | Structure | EMW | (M⁺ + H) | (min) |
| 162 | 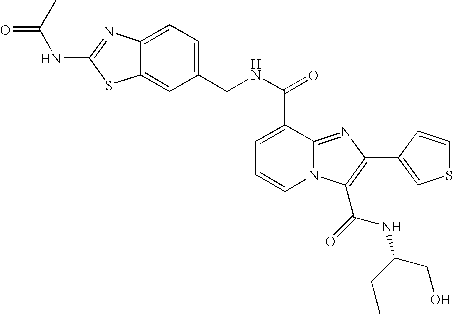 | 562.15 | 563.0 | 3.978 |

### Example 2C

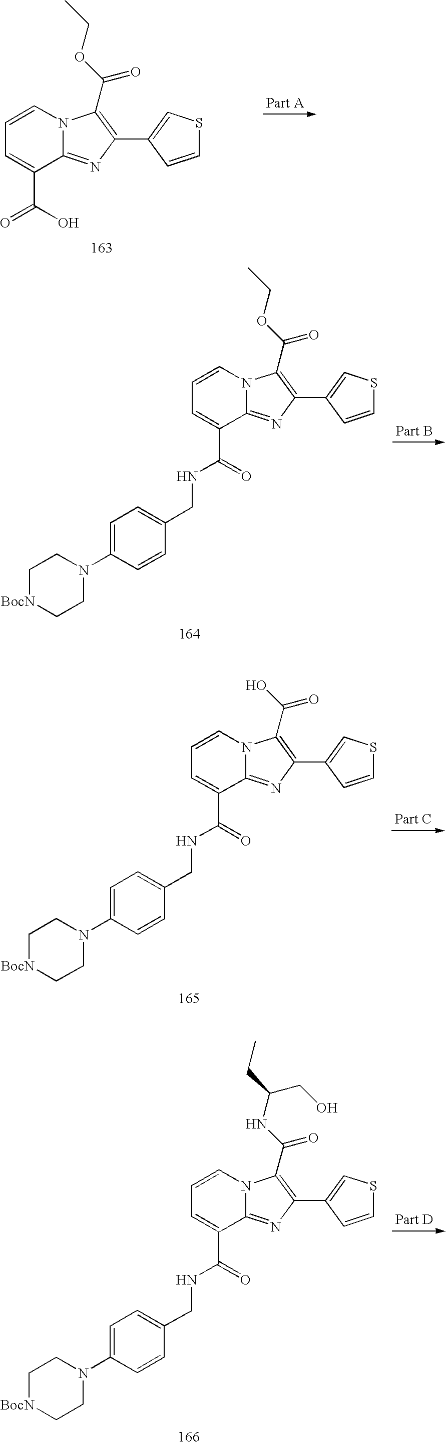

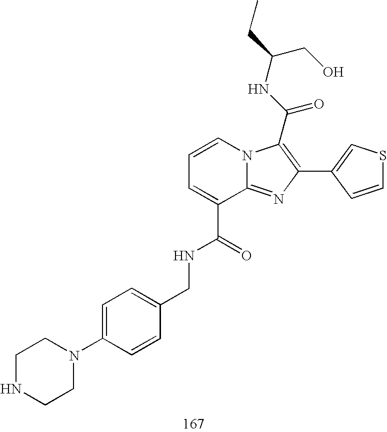

### Part A:

2-Thiphene-3-yl-imidazo[1,2-a]pyridine-3,8-dicarboxylic acid 163 (0.05 mmol) dissolved in in dichloromethane (5 mL) and cooled to −20° C. To this (1-(3-dimethylaminopropyl)-3-ethylcarbodiimide (1.2 equivalents, 0.06 mmol) was added. Followed by Diisopropyl ethyl amine (3 equivalents) was added and the solution stirred at −20° C. for 15 minutes. To the activated acid was added with 0.05 mmol solution of Amine (pre dissolved in to DCM or NMP; 0.5 mL). The solution was shaken at −5° C. for 14 hrs. LCMS analysis showed the completion of the reaction. HPLC-LC-MS mass calculated for formula C₃₁H₃₅N₅O₅S, 589.23; and observe m/z M⁺+H 590.0

### Part B:

8-[4-(4-tert-Butoxycarbomoyl-piperazin-1-yl)-2-thiophen-3-yl-imidazo[1,2-a]pyridine-3-carboxylic acid ethyl ester 164 (0.040 g) was dissolved in THF:Water (1:!; 5 mL) and LiOH (0.004 g) added and stirred at room temperature for 5 hrs. The solvent was evaporated and neutralized to pH 4 with dil.HCl. Extracted in to EtOAc. EtOAC is evaporated and dried. HPLC LC-MS mass calculated for formula, C₂₉H₃1N₅O₅S, 561.20; and observed M⁺+H 562.2

This has been used in the next step with out any further purification.

### Part C:

To the above solution, one equivalent of (1-(3-dimethylaminopropyl)-3-ethylcarbodiimide (0.05 mmol) was added in each reaction vial followed by diisopropyl ethyl amine (5 equivalents) and S—(S)-(+)-2-amino-1-butanol (0.05 mmol). The reaction mixture stirred at room temperature for overnight. LCMS analysis showed completion of reaction.

The dichloromethane/N-methylpyrrolidine solution was concentrated under vacuum. Extracted in to ethyl acetate (3×2 mL). The organic extracts were dried under vacuum and re dissolved in methanol-acetonitrile and subjected to Prep. LC purification to get the desired product in 95% purity. HPLC LC-MS mass calculated for molecular formula, C₃₃H₄₀N₆O₅S, 632.27, and observed M⁺+H=637.2

### Part D:

The above purified product was treated with 4N hydrochloride in dioxane for 1 hr. The dioxane solution evaporated under vacuum and redissolved in water-acetonitrile lyophilized to get the hydrochloride salt of the title compound.

| | | | | Ret. |
|---|---|---|---|---|
| Compd | | | MS m/z | Time |
| # | Structure | EMW | (M⁺+ H) | (min) |
| 167 | 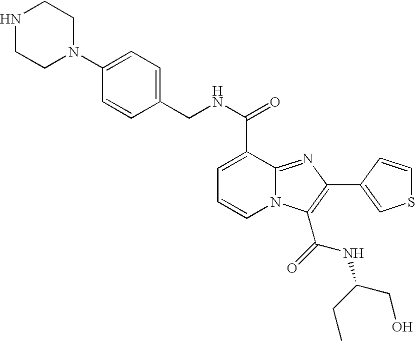 | 532.23 | 533.1 | 3.22 |

### Example 3A

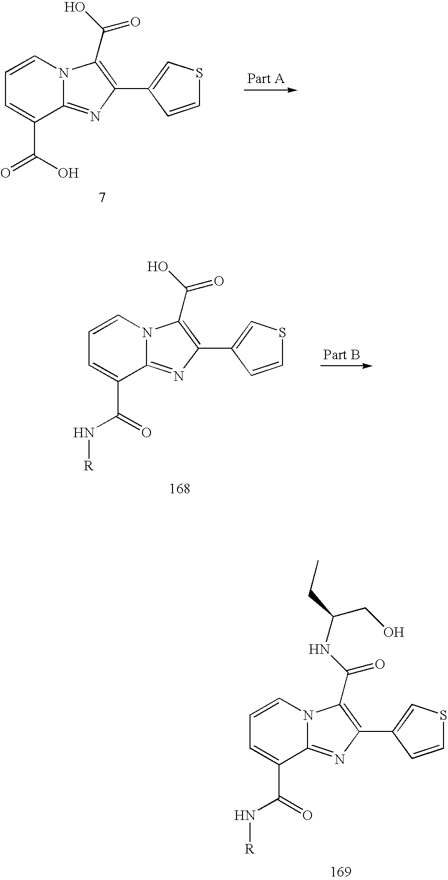

### Part A:

2-Thiphene-3-yl-imidazo[1,2-a]pyridine-3,8-dicarboxylic acid 7 (0.144 g, 0.5 mmol) dissolved in in dichloromethane (5 mL) and cooled to −20° C. To this (1-(3-dimethylaminopropyl)-3-ethylcarbodiimide (0.093 g; 1.2 equivalents; 0.6 mmol) was added. Followed by Diisopropyl ethyl amine (3 equivalents., 0.315 mL) was added and the solution was stirred at −20° C. for 15 minutes.

The activated acid was distributed equally in to, 4 ml Vials. Each vial was added with 0.025 mmol solution of Amine (pre dissolved in to DCM or NMP; 0.5 mL). The solution was shaken at −5° C. for 14 hrs. LCMS analysis showed the completion of the reaction.

### Part B:

The 8-aralkyl/aryl carbornyl-2-thiophen-3yl-imidazo[1,2-a]pyridine-3-carboxylic acid 168 obtained in the above step was used for this step with out any purification. The reaction mixture was warmed up to room temperature and to the above solution, one equivalent of (1-(3-dimethylaminopropyl)-3-ethylcarbodiimide (0.03 mmol) was added in each reaction vial followed by diisopropyl ethyl amine (5 equivalents) and S—(S)-(+)-2-amino-1-butanol (0.027 mmol). The reaction mixture stirred at room temperature for overnight. LCMS analysis showed completion of reaction.

The dichloromethane/N-methylpyrrolidine solution was concentrated under vacuum. Extracted in to ethyl acetate (3×2 mL). The organic extracts were dried under vacuum and re dissolved in methanol-acetonitrile and subjected to Prep. LC purification to get the products in Table 8.

TABLE 8

| | | | | Ret. |
|---|---|---|---|---|
| Compd | | | MS m/z | Time |
| # | Structure | EMW | (M⁺+ H) | (min) |
| 170 | 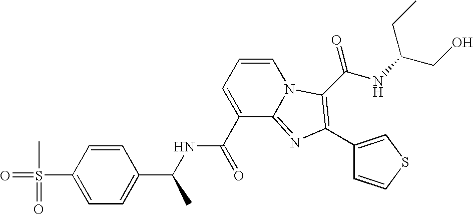 | 540.150 | 541.2 | 4.0 |
| 171 | 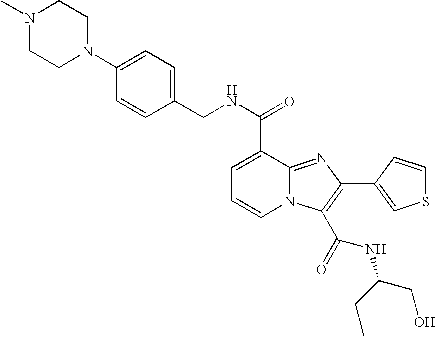 | 546.2416 | 547.2 | 3.25 |
| 172 | 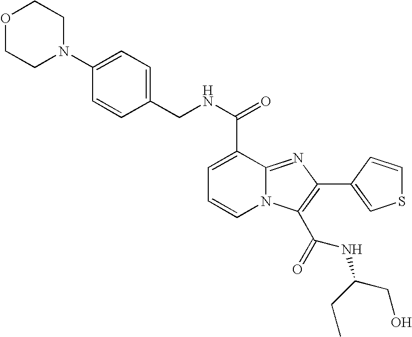 | 533.209 | 534.1 | 3.9 |
| 173 | 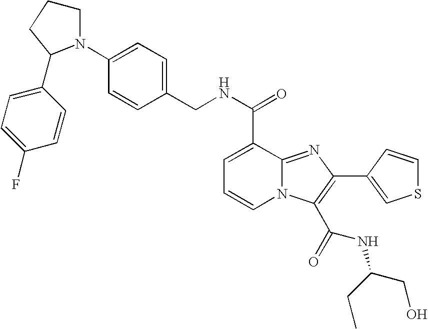 | 611.2369 | 612.20 | 5.8 |
| 174 | 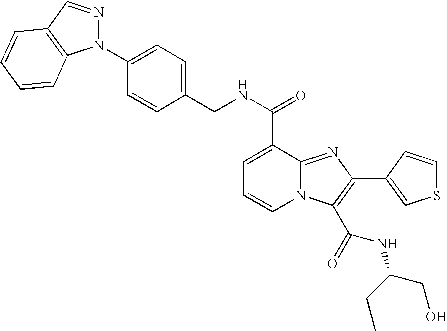 | 564.1946 | 565.1 | 5.2 |
| 175 | 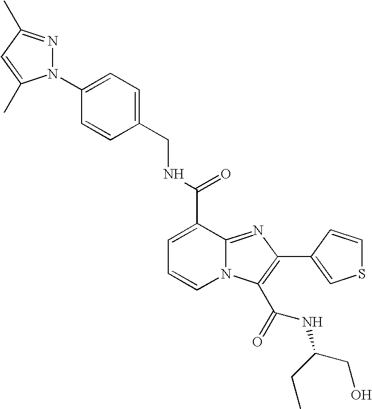 | 542.2103 | 543.1 | 4.5 |
| 176 | 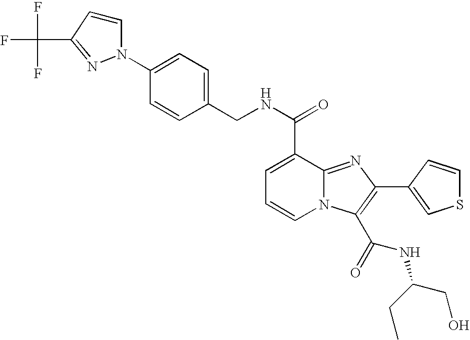 | 582.1664 | 583.2 | 5.3 |
| 177 | 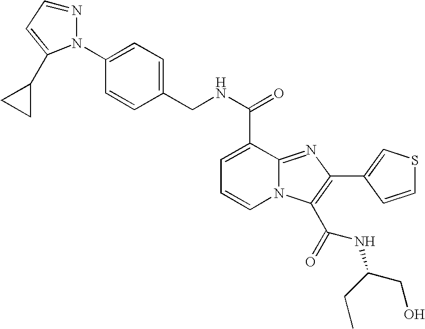 | 554.2103 | 555.1 | 4.85 |
| 178 | 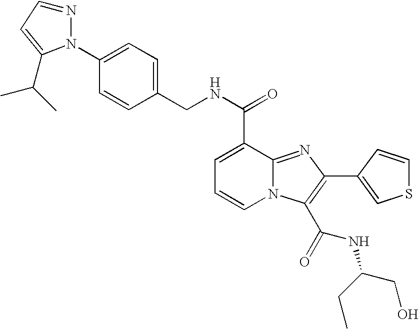 | 556.226 | 557.2 | 4.95 |
| 179 | 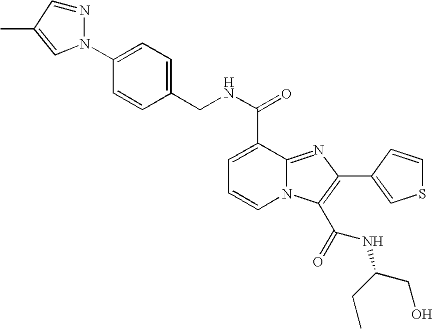 | 528.1946 | 529.2 | 4.75 |
| 180 | 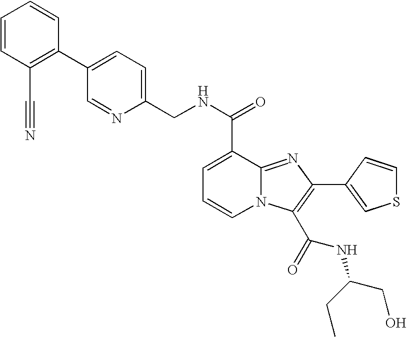 | 550.179 | 551.2 | 4.25 |
| 181 | 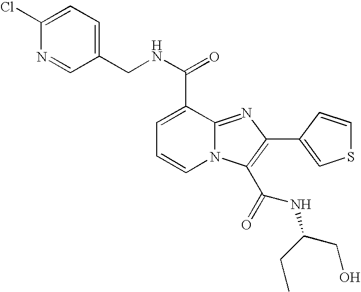 | 483.1135 | 484.1 | 4.2 |
| 182 | 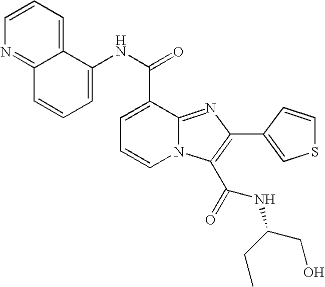 | 485.1524 | 486.1 | 3.65 |
| 183 | 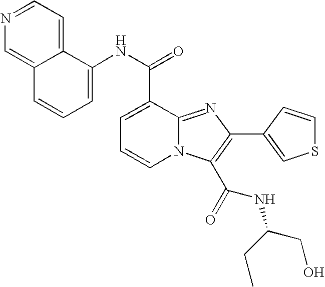 | 485.1524 | 486.1 | 3.55 |
| 184 | 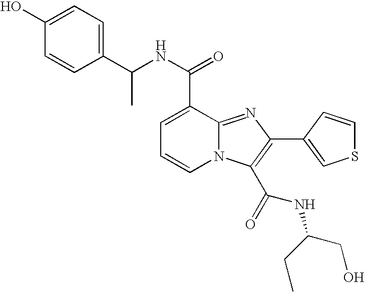 | 478.1677 | 479.1 | 3.85 |
| 185 | 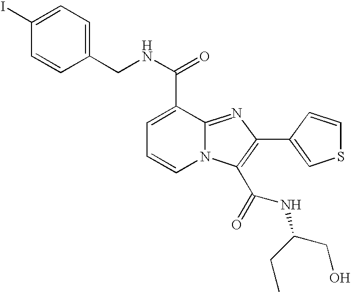 | 574.0538 | 575.0 | 5.2 |
| 186 | 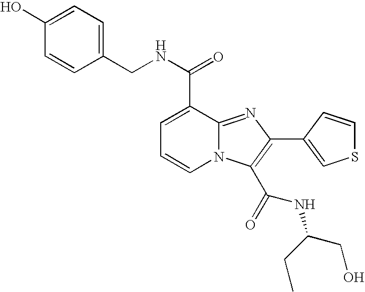 | 464.152 | 465.1 | 3.72 |
| 187 | 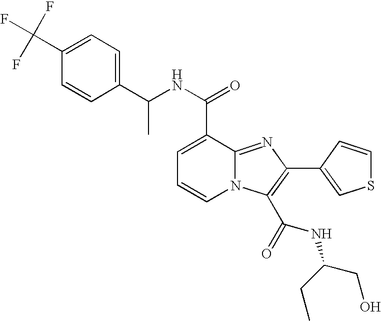 | 530.1602 | 531.0 | 5.39 |
| 188 | 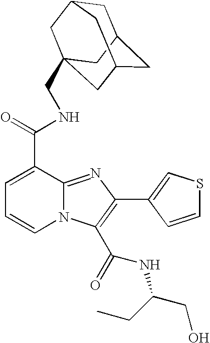 | 506.2354 | 507.2 | 5.92 |
| 189 | 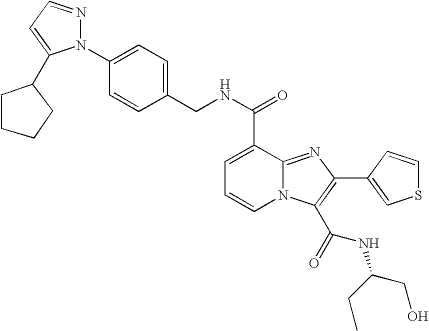 | 582.2416 | 583.2 | 5.30 |
| 190 | 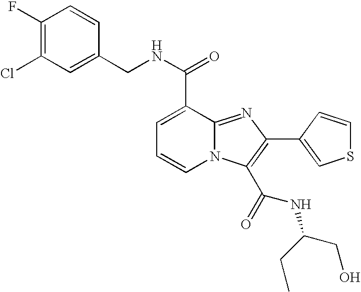 | 500.10 | 501.1 | 5.0 |
| 191 | 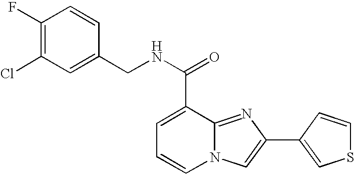 | 385.04 | 386.0 | 4.25 |
| 192 | 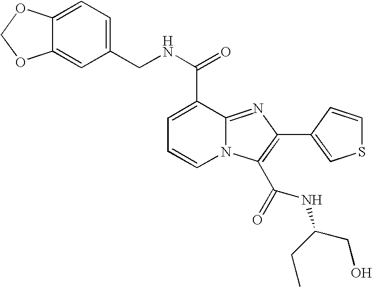 | 492.15 | 493.1 | 4.4 |
| 193 | 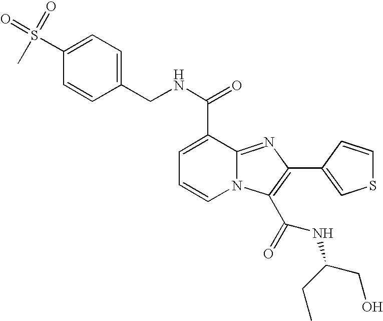 | 526.13 | 527.0 | 1.5 |
| 194 | 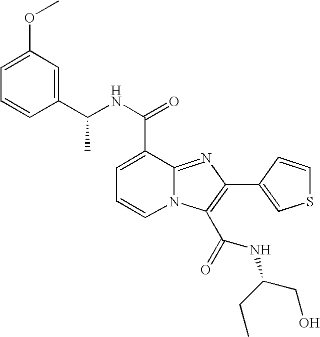 | 492.18 | 493.1 | 4.75 |
| 195 | 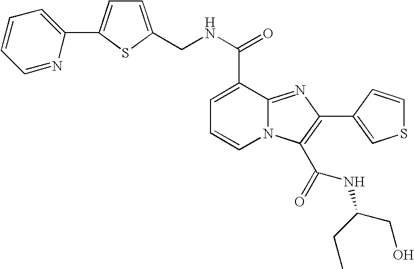 | 531.14 | 532.0 | 4.0 |
| 196 | 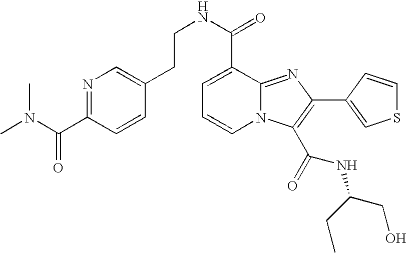 | 534.20 | 535.2 | 3.5 |
| 197 | 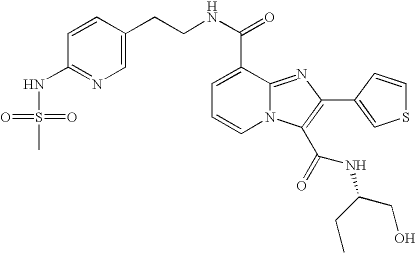 | 556.15 | 557.2 | 3.5 |
| 198 | 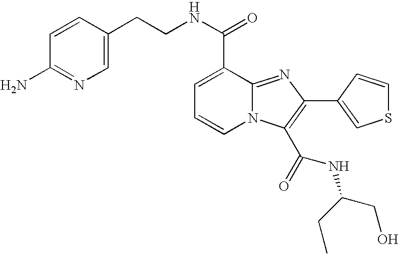 | 478.17 | 479.1 | 2.9 |
| 199 | 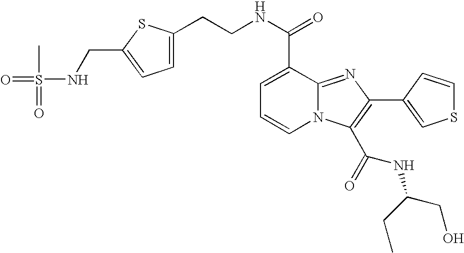 | 575.13 | 576.1 | 3.9 |
| 200 | 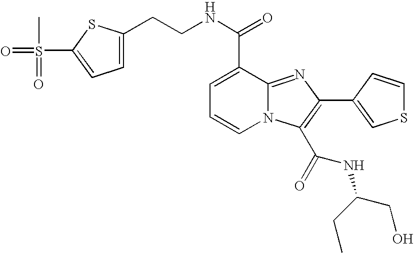 | 546.10 | 547.0 | 3.9 |
| 201 | 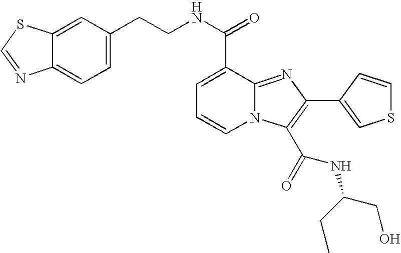 | 519.14 | 520.1 | 4.25 |
| 202 | 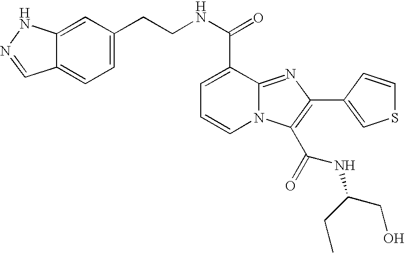 | 502.17 | 503.0 | 3.85 |
| 203 | 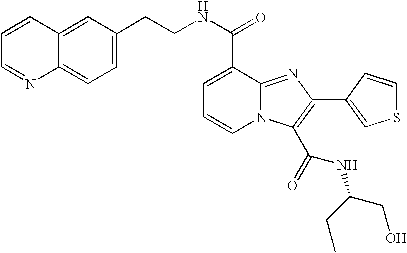 | 513.18 | 514.1 | 3.15 |
| 204 | 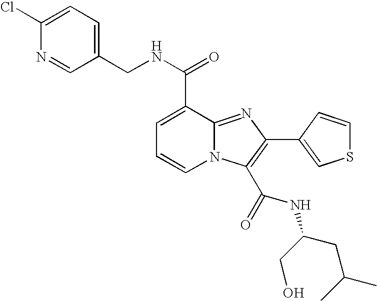 | 511.14 | 512.0 | 4.75 |
| 205 | 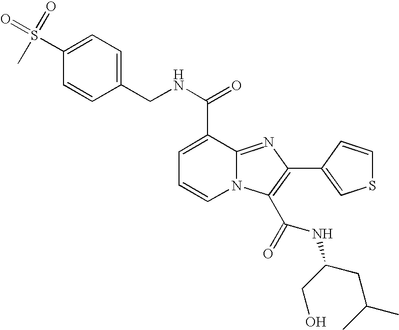 | 554.17 | 555.1 | 4.4 |
| 206 | 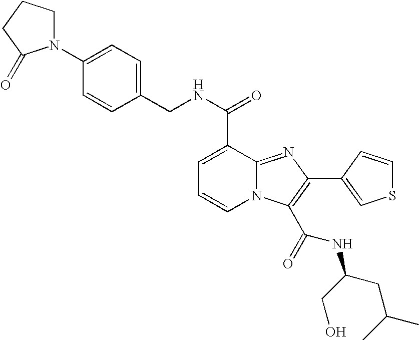 | 559.23 | 560.2 | 4.6 |
| 207 | 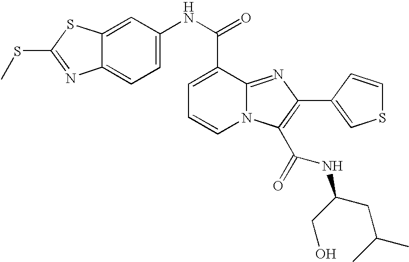 | 565.13 | 566.0 | 5.9 |
| 208 | 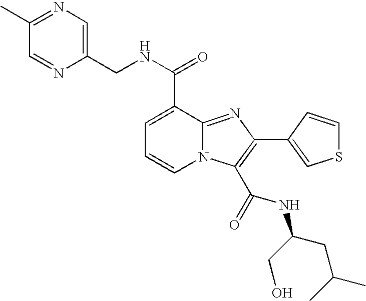 | 492.19 | 493.1 | 4.3 |
| 209 | 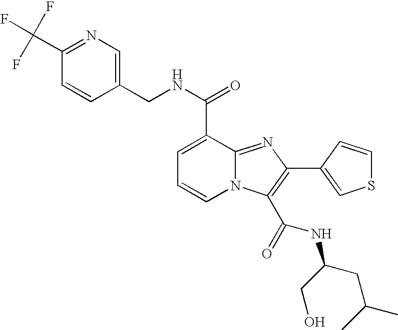 | 545.17 | 546.1 | 5.0 |
| 210 | 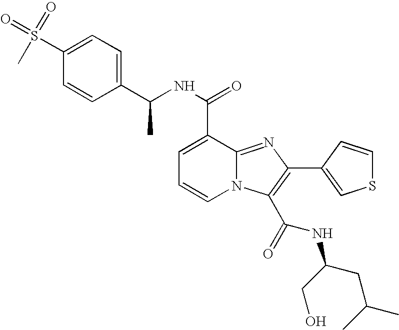 | 568.18 | 569.0 | 4.4 |
| 211 | 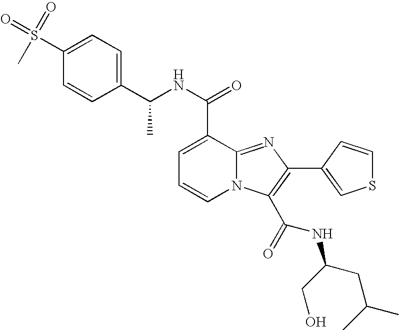 | 568.18 | 569.0 | 4.55 |
| 212 | 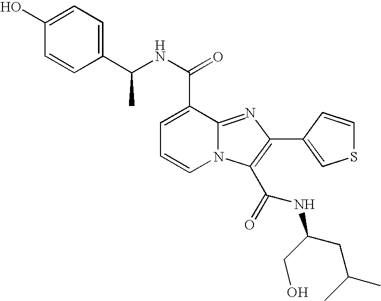 | 506.19 | 507.0 | 4.95 |
| 213 | 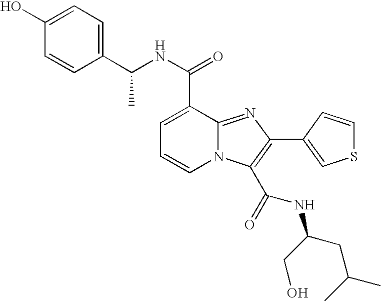 | 506.19 | 507.0 | 4.74 |
| 214 | 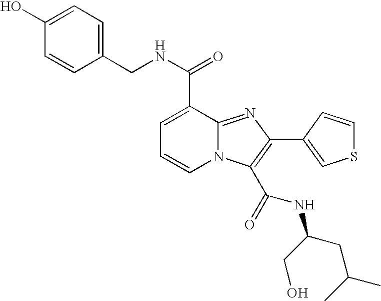 | 492.18 | 493.0 | 4.6 |
| 215 | 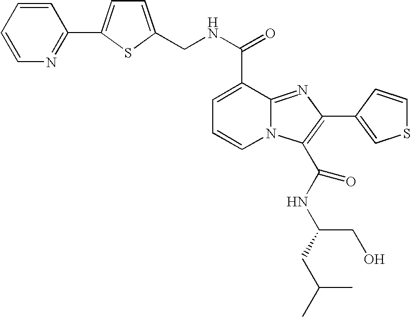 | 559.17 | 560.0 | 4.62 |
| 216 | 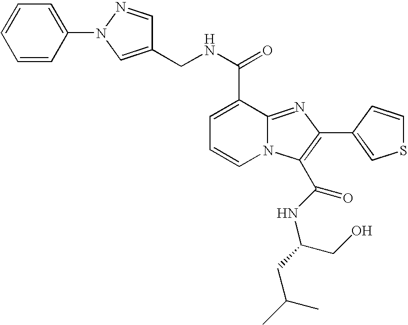 | 542.21 | 543.0 | 5.35 |
| 217 | 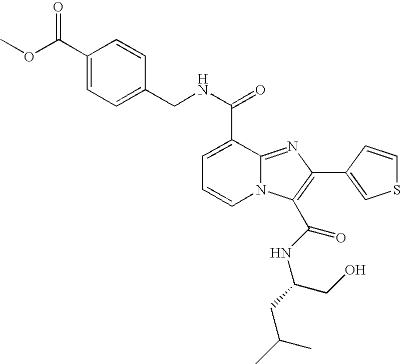 | 534.19 | 535.0 | 5.39 |
| 218 | 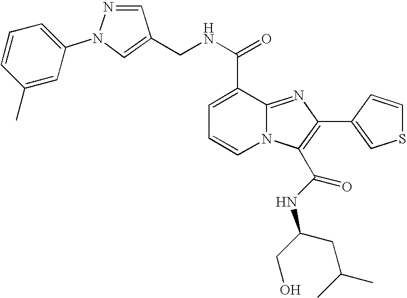 | 556.23 | 557.0 | 5.73 |
| 219 | 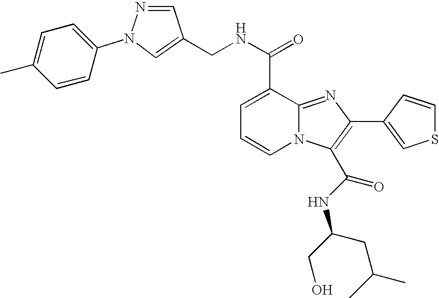 | 556.23 | 557.0 | 5.71 |
| 220 | 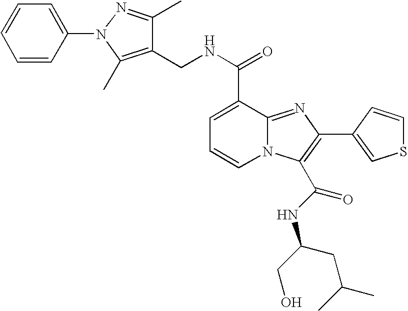 | 570.24 | 571.0 | 5.42 |
| 221 | 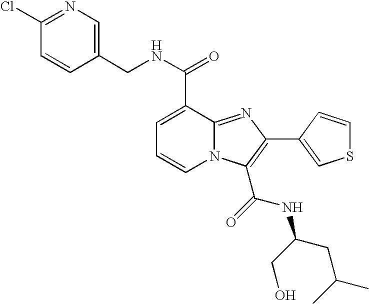 | 511.14 | 512.0 | 4.75 |
| 222 | 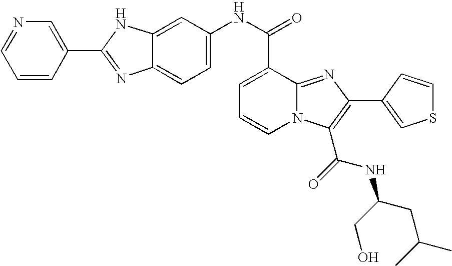 | 579.21 | 580.2 | 4.10 |
| 223 | 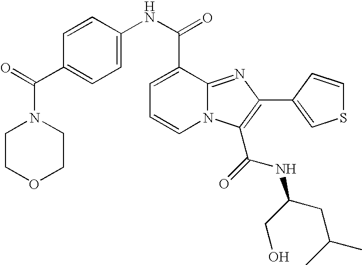 | 575.22 | 576.2 | 4.65 |
| 224 | 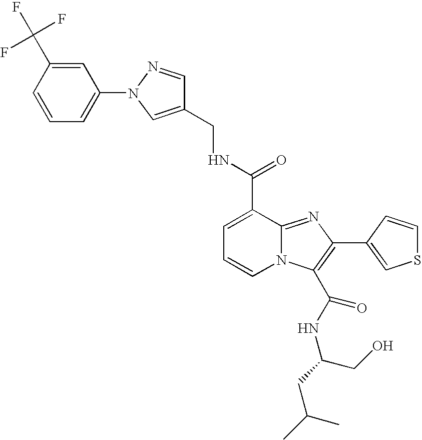 | 610.19 | 611.0 | 6.06 |
| 225 | 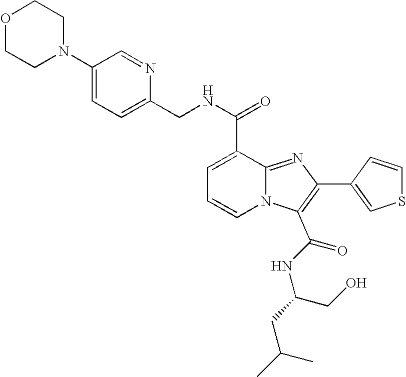 | 562.23 | 563.0 | 3.77 |
| 226 | 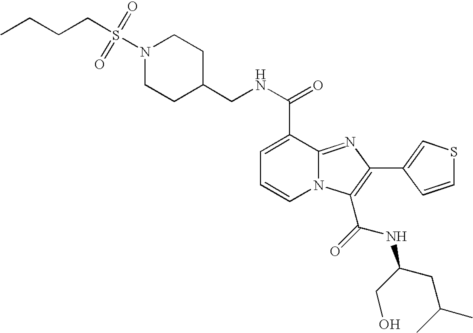 | 603.25 | 604.2 | 5.1 |
| 227 | 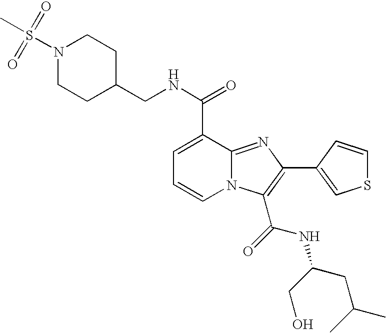 | 561.20 | 562.2 | 4.35 |
| 228 | 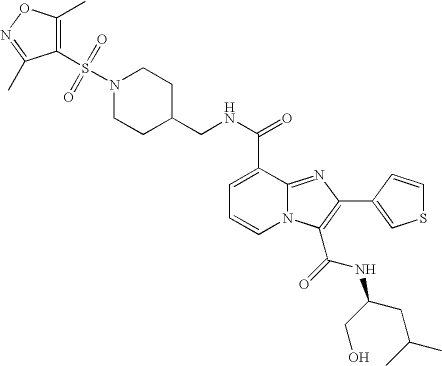 | 642.23 | 643.2 | 5.0 |
| 229 | 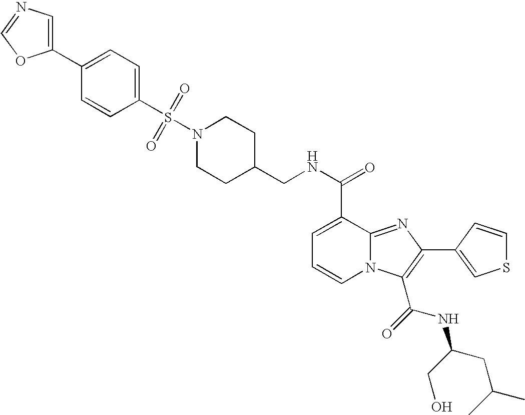 | 690.23 | 691.20 | 4.95 |
| 230 | 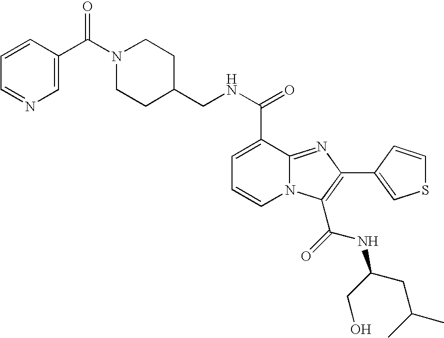 | 588.25 | 589.2 | 3.9 |
| 231 | 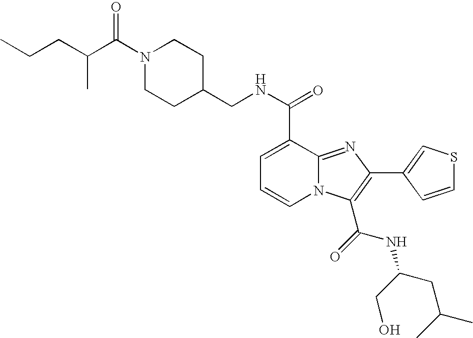 | 581.30 | 581.2 | 5.15 |
| 232 | 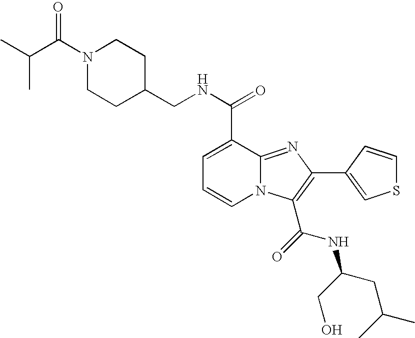 | 553.27 | 554.2 | 4.70 |
| 233 | 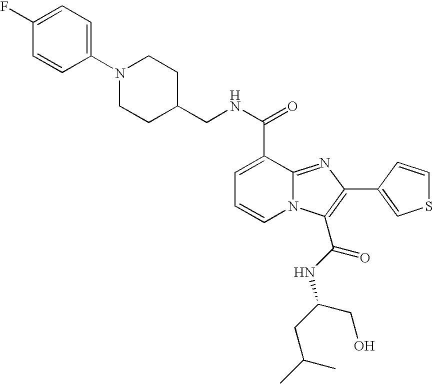 | 577.25 | 578.0 | 4.35 |
| 234 | 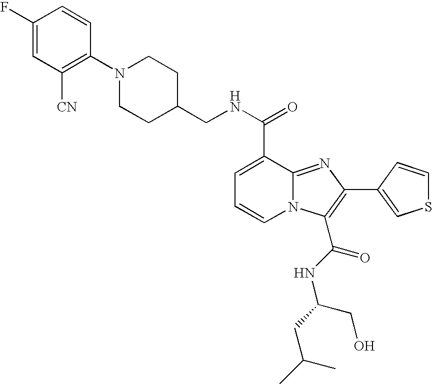 | 602.24 | 603 | 6.12 |
| 235 | 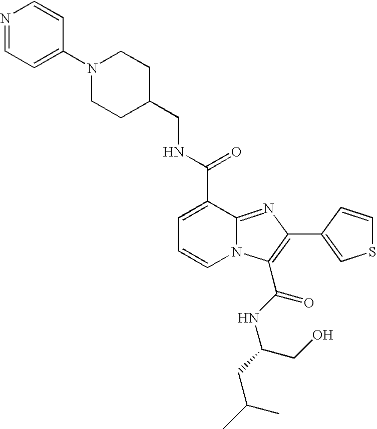 | 560.25 | 561.0 | 3.81 |
| 236 | 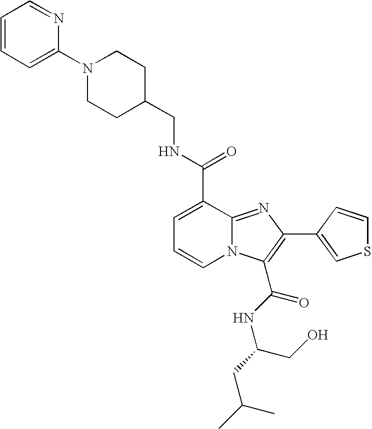 | 560.25 | 561.0 | 3.49 |
| 237 | 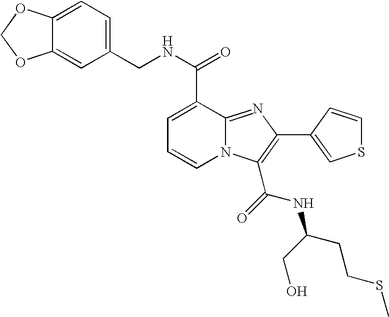 | 538.13 | 539.0 | 4.5 |
| 238 | 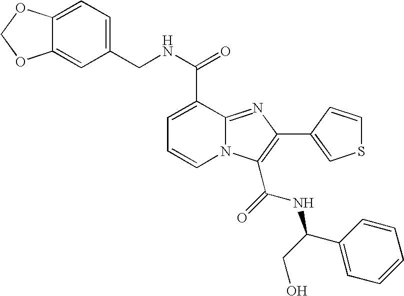 | 540.14 | 541.2 | 4.75 |
| 239 | 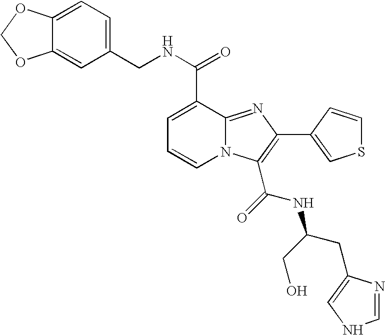 | 544.15 | 545.0 | 3.3 |
| 240 | 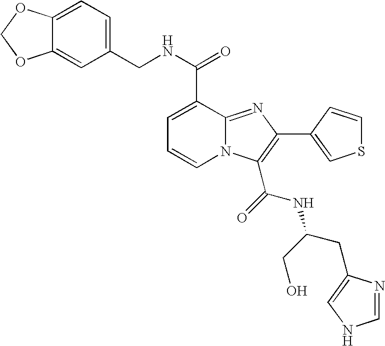 | 544.15 | 545.0 | 3.3 |
| 241 | 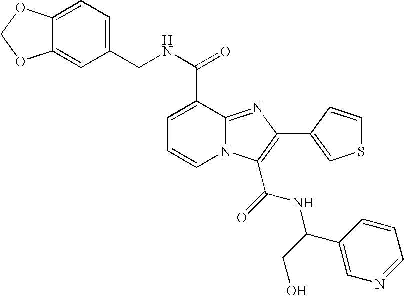 | 541.14 | 542.0 | 3.45 |
| 242 | 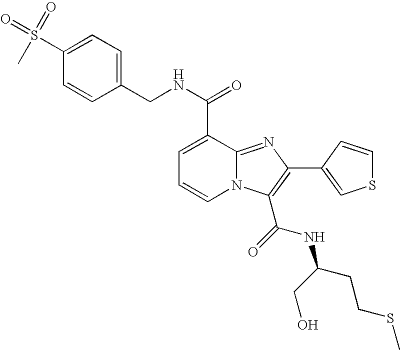 | 572.12 | 573.1 | 3.90 |
| 243 | 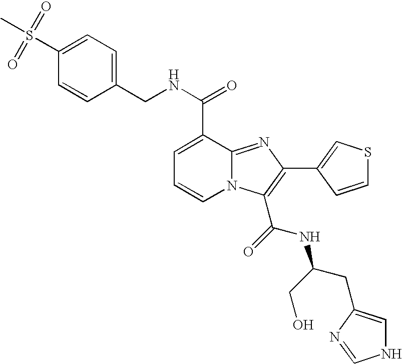 | 578.14 | 579.1 | 2.75 |
| 244 | 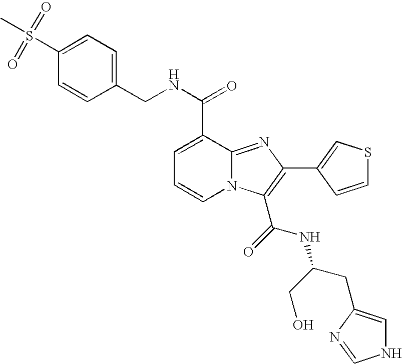 | 578.14 | 579.1 | 2.75 |
| 245 | 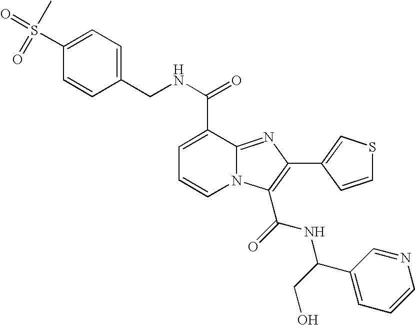 | 575.13 | 576.1 | 2.85 |

### Example-3B

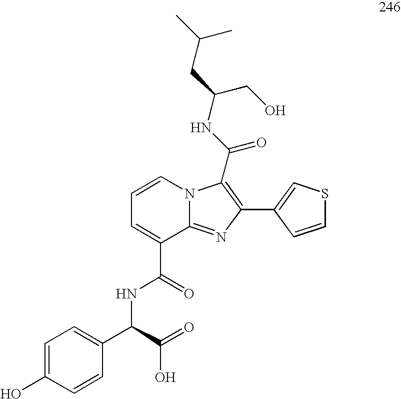

### Part A:

The general procedure used for coupling reaction is as described in preparative Example 3-Part A

### Part B:

The general procedure used for coupling reaction is as described in preparative Example 3-Part B

### Part C:

The general procedure used for coupling reaction is as described in preparative Example 2C-Part B

| | | | | Ret. |
|---|---|---|---|---|
| Compd | | | MS m/z | Time |
| # | Structure | EMW | (M⁺ + H) | (min) |
| 246 | 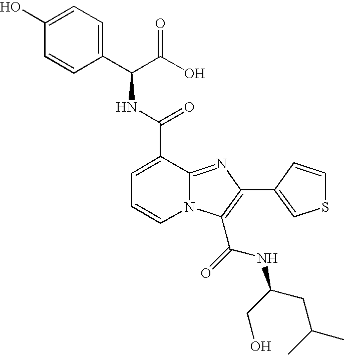 | 536.17 | 537.2 | 4.06 |

### Example 4A

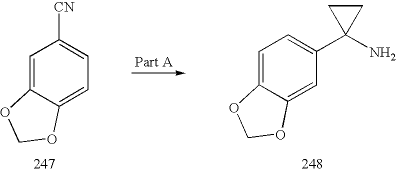

### Part A:

Piperonylonitrile 247 (0.735 g, 0.5 mmol,) was dissolved in dry ether, cooled to −78° C. and kept under inert atmosphere. Ethyl magnesium bromide (1.2 equivalents) was added to the above solution by syringe maintaining the temperature at −78° C. After the addition, the reaction stirred at −78° C. for 1 hour and allowed the reaction mixture to warm up to room temperature. Stirring continued at r.t for another 2 hours. LCMS analysis showed the formation of product. The reaction was quenched with water and reaction mixture extracted with ether, Ether layer was washed with water, brine and dried with anhydrous MgSO₄. Evaporation of ether gave crude which on passing through the silica gel column eluting with Hexane/Ethyl acetate provided with the product, 1-benzo[1,3]dioxol-5-yl-cyclopropylamine. Calculated M.W.=177.19, and observed M⁺+H 178.1

### Example 4B

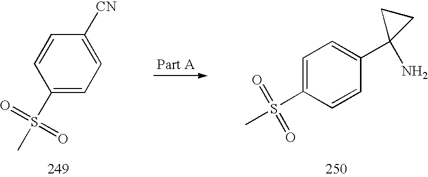

### Part A:

Compound 250 was prepared from 249 using methods described in Example 4A. mass calculated for compound 253 is 211.06, observed LCMS m/z 212.21

### Example 4C

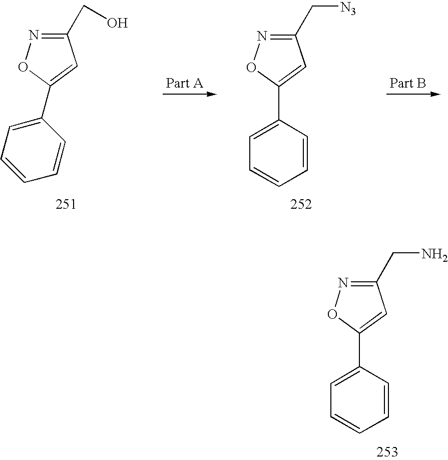

### Part A:

(5-Phenyl-isoxazol-3-yl)methanol 251 (0.175 g, 1 mmol) was dissolved in THF (10 mL) and to this, DPPA (1.1 eq, 1.1 mmol) and DBU (1.5 eq, 1.5 mm) was added. and the solution was stirred under reflux for 14 hours. The THF was removed under vacuum and the crude thus obtained showed formation of product from the LCMS analysis. The crude was passed through the silical gel column to give the 3-azido methyl-5-phenyl-isoxazole 252. mass calculated for compound 252 is 200.19, observed LCMS m/z 201.24.

### Part B:

3-azido methyl-5-phenyl-isoxazole 252 obtained in the above step was dissolved in dioxane and resin bound triphenylphosphine (excess) was added and stirred at room temperature. After 2 hours, a mixture of dixane/water (0.50 mL) was added and stirring continued for 2 more hours. Filter off the resin and the evaporated the dioxane under vacuum resulted in the desired amine, (5-Phenyl-isoxazol-3-yl)methylamine 253, mass calculated for compound 253 is 174.19, observed LCMS m/z 175.25 which was used in the next step with out purification.

### Example 4D

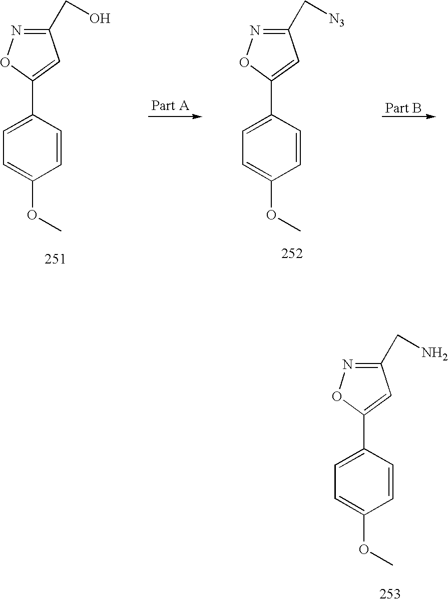

### Part A:

Compound 255 was prepared from 254 using methods described in Example 4C. mass calculated for compound 255 is 230.08, observed LCMS m/z 239.1

### Part B:

Compound 256 was prepared from 255 using methods described in Example 4C. mass calculated for compound 256 is 204.1, observed LCMS m/z 205.1

### Example 4E

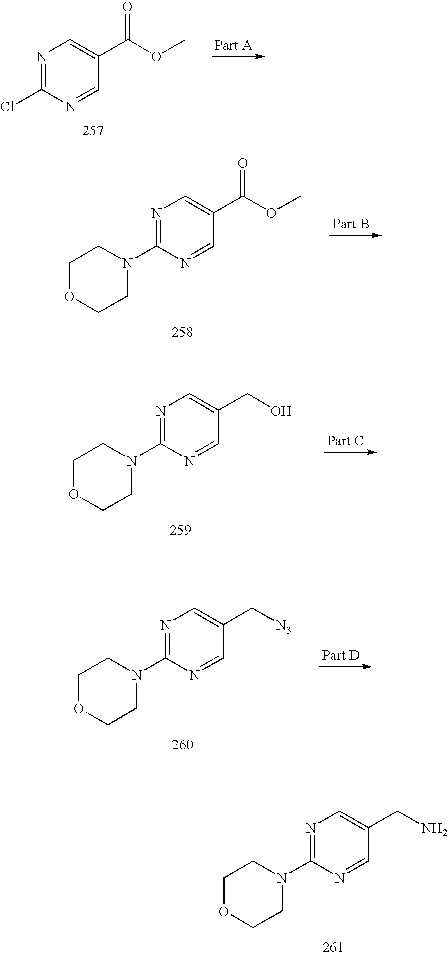

### Part A:

2-chloro-5-carboxymethylpyrimidine 257 (0.5 g) was dissolved in Morpholine and heated at 100° C. for 14 hours. Removal of excess morpholine and passing through the column provided the product, 2-morpholino-5-carboxymethylpyrimidine 258. Mass calculated for compound 258 is 223.22, observed LCMS m/z 224.1

### Part B:

2-morpholino-5-carboxymethylpyrimidine 258 (0.4 g) was dissolved in MeOH and NaBH₄ (1.5 equivalents) was added and reaction stirred at room temperature for 12 hours. Solvent was evaporated and diluted with ethyl acetate, washed with water, brine, dried over anhydrous magnesium sulfate. Filtered, evaporated and passed through the column to afford the product corresponding alcohol 259. Mass calculated for compound 259 is 195.21, observed LCMS m/z 196.1

### Part C & Part D:

Follwing the general procedure described in the preparative Example 4C, Part A and Part B, the title compound was prepared. Mass calculated for compound 261 is 194.23, observed LCMS m/z 195.2

### Example 4F

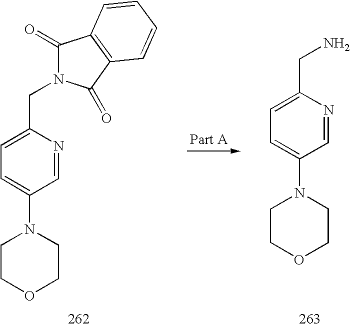

### Part A:

2-(5-Morpholino-4-yl-pyridine-2-yl-methyl)isoindole-1,3-dione 262 (0.200 g) was dissolved in methanol and excess hydrazine hydrate was added and refluxed for two hours. After concentration of solvent, the residue was passed through the Prep LC to get the desired product 263. Mass calculated for compound 263 is 193.24, observed LCMS m/z 194.1

### Example 4F

1-(Tetrahydro-pyran-2-yl-1H-indazo-5-yl}-methylamine: synthesized as described in the reference. JOC, 62, 5627 (1997).

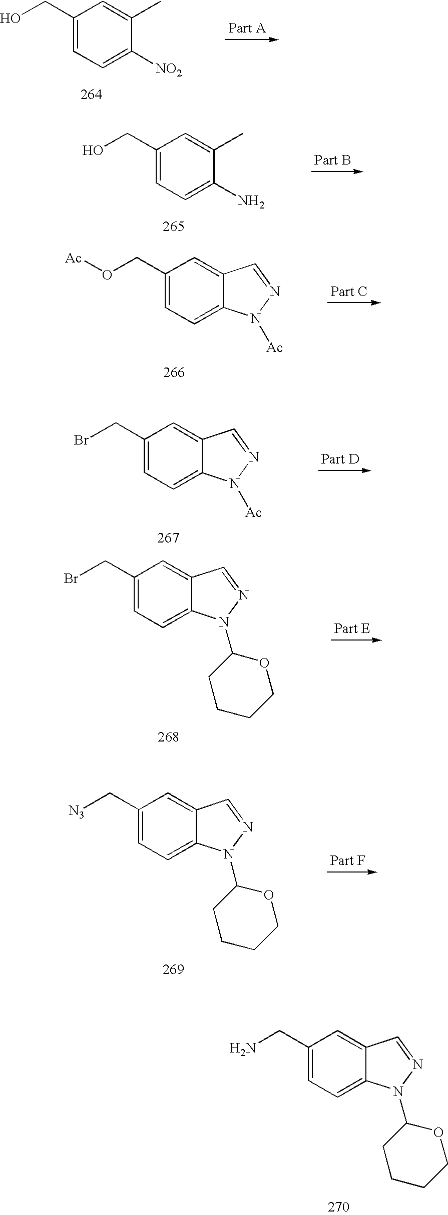

### Part A:

A mixture of 3-methyl-4-nitro benzyl alcohol 264 (2.10 g, 12.6 mmol) and 10% Palladium on carbon (0.2 g) in 25 mL of EtOH was hydrogenated at room temperature. After completion of the reaction, the catalyst was removed by filtration. The solvent was evaporated and residue dried in a vacuum to give title compound as yellow solid 1.7 g, 97%), ¹H NMR (CDCl₃), δ 7.06 (s, 1H), 7.03 (d, J=8.0 Hz, 1H), 6.66 (d, J=7.7 Hz, 1H), 4.53 (s, 1H), 3.62 (br, 2H), 2.17 (s, 3H); mass calculated for compound 265 is 137.17, observed LCMS m/z 138.2 (M+H).

### Part B:

A mixture of product 265 from part A (1.65 g, 12 mmol), acetic anhydride (3.4 mL, 36 mmol) and potassium acetate (2.37 g, 24 mmol) in 50 mL of CHCl₃ was stirred at room temperature and then refluxed for 2 hours and stirred at room temperature for overnight. Then n-amyl nitrite (3.2 g, 27 mmol) and 18-crown-6 (0.16 g, 0.6 mmol) were added and the mixture was heated at reflux for 28 hours. After being cooled to room temperature the reaction mixture was added to acetic anhydride (1 mL) and stirred at room temperature overnight. The reaction mixture diluted with CH₂Cl₂ (50 mL), washed with water, brine and dried (Na₂SO₄) and the solvent evaporated to give dark brown solid. Chromatography (silica gel, 15% EtOAc/Hexane) gve the title product 1.7 g, 58%): ¹H NMR (CDCl₃) δ 8.44 (d, J=8.8 Hz, 1H), 8.13 (d, J=0.8 Hz, 1H), 7.75 (d, J=0.7 Hz, 1H), 7.56 (dd, J=8.8, 1.5 Hz, 1H), 5.23 (s, 2H), 2.79 (s, 3H), 2.12 (s, 3H), mass calculated for compound 266 is 232.23, observed LCMS m/z 233.2 (M+H).

### Part C:

A mixture of the above compound 266 (1.0 g, 4.3 mmol) in 10 mL of 48% HBr was stirred at room temperature for 16 hours. The solid was collected on Buchner funnel, washed with 48% HBr and dried in a vacuum desiccator with P₂O₅ and NaOH to give the title compound as a light tan solid (1.15 g, 92%), which was used in the next step with out further purification. mass calculated for compound 267 is 209.97, observed LCMS m/z 211.2 (M+H).

### Part D:

The mixture above compound 267 (1.6 g, 5.7 mmol) and 3,4-dihydro-2H-pyran (1 g, 11.3 mmol, 2 equivalents) in THF (40 mL) was refluxed for 2 hours and stirred at room temperature for overnight. The reaction mixture diluted to 100 mL with CH₂Cl₂, washed with water, saturated NaHCO₃, water, brine and dried over MgSO₄ and the solvent evaporated. Chromatography (silica gel, EtOAc/Hexane 0-20%) gave title compound as beige solid (1.3 g, 79%), mass calculated for compound 268 is 293.03, observed LCMS m/z 294.0 (M+H).

### Part E:

A solution of 5-(Bromomethyl)-1-(2(tetrahydropyranyl) indazole 268 (1 g, 4 mmol) in dry DMF was treated with sodium azide (0.78 g, 12 mmol.) in one portion and heated to 90° C. for 30 min. The reaction mixture cooled to room temperature, poured in to water (50 mL) and extracted with ether (150 mL), the organic phase washed with brine, dried over MgSO₄, filtered and evaporated to give title compound azide 269. No further purification is needed. mass calculated for compound 269 is 257.12, observed LCMS m/z 258.2 (M+H).

### Part F:

A solution of azide 269 from the above step (1 g) in THF was cooled to 0° C. in ice bath and treated with LAH (10 mL, 1.0 M in THF) via syringe over 10 min. After 1 hour, the reaction mixture was quenched by drop wise addition of 1.0 M solution of NaOH (1.5 mL). The reaction mixture allowed to warm up to room temperature, diluted with EtOAc (60 mL) dried with (Na₂SO₄) and filtered (celite). The organic layer evaporated to give essentially pure amine 270. mass calculated for compound 270 is 231.13, observed LCMS m/z 232.1 (M+H).

The compounds in Table 9 are made using the methods described in Example 3 Part A and B.

TABLE 9

| | | | | Ret. |
|---|---|---|---|---|
| Compd | | | MS m/z | Time |
| # | Structure | EMW | (M⁺ + H) | (min) |
| 271 | 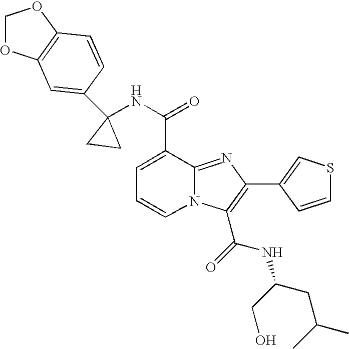 | 546.19 | 547.2 | 5.58 |
| 272 | 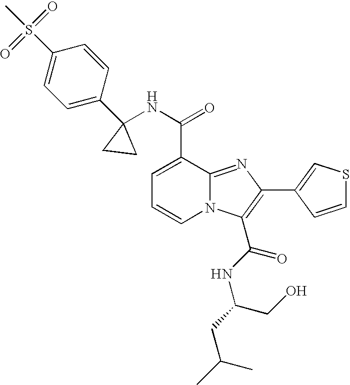 | 580.18 | 581.0 | 4.97 |
| 273 | 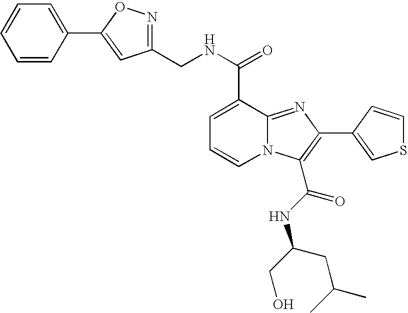 | 543.19 | 544.0 | 5.72 |
| 274 | 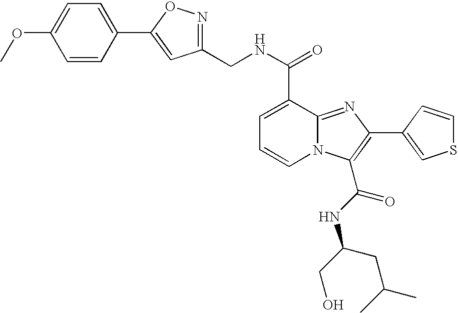 | 573.20 | 574.0 | 5.67 |
| 275 | 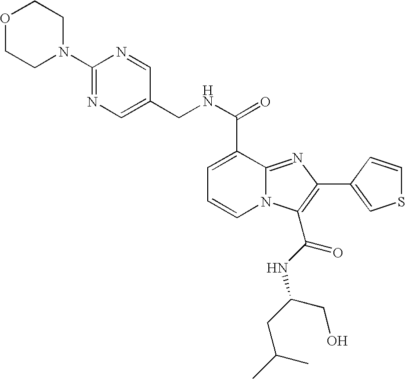 | 563.23 | 564.0 | 4.62 |
| 276 | 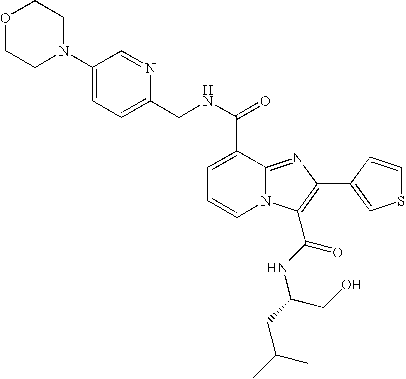 | 562.24 | 563.0 | 3.77 |
| 277 | 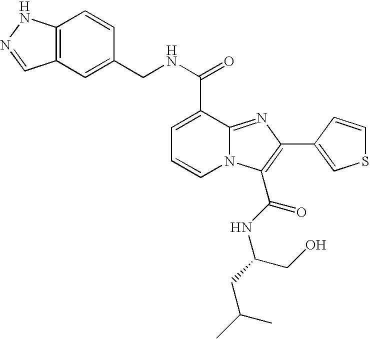 | 516.19 | 517.0 | 4.46 |

### Example 4H

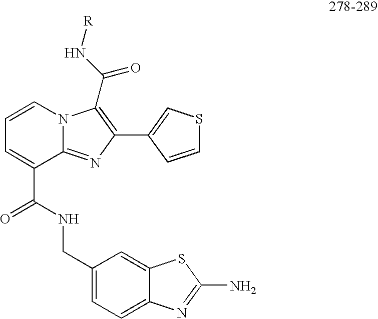

(wherein R is the remaining moiety of the Q^{B} group in formula 1.0)

The compounds in Table 10 are made using the methods described in the Example 3, parts A and B and Example 2C part D.

TABLE 10

| | | | | Ret. |
|---|---|---|---|---|
| Compd | | | MS m/z | Time |
| # | Structure | EMW | (M⁺ + H) | (min) |
| 278 | 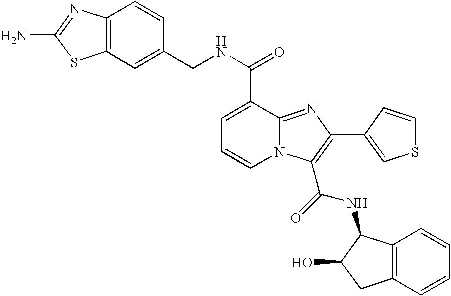 | 580.13 | 581.0 | 3.75 |
| 279 | 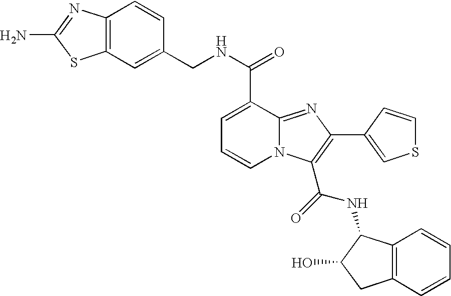 | 580.13 | 581.0 | 3.75 |
| 280 | 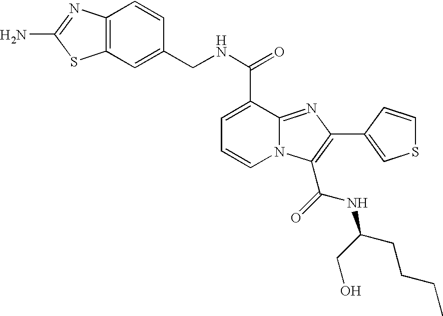 | 548.16 | 549.2 | 3.8 |
| 281 | 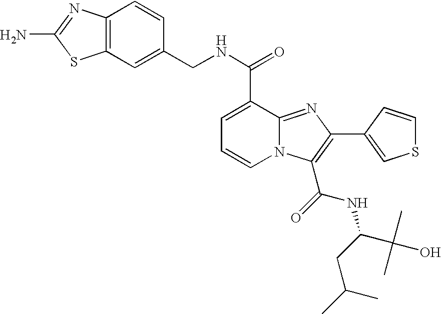 | 576.19 | 577.2 | 4.05 |
| 282 | 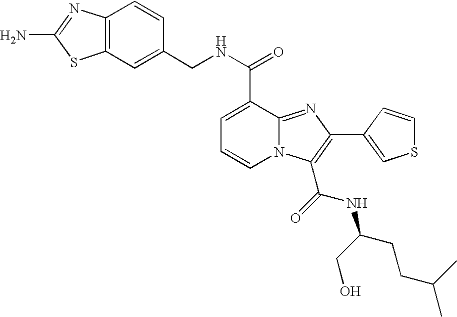 | 562.18 | 563.2 | 4.17 |
| 283 | 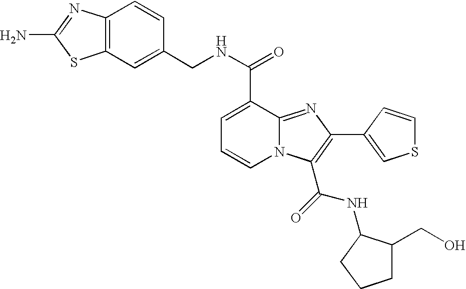 | 546.15 | 547.0 | 3.85 |
| 284 | 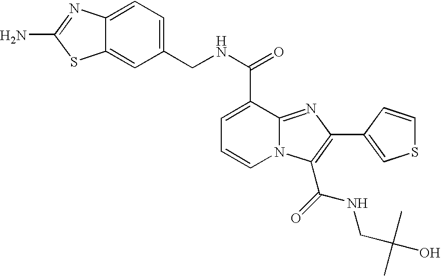 | 520.13 | 521.0 | 3.34 |
| 285 | 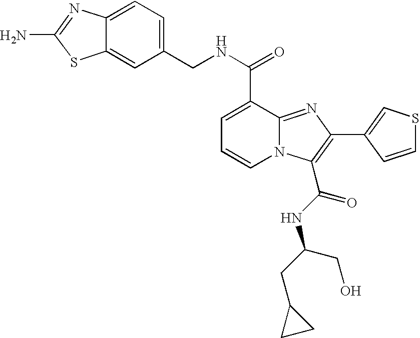 | 546.15 | 546.1 | 3.45 |
| 286 | 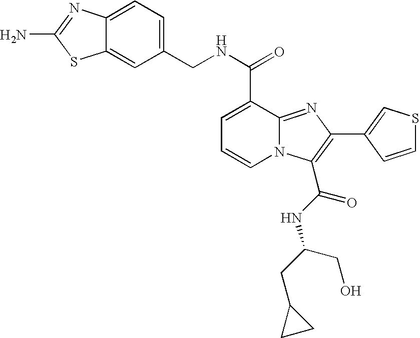 | 546.15 | 546.1 | 3.45 |
| 287 | 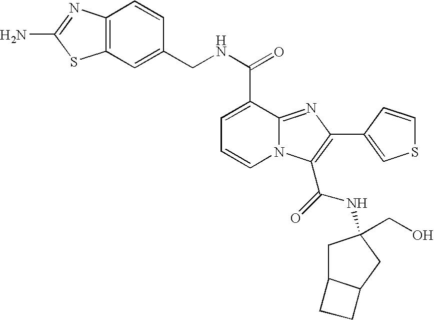 | 572.16 | 573.1 | 3.85 |
| 288 | 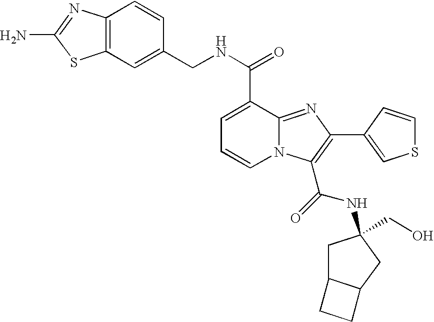 | 572.16 | 573.1 | 3.87 |
| 289 | 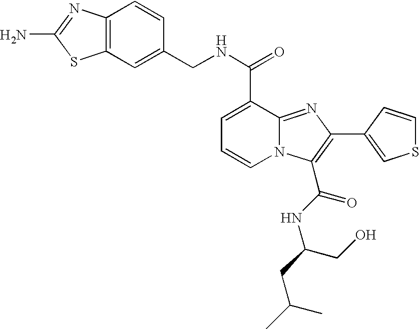 | 548.16 | 549.0 | 5.58 |

### Example 5A

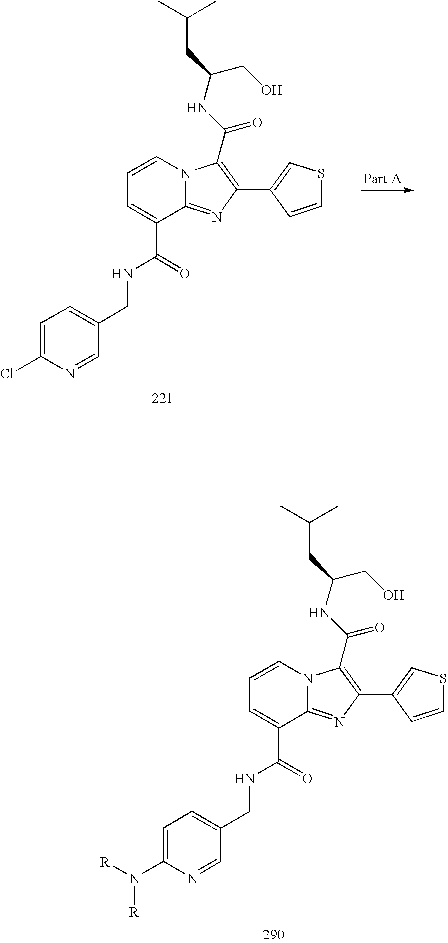

(wherein the R groups are identified in Table 11)

The compound 221 prepared using methods described in Example 3A was dissolved in to NMP (5 mL) and distributed equally in to 4 mL vials. The required amine was added in excess and the mixture was heated in a sealed tube at 100° C. for 72 hours or until LCMS analysis showed the completion of the reaction.

The crude material was subjected to HPLC purification to get pure products in various yields. The products obtained are given in Table 11.

TABLE 11

| | | | MS | Ret. |
|---|---|---|---|---|
| Compd | | | m/z | Time |
| # | Structure | EMW | (M⁺ + H) | (min) |
| 291 | 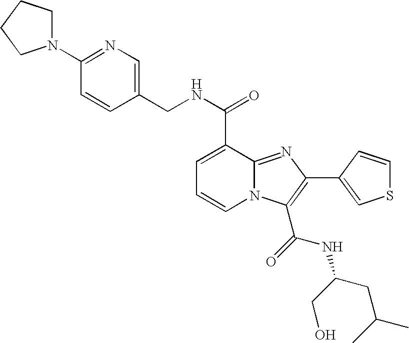 | 546.24 | 547.2 | 3.82 |
| 292 | 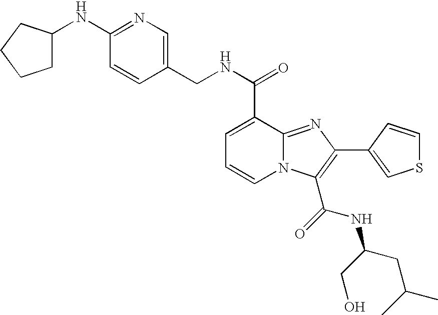 | 560.26 | 561.3 | 1.32 |
| 293 | 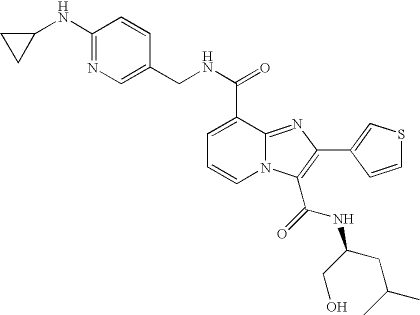 | 532.23 | 533.2 | 1.32 |
| 294 | 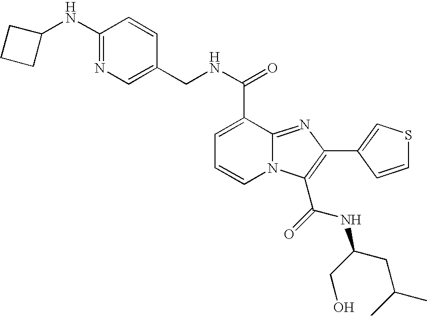 | 546.24 | 547.2 | 1.25 |
| 295 | 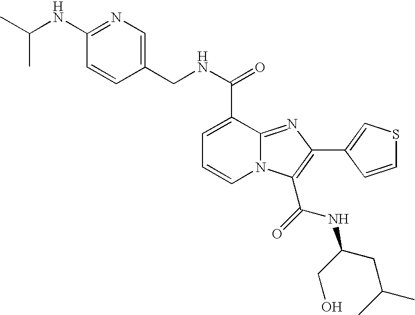 | 534.24 | 535.2 | 1.25 |
| 296 | 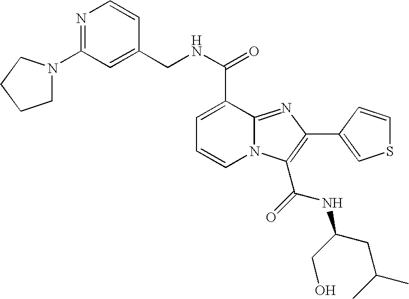 | 546.24 | 547.2 | 3.9 |
| 297 | 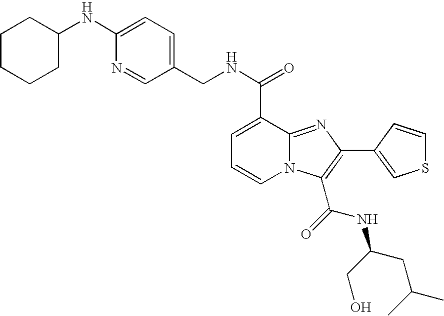 | 574.27 | 575.2 | 4.15 |
| 298 | 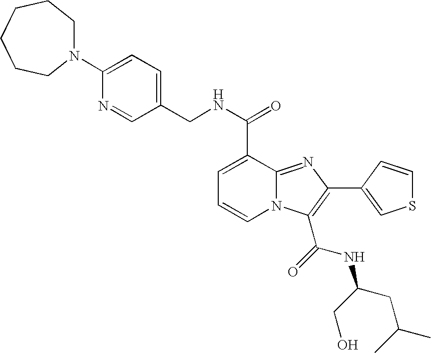 | 574.27 | 575.2 | 4.0 |
| 299 | 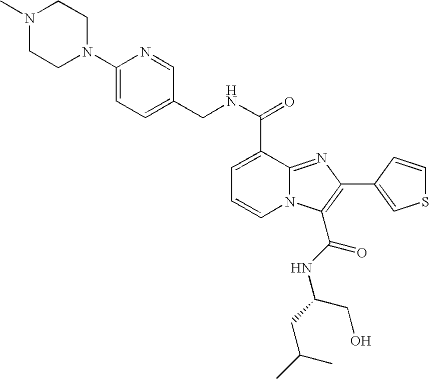 | 575.27 | 576.2 | 3.2 |
| 300 | 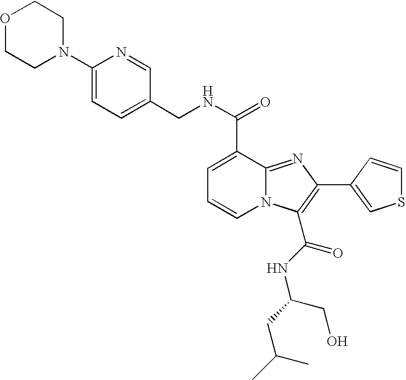 | 562.24 | 563.2 | 3.45 |
| 301 | 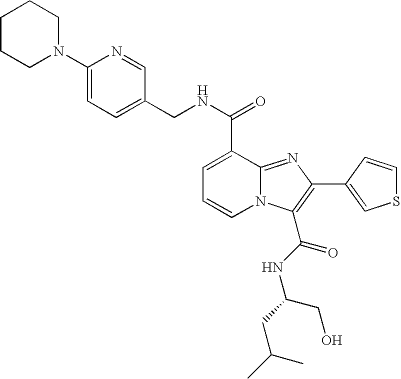 | 560.26 | 561.3 | 3.8 |
| 302 | 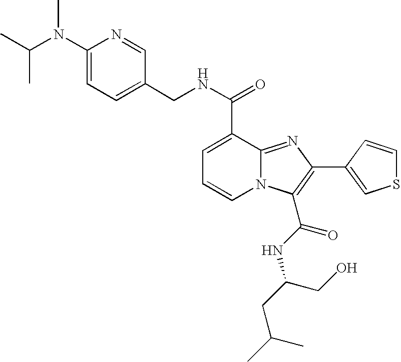 | 548.26 | 549.0 | 4.07 |
| 303 | 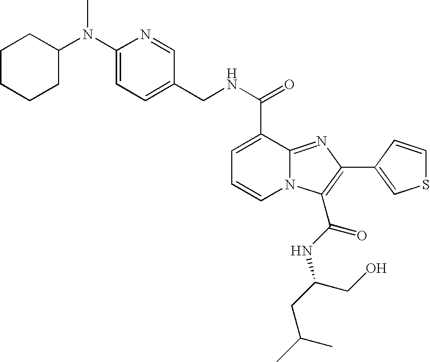 | 588.29 | 589.0 | 4.07 |

### Example 5B

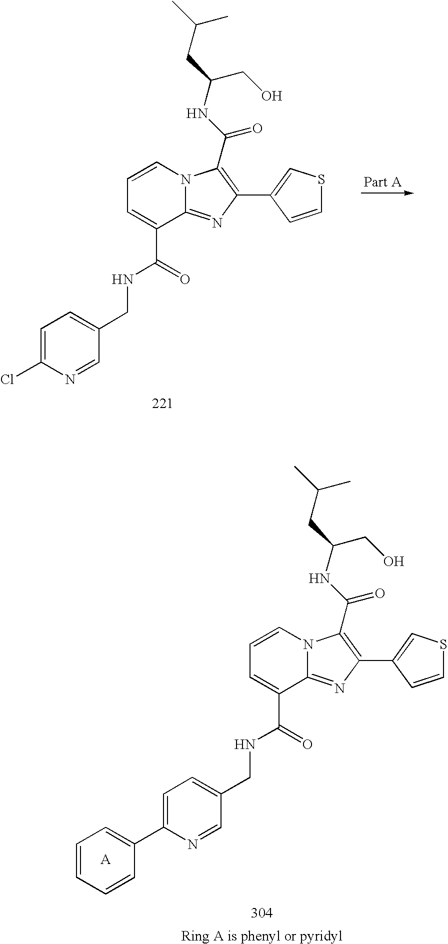

(Ring A is phenyl or Pyridyl as identified in Table 12)

### Part A:

The compound 221 (0.15 mmol) is taken in DMF (1 mL) and added with 0.015 mmol, of Pd(dppf)₂Cl₂, appropriate boronic acid (0.18 mmol; 1.2 equivalents) and K₃PO₄ (0.70 mg; 2.5 mmol) were added. The reaction mixture purged with argon and heated at 80° C. for 14 hrs. LC MS analysis showed completion of the reaction.

The reaction mixture poured in to water, extracted with Ethyl acetate. The organic layer washed with brine, dried over anh. MgSO₄, filtered, evaporated and subjected to HPLC purification to give the 90% pure title compound. The compounds obtained are identified in Table 12.

TABLE 12

| | | | MS | Ret. |
|---|---|---|---|---|
| Compd | | | m/z | Time |
| # | Structure | EMW | (M⁺ + H) | (min) |
| 305 | 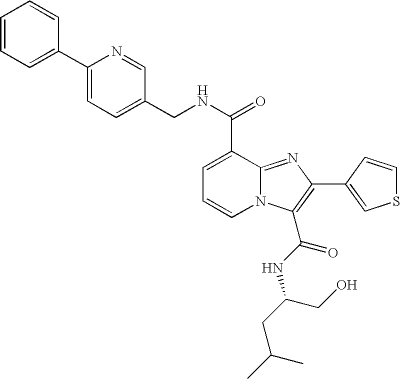 | 553.21 | 554.0 | 4.57 |
| 306 | 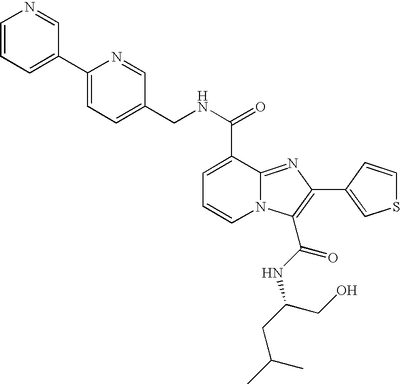 | 554.21 | 555.0 | 3.79 |
| 307 | 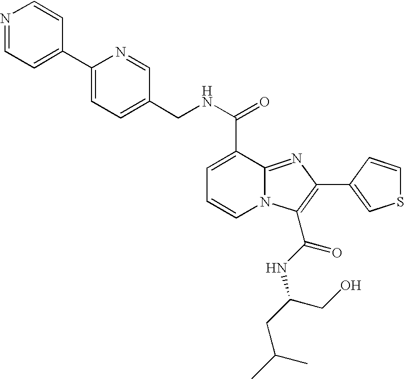 | 554.21 | 555.1 | 3.82 |

### Example 6A

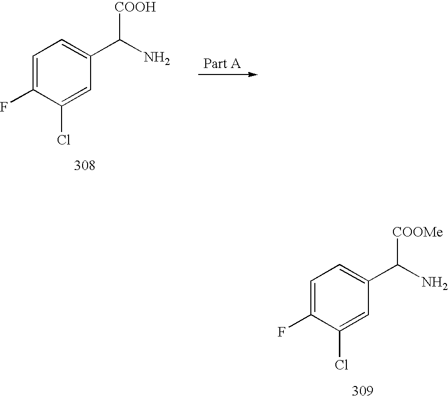

### Part A:

To a solution of compound 308 (0.15 mmol) in acetonitrile (2 mL) and methanol (2 mL) was added (trimethylsilyl)diazomethane (2M, 0.11 mL, 0.22 mmol). The reaction mixture was stirred at room temperature for 30 minutes. LC-MS analysis of the reaction indicated that the reaction was complete. The volatiles were removed in vacuo to afford compound 309 as a white solid. HPLC-MS t_{R}=0.82 min (UV_{254 nm}); mass calculated for formula C₉H₉CIFNO₂ 217.0, observed LCMS m/z 218.1 (M+H).

### Example 6B

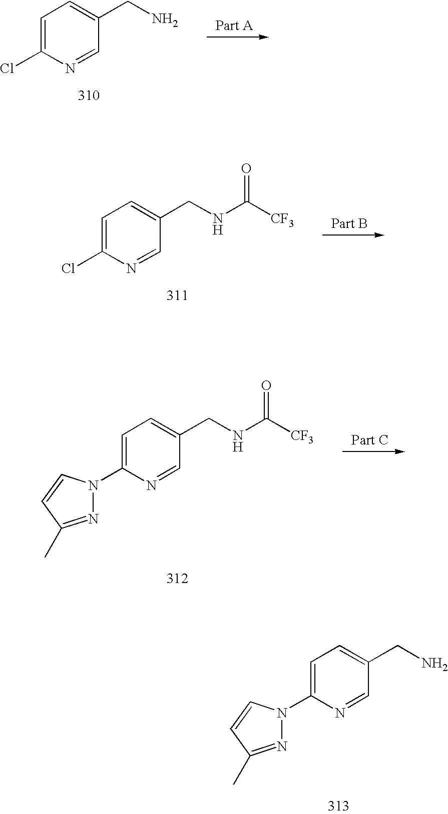

### Part A:

To a solution of 2-chloro-5-aminomethylpyridine 310 (1 g, 7.0 mmol) in dichloromethane (20 mL) at 0° C. (ice-bath) was added trifluoroacetic anhydride (1.2 mL, 8.5 mmol) in dichloromethane (10 mL). The reaction mixture was stirred at room temperature for 1 hour. LC-MS analysis of the reaction indicated that the reaction was complete. The volatiles were removed in vacuo to afford compound 311 (100% yield) as a white solid. HPLC-MS t_{R}=1.37 min (UV_{254 nm}); mass calculated for formula C₈H₆ClF₃N₂O 238.0, observed LCMS m/z 239.0 (M+H).

### Part B:

A mixture of compound 311 (0.180 g, 0.76 mmol) and 3-methylpyrazole (2 mL) was heated at 110° C. for 72 hours. Once the reaction mixture was cooled to room temperature, LC-MS analysis indicated that the reaction was complete. The volatiles were removed in vacuo, and the crude product was purified by flash column chromatography (SiO₂, ethyl acetate/methanol—9:1) to afford compound 312 as a white solid (35% yield). HPLC-MS t_{R}=1.57 min (UV_{254 nm}), mass calculated for formula C₁₂H₁₁F₃N₄O 284.1, observed LCMS m/z 285.0 (M+H).

### Part C:

A mixture of compound 312 (0.007 g, 0.03 mmol) and NaOH (1M, 0.3 mL, 0.3 mmol) in methanol (3 mL) was stirred at room temperature for 16 hours. LC-MS analysis of the reaction indicated that the reaction was incomplete. NaOH (1M, 0.6 mL, 0.6 mmol) was added and the reaction mixture heated at 55° C. for 16 hours. Once the reaction mixture was cooled to room temperature, LC-MS analysis indicated that full hydrolysis had occurred. The volatiles were removed in vacuo, and the crude dried to afford compound 30 as white paste (100% yield). HPLC-MS t_{R}=0.72 min (UV_{254 nm}); mass calculated for formula C₁₀H₁₂N₄ 188.1, observed LCMS m/z 189.1 (M+H).

### Example 6C

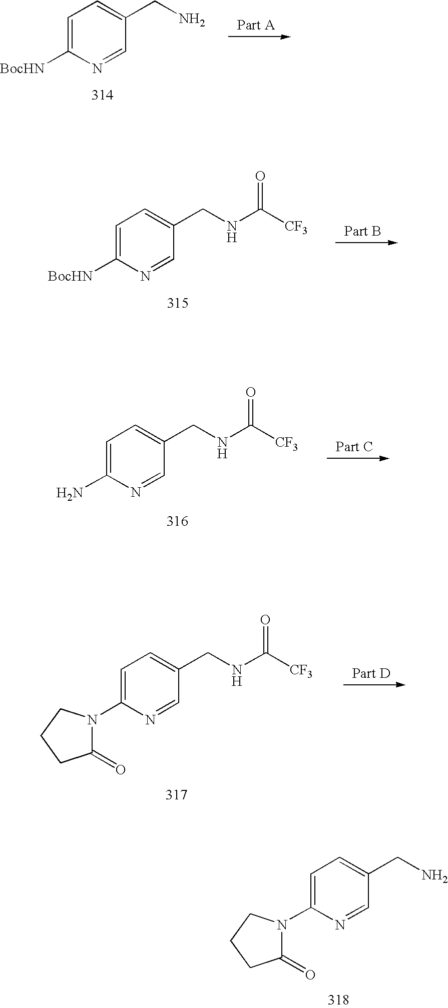

### Part A:

Compound 314 was prepared using procedures described in Example 6B, Part A. HPLC-MS t_{R}=1.59 min (UV_{254 nm}); mass calculated for formula C₁₃H₁₆F₃N₃O₃ 319.1, observed LCMS m/z 320.1 (M+H).

### Part B:

Compound 316 was prepared using procedures described in Example 6B, Part B. HPLC-MS t_{R}=0.40 min (UV_{254 nm}); mass calculated for formula C₈H₈F₃N₃O 219.1, observed LCMS m/z 220.1 (M+H).

### Part C:

A mixture of compound 316 (0.10 g, 0.46 mmol) and vinylacetic acid (5 mL) was heated at 110° C. for 96 hours. Once the reaction mixture was cooled to room temperature, LC-MS analysis indicated that the reaction was complete. The volatiles were removed in vacuo, and the crude was purified by Prep-LC to afford compound 317 as a white solid. HPLC-MS t_{R}=0.49 min (UV_{254 nm}); mass calculated for formula C₁₂H₁₂F₃N₃O₂ 287.1, observed LCMS Mk 288.1 (M+H).

### Part D:

Compound 318 was prepared using procedures described in Example 6B, Part C. HPLC-MS t_{R}=0.18 min (UV_{254 nm}); mass calculated for formula C₁₀H₁₃N₃O 191.1, observed LCMS m/z 192.1 (M+H).

### Example 6D

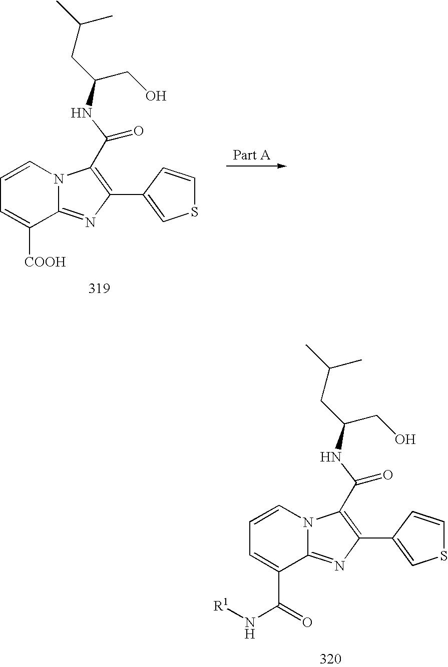

(wherein R¹ is identified in Table 13)

### Part A:

Compounds 321 and 322 are isomers and were prepared from compound 309 using the coupling conditions described in Example 1B, Part I. Purification by Prep-LC allowed isolation of both diastereomers. Compounds 323 and 324 were prepared from compounds 314 and 318 respectively, using the coupling conditions described in Example 1B, Part I.

The compounds in Table 13 were synthesized using this procedure.

TABLE 13

| | | | | Ret. |
|---|---|---|---|---|
| Compd | | | MS m/z | Time |
| # | Structure | EMW | (M⁺ + H) | (min) |
| 321 | 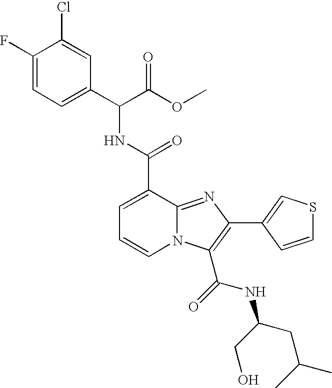 | 586.1 | 587.1 | 5.98 |
| 322 | 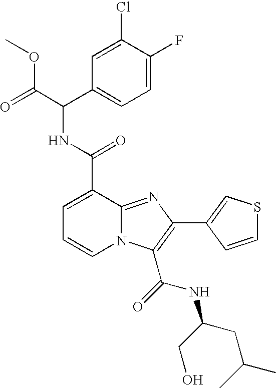 | 586.1 | 587.1 | 6.13 |
| 323 | 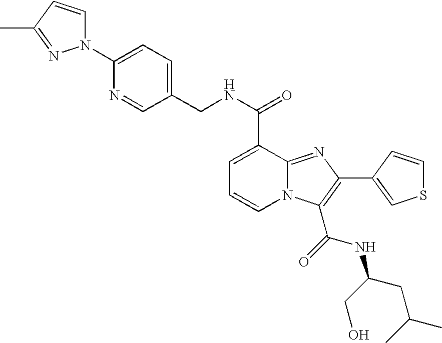 | 557.2 | 558.2 | 5.47 |
| 324 | 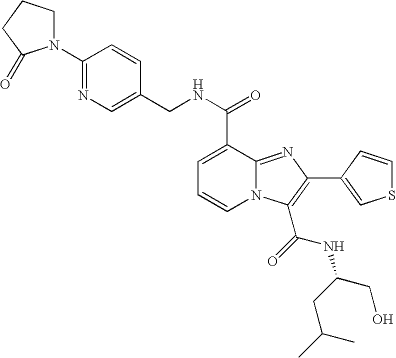 | 560.2 | 561.2 | 3.49 |

### Example 6E

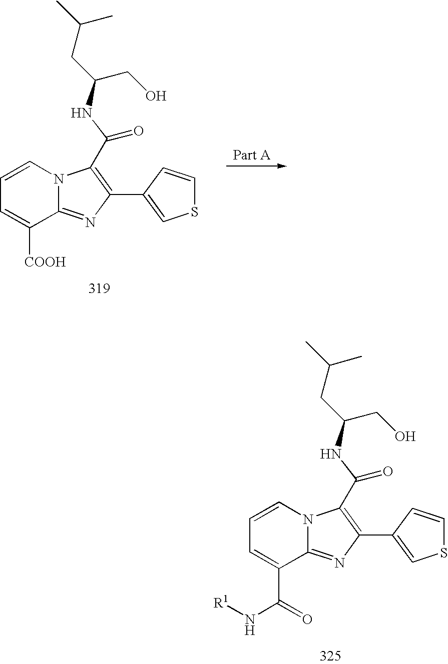

(wherein R¹ is identified in Table 14)

### Part A:

To a mixture of compound 319 (0.1 mmol) and HATU (0.046 g, 0.12 mmol) in DMF (2 mL) was added amine building block (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and washed successively with saturated NaHCO₃ (×1), water (×1), brine (×1), dried over magnesium sulfate and concentrated. The crude was redissolved in dioxane (1 mL), and a solution of 4 N HCl in dioxane (2 mL) and water (0.2 mL) was added at 0° C. (ice-bath). The reaction mixture was stirred at room temperature for 3 hours. LC-MS analysis of the reaction indicated that the reaction was complete. The volatiles were removed in vacuo, acetonitrile was added, concentrated and dried to afford compounds. Purification by Prep-LC and conversion to the hydrochloride salt afforded compounds as white solids.

The compounds in Table 14 were synthesized using this procedure.

TABLE 14

| | | | | Ret. |
|---|---|---|---|---|
| Compd | | | MS m/z | Time |
| # | Structure | EMW | (M⁺ + H) | (min) |
| 326 | 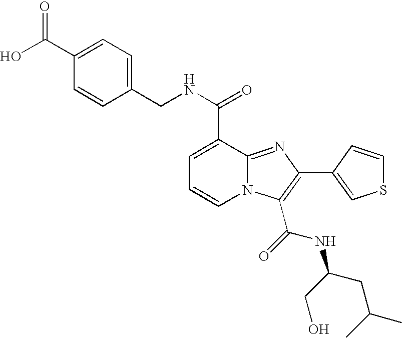 | 520.2 | 521.2 | 4.53 |
| 327 | 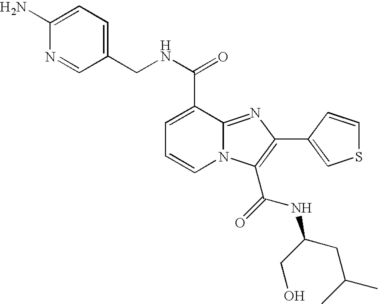 | 492.2 | 493.2 | 3.50 |
| 328 | 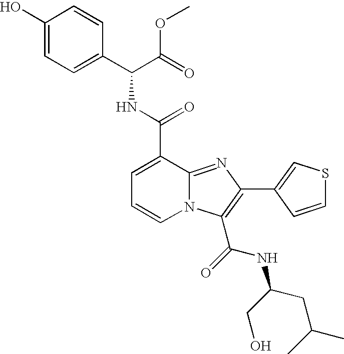 | 550.2 | 551.2 | 4.72 |

### Example 7A

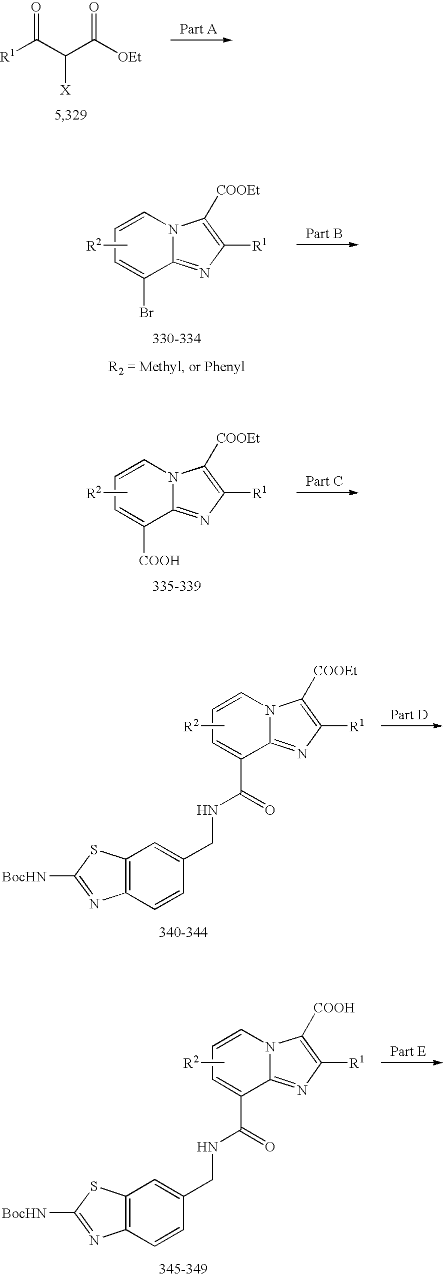

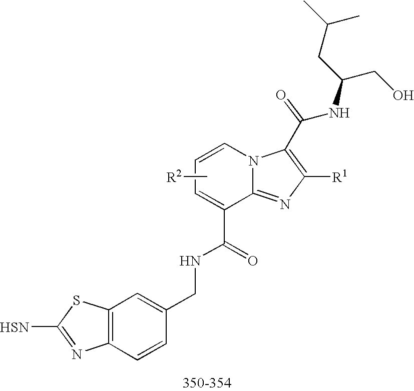

(R¹ and R² are identified in Table 15)

Compound 5 was prepared using procedures described in Example 1B.

### Part A:

A mixture of compound 5 (0.148 g, 0.53 mmol) and 2-amino-3-bromo-5-methylpyridine (0.100 g, 0.53 mmol) in ethanol (5 mL) was heated at relux for 60 hours. After cooling to room temperature, the reaction was monitored by LC-MS. The volatiles were removed in vacuo, ethyl acetate was added, and the organic solution washed successively with saturated NaHCO₃(×1), water (×1), brine (×1), dried over magnesium sulfate and concentrated. The crude was purified by preparative Thin Layer Chromatography (SiO₂, ethyl acetate/hexanes—1:1) to afford compound 330 as a white solid. HPLC-MS t_{R}=2.25 min (UV_{254 nm}); mass calculated for formula C₁₅H₁₃BrN₂O₂S 363.99, observed LCMS m/z 365.0 (M+H).

Compound 331 was prepared from the reaction of ethyl 2-chloroacetoacetate 332 and 2-amino-3-bromo-5-methylpyridine. HPLC-MS t_{R}=1.78 min (UV_{254 nm}); mass calculated for formula C₁₂H₁₃BrN₂O₂ 296.0, observed LCMS m/z 297.0 (M+H).

Compound 332 was prepared from the reaction of compound 5 and 2-amino-3-bromo-5-phenylpyridine. HPLC-MS t_{R}=2.55 min (UV_{254 nm}); mass calculated for formula C₂₀H₁₅BrN₂O₂S 426.0, observed LCMS m/z 427.0 (M+H).

Compound 333 was prepared from the reaction of ethyl 2-chloroacetoacetate 332 and 2-amino-3-bromo-5-phenylpyridine. HPLC-MS t_{R}=2.26 min (UV_{254 nm}); mass calculated for formula C₁₇H₁₅BrN₂O₂ 358.0, observed LCMS m/z 359.0 (M+H).

Compound 334 was prepared from the reaction of ethyl 2-chloroacetoacetate 332 and 2-amino-3-bromo-6-methylpyridine. HPLC-MS t_{R}=1.61 min (UV_{254 nm}); mass calculated for formula C₁₂H₁₃BrN₂O₂ 296.0, observed LCMS m/z 297.0 (M+H).

### Part B:

A saturated solution of carbon monoxide in a 20 ml scintillation vial was pre-prepared by adding acetic anhydride (0.032 mL, 0.34 mmol) and diisopropylethylamine (0.046 mL, 0.34 mmol) to a solution of sodium formate (0.034 g, 0.51 mmol) in de-gassed DMF (2 mL). The reaction mixture was stirred at room temperature for 1 hour. In another flask, palladium (II) acetate (0.00113 g, 0.005 mmol) was added to a solution of 1,3-bis(diphenylphosphino)propane (0.00207 g, 0.005 mmol) in de-gassed DMF (2 mL) and stirred at room temperature for 30 minutes. Lithium chloride (0.021 g, 0.51 mmol) was added and the solution sonicated to ensure there was no precipitation. Compound 330 (0.061 g, 0.17 mmol) was added and the reaction mixture quickly transferred to the saturated solution of carbon monoxide. The vial was capped and the reaction mixture heated at 80° C. for 16 hours. The vial was cooled to room temperature, and the reaction monitored by LC-MS. The precipitates were removed by filtration, the filtrate concentrated, and the crude re-dissolved in acetonitrile (1 mL). The solution was acidified to pH 4.0 with 1.0 M HCl, concentrated and dried to afford compound 335 which was used as crude in the next step. HPLC-MS t_{R}=1.85 min (UV_{254 nm}); mass calculated for formula C₁₆H₁₄N₂O₄S 330.1, observed LCMS m/z 331.0 (M+H).

Compound 336 was prepared from compound 331. HPLC-MS t_{R}=1.01 min (UV_{254 nm}); mass calculated for formula C₁₃H₁₄N₂O₄ 262.1, observed LCMS m/z 263.1 (M+H).

Compound 337 was prepared from compound 332. HPLC-MS t_{R}=2.28 min (UV_{254 nm}); mass calculated for formula C₂₁H₁₆N₂O₄S 392.1, observed LCMS m/z 393.1 (M+H).

Compound 338 was prepared from compound 333. HPLC-MS t_{R}=1.55 min (UV_{254 nm}); mass calculated for formula C₁₈H₁₆N₂O₄ 324.1, observed LCMS m/z 325.1 (M+H).

Compound 339 was prepared from compound 334. HPLC-MS t_{R}=0.95 min (UV_{254 nm}); mass calculated for formula C₁₃H₁₄N₂O₄ 262.1, observed LCMS m/z 263.2 (M+H).

### Part C:

To a mixture of compound 335 (0.1 mmol) and HATU (0.046 g, 0.12 mmol) in DMF (2 mL) was added (6-Aminomethyl-benzothiazol-2-yl)-carbamic acid tert-butyl ester (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and the organic solution washed successively with saturated NaHCO₃ (×1), water (×1), brine (×1), dried over magnesium sulfate and concentrated. The crude was purified by preparative Thin Layer Chromatography (SiO₂, ethyl acetate) to afford compound 340 as a white solid. HPLC-MS t_{R}=2.40 min (UV_{254 nm}); mass calculated for formula C₂₉H₂₉N₅O₅S₂ 591.2, observed LCMS m/z 592.0 (M+H).

Compound 341 was prepared from compound 336. HPLC-MS t_{R}=2.31 min (UV_{254 nm}); mass calculated for formula C₂₆H₂₉N₅O₅S 523.2, observed LCMS m/z 524.2 (M+H).

Compound 342 was prepared from compound 337. HPLC-MS t_{R}=2.50 min (UV_{254 nm}); mass calculated for formula C₃₄H₃₁N₅O₅S₂ 653.2, observed LCMS m/z 654.1 (M+H).

Compound 343 was prepared from compound 338. HPLC-MS t_{R}=2.44 min (UV_{254 nm}); mass calculated for formula C₃₁H₃₁N₅O₅S 585.2, observed LCMS m/z 586.2 (M+H).

Compound 344 was prepared from compound 339. HPLC-MS t_{R}=1.54 min (UV_{254 nm}); mass calculated for formula C₂₆H₂₉N₅O₅S 523.2, observed LCMS m/z 524.2 (M+H).

### Part D:

A mixture of compound 340 (0.010 g, 0.017 mmol) and LiOH (1M, 51 uL, 0.051 mmol) in THF (2 mL) and water (1 mL) was heated at 55° C. for 16 hours. LC-MS analysis of the reaction indicated that the reaction was complete. Hexanes (1 mL) were added to form a biphasic solution. The aqueous phase was separated, acidified to pH 4.0 with 1N HCl, concentrated and lyophilized with acetonitrile and water (1:1) to afford compound 345 as a white solid. HPLC-MS t_{R}=1.95 min (UV_{254 nm}); mass calculated for formula C₂₇H₂₅N₅O₅S₂ 563.1, observed LCMS m/z 564.1 (M+H).

Compound 346 was prepared from compound 341. HPLC-MS t_{R}=1.74 min (UV_{254 nm}); mass calculated for formula C₂₄H₂₅N₅O₅S 495.2, observed LCMS m/z 496.1 (M+H).

Compound 347 was prepared from compound 342. HPLC-MS t_{R}=2.07 min (UV_{254 nm}); mass calculated for formula C32H₂₇N₅O₅S₂ 625.1, observed LCMS m/z 626.0 (M+H).

Compound 348 was prepared from compound 343. HPLC-MS t_{R}=1.93 min (UV_{254 nm}); mass calculated for formula C₂₉H₂₇N₅O₅S 557.2, observed LCMS m/z 558.1 (M+H).

Compound 349 was prepared from compound 344. HPLC-MS t_{R}=1.22 min (UV_{254 nm}); mass calculated for formula C₂₄H₂₅N₅O₅S 495.2, observed LCMS m/z 496.1 (M+H).

### Part E:

To a mixture of compound 345 (0.1 mmol) and HATU (0.046 g, 0.12 mmol) in DMF (2 mL) was added L-leucinol (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and washed successively with saturated NaHCO₃ (×1), water (×1), brine (×1), dried over magnesium sulfate and concentrated. The crude was redissolved in dioxane (1 mL), and a solution of 4 N HCl in dioxane (2 mL) and water (0.2 mL) was added at 0° C. (ice-bath). The reaction mixture was stirred at room temperature for 3 hours. LC-MS analysis of the reaction indicated that hydrolysis was complete. The volatiles were removed in vacuo, acetonitrile was added, concentrated and dried to afford compounds. Purification by Prep-LC and conversion to the hydrochloric salt afforded compounds 350-354 (Table 15) as white solids.

The ligands in Table 15 were synthesized using this procedure.

TABLE 15

| | | | MS | Ret. |
|---|---|---|---|---|
| Compd | | | m/z | Time |
| # | Structure | EMW | (M⁺ + H) | (min) |
| 350 | 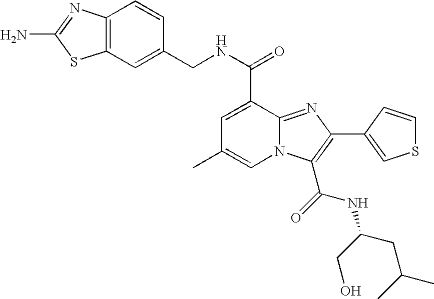 | 562.2 | 563.1 | 3.93 |
| 351 | 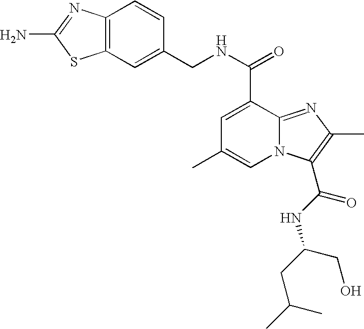 | 494.2 | 495.1 | 2.83 |
| 352 | 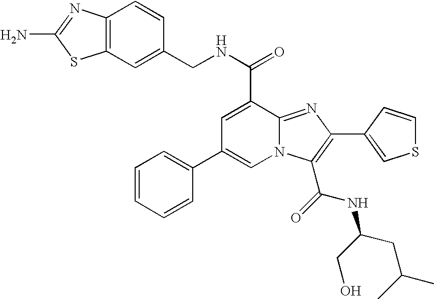 | 624.2 | 625.2 | 4.42 |
| 353 | 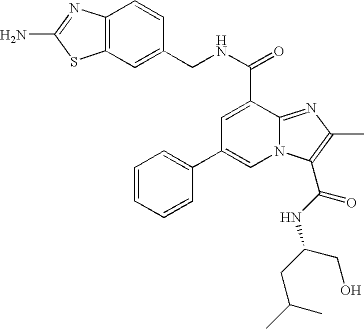 | 556.2 | 557.2 | 3.72 |
| 354 | 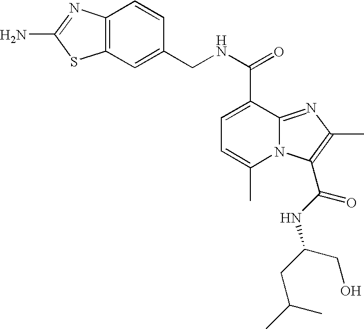 | 494.2 | 494.6 | 2.23 |

### Example 9A

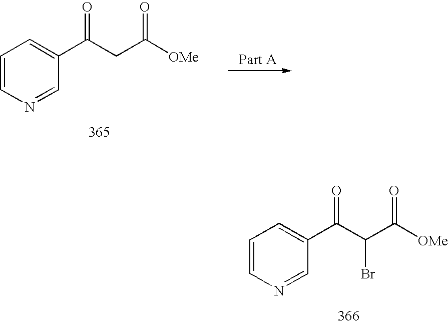

### Part A:

Compound 366 was prepared from methyl nicotinoylacetate 365 using procedures described in Example 1B, Part B. HPLC-MS t_{R}=1.15 min (UV_{254 nm}); mass calculated for formula C₉H₈BrNO₃ 257.0, observed LCMS m/z 258.0 (M+H).

### Example 9B

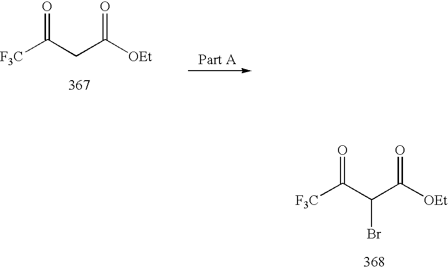

### Part A:

Compound 368 was prepared from ethyl 4,4,4-trifluoroacetoacetate 367 using procedures described in Example 1B, Part B. HPLC-MS t_{R}=1.30 min (UV_{254 nm}); mass calculated for formula C₆H₆BrF₃O₃ 261.9, observed LCMS m/z 263.0 (M+H).

### Example 9C

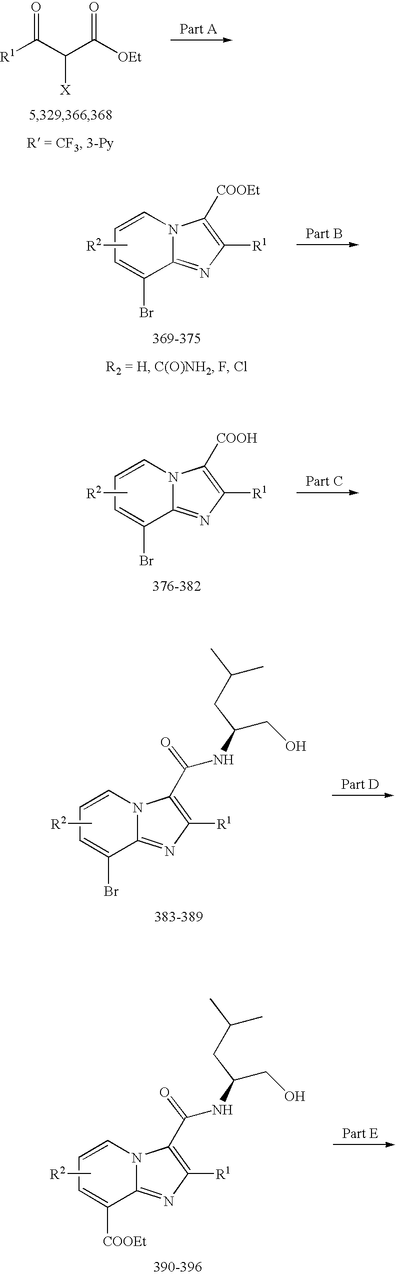

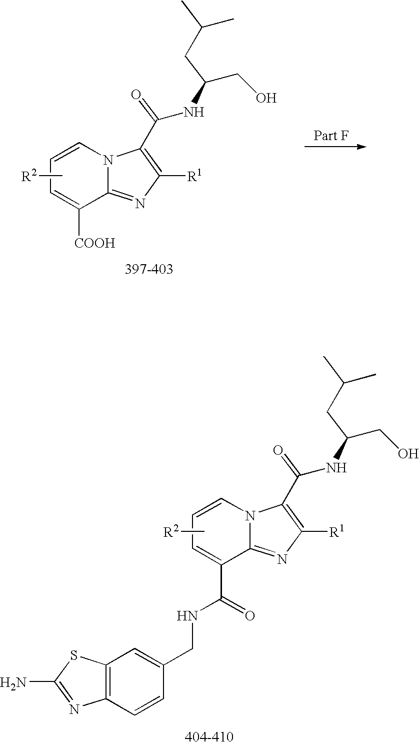

(wherein R¹ is identified in Table 16)

Compound 5 was prepared using procedures described in Example 1B.

### Part A:

A mixture of compound 5 (0.148 g, 0.53 mmol) and 2-amino-3-bromo-5-chloropyridine (0.110 g, 0.53 mmol) in ethanol (5 mL) was heated at reflux for 60 hours. After cooling to room temperature, the reaction was monitored by LC-MS. The volatiles were removed in vacuo, ethyl acetate was added, and the organic solution washed successively with saturated NaHCO₃(×1), water (×1), brine (×1), dried over magnesium sulfate and concentrated. The crude was purified by preparative Thin Layer Chromatography (SiO₂, ethyl acetate/hexanes—1:1) to afford compound 369 as a white solid. HPLC-MS t_{R}=2.40 min (UV_{254 nm}); mass calculated for formula C₁₄H₁₀BrClN₂O₂S 383.9, observed LCMS m/z 384.9 (M+H).

Compound 370 was prepared from the reaction of ethyl 2-chloroacetoacetate 329 and 2-amino-3-bromo-5-chloropyridine. HPLC-MS t_{R}=2.07 min (UV_{254 nm}), mass calculated for formula C₁₁H₁₀BrClN₂O₂ 316.0, observed LCMS m/z 317.0 (M+H).

Compound 371 was prepared from the reaction of ethyl 2-chloroacetoacetate 329 and 6-amino-5-bromo-nicotinonitrile. HPLC-MS t_{R}=1.74 min (UV_{254 nm}); mass calculated for formula C₁₂H₁₀BrN₃O₂ 307.0, observed LCMS m/z 308.0 (M+H).

Compound 372 was prepared from the reaction of compound 5 and 2-amino-3-bromo-5-fluoropyridine. HPLC-MS t_{R}=2.29 min (UV_{254 nm}); mass calculated for formula C₁₄H₁₀BrFN₂O₂S 368.0, observed LCMS m/z 369.0 (M+H).

Compound 373 was prepared from the reaction of ethyl 2-chloroacetoacetate 329 and 2-amino-3-bromo-5-fluoropyridine. HPLC-MS t_{R}=1.84 min (UV_{254 nm}); mass calculated for formula C₁₁H₁₀BrFN₂O₂ 300.0, observed LCMS m/z 301.0 (M+H).

Compound 374 was prepared from the reaction of compound 366 and 2-amino-3-bromo-pyridine. HPLC-MS t_{R}=1.11 min (UV_{254 nm}); mass calculated for formula C74H₁₀BrN₃O₂ 331.0, observed LCMS m/z 332.0 (M+H).

Compound 375 was prepared from the reaction of compound 367 and 2-amino-3-bromo-pyridine. HPLC-MS t_{R}=2.03 min (UV_{254 nm}); mass calculated for formula C₁₁H₈BrF₃N₂O₂ 336.0, observed LCMS m/z 337.0 (M+H).

### Part B:

Compound 376 was prepared from compound 369 using procedures described in Example 7A, Part D. HPLC-MS t_{R}=1.80 min (UV_{254 nm}); mass calculated for formula C₁₂H₆BrClN₂O₂S 355.9, observed LCMS m/z 357.0 (M+H).

Compound 377 was prepared from compound 370. HPLC-MS t_{R}=1.32 min (UV_{254 nm}); mass calculated for formula C₉H₆BrClN₂O₂ 287.9, observed LCMS m/z 289.0 (M+H).

Compound 378 was prepared from compound 371. HPLC-MS t_{R}=0.77 min (UV_{254 nm}); mass calculated for formula C₁₀H₈BrN₃O₃ 297.0, observed LCMS m/z 298.0 (M+H).

Compound 379 was prepared from compound 372. HPLC-MS t_{R}=1.63 min (UV_{254 nm}); mass calculated for formula C₁₂H₆BrFN₂O₂S 339.9, observed LCMS m/z 340.9 (M+H).

Compound 380 was prepared from compound 373. HPLC-MS t_{R}=1.08 min (UV_{254 nm}); mass calculated for formula C₉H₆BrFN₂O₂ 272.0, observed LCMS m/z 273.0 (M+H).

Compound 381 was prepared from compound 374. HPLC-MS t_{R}=1.41 min (UV_{254 nm}); mass calculated for formula C₁₃H₈BrN₃O₂ 317.0, observed LCMS m/z 318.0 (M+H).

Compound 382 was prepared from compound 375. HPLC-MS t_{R}=1.41 min (UV_{254 nm}); mass calculated for formula C₉H₄BrF₃N₂O₂ 307.9, observed LCMS m/z 309.0 (M+H).

### Part C:

To a mixture of compound 376 (0.1 mmol) and HATU (0.046 g, 0.12 mmol) in DMF (2 mL) was added L-Leucinol (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and the organic solution washed successively with saturated NaHCO₃ (×1), water (×1), brine (×1), dried over magnesium sulfate and concentrated. The crude was purified by preparative Thin Layer Chromatography (SiO₂, ethyl acetate/methanol—9:1) to afford compound 383 as a white solid. HPLC-MS t_{R}=2.07 min (UV_{254 nm}); mass calculated for formula C₁₈H₁₉BrClN₃O₂S 455.0, observed LCMS m/z 456.0 (M+H).

Compound 384 was prepared from compound 377. HPLC-MS t_{R}=1.70 min (UV_{254 nm}); mass calculated for formula C₁₅H₁₉BrClN₃O2 387.0, observed LCMS m/z 388.0 (M+H).

Compound 385 was prepared from compound 378. HPLC-MS t_{R}=0.69 min (UV_{254 nm}); mass calculated for formula C₁₆H₂₁BrN₄O₃ 396.1, observed LCMS m/z 397.1 (M+H).

Compound 386 was prepared from compound 379. HPLC-MS t_{R}=1.90 min (UV_{254 nm}); mass calculated for formula C₁₈H₁₉BrFN₃O₂S 439.0, observed LCMS m/z 440.0 (M+H).

Compound 387 was prepared from compound 380. HPLC-MS t_{R}=1.54 min (UV_{254 nm}); mass calculated for formula C₁₅H₁₉BrFN₃O₂ 371.1, observed LCMS m/z 372.0 (M+H).

Compound 388 was prepared from compound 381. HPLC-MS t_{R}=1.27 min (UV_{254 nm}); mass calculated for formula C₁₉H₂₁BrN₁₄O₂ 416.1, observed LCMS m/z 417.1 (M+H).

Compound 389 was prepared from compound 382. HPLC-MS t_{R}=1.72 min (UV_{254 nm}); mass calculated for formula C₁₅H₁₇BrF₃N₃O₂ 407.0, observed LCMS m/z 408.0 (M+H).

### Part D:

Carbon monoxide (˜1.5 mL) was condensed into an evacuated ACE pressure tube (35 mL) at −78° C. (liquid nitrogen). A solution of compound 383 (0.58 mmol) in ethanol (7 mL) was transferred to the reaction tube, Pd(DPPF)Cl₂. DCM (10 mol %) was added, the pressure tube capped, and the reaction mixture warmed slowly to room temperature and then finally heated at 80° C. for 16 hours. The reaction mixture was cooled to 0° C. (ice-bath), and the pressure released by uncapping the pressure tube. LC-MS analysis of the reaction indicated that the reaction was complete. The precipitates were filtered and the volatiles removed in vacuo. The crude was purified by preparative Thin Layer Chromatography (SiO₂, ethyl acetate/methanol—9:1) to afford compound 390. HPLC-MS t_{R}=1.99 min (UV_{254 nm}); mass calculated for formula C₂₁H₂₄ClN₃O₄S 449.1, observed LCMS m/z 450.1 (M+H).

Compound 391 was prepared from compound 384. HPLC-MS t_{R}=1.46 min (UV_{254 nm}); mass calculated for formula C₁₈H₂₄ClN₃O₄ 381.1, observed LCMS m/z 382.1 (M+H).

Compound 392 was prepared from compound 385. HPLC-MS t_{R}=1.06 min (UV_{254 nm}); mass calculated for formula C₁₉H₂₆N₄O₅ 390.2, observed LCMS m/z 391.1 (M+H).

Compound 393 was prepared from compound 386. HPLC-MS t_{R}=1.84 min (UV_{254 nm}); mass calculated for formula C₂₁H₂₄FN₃O₄S 433.1, observed LCMS m/z 434.1 (M+H).

Compound 394 was prepared from compound 397. HPLC-MS t_{R}=1.28 min (UV_{254 nm}); mass calculated for formula C₁₈H₂₄FN₃O₄ 365.2, observed LCMS m/z 366.1 (M+H).

Compound 395 was prepared from compound 388. HPLC-MS t_{R}=1.14 min (UV_{254 nm}); mass calculated for formula C₂₂H₂₆N₄O₄ 410.2, observed LCMS m/z 411.1 (M+H).

Compound 396 was prepared from compound 389. HPLC-MS t_{R}=1.73 min (UV_{254 nm}); mass calculated for formula C₁₈H₂₂F₃N₃O₄ 401.2, observed LCMS m/z 402.1 (M+H).

### Part E:

Compound 397 was prepared from compound 390 using procedures described in Example 2A, Part D. HPLC-MS t_{R}=1.69 min (UV_{254 nm}); mass calculated for formula C₁₉H₂₀ClN₃O₄S 421.1, observed LCMS m/z 422.1 (M+H).

Compound 398 was prepared from compound 391. HPLC-MS t_{R}=1.09 min (UV_{254 nm}); mass calculated for formula C₁₆H₂₀ClN₃O₄ 353.1, observed LCMS m/z 354.1 (M+H).

Compound 399 was prepared from compound 392. HPLC-MS t_{R}=0.79 min (UV_{254 nm}); mass calculated for formula C₁₇H₂₂N₁₄O₅ 362.2, observed LCMS m/z 363.1 (M+H).

Compound 400 was prepared from compound 393. HPLC-MS t_{R}=1.52 min (UV_{254 nm}); mass calculated for formula C₁₉H₂₀FN₃O₄S 405.1, observed LCMS m/z 406.1 (M+H).

Compound 401 was prepared from compound 394. HPLC-MS t_{R}=1.00 min (UV_{254 nm}); mass calculated for formula C₁₆H₂₀FN₃O₄ 337.1, observed LCMS m/z 338.1 (M+H).

Compound 402 was prepared from compound 395. HPLC-MS t_{R}=1.04 min (UV_{254 nm}); mass calculated for formula C₂₀H₂₂N₄O₄ 382.2, observed LCMS m/z 383.1 (M+H).

Compound 403 was prepared from compound 396. HPLC-MS t_{R}=1.46 min (UV_{254 nm}); mass calculated for formula C₁₆H₁₈F₃N₃O₄ 373.1, observed LCMS m/z 374.0 (M+H).

### Part F:

To a mixture of mono-acid (0.1 mmol) and HATU (0.046 g, 0.12 mmol) in DMF (2 mL) was added amine building block (1.2 equivalents) and diisopropylamine (3 equivalents). The reaction mixture was stirred at room temperature for 3 hours. LC-MS analysis of the reaction indicated that the reaction was complete. The volatiles were removed in vacuo, ethyl acetate was added, and washed successively with saturated NaHCO₃(×1), water (×1), brine (×1), dried over magnesium sulfate and concentrated. For compounds 404-405 and 407-410, the crude was redissolved in dioxane (1 mL), and a solution of 4 N HCl in dioxane (2 mL) and water (0.2 mL) was added at 0° C. (ice-bath). The reaction mixture was stirred at room temperature for 3 hours. LC-MS analysis of the reaction indicated that hydrolysis was complete. The volatiles were removed in vacuo, acetonitrile was added, concentrated and dried to afford compounds. Purification by Prep-LC and conversion to the hydrochloride salt afforded compounds 404-410 as white solids.

The compounds in Table 16 were synthesized using this procedure.

TABLE 16

| | | | | Ret. |
|---|---|---|---|---|
| Compd | | | MS m/z | Time |
| # | Structure | EMW | (M⁺ + H) | (min) |
| 404 | 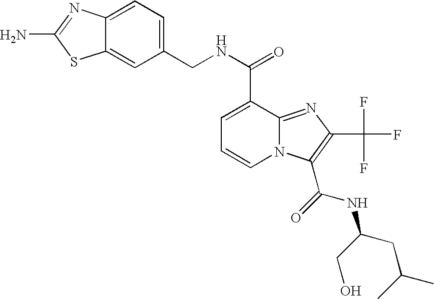 | 534.2 | 535.2 | 3.74 |
| 405 | 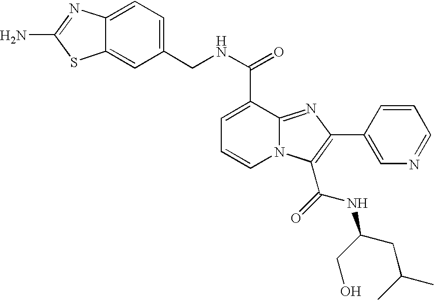 | 543.2 | 544.2 | 2.84 |
| 406 | 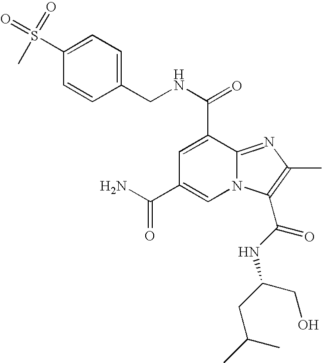 | 529.2 | 530.2 | 3.5 |
| 407 | 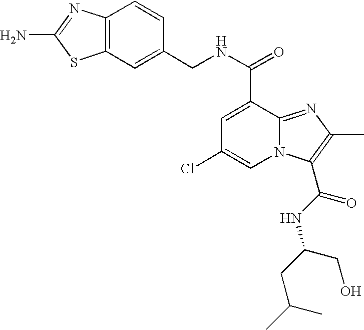 | 514.2 | 515.2 | 3.78 |
| 408 | 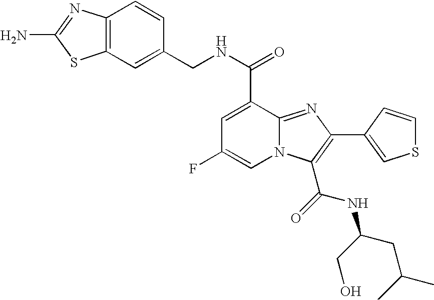 | 566.2 | 567.2 | 4.16 |
| 409 | 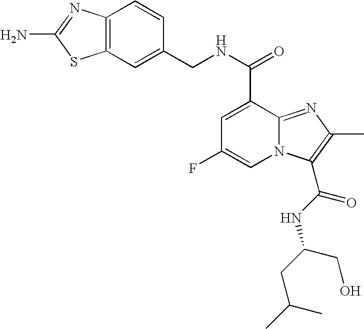 | 498.2 | 499.2 | 3.51 |
| 410 | 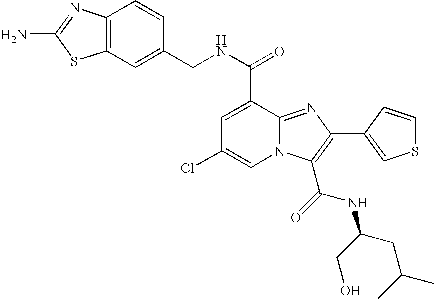 | 582.1 | 583.2 | 4.41 |

### Example 10A

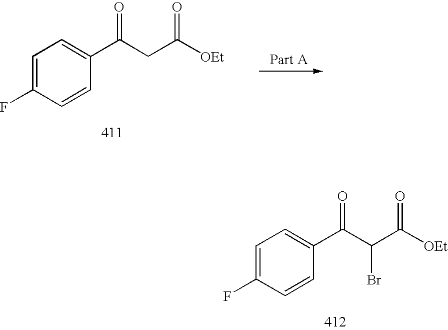

### Part A:

Compound 412 was prepared from ethyl 4-fluorobenzoylacetate 411 using procedures described in Example 1B, Part B. HPLC-MS t_{R}=1.86 min (UV_{254 nm}); mass calculated for formula C₁₁H₁₀BrFO₃ 288.0, observed LCMS m/z 289.0 (M+H).

### Example 10B

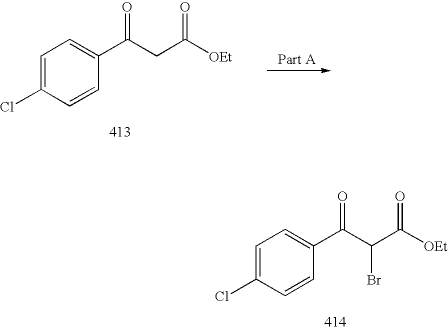

### Part A:

Compound 414 was prepared from ethyl 4-chlorobenzoylacetate 413 using procedures described in Example 1B, Part B. HPLC-MS t_{R}=2.04 min (UV_{254 nm}); mass calculated for formula C₁₁H₁₀BrClO₃ 304.0, observed LCMS m/z 305.0 (M+H).

### Example 10C

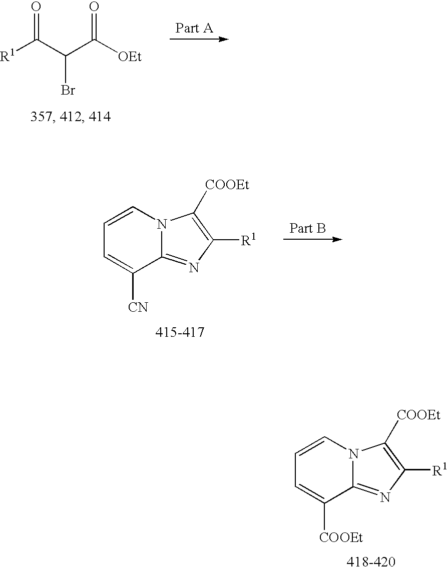

### Part A:

A mixture of compound 357 (2 mmol) and 2-amino-3-cyanopyridine (0.200 g, 1.67 mmol) in ethanol (8 mL) was heated at reflux for 60 hours. After cooling to room temperature, the reaction was monitored by LC-MS. The volatiles were removed in vacuo, ethyl acetate was added, and the organic solution washed successively with saturated NaHCO₃ (×1), water (×1), brine (×1), dried over magnesium sulfate and concentrated. The crude was purified by preparative Thin Layer Chromatography (SiO₂, ethyl acetate/hexanes—1:1) to afford compound 415 as a white solid. HPLC-MS t_{R}=1.92 min (UV_{254 nm}); mass calculated for formula C₁₇H₁₃N₃O₂ 291.1, observed LCMS m/z 292.0 (M+H).

Compound 416 was prepared from the reaction of compound 412 and 2-amino-3-cyanopyridine. HPLC-MS t_{R}=1.96 min (UV_{254 nm}); mass calculated for formula C₁₇H₁₂ClN₃O₂ 309.1, observed LCMS m/z 310.1 (M+H).

Compound 417 was prepared from the reaction of compound 414 and 2-amino-3-cyanopyridine. HPLC-MS t_{R}=2.08 min (UV_{254 nm}); mass calculated for formula C₁₇H₁₂ClN₃O₂ 325.1, observed LCMS m/z 326.0 (M+H).

### Part B:

A mixture of compound 415 (0.090 g, 0.31 mmol) and chlorotrimethylsilane (0.393 mL, 3.1 mmol) in ethanol (5 mL) was heated at 60° C. for 16 hours. After cooling to room temperature, the reaction was monitored by LC-MS. After cooling to room temperature, the reaction was monitored by LC-MS. The volatiles were removed in vacuo, ethyl acetate was added, and the organic solution washed successively with saturated NaHCO₃ (×1), water (×1), brine (×1), dried over magnesium sulfate and concentrated. The crude was purified by preparative Thin Layer Chromatography (SiO₂, ethyl acetate) to afford compound 418 as a white solid. HPLC-MS t_{R}=1.82 min (UV_{254 nm}); mass calculated for formula C₁₉H₁₈N₁₂O₄ 338.1, observed LCMS m/z 339.1 (M+H).

Compound 419 was prepared from compound 416. HPLC-MS t_{R}=1.91 min (UV_{254 nm}); mass calculated for formula C₁₉H₁₇FN₂O₄ 356.1, observed LCMS m/z 357.1 (M+H).

Compound 420 was prepared from compound 417. HPLC-MS t_{R}=2.16 min (UV_{254 nm}); mass calculated for formula C₁₉H₁₇ClN₂O₄ 372.1, observed LCMS m/z 373.0 (M+H).

### Example 10D

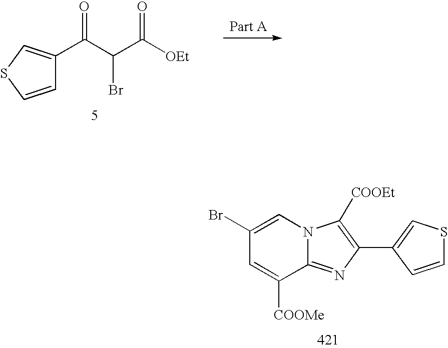

Compound 5 was prepared using procedures described in Example 1B.

### Part A:

Compound 421 was prepared from the reaction of compound 5 and methyl 2-amino-5-bromonicotinate using procedures described in Example 1B, Part C. HPLC-MS t_{R}=2.15 min (UV_{254 nm}); mass calculated for formula C₁₆H₁₃BrN₂O₄S 408.0, observed LCMS m/z 409.0 (M+H).

### Example 10E

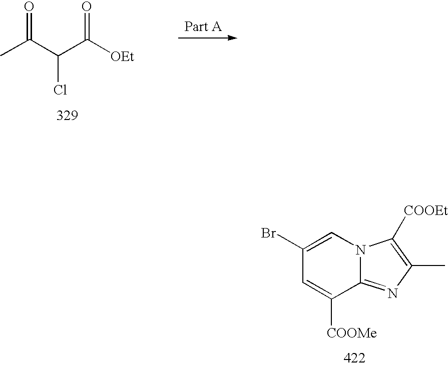

### Part A:

Compound 422 was prepared from the reaction of ethyl 2-chloroacetoacetate 329 and methyl 2-amino-5-bromonicotinate using procedures described in Example 1B, Part C. HPLC-MS t_{R}=1.72 min (UV_{254 nm}); mass calculated for formula C₁₃H₁₃BrN₂O₄ 340.0, observed LCMS m/z 341.0 (M+H).

### Example 10F

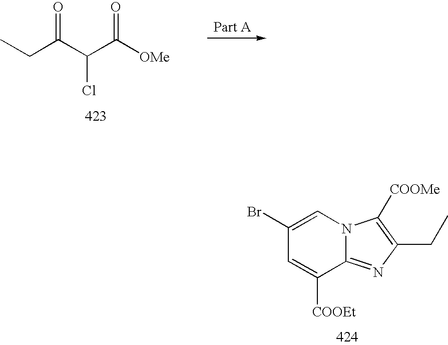

### Part A:

Compound 424 was prepared from the reaction of methyl 2-chloro-3-oxopentanoate 423 and compound 2 using procedures described in Example 1B, Part C. HPLC-MS t_{R}=1.20 min (UV_{254 nm}); mass calculated for formula C₁₄H₁₆N₂O₄ 276.1, observed LCMS m/z 277.1 (M+H).

### Example 10G

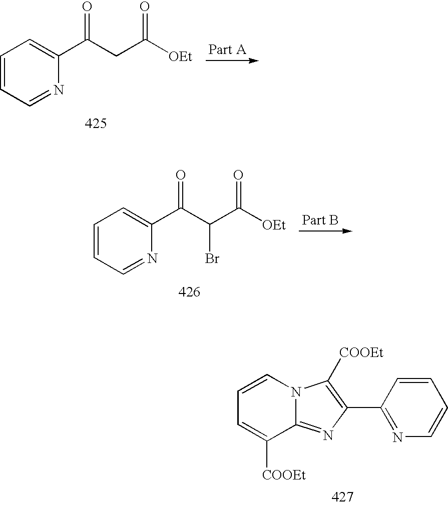

### Part A:

Compound 426 was prepared from ethyl picolinoylacetate 425 using procedures described in Example 1B, Part B. HPLC-MS t_{R}=1.94 min (UV_{254 nm}); mass calculated for formula C₁₀H₁₀BrNO₃ 271.0, observed LCMS m/z 272.0 (M+H).

### Part B:

Compound 427 was prepared from the reaction of compound 426 and compound 2 using procedures described in Example 1B, Part C. HPLC-MS t_{R}=0.67 min (UV_{254 nm}); mass calculated for formula C₁₈H₁₇N₃O₄ 339.1, observed LCMS m/z 340.0 (M+H).

### Example 10H

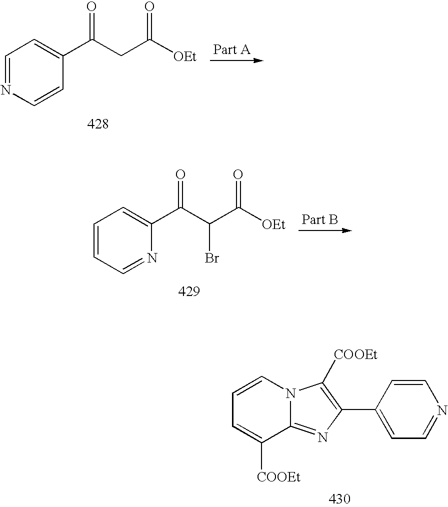

### Part A:

Compound 429 was prepared from ethyl isonicotinoylacetate 428 using procedures described in Example 1B, Part B. HPLC-MS t_{R}=1.49 min (UV_{254 nm}); mass calculated for formula C₁₀H₁₀BrNO₃ 271.0, observed LCMS m/z 272.0 (M+H).

### Part B:

Compound 430 was prepared from the reaction of compound 429 and compound 2 using procedures described in Example 1B, Part C. HPLC-MS t_{R}=0.66 min (UV_{254 nm}); mass calculated for formula C₁₈H₁₇N₃O₄ 339.1, observed LCMS m/z 340.0 (M+H).

### Example 10I

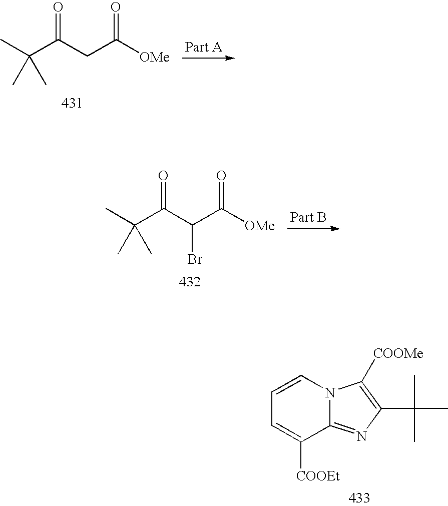

### Part A:

Compound 432 was prepared from methyl 4,4-dimethyl-3-oxopentanoate 431 using procedures described in Example 1B, Part B. HPLC-MS t_{R}=1.70 min (UV₂₅₄ nm); mass calculated for formula C₈H₁₃BrO₃ 236.0, observed LCMS m/z 237.0 (M+H).

### Part B:

Compound 433 was prepared from the reaction of compound 432 and compound 2 using procedures described in Example 1B, Part C. HPLC-MS t_{R}=1.89 min (UV_{254 nm}); mass calculated for formula C₁₆H₂₀N₂O₄ 304.1, observed LCMS m/z 305.1 (M+H).

### Example 10J

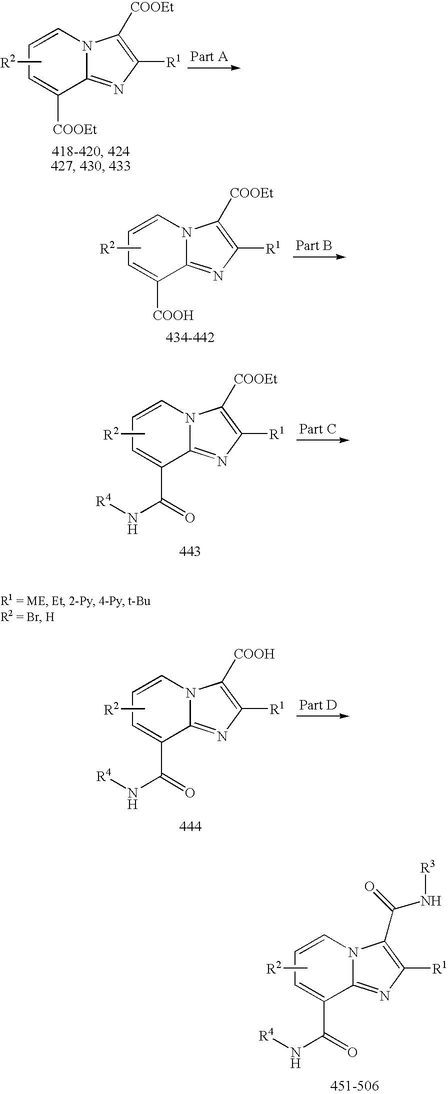

(wherein R¹⁻⁴ are identified in Table 17)

### Part A:

A mixture of compound 418 (0.09 mmol) and LiOH (1M, 0.18 mL, 0.18 mmol) in THF (3 mL) and water (1 mL) was stirred at room temperature for 3 hours. LC-MS analysis of the reaction indicated that the reaction was complete. Hexanes (1 mL) were added to form a biphasic solution. The aqueous phase was separated, acidified to pH 4.0 with 1N HCl, concentrated and lyophilized with acetonitrile and water (1:1) to afford compound 434 as a white solid (100% yield). HPLC-MS t_{R}=1.74 min (UV_{254 nm}); mass calculated for formula C₁₇H₁₄N₂O₄ 310.1, observed LCMS m/z 311.1 (M+H).

Compound 435 was prepared from compound 419. HPLC-MS t_{R}=1.81 min (UV_{254 nm}); mass calculated for formula C₁₇H₁₃FN₂O₄ 328.1, observed LCMS m/z 329.0 (M+H).

Compound 436 was prepared from compound 420. HPLC-MS t_{R}=2.01 min (UV_{254 nm}); mass calculated for formula C₁₇H₁₃ClN₂O₄ 344.1, observed LCMS m/z 345.0 (M+H).

Compound 437 was prepared from compound 421. HPLC-MS t_{R}=2.08 min (UV_{254 nm}); mass calculated for formula C₁₅H₁₁BrN₂O₄S 394.0, observed LCMS m/z 394.9 (M+H).

Compound 438 was prepared from compound 422. HPLC-MS t_{R}=1.30 min (UV_{254 nm}); mass calculated for formula C₁₂H₁₁BrN₂O₄ 326.0, observed LCMS m/z 327.0 (M+H).

Compound 439 was prepared from compound 424. HPLC-MS t_{R}=0.87 min (UV_{254 nm}); mass calculated for formula C₁₂H₁₂N₂O₄ 248.1, observed LCMS m/z 249.1 (M+H).

Compound 440 was prepared from compound 427. HPLC-MS t_{R}=1.07 min (UV_{254 nm}); mass calculated for formula C₁₆H₁₃N₃O₄ 311.1, observed LCMS m/z 312.0 (M+H).

Compound 441 was prepared from compound 430. HPLC-MS t_{R}=0.95 min (UV_{254 nm}); mass calculated for formula C₁₆H₁₃N₃O₄ 311.1, observed LCMS m/z 312.0 (M+H).

Compound 442 was prepared from compound 433. HPLC-MS t_{R}=1.84 min (UV_{254 nm}); mass calculated for formula C₁₅H₁₈N₂O₄ 290.1, observed LCMS m/z 291.1 (M+H).

### Part B:

Compounds 443 were prepared using coupling procedures described in Example 1B, Part G.

### Part C:

The esters were saponified to form compounds 444 using procedures described in Example 7A, Part D.

The compounds in Table 17 were synthesized using this procedure.

TABLE 17

| | | | | Ret. |
|---|---|---|---|---|
| Compd | | | MS m/z | Time |
| # | Structure | EMW | (M⁺ + H) | (min) |
| 444 | 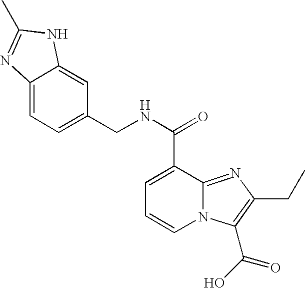 | 377.1 | 378.1 | 2.36 |
| 445 | 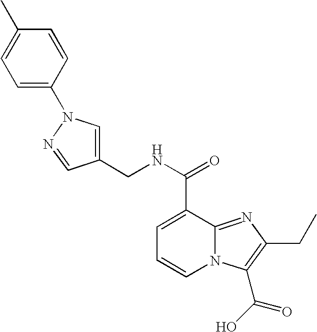 | 403.2 | 404.2 | 4.20 |
| 446 | 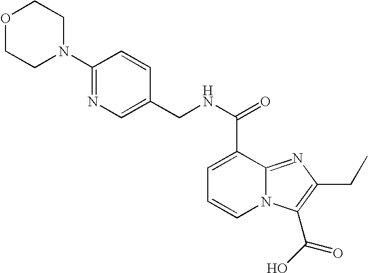 | 409.2 | 410.2 | 2.20 |
| 447 | 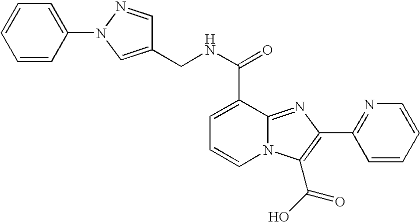 | 438.1 | 439.1 | 4.27 |
| 448 | 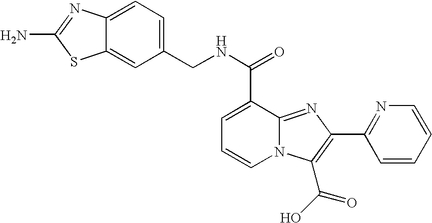 | 444.1 | 445.1 | 2.74 |
| 449 | 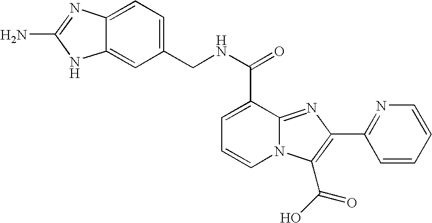 | 426.1 | 427.1 | 2.57 |
| 450 | 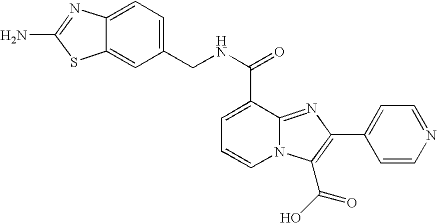 | 444.1 | 445.1 | 2.05 |

### Part D:

Compounds 451-506 (Table 18) were prepared using procedures described in Example 6E, Part A.

TABLE 18

| | | | MS | Ret. |
|---|---|---|---|---|
| Compd | | | m/z | Time |
| # | Structure | EMW | (M⁺ + H) | (min) |
| 451 | 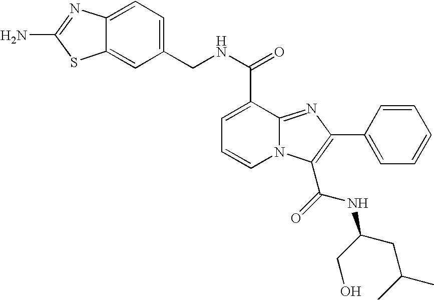 | 542.2 | 543.1 | 3.85 |
| 452 | 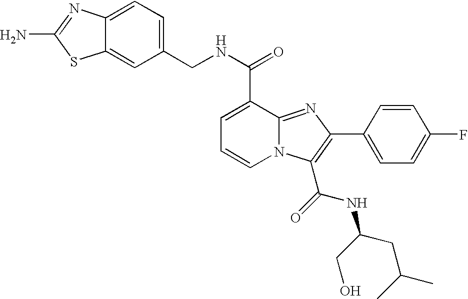 | 560.2 | 561.1 | 3.95 |
| 453 | 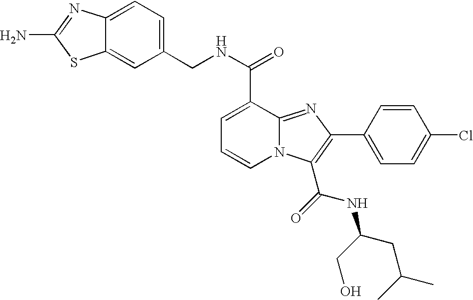 | 576.2 | 577.1 | 4.05 |
| 454 | 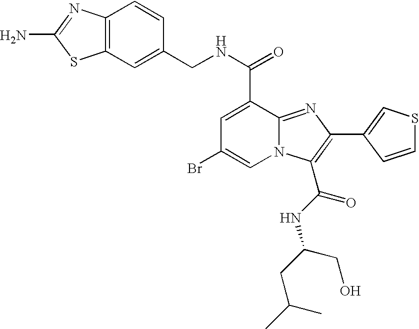 | 626.1 | 627.0 | 4.11 |
| 455 | 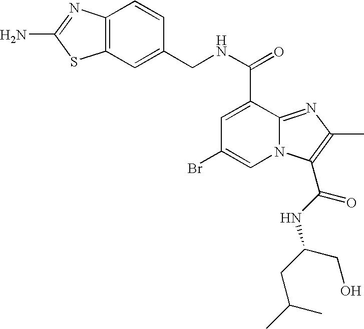 | 558.1 | 559.0 | 3.58 |
| 456 | 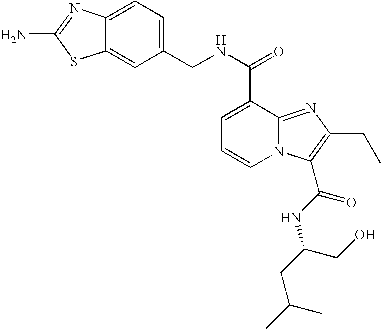 | 494.2 | 495.1 | 3.17 |
| 457 | 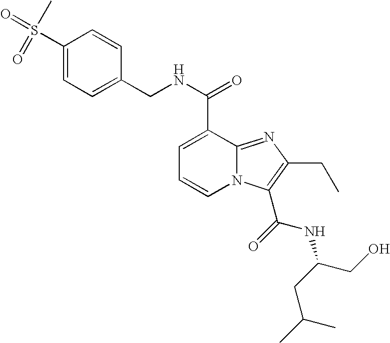 | 500.2 | 501.1 | 3.78 |
| 458 | 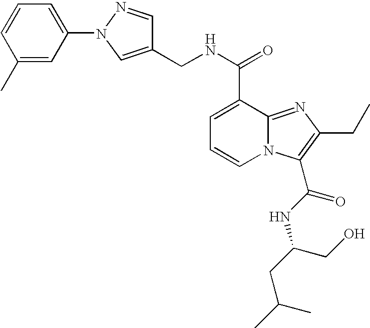 | 502.3 | 503.3 | 4.88 |
| 459 | 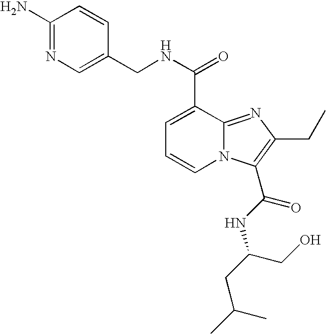 | 438.2 | 439.2 | 2.88 |
| 460 | 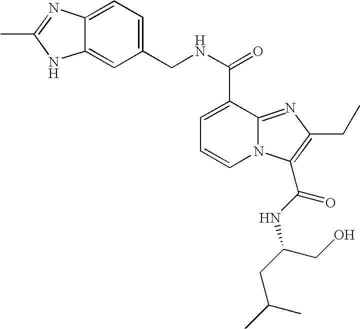 | 476.3 | 477.3 | 3.01 |
| 461 | 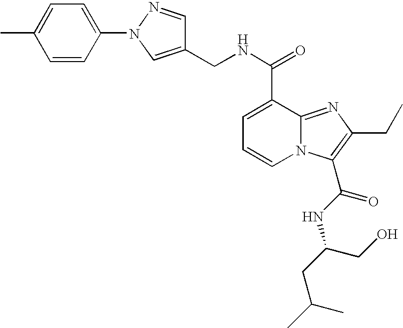 | 502.3 | 503.3 | 4.79 |
| 462 | 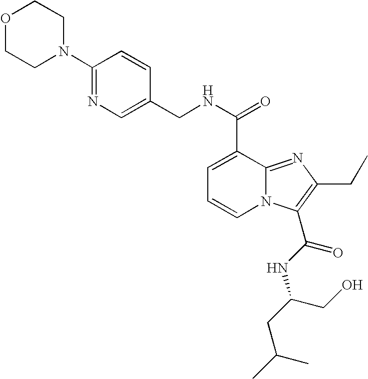 | 508.3 | 509.3 | 2.97 |
| 463 | 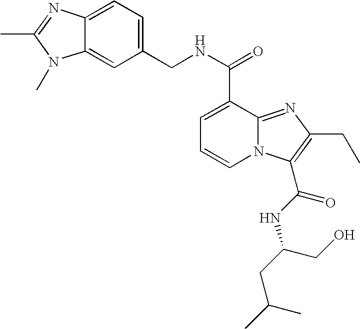 | 490.3 | 491.3 | 3.08 |
| 464 | 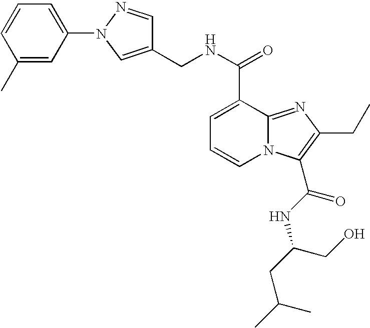 | 502.3 | 503.3 | 4.88 |
| 465 | 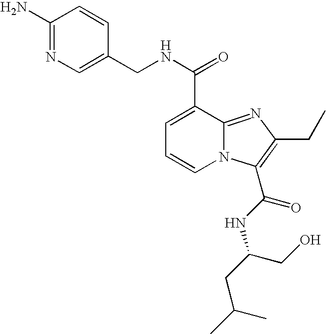 | 438.2 | 439.2 | 2.75 |
| 466 | 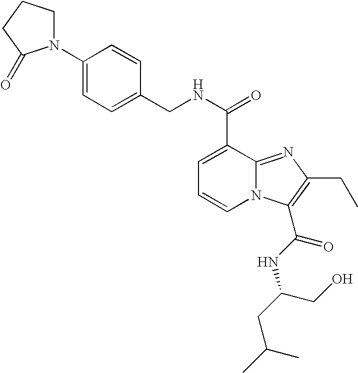 | 505.3 | 506.3 | 3.93 |
| 467 | 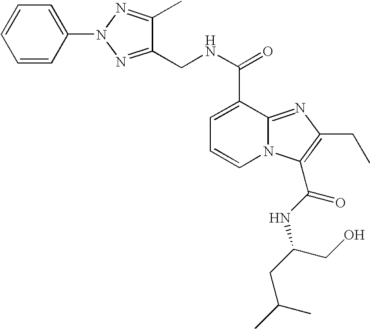 | 503.3 | 504.3 | 5.24 |
| 468 | 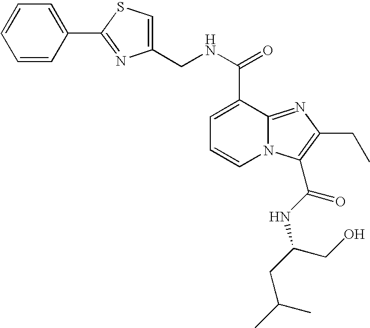 | 505.2 | 506.2 | 5.11 |
| 469 | 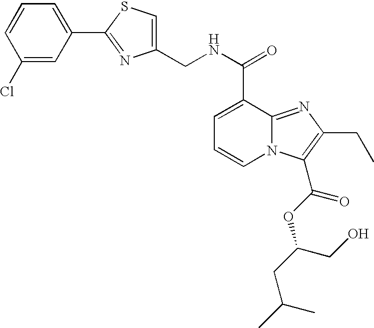 | 539.2 | 540.2 | 5.63 |
| 470 | 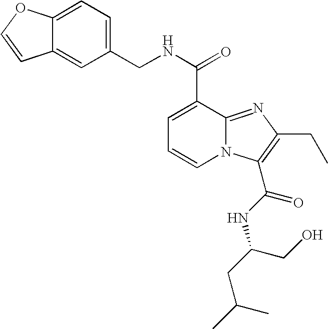 | 462.2 | 463.2 | 4.77 |
| 471 | 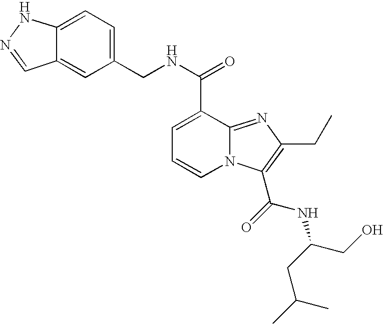 | 462.2 | 463.2 | 3.57 |
| 472 | 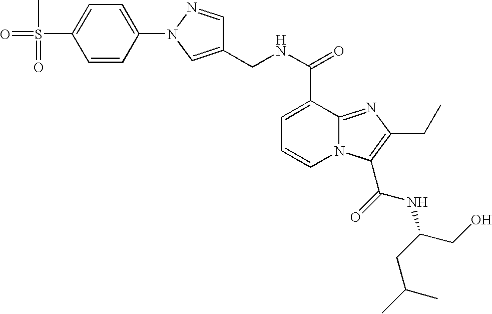 | 566.2 | 567.2 | 3.77 |
| 473 | 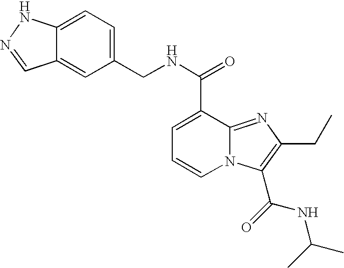 | 404.2 | 405.2 | 3.30 |
| 474 | 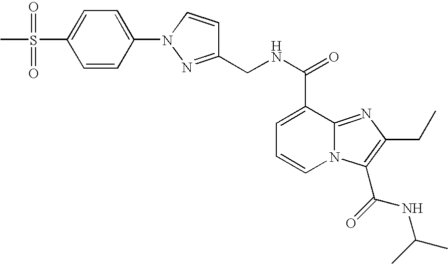 | 508.2 | 509.2 | 3.55 |
| 475 | 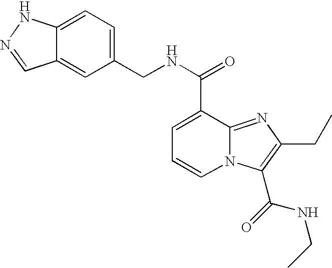 | 390.2 | 391.2 | 2.97 |
| 476 | 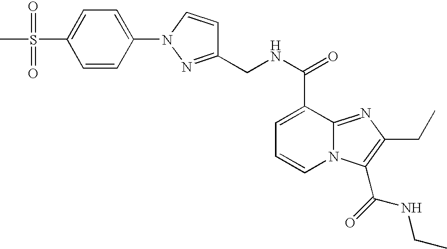 | 494.2 | 495.2 | 3.25 |
| 477 | 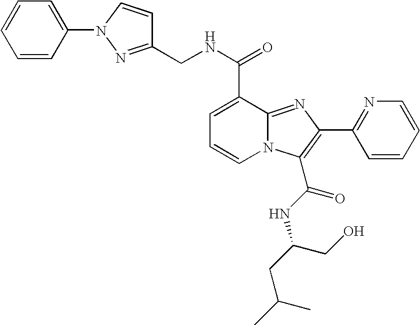 | 537.2 | 538.2 | 5.54 |
| 478 | 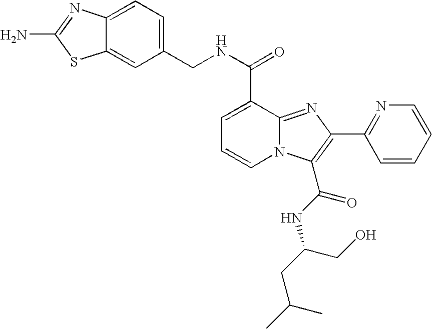 | 543.2 | 544.2 | 3.88 |
| 479 | 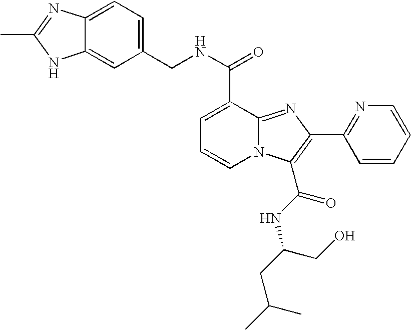 | 525.2 | 526.2 | 3.71 |
| 480 | 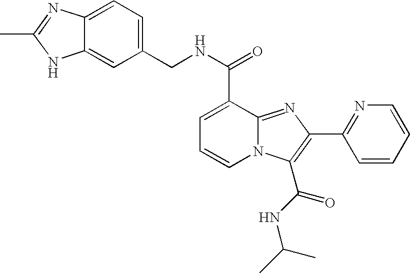 | 467.2 | 468.2 | 3.78 |
| 481 | 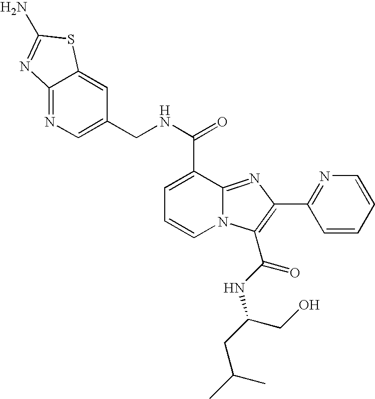 | 544.2 | 545.2 | 3.59 |
| 482 | 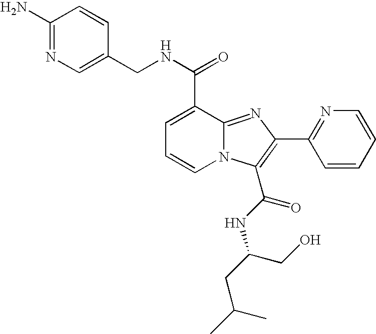 | 487.2 | 488.2 | 3.53 |
| 483 | 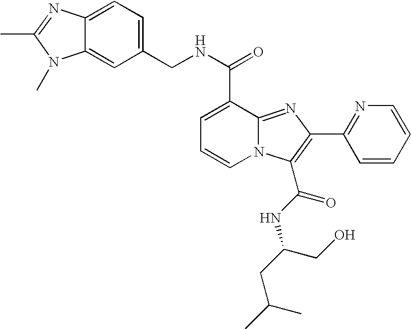 | 539.3 | 540.3 | 3.81 |
| 484 | 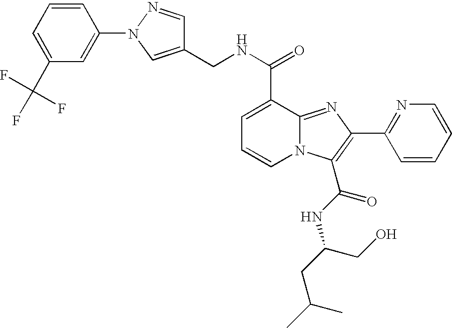 | 605.2 | 606.2 | 6.16 |
| 485 | 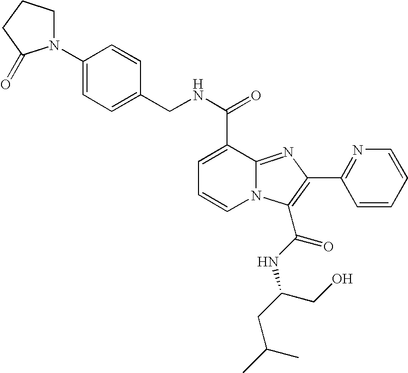 | 554.3 | 555.3 | 4.97 |
| 486 | 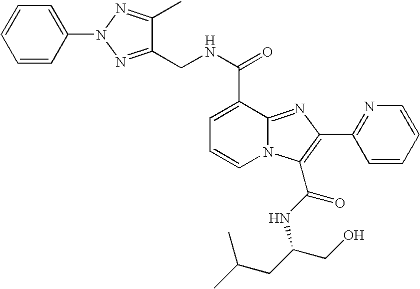 | 552.3 | 553.3 | 6.11 |
| 487 | 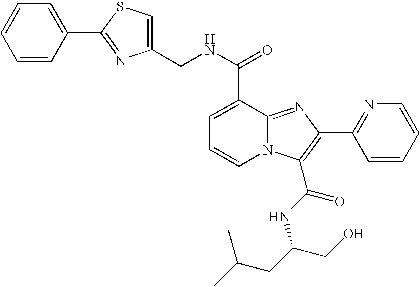 | 554.2 | 555.2 | 5.98 |
| 488 | 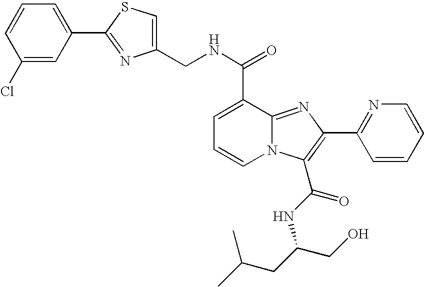 | 588.2 | 589.2 | 6.41 |
| 489 | 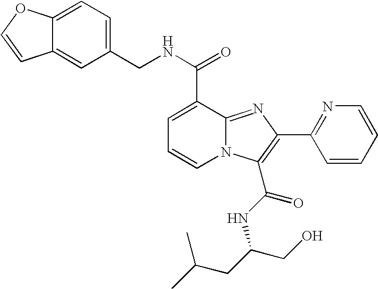 | 511.2 | 512.2 | 5.64 |
| 490 | 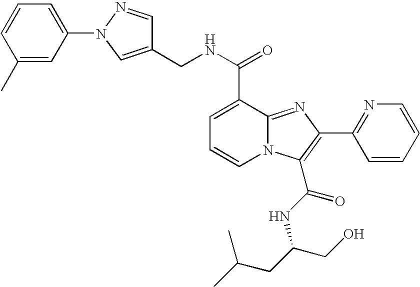 | 551.3 | 552.3 | 5.74 |
| 491 | 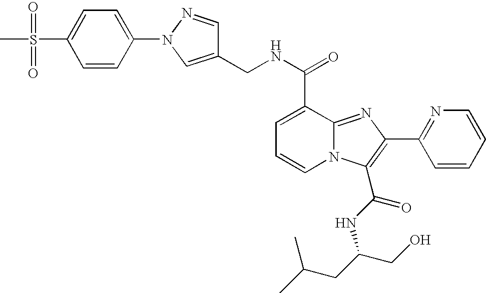 | 615.2 | 616.2 | 4.71 |
| 492 | 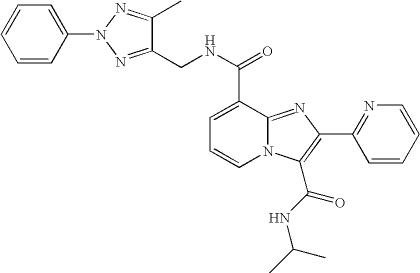 | 494.2 | 495.2 | 6.44 |
| 493 | 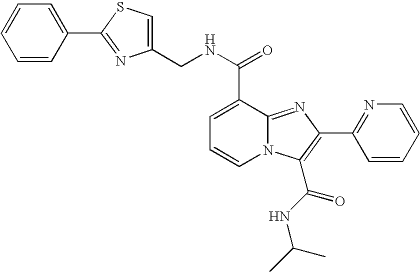 | 496.2 | 497.2 | 6.26 |
| 494 | 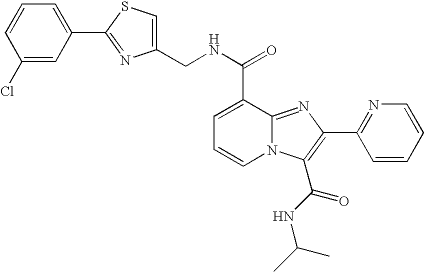 | 530.1 | 531.1 | 6.78 |
| 495 | 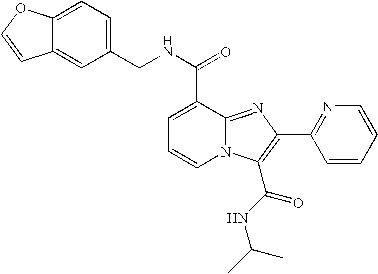 | 453.2 | 454.2 | 5.91 |
| 496 | 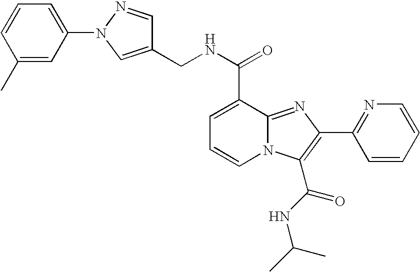 | 493.2 | 494.2 | 6.04 |
| 497 | 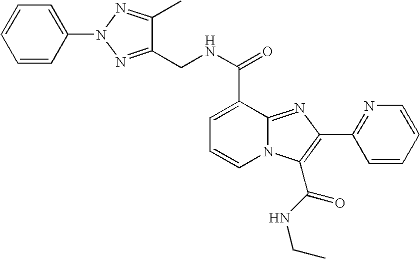 | 480.2 | 481.2 | 6.04 |
| 498 | 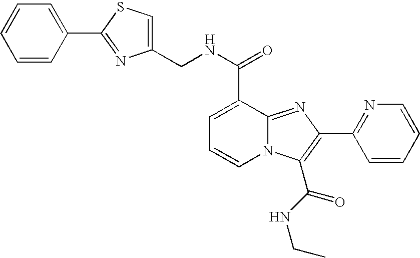 | 482.2 | 483.2 | 5.89 |
| 499 | 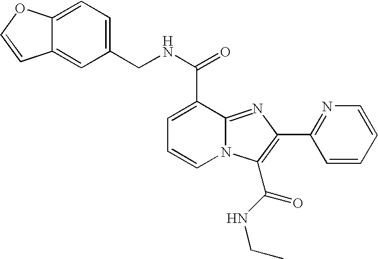 | 439.2 | 440.2 | 5.53 |
| 500 | 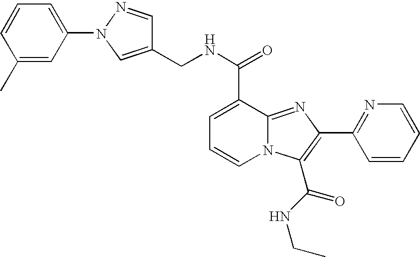 | 479.2 | 480.2 | 5.63 |
| 501 | 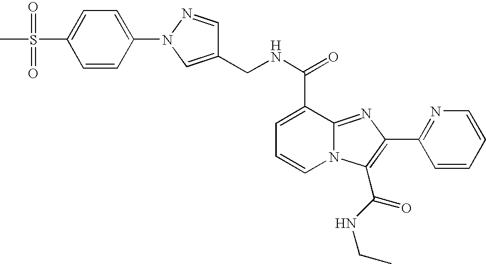 | 543.2 | 544.2 | 4.49 |
| 502 | 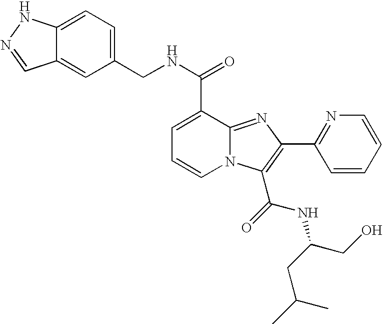 | 511.2 | 512.2 | 4.55 |
| 503 | 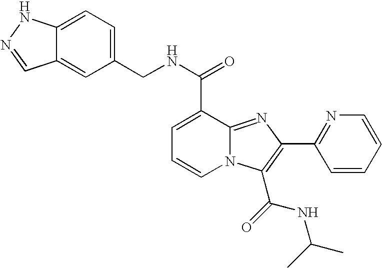 | 453.2 | 454.2 | 4.68 |
| 504 | 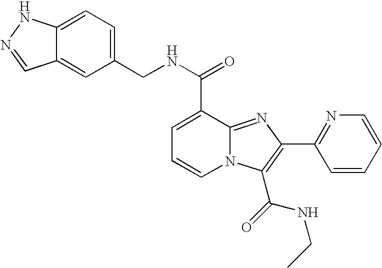 | 439.2 | 440.2 | 4.30 |
| 505 | 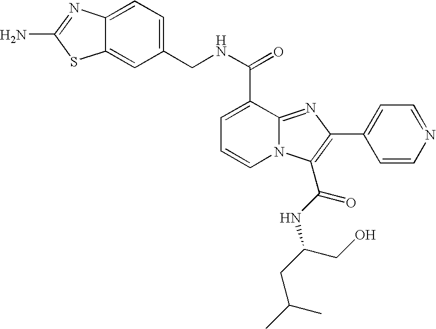 | 543.2 | 544.2 | 2.69 |
| 506 | 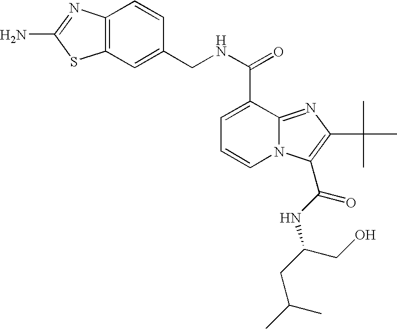 | 522.2 | 523.2 | 3.56 |

### Example 11

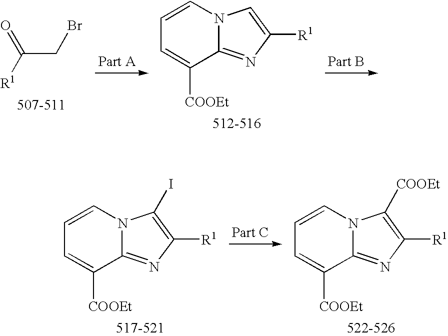

### Part A:

A mixture of 2-bromo-1-(thienyl)-1-ethanone 507 0.410 g, 2 mmol) and compound 2 (0.166 g, 1 mmol) in ethanol (5 mL) was heated at reflux for 48 hours. After cooling to room temperature, the reaction was monitored by LC-MS. The volatiles were removed in vacuo, ethyl acetate was added, and the organic solution washed successively with saturated NaHCO₃ (×1), water (×1), brine (×1), dried over magnesium sulfate and concentrated. The crude was purified by preparative Thin Layer Chromatography (SiO₂, dichloromethane/ethyl acetate—4:1) to afford compound 512 as a white solid. HPLC-MS t_{R}=1.07 min (UV_{254 nm}); mass calculated for formula C₁₄H₁₂N₂O₂S 272.1, observed LCMS m/z 273.0 (M+H).

Compound 513 was prepared from the reaction of chloroacetaldehyde 508 and compound 2. HPLC-MS t_{R}=0.21 min (UV_{254 nm}); mass calculated for formula C₁₀H₁₀N₂O₂ 190.1, observed LCMS m/z 191.1 (M+H).

Compound 514 was prepared from the reaction of 3,5-difluorophenacyl bromide 509 and compound 2. HPLC-MS t_{R}=1.47 min (UV_{254 nm}); mass calculated for formula C₁₆H₁₂F₂N₂O₂ 302.1, observed LCMS m/z 303.1 (M+H).

Compound 515 was prepared from the reaction of 2-fluorophenacyl bromide 510 and compound 2. HPLC-MS t_{R}=1.19 min (UV_{254 nm}); mass calculated for formula C₁₆H₁₃FN₂O₂ 284.1, observed LCMS m/z 285.0 (M+H).

Compound 516 was prepared from the reaction of 3-fluorophenacyl bromide 511 and compound 2. HPLC-MS t_{R}=1.19 min (UV_{254 nm}); mass calculated for formula C₁₆H₁₃FN₂O₂ 284.1, observed LCMS m/z 285.0 (M+H).

### Part B:

Compound 512 (0.100 g, 0.37 mmol) was dissolved in ethanol (5 mL). To this solution was added N-iodosuccinimide (0.125 g, 0.56 mmol) and the reaction mixture stirred at room temperature for 1 hour. The reaction was monitored by LC-MS. If necessary, excess N-iodosuccinimide (0.0832 g, 0.37 mmol) was added and the reaction mixture stirred for an additional hour. The volatiles were removed in vacuo. Ethyl acetate was added and the organic solution was washed successively with saturated NaHCO₃ (×1), water (×1), brine (×1), dried over magnesium sulfate and concentrated to afford compound 517, which was taken forward as crude to the next step. HPLC-MS t_{R}=1.98 min (UV_{254 nm}); mass calculated for formula C₁₄H₁₁IN₂O₂S 398.0, observed LCMS m/z 399.0 (M+H).

Compound 518 was prepared from the reaction of compound 513 and N-bromosuccinimide. HPLC-MS t_{R}=0.64 min (UV_{254 nm}); mass calculated for formula C₁₀H₉BrN₂O₂ 268.0, observed LCMS m/z 269.0 (M+H).

Compound 519 was prepared from the reaction of compound 514 and N-iodosuccinimide. HPLC-MS t_{R}=2.14 min (UV_{254 nm}); mass calculated for formula C₁₆H₁₁F₂IN₂O₂ 428.0, observed LCMS m/z 429.0 (M+H).

Compound 520 was prepared from the reaction of compound 515 and N-iodosuccinimide. HPLC-MS t_{R}=1.64 min (UV_{254 nm}), mass calculated for formula C₁₆H₁₂FIN₂O₂ 410.0, observed LCMS m/z 411.0 (M+H).

Compound 521 was prepared from the reaction of compound 516 and N-iodosuccinimide. HPLC-MS t_{R}=1.98 min (UV_{254 nm}), mass calculated for formula C₁₆H₁₂FIN₂O₂ 410.0, observed LCMS m/z 411.0 (M+H).

### Part C:

To a mixture of compound 517 (0.063 g, 0.16 mmol), molybdenum hexacarbonyl (0.084 g, 0.32 mmol), diisopropylethylamine (0.030 mL, 0.18 mmol) in ethanol (3 mL) was added Pd(DPPF)Cl₂. DCM (10 mol %). The reaction vessel was flushed with argon, capped and heated at 80° C. for 16 hours. After cooling to room temperature, the reaction was shown to be incomplete by LC-MS. Excess molybdenum hexacarbonyl (0.084 g, 0.32 mmol) was added and the reaction mixture heated for another 16 hours. The precipitates were removed by filtration, the filtrate concentrated and purified by preparative thin layer chromatography (SiO₂, dichloromethane/ethyl acetate—15:1) to afford compound 522 as a yellow solid. HPLC-MS t_{R}=2.05 min (UV_{254 nm}); mass calculated for formula C₁₇H₁₆N₂O₄S 344.1, observed LCMS m/z 345.1 (M+H).

Compound 523 was prepared from compound 518. HPLC-MS t_{R}=1.22 min (UV_{254 nm}); mass calculated for formula C₁₃H₁₄N₂O₄ 262.1, observed LCMS m/z 263.1 (M+H).

Compound 524 was prepared from compound 519. HPLC-MS t_{R}=2.83 min (UV_{254 nm}); mass calculated for formula C₁₉H₁₆F₂N₂O₄ 374.1, observed LCMS m/z 375.1 (M+H).

Compound 525 was prepared from compound 520. HPLC-MS t_{R}=1.96 min (UV_{254 nm}); mass calculated for formula C₁₉H₁₇FN₂O₄ 356.1, observed LCMS m/z 357.1 (M+H).

Compound 526 was prepared from compound 521. HPLC-MS t_{R}=2.74 min (UV_{254 nm}); mass calculated for formula C₁₉H₁₇FN₂O₄ 356.1, observed LCMS m/z 357.1 (M+H).

### Example 11B

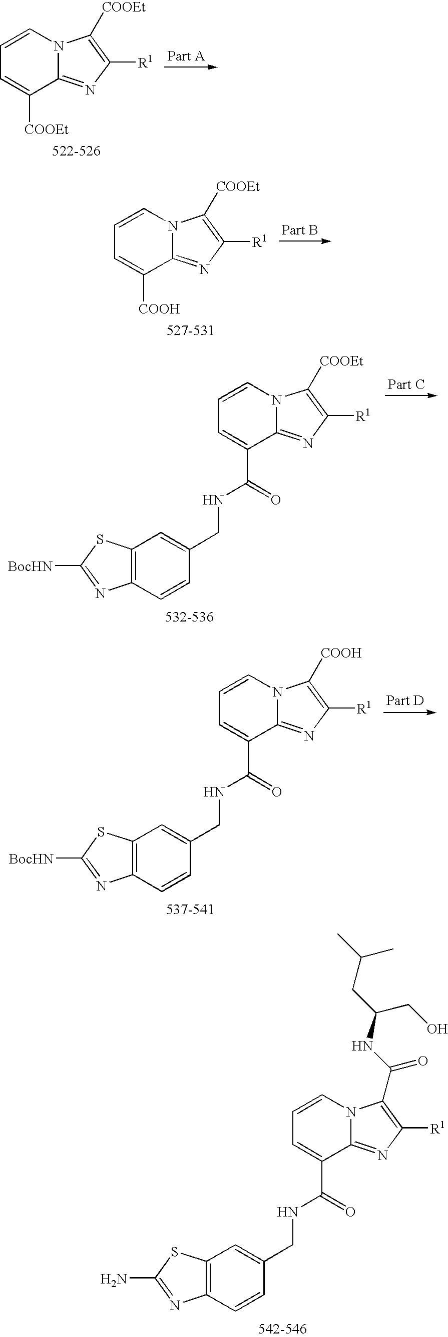

(wherein R¹ is identified in Table 19)

### Part A:

Compound 527 was prepared from the saponification of compound 522 using procedures described in Example 5K, Part A. HPLC-MS t_{R}=1.91 min (UV_{254 nm}); mass calculated for formula C₁₅H₁₂N₂O₄S 316.1, observed LCMS m/z 317.1 (M+H).

Compound 528 was prepared from compound 523. HPLC-MS t_{R}=0.77 min (UV_{254 nm}); mass calculated for formula C₁₁H₁₀N₂O₄ 234.1, observed LCMS m/z 235.1 (M+H).

Compound 529 was prepared from compound 524. HPLC-MS t_{R}=2.63 min (UV_{254 nm}); mass calculated for formula C₁₇H₁₂F₂N₂O₄ 346.1, observed LCMS m/z 347.0 (M+H).

Compound 530 was prepared from compound 525. HPLC-MS t_{R}=1.78 min (UV_{254 nm}); mass calculated for formula C₁₇H₁₃FN₂O₄ 328.1, observed LCMS m/z 329.0 (M+H).

Compound 531 was prepared from compound 526. HPLC-MS t_{R}=2.46 min (UV_{254 nm}); mass calculated for formula C₁₇H₁₃FN₂O₄ 328.1, observed LCMS m/z 329.0 (M+H).

### Part B:

Compound 532 was prepared from the coupling of compound 527 and (6-aminomethyl-benzothiazol-2-yl)-carbamic acid tert-butyl ester using procedures described in Example 3A, Part C. HPLC-MS t_{R}=2.33 min (UV_{254 nm}); mass calculated for formula C₂₈H₂₇N₅O₅S₂ 577.1, observed LCMS m/z 578.0 (M+H).

Compound 533 was prepared from compound 528. HPLC-MS t_{R}=2.13 min (UV_{254 nm}); mass calculated for formula C₂₄H₂₅N₅O₅S 495.2, observed LCMS m/z 496.1 (M+H).

Compound 534 was prepared from compound 529. HPLC-MS t_{R}=2.42 min (UV_{254 nm}); mass calculated for formula C₃₀H₂₇F₂N₅O₅S 607.2, observed LCMS m/z 608.0 (M+H).

Compound 535 was prepared from compound 530. HPLC-MS t_{R}=2.33 min (UV_{254 nm}); mass calculated for formula C₃₀H₂₈FN₅O₅S 589.2, observed LCMS m/z 590.0 (M+H).

Compound 536 was prepared from compound 531. HPLC-MS t_{R}=3.63 min (UV_{254 nm}); mass calculated for formula C₃₀H₂₈FN₅O₅S 589.2, observed LCMS m/z 590.0 (M+H).

### Part C:

Compound 537 was prepared from the saponification of compound 532 using procedures described in Example 3A, Part D. HPLC-MS t_{R}=1.91 min (UV_{254 nm}); mass calculated for formula C₂₆H₂₃N₅O₅S₂ 549.1, observed LCMS m/z 550.0 (M+H).

Compound 538 was prepared from compound 533. HPLC-MS t_{R}=1.64 min (UV_{254 nm}); mass calculated for formula C₂₂H₂₁N₅O₅S 467.1, observed LCMS m/z 468.1 (M+H).

Compound 539 was prepared from compound 534. HPLC-MS t_{R}=1.99 min (UV_{254 nm}); mass calculated for formula C₂₈H₂₃F₂N₅O₅S 579.1, observed LCMS m/z 580.0 (M+H).

Compound 540 was prepared from compound 535. HPLC-MS t_{R}=1.90 min (UV_{254 nm}); mass calculated for formula C₂₈H₂₄FN₅O₅S 561.1, observed LCMS m/z 562.0 (M+H).

Compound 541 was prepared from compound 536. HPLC-MS t_{R}=1.95 min (UV_{254 nm}); mass calculated for formula C₂₈H₂₄FN₅O₅S 561.1, observed LCMS m/z 562.0 (M+H).

### Part D:

Compounds 542-546 (Table 19) were prepared using coupling procedures described in Example 7A, Part E.

TABLE 19

| | | | | Ret. |
|---|---|---|---|---|
| | | | MS m/z | Time |
| compd # | Structure | EMW | (M⁺+ H) | (min) |
| 542 | 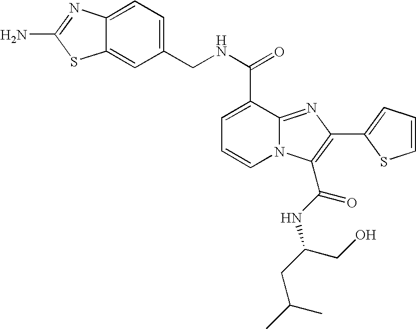 | 548.2 | 549.2 | 3.81 |
| 543 | 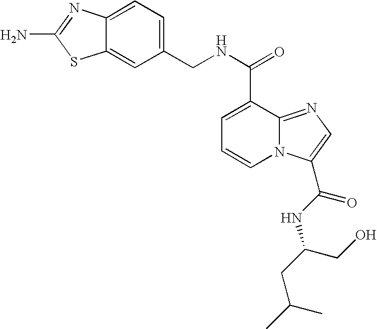 | 466.2 | 467.2 | 3.01 |
| 544 | 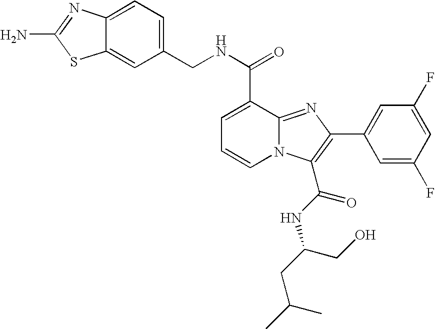 | 578.2. | 579.2 | 4.07 |
| 545 | 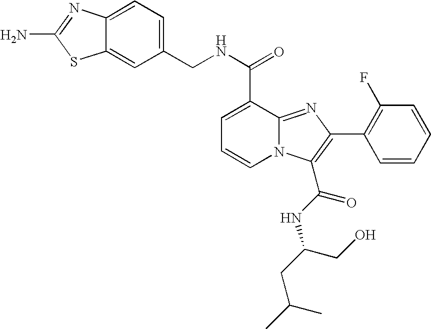 | 560.2 | 561.1 | 3.66 |
| 546 | 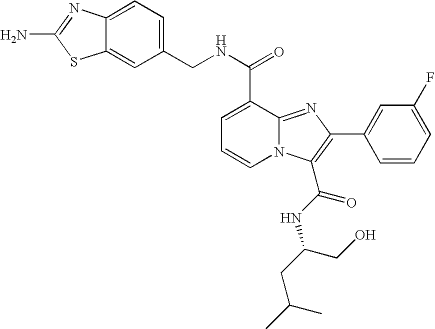 | 560.2 | 561.0 | 3.76 |

### Example 12A

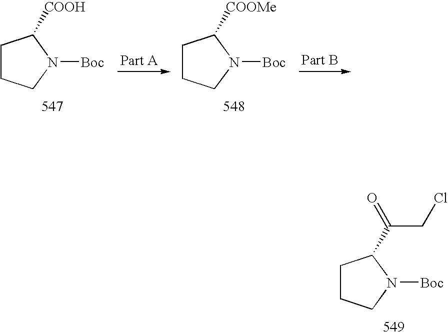

### Part A:

To a solution of Boc-D-proline 547 (25.0 g, 0.116 mol) in methanol (50 mL) and acetonitrile (50 mL) was added (trimethylsilyl)diazomethane (2M, 116 mL, 0.232 mol), and the reaction mixture stirred at room temperature for 16 hours. The reaction was monitored by Thin Layer Chromatography (hexanes/ethyl acetate—4:1). Acetic acid (5 mL) was added to quench the excess (trimethylsilyl)diazomethane. The volatiles were removed in vacuo, and the crude product purified by flash column chromatography to afford compound 548 as a colorless oil.

### Part B:

A fresh solution of lithium diisopropylamide (LDA) was prepared by adding n-butyllithium (2.5M, 87 mL, 0.218 mol) to a stirred solution of diisopropylamine (32 mL, 0.229 mol) in THF (50 mL) at −78° C., under an inert atmosphere. The LDA solution was warmed to −20° C. (salt ice-bath) with stirring for 1 hour. Chloroiodomethane (16 mL, 0.218 mol) was added to a solution of compound 548 (10.0 g, 0.044 mol) in THF (50 mL), and cooled to −78° C. The LDA solution was transferred via cannula to the reaction mixture over a period of 90 minutes, and then the mixture was stirred for an additional 1 hour at −78° C. A solution of acetic acid (7.5 mL) in THF (20 mL) was added slowly to the reaction, maintaining the temperature below −70° C. The reaction mixture was stirred for an additional 10 minutes at −70° C. and then warmed to room temperature. Ethyl acetate (100 mL) was added and the precipitates removed by filtration. The filtrate was washed with water (×1), saturated Na₂HPO₄ (×1), saturated NaHCO₃ (×1), water (×1), brine (×1), dried over magnesium sulfate and concentrated. The crude was purified by flash column chromatography (SiO₂, hexanes/ethyl Acetate—4:1) to afford compound 549 as a deep red oil.

### Example 12B

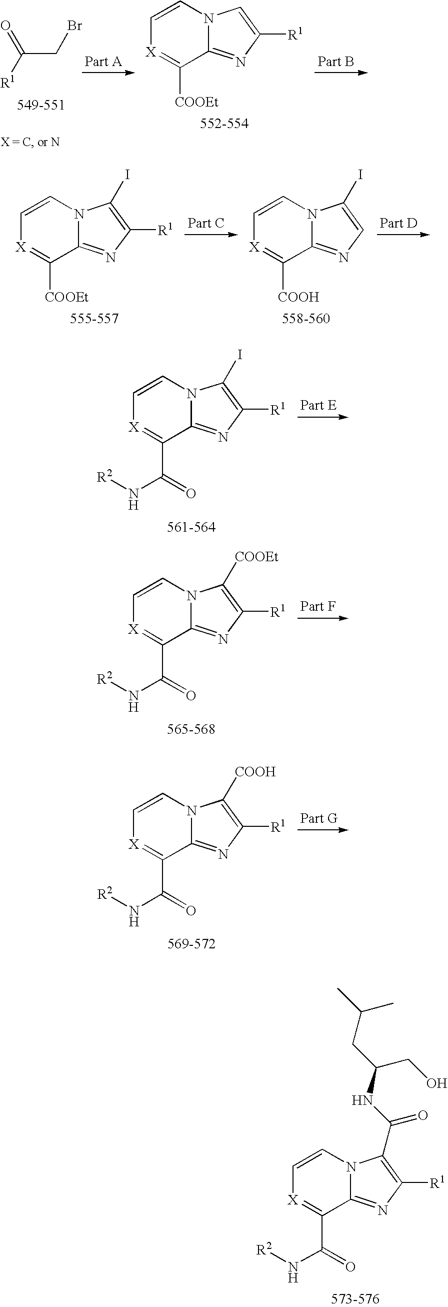

### Part A:

Compound 552 was prepared from the reaction of (R)-1-boc-2-(2′-chloroacetyl)-pyrrolidine 549 and compound 2 using procedures described in Example 11A, Part A. HPLC-MS t_{R}=0.63 min (UV_{254 nm}); mass calculated for formula C₁₉H₂₅N₃O₄ 359.2, observed LCMS m/z 360.1 (M+H).

Compound 553 was prepared from the reaction of (S)-1-boc-2-(2′-chloroacetyl)-pyrrolidine 550 and compound 2. HPLC-MS t_{R}=0.68 min (UV_{254 nm}); mass calculated for formula C₁₉H₂₅N₃O₄ 359.2, observed LCMS m/z 360.2 (M+H).

Compound 554 was prepared from the reaction of 1-bromo-2-butanone 551 and methyl 3-amino-2-pyrazolecarboxylate. HPLC-MS t_{R}=0.73 min (UV_{254 nm}); mass calculated for formula C₁₁H₁₃N₃O₂ 219.1, observed LCMS m/z 220.1 (M+H).

### Part B:

Compound 555 was prepared from compound 552 using procedures described in Example 6A, Part B. HPLC-MS t_{R}=1.60 min (UV_{254 nm}); mass calculated for formula C₁₉H₂₄IN₃O₄ 485.1, observed LCMS m/z 486.0 (M+H).

Compound 556 was prepared from compound 553. HPLC-MS t_{R}=1.67 min (UV_{254 nm}); mass calculated for formula C₁₉H₂₄IN₃O₄ 485.1, observed LCMS m/z 486.0 (M+H).

Compound 557 was prepared from compound 554. HPLC-MS t_{R}=1.45 min (UV_{254 nm}); mass calculated for formula C₁₁H₁₂IN₃O₂ 345.0, observed LCMS m/z 346.0 (M+H).

### Part C:

Compound 558 was prepared from the saponification of compound 555 using procedures described in Example 5K, Part A. HPLC-MS t_{R}=1.42 min (UV_{254 nm}); mass calculated for formula C₁₇H₂₀IN₃O₄ 457.0, observed LCMS m/z 458.0 (M+H).

Compound 559 was prepared from compound 556. HPLC-MS t_{R}=1.42 min (UV_{254 nm}); mass calculated for formula C₁₇H₂₀IN₃O₄ 457.0, observed LCMS m/z 458.0 (M+H).

Compound 560 was prepared from compound 557. HPLC-MS t_{R}=0.79 min (UV_{254 nm}); mass calculated for formula C₉H₈IN₃O₂ 317.0, observed LCMS m/z 318.0 (M+H).

### Part D:

Compounds 561 was prepared from the coupling of compound 558 and (6-aminomethyl-benzothiazol-2-yl)-carbamic acid tert-butyl ester using procedures described in Example 3A, Part C. HPLC-MS t_{R}=2.31 min (UV_{254 nm}); mass calculated for formula C₃₀H₃₅IN₆O₅S 718.0, observed LCMS m/z 719.0 (M+H).

Compound 562 was prepared from the coupling of compound 558 and (6-aminomethyl-benzothiazol-2-yl)-carbamic acid tert-butyl ester. HPLC-MS t_{R}=2.33 min (UV_{254 nm}); mass calculated for formula C₃₀H₃₅IN₆O₅S 718.0, observed LCMS m/z 719.0 (M+H).

Compound 563 was prepared from the coupling of compound 559 and 1-(4-aminomethylphenyl)pyrrolidin-2-one. HPLC-MS t_{R}=2.00 min (UV_{254 nm}); mass calculated for formula C₂₈H₃₂IN₅O₄ 629.1, observed LCMS m/z 630.0 (M+H).

Compound 564 was prepared from the coupling of compound 560 and (6-aminomethyl-benzothiazol-2-yl)-carbamic acid tert-butyl ester. HPLC-MS t_{R}=1.92 min (UV_{254 nm}); mass calculated for formula C₂₂H₂₃IN₆O₃S 578.1, observed LCMS m/z 579.0 (M+H).

### Part E:

Compound 565 was prepared from compound 561 using carbonylation procedures described in Example 6A, Part C. HPLC-MS t_{R}=2.35 min (UV_{254 nm}); mass calculated for formula C33H₄₀N₆O₇S 664.3, observed LCMS m/z 665.2 (M+H).

Compound 566 was prepared from compound 562. HPLC-MS t_{R}=2.35 min (UV_{254 nm}); mass calculated for formula C₃₃H₄₀N₆O₇S 664.3, observed LCMS m/z 665.2 (M+H).

Compound 567 was prepared from compound 563. HPLC-MS t_{R}=2.06 min (UV_{254 nm}); mass calculated for formula C₃₁H₃₇N₅O₆ 575.3, observed LCMS m/z 576.2 (M+H).

Compound 568 was prepared from compound 564. HPLC-MS t_{R}=2.03 min (UV_{254 nm}); mass calculated for formula C₂₅H₂₈N₁₆O₅S 524.2, observed LCMS m/z 525.1 (M+H).

### Part F:

Compound 569 was prepared from the saponification of compound 565 using procedures described in Example 7A, Part D. HPLC-MS t_{R}=1.95 min (UV_{254 nm}); mass calculated for formula C₃₁H₃₆N₆O₇S 636.2, observed LCMS m/z 637.2 (M+H).

Compound 570 was prepared from compound 566. HPLC-MS t_{R}=1.94 min (UV_{254 nm}); mass calculated for formula C₃₁H₃₆N₆O₇S 636.2, observed LCMS m/z 637.1 (M+H).

Compound 571 was prepared from compound 567. HPLC-MS t_{R}=1.59 min (UV_{254 nm}); mass calculated for formula C₂₉H₃₃N₅O₆ 547.2, observed LCMS m/z 548.2 (M+H).

Compound 572 was prepared from compound 568. HPLC-MS t_{R}=1.61 min (UV_{254 nm}); mass calculated for formula C₂₃H₂₄N₆O₅S 496.2, observed LCMS m/z 497.0 (M+H).

### Part G:

Compounds 573-576 (Table 20) were prepared using coupling procedures described in Example 7A, Part E.

TABLE 20

| | | | | Ret. |
|---|---|---|---|---|
| | | | MS m/z | Time |
| Compd # | Structure | EMW | (M⁺+ H) | (min) |
| 573 | 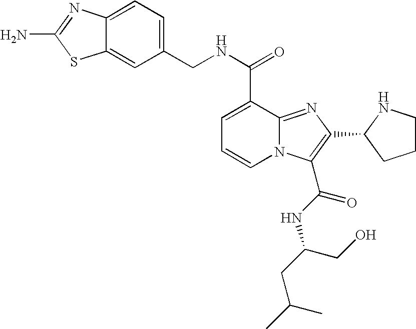 | 535.2 | 536.2 | 2.62 |
| 574 | 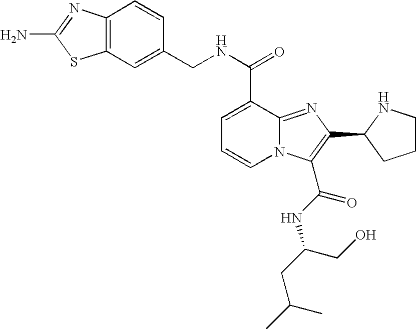 | 546.3 | 547.3 | 3.21 |
| 575 | 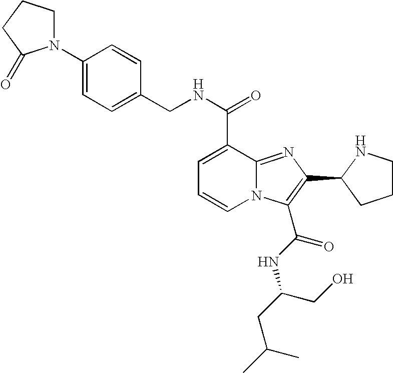 | 535.2 | 536.2 | 2.58 |
| 576 | 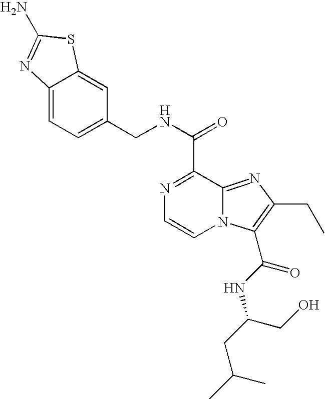 | 495.2 | 496.1 | 2.80 |

### Example 13

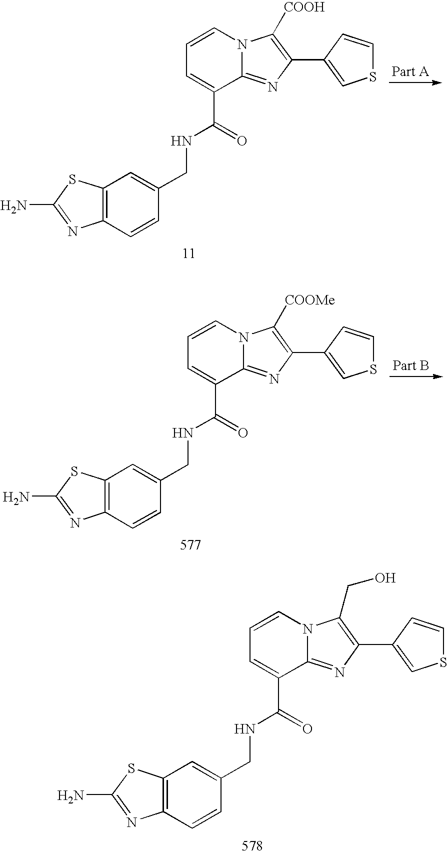

Compound 11 was prepared using procedures described in Example 1B, Part A-H.

### Part A:

To a solution of compound 11 (0.15 mmol) in acetonitrile (2 mL) and methanol (2 mL) was added (trimethylsilyl)diazomethane (2M, 0.11 mL, 0.22 mmol). The reaction mixture was stirred at room temperature for 30 minutes. LC-MS analysis of the reaction indicated that the reaction was complete. The volatiles were removed in vacuo to afford compound 577 (100% yield). HPLC-MS t_{R}=3.80 min (UV_{254 nm}); mass calculated for formula C₂₂H₁₇N₅O₃S₂ 463.1, observed LCMS m/z 464.0 (M+H).

### Part B:

Compound 577 (0.010 g, 0.02 mmol) was dissolved in a mixture of THF (0.5 mL) and methanol (0.5 mL). Lithium borohydride (0.0014 g, 0.07 mmol) was added, and the reaction mixture heated at 55° C. for 1 hour. On cooling to room temperature, the reaction was monitored by LC-MS. The volatiles were removed in vacuo. Ethyl acetate was added, and the organic solution washed with saturated NaHCO₃ (×1), brine (×1), dried over magnesium sulfate and concentrated to afford compound 578 which was purified by PrepLC.

The following ligand was synthesized using this procedure:

| | | | | Ret. |
|---|---|---|---|---|
| | | | MS m/z | Time |
| compd # | Structure | EMW | (M⁺+ H) | (min) |
| 578 | 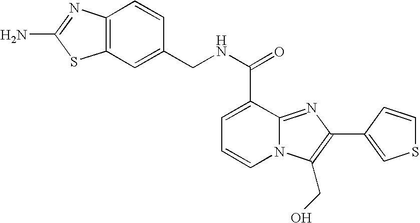 | 435.1 | 436.0 | 2.51 |

### Example 14A

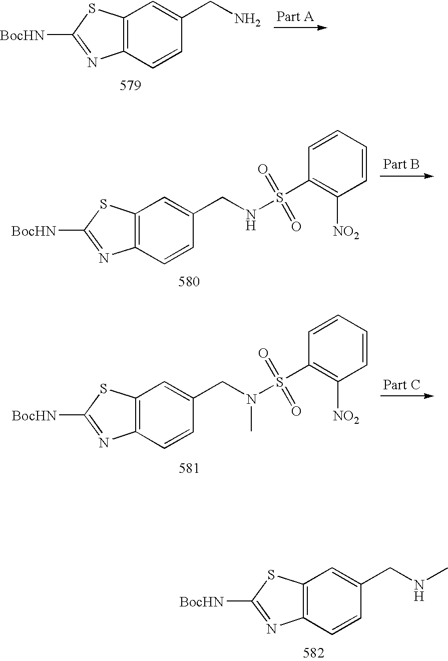

### Part A:

(6-aminomethyl-benzothiazol-2-yl)-carbamic acid tert-butyl ester 579 (0.100 g, 0.36 mmol) was dissolved in a mixture of dichloromethane (6 mL) and pyridine (2 mL). 2-Nitrobenzenesulfonyl chloride (0.087 g, 0.4 mmol) was added, and the reaction mixture stirred at room temperature for 4 hours. The reaction was monitored by LC-MS. The volatiles were removed in vacuo. Ethyl acetate was added, and the organic solution washed with saturated NaHCO₃ (×1), brine (×1), dried over magnesium sulfate and concentrated to afford compound 580 which was taken forward as crude to the next step. HPLC-MS t_{R}=1.86 min (UV_{254 nm}); mass calculated for formula C₁₉H₂₀N₄O₆S₂ 464.1, observed LCMS m/z 465.0 (M+H).

### Part B:

A mixture of compound 580 (0.36 mmol), potassium carbonate (50 mg, 0.36 mmol) and iodomethane (0.051 g, 0.36 mmol) in DMF (2 mL) was stirred at room temperature for 16 hours. The reaction was monitored by LC-MS. The volatiles were removed in vacuo. Ethyl acetate was added, and the organic solution washed with brine (×1), dried over magnesium sulfate and concentrated to afford compound 581 which was taken forward as crude to the next step. HPLC-MS t_{R}=2.15 min (UV₂₅₄ nm); mass calculated for formula C₂₀H₂₂N₄O₆S₂ 478.1, observed LCMS m/z 479.0 (M+H).

### Part C:

A mixture of compound 581 (0.030 g, 0.06 mmol), potassium carbonate (0.0095 g, 0.07 mmol) and benzenethiol (0.007 mL, 0.075 mmol) in DMF (2 mL) was stirred at room temperature for 16 hours. The reaction was monitored by LC-MS. Excess benzenthiol (0.014 mL, 0.15 mmol) was added and the reaction mixture stirred at room temperature for an addition 16 hours. The volatiles were removed in vacuo to afford compound 582 which was taken forward as crude to the next step. HPLC-MS t_{R}=1.17 min (UV_{254 nm}); mass calculated for formula C₁₄H₁₉N₃O₂S 293.1, observed LCMS m/z 294.1 (M+H).

### Example 14B

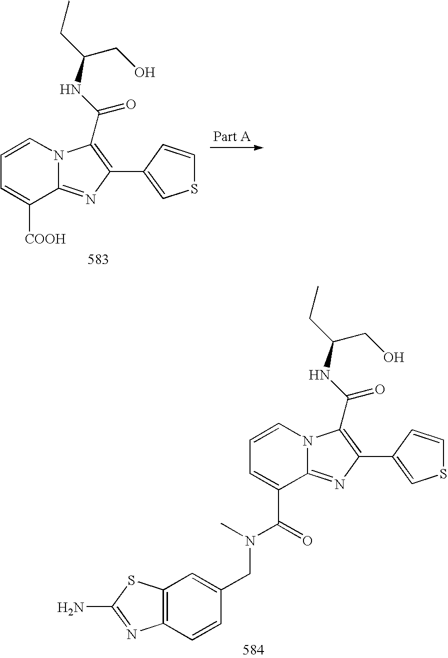

Compound 583 was prepared using procedures described in Example 13, Part A-D. Example 6. HPLC-MS t_{R}=0.99 min (UV_{254 nm}); mass calculated for formula C₁₇H₁₇N₃O₄S 359.1, observed LCMS m/z 360.1 (M+H).

### Part A:

Compound 584 was prepared using the coupling procedures described in Example 6D, Part A.

| | | | | Ret. |
|---|---|---|---|---|
| | | | MS m/z | Time |
| Compd # | Structure | EMW | (M⁺+ H) | (min) |
| 584 | 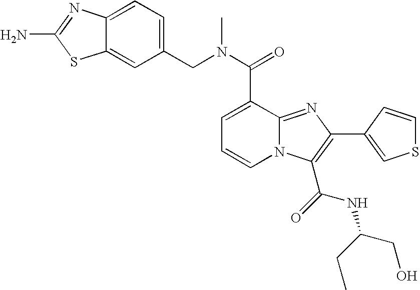 | 534.2 | 535.1 | 3.32 |

### Example 15

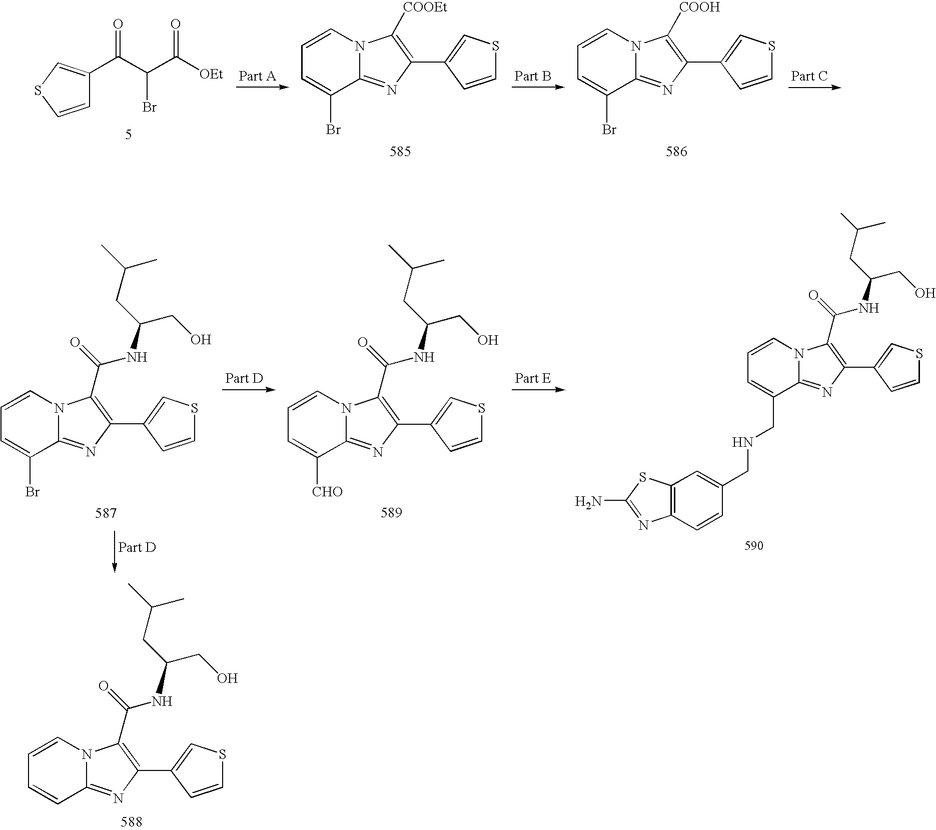

Compound 5 was prepared using procedures described in Example 1B.

### Part A:

Compound 585 was prepared from compound 5 and 2-amino-3-bromopyrdine using procedures described in Example 7A, Part A. HPLC-MS t_{R}=2.10 min (UV₂₅₄ nm); mass calculated for formula C₁₄H₁₁BrN₂O₂S 350.0, observed LCMS m/z 351.0 (M+H).

### Part B:

Compound 586 was prepared from compound 585 using procedures described in Example 7A, Part D. HPLC-MS t_{R}=1.47 min (UV_{254 nm}); mass calculated for formula C₁₂H₇BrN₂O₂S 321.9, observed LCMS m/z 322.9 (M+H).

### Part C:

Compound 587 was prepared from compound 586 using procedures described in Example 9C, Part C. HPLC-MS t_{R}=1.71 min (UV_{254 nm}); mass calculated for formula C₁₈H₂₀BrN₃O₂S 421.0, observed LCMS m/z 422.0 (M+H).

### Part D:

Compound 587 (0.022 g, 0.052 mmol) was dissolved in a mixture of DMF (1 mL) and THF (2 mL). n-Butyllithium (2.5M, 0.053 mL, 0.16 mmol) was added, and the reaction mixture stirred at room temperature for 1 hour. The reaction was monitored by LC-MS, indicating formation of the desired aldehyde, but also de-bromination side product. The volatiles were removed in vacuo. Ethyl acetate was added, and the organic solution washed with saturated NaHCO₃ (×1), brine (×1), dried over magnesium sulfate and concentrated to afford a mixture of compounds 588 and 589 which were taken forward to the next step.

### Part E:

Compound 589 (0.052 mmol) was dissolved in 1,2-dichloroethane (2 mL). (6-aminomethyl-benzothiazol-2-yl)-carbamic acid tert-butyl ester 578 (0.022 g, 0.078 mmol), acetic acid (0.200 mL) and sodium triacetoxyborohydride (0.0121 g, 0.06 mmol) was added, and the reaction mixture stirred at room temperature for 16 hours. The reaction was monitored by LC-MS, quenched by the addition of saturated NaHCO₃, extracted into dichloromethane, dried over magnesium sulfate and concentrated. The crude was redissolved in dioxane (1 mL), and a solution of 4 N HCl in dioxane (2 mL) and water (0.2 mL) was added at 0° C. (ice-bath). The reaction mixture was stirred at room temperature for 3 hours. LC-MS analysis of the reaction indicated that the reaction was complete. The volatiles were removed in vacuo, acetonitrile was added, concentrated and dried. Purification by Prep-LC and conversion to the hydrochloric salt afforded compounds 588 and 590 (Table 21) as white solids.

TABLE 21

| | | | | Ret. |
|---|---|---|---|---|
| | | | MS m/z | Time |
| Compd # | Structure | EMW | (M⁺+ H) | (min) |
| 588 | 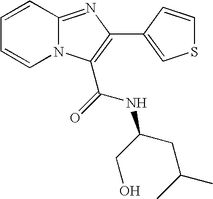 | 343.1 | 344.1 | 2.99 |
| 590 | 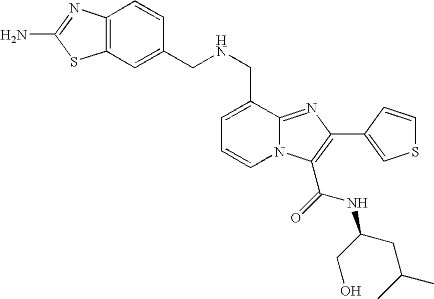 | 534.2 | 535.2 | 2.79 |

### Example 16A

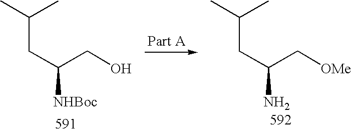

### Part A:

A mixture of N-(tert-butoxycarbonyl)-L-leucinol (0.500 g, 2.3 mmol), silver oxide (2.67 g, 11.5 mmol) and iodomethane (1.43 mL, 23 mmol) in acetonitrile (20 mL) was stirred at room temperature for 72 hours. The reaction was monitored by LC-MS. The precipitates were removed by filtration, the filtrate concentrated and the crude purified by flash column chromatography (SiO₂, dichloromethane/ethyl acetate—10:1) to afford compound 592 as the BOC protected amine. A mixture of trifluoroactic acid (1.8 mL) and water (0.2 mL) was added and the reaction mixture stirred at room temperature for 15 minutes. The volatiles were removed in vacuo, to afford compound 592 as a colorless oil. HPLC-MS t_{R}=0.69 min (UV_{254 nm}); mass calculated for formula C₇H₁₇NO 131.1, observed LCMS m/z 132.1 (M+H).

### Example 16B

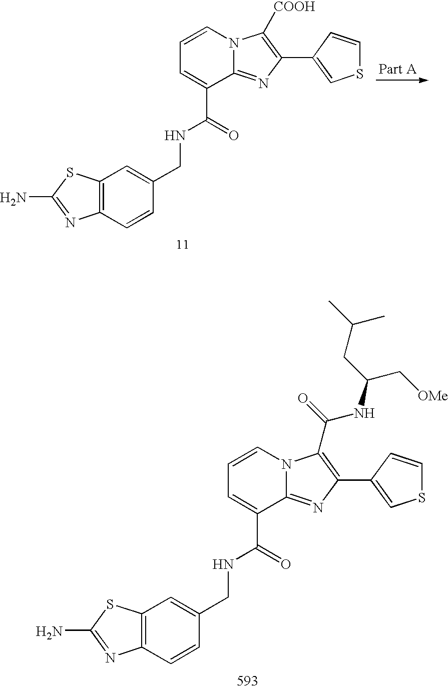

Compound 11 was prepared using procedures described in Example 1B.

### Part A:

Compound 593 was prepared from the coupling of compound 11 and compound 592 using procedures described in Example 1B, Part I.

| | | | | Ret. |
|---|---|---|---|---|
| | | | MS m/z | Time |
| Compd # | Structure | EMW | (M⁺+ H) | (min) |
| 593 | 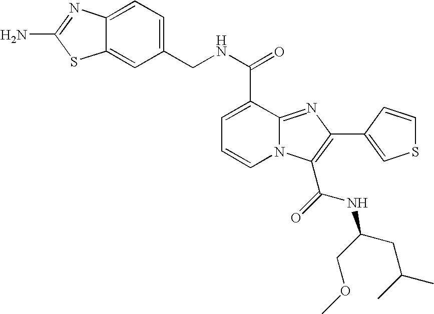 | 562.2 | 563.2 | 4.26 |

### Example 17

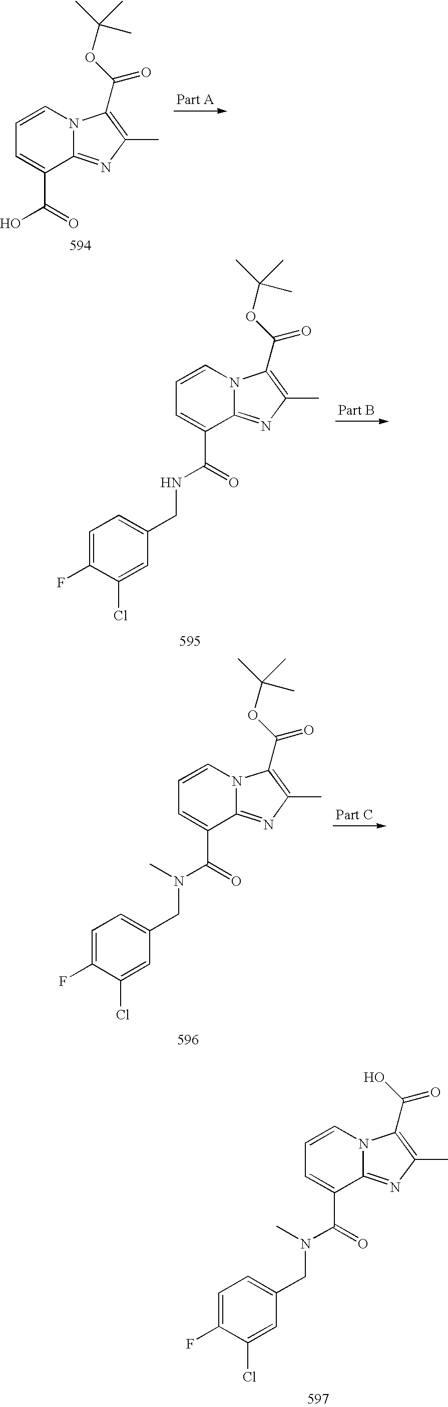

### Part A:

2-methyl-imidazo[1,2-a]pyridine-3,8-dicarboxylic acid-3-tert-butyl ester 594 (0.138 g, 0.5 mmol) was dissolved in dichloromethane. 1-(3-dimethylaminopropyl)-3-ethylcarbodiimide (0.093 g, 0.6 mmol;) was added followed by DIEA (1.5 mmol, 0.262 mL) and 3-chloro-4-fluoro benzyl amine (0.087 g, 0.55 mmol) was added. The reaction mixture stirred at room temperature for 10 hrs and LCMS analysis showed the completion of the reaction.

Reaction mixture diluted with water and extracted with EtOAc. The EtOAc layer separated, dried over anh. MgSO₄, filtered and evaporation of EtOAc gave crude 8-(3-Chloro-4-fluoro-benzylcarbomoyl)-2-methyl-imidazo[1,2-a]pyridine-3-carboxylic acid tert-butyl ester 595. Column chromatography of this material by eluting with Hexan/EtOAc gave pure product, 75%; 0.310 g; M⁺+H 418.2)

### Part B:

8-(3-Chloro-4-fluoro-benzylcarbomoyl)-2-methyl[1,2-a]pyridine-3-carboxylic acid-tert-butyl ester 595 (0.0417 g, 0.1 mmol) was dissolved in dry THF was added to the flask containing NaH (60%; 0.005 g) in THF. The reaction mixture cooled to 0° C. After 10 minutes the MeI (1.2 eq. 0.017 mL) was added. The reaction mixture warmed to room temperature stirred at room temperature for 2 hours. LC MS analysis showed N-methylation was complete. 5 mL of water was added to the solution and extracted in to EtOAC (50 mL). Organic layer dried with anh. MgSO₄, filtered, and evaporated to dryness to give the 8-(3-Chloro-4-fluoro-benzyl methylcarbomoyl)-2-methyl[1,2-a]pyridine-3-carboxylic acid-tert-butyl ester 596 in quantitative yield (0.043 g).

### Part C:

8-(3-Chloro-4-fluoro-benzyl)methyl-carbamoyl)-2-methyl[1,2-a]pyridine-3-carboxylic acid tert-butyl ester 596 (0.040 g) was treated with 4N HCl in dioxane for 2 hours obtain free carboxylic acid 597. The resulting solution was concentrated under vacuum to dryness and purified by prep. LC.

| | | | | Ret. |
|---|---|---|---|---|
| | | | MS m/z | Time |
| Compd # | Structure | EMW | (M⁺+ H) | (min) |
| 597 | 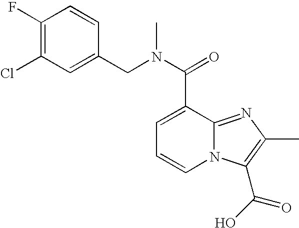 | 375.07 | 376.0 | 3.3 |

### Example 18

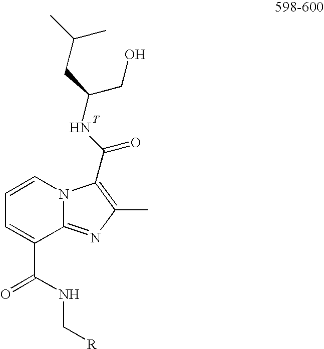

(wherein R is identified in Table 22)

The compounds in Table 22 are prepared using methods described in Example 2C

TABLE 22

| | | | MS m/z |
|---|---|---|---|
| compd # | Structure | EMW | (M⁺+ H) |
| 598 | 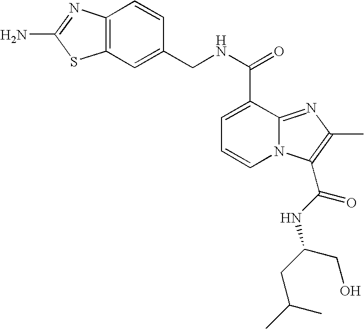 | 480.19 | 481.1 |
| 599 | 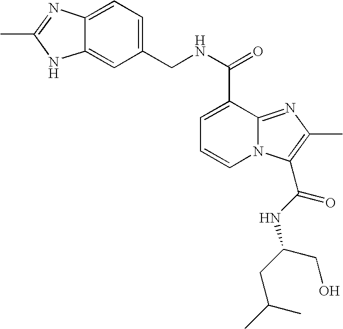 | 462.24 | 463.0 |
| 600 | 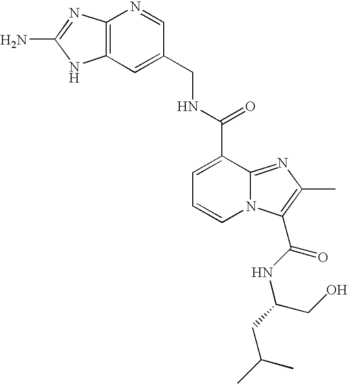 | 481.19 | 482.1 |

### Example 19

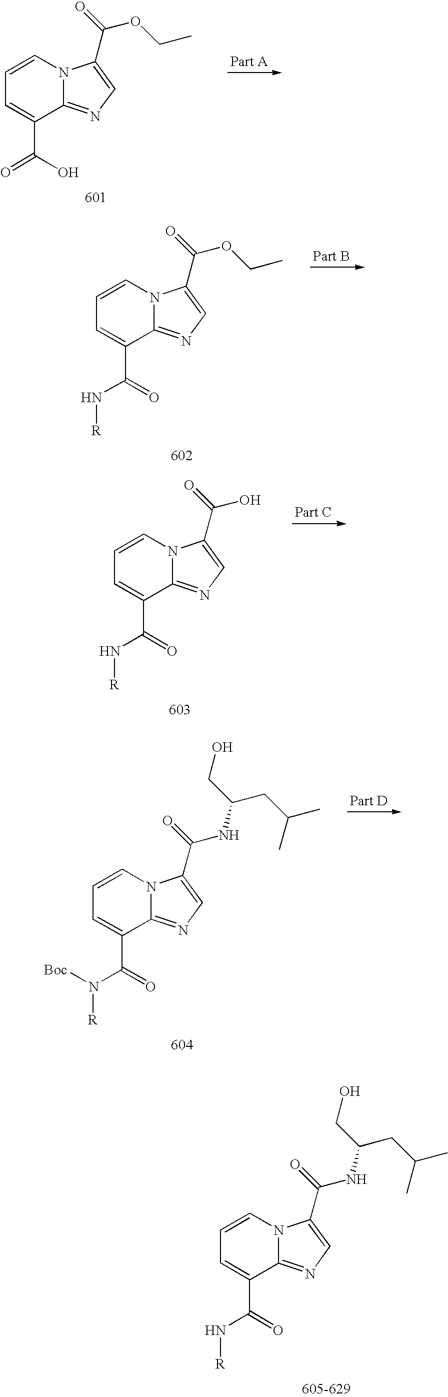

(wherein R is identified in Table 23)

### Part A:

Compound 602 was prepared using the peptide coupling condition described in Example 1B.

### Part B:

Compound 603 was prepared using the hydrolysis conditions described in Example 1B.

### Part C:

Compound 604 was prepared using the peptide coupling condition described in Example 1B

### Part D:

Compound 605 was prepared using the deprotecting condition described in Example 1B

The compounds in Table 23 were synthesized using essentially similar procedures and conditions as described in Example 19.

TABLE 23

| | | | | Ret. |
|---|---|---|---|---|
| | | | MS m/z | Time |
| Compd # | Structure | EMW | (M⁺+ H) | (min) |
| 605 | 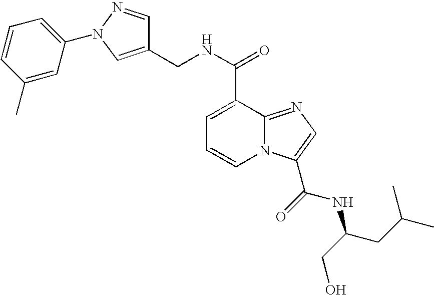 | 474.2 | 475.1 | 1.84 |
| 606 | 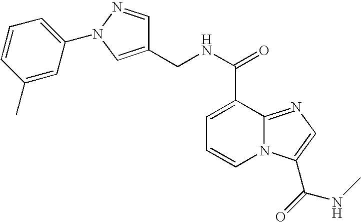 | 388.2 | 389.2 | 1.58 |
| 607 | 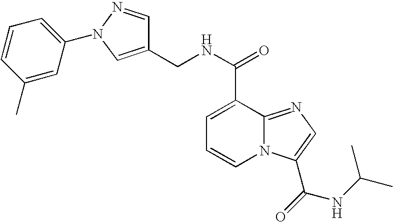 | 416.2 | 417.1 | 1.84 |
| 608 | 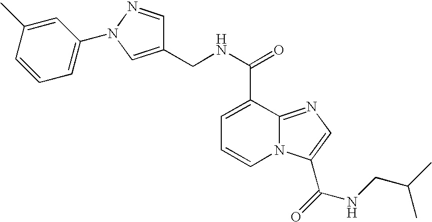 | 430.2 | 431.2 | 1.97 |
| 609 | 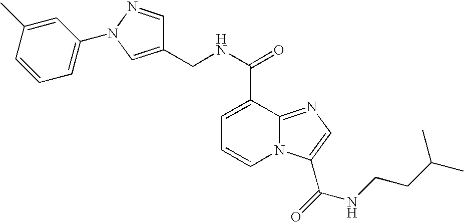 | 444.2 | 445.1 | 2.09 |
| 610 | 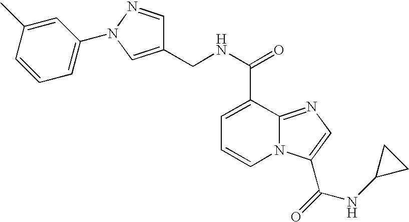 | 414.2 | 415.1 | 1.72 |
| 611 | 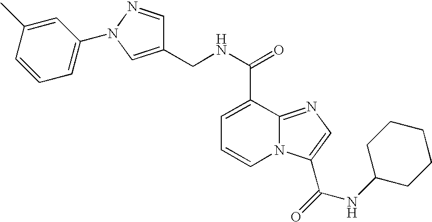 | 456.2 | 457.1 | 2.10 |
| 612 | 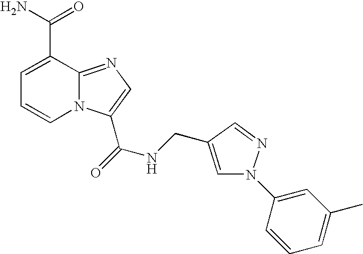 | 374.1 | 375.1 | 1.46 |
| 613 | 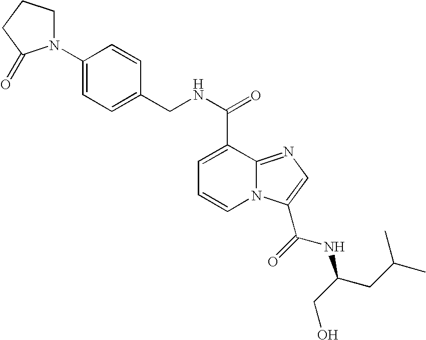 | 477.2 | 478.1 | 1.52 |
| 614 | 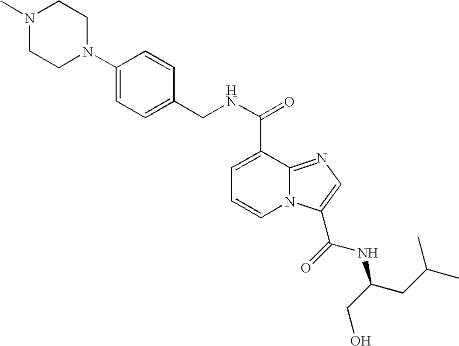 | 492.3 | 493.3 | 1.17 |
| 615 | 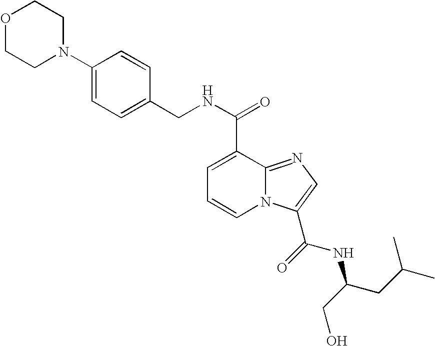 | 479.3 | 480.2 | 1.61 |
| 616 | 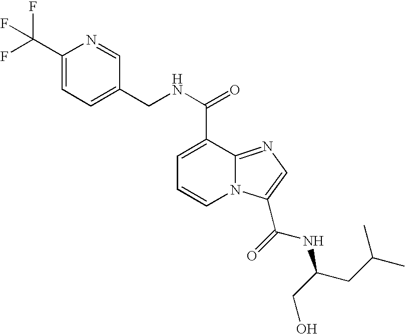 | 463.2 | 464.2 | 1.72 |
| 617 | 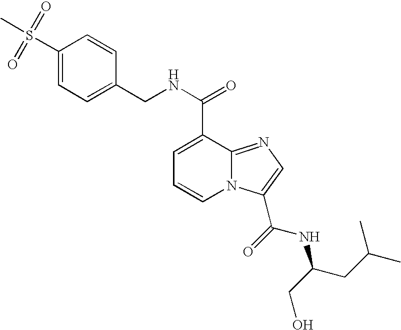 | 472.2 | 473.2 | 1.46 |
| 618 | 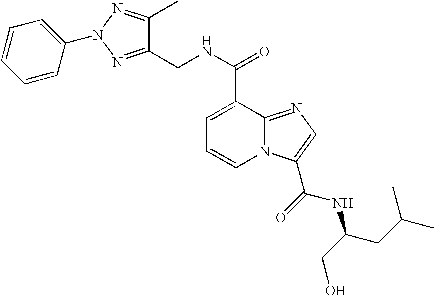 | 475.2 | 476.2 | 1.94 |
| 619 | 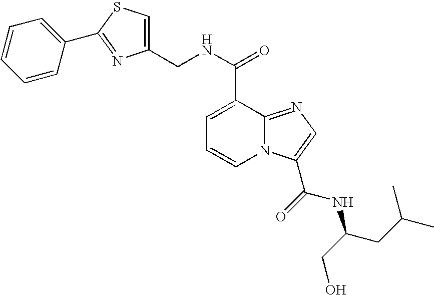 | 477.2 | 478.1 | 1.90 |
| 620 | 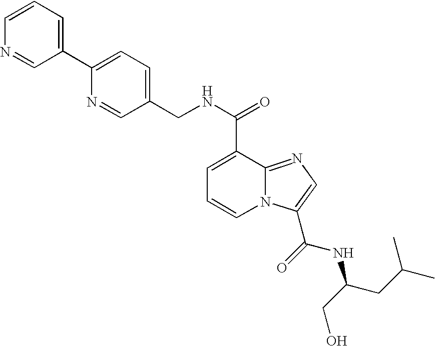 | 472.2 | 473.2 | 1.23 |
| 621 | 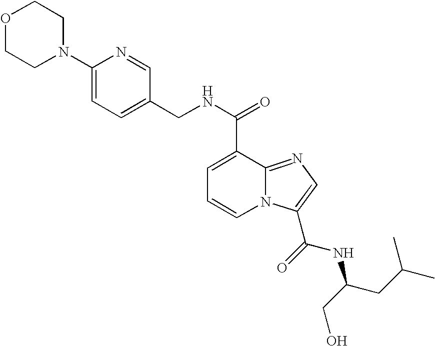 | 480.2 | 481.3 | 1.15 |
| 622 | 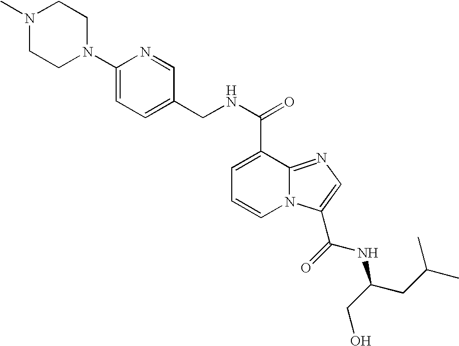 | 493.3 | 494.2 | 1.07 |
| 623 | 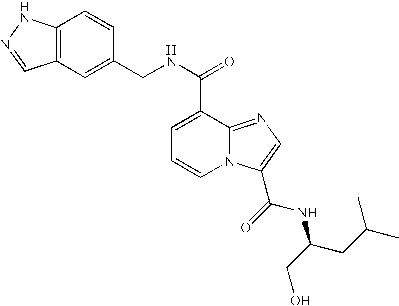 | 434.2 | 435.1 | 1.43 |
| 624 | 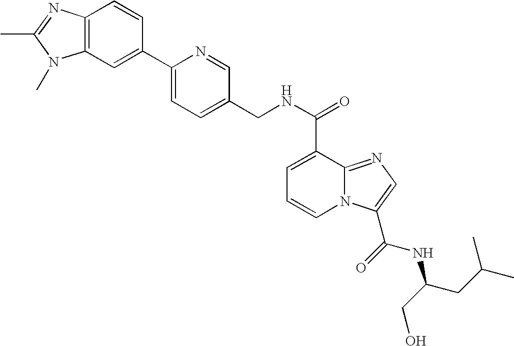 | 539.3 | 540.1 | 1.18 |
| 625 | 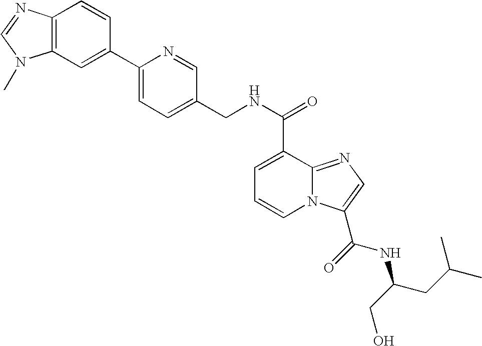 | 525.3 | 526.2 | 1.18 |
| 626 | 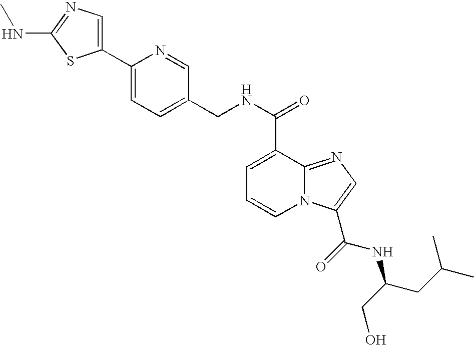 | 507.2 | 508.1 | 1.24 |
| 627 | 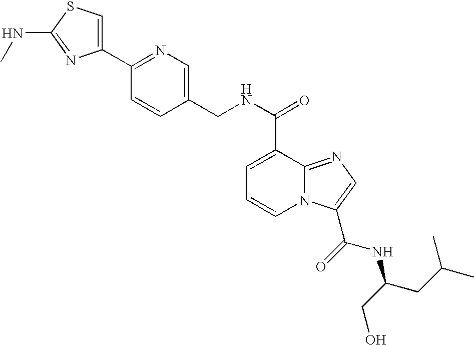 | 507.2 | 508.1 | 1.24 |
| 628 | 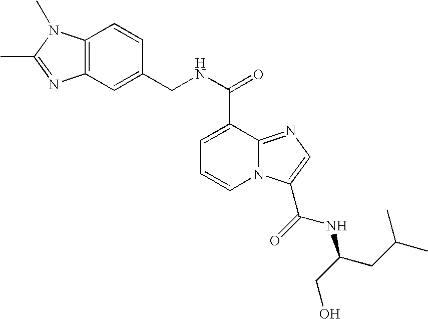 | 462.2 | 463.1 | 1.13 |
| 629 | 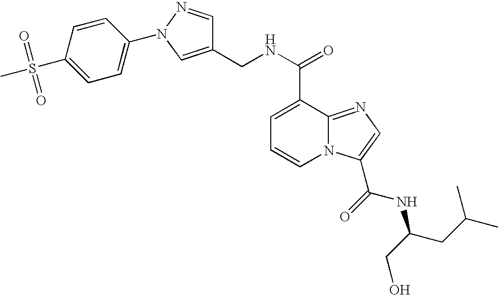 | 538.2 | 539 | 1.50 |

### Example 20

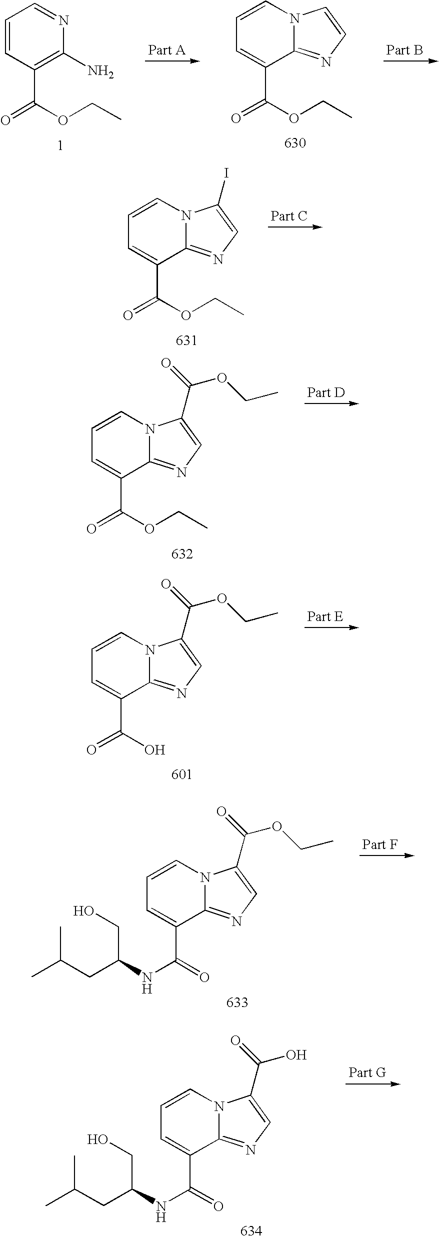

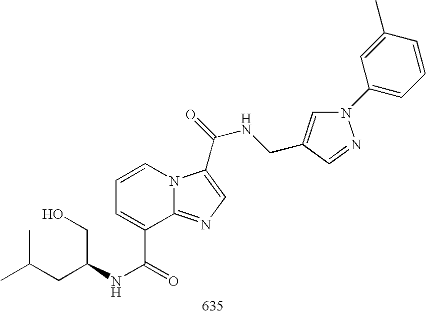

### Part A:

Compound 630 was prepared using the same conditions described in Example 8 part C with chloroacetaldehyde. HPLC-MS t_{R}=0.22 min (UV_{254 nm}); mass calculated for formula C₁₀H₁₀N₂O₂ 190.1, observed LCMS m/z 191.1 (M+H).

### Part B:

Compound 630 (1.84 g, 9.7 mmol) was dissolved in EtOH (10 mL) and NIS (2.38 g, 10.6 mmol) was added at room temperature. The resulting mixture was allowed to stir for 1 hour and then concentrated. The residue was diluted with EtOAc (150 mL) and washed with NaHCO₃ (sat. aq., 50 mL×3), brine and dried over Na₂SO₄. After concentration, the crude compound 631 was used in the next step directly without further purification. HPLC-MS t_{R}=1.25 min (UV_{254 nm}); mass calculated for formula C₁₀H₉IN₂O₂ 316.0, observed LCMS m/z 317.0 (M+H).

### Part C:

Under Argon, the flask was charged with compound 631 (crude, ˜9.7 mmol), Pd(dppf)Cl2 (0.900 g, 1.1 mmol), and Mo(CO)₆ (5.28 g, 20 mmol). DIEA (2 mL, 12 mmol) and EtOH (20 mL) was added and the flask was sealed under Argon flow. The mixture was heated up to 80° C. and stirred over night. After cooling to room temperature, the mixture was concentrated and diluted with EtOAc (250 mL) and washed with water, brine and dried over Na₂SO₄. After concentration, the residue was purified with column (silica gel, Hexane/EtOAc=40/60) gave the product 632 (1.0 g) as white solid. HPLC-MS t_{R}=1.22 min (UV_{254 nm}); mass calculated for formula C₁₃H₁₄N₂O₄ 262.1, observed LCMS m/z 263.1 (M+H).

### Part D:

Compound 601 was prepared using the hydrolysis conditions described in Example 8 part E. HPLC-MS t_{R}=0.77 min (UV_{254 nm}); mass calculated for formula C₁₁H₁₀N₂O₄ 234.1, observed LCMS m/z 235.1 (M+H).

### Part E:

Compound 633 was prepared using the peptide coupling conditions described in Example 1B. HPLC-MS t_{R}=1.81 min (UV_{254 nm}); mass calculated for formula C₁₇H₂₃N₃O₄ 333.2, observed LCMS m/z 334.1 (M+H).

### Part F:

Compound 634 was prepared using the hydrolysis conditions described in Example 8 part G. HPLC-MS t_{R}=1.18 min (UV_{254 nm}); mass calculated for formula C₁₅H₁₉N₃O₄ 305.1, observed LCMS m/z 306.1 (M+H).

### Part G:

Compound 635 was prepared using the peptide coupling conditions described in Example 1B.

| | | | | Ret. |
|---|---|---|---|---|
| | | | MS m/z | Time |
| Compd # | Structure | EMW | (M⁺+ H) | (min) |
| 635 | 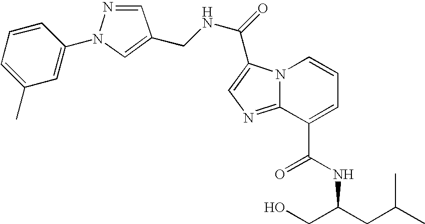 | 474.2 | 475.1 | 1.83 |

### Example 21

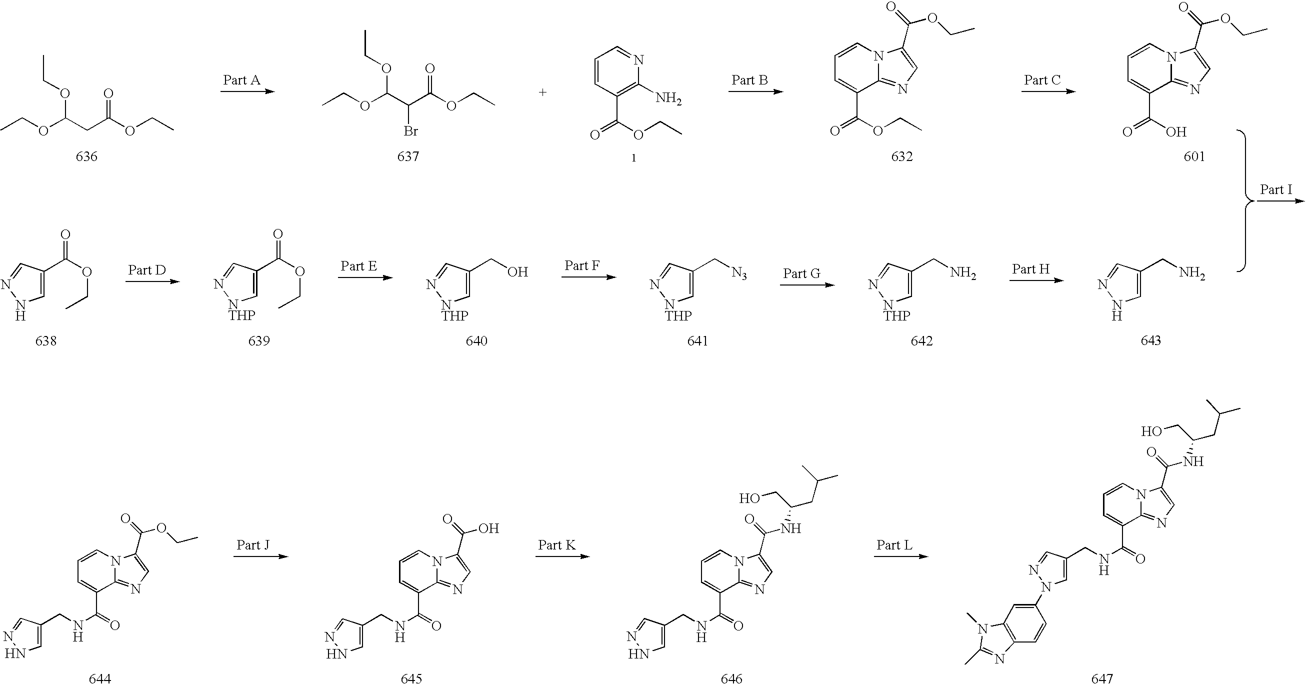

### Part A:

A few drops of bromine and pyridine (0.050 mL) were added to a well-stirred mixture of ethyl 3,3-diethoxylpropionate (15 g, 78.9 mmol), CCl₄ (50 mL) and dry precipitated Calcium carbonate (12 g, 120 mmol). After stirring for 15 min., the remained bromine (13.5 g, 84 mmol) was added dropwise during a period of 1 hour at 12-15° C. Carbon dioxide evolved regularly and the mixture thicked considerably. Stirring was continued for two hours at 12-15° C. after complete addition of bromine. The mixture was then poured into ice-water and the excess calcium carbonate was removed by filtration through celite. The CCl₄ layer was removed and after washing with water, NaHCO₃ (sat. aq.), brine and dried over Na₂SO₄, did concentration to remove CCl₄. The crude product 637 was used in the next step directly without purification.

### Part B:

Compound 632 was prepared using the same conditions described in Example 8 part C. HPLC-MS t_{R}=1.21 min (UV_{254 nm}); mass calculated for formula C₁₃H₁₄N₂O₄ 262.1, observed LCMS m/z 263.1 (M+H).

### Part C:

Compound 601 was prepared using the hydrolysis conditions described in Example 8 part E. HPLC-MS t_{R}=0.77 min (UV_{254 nm}); mass calculated for formula C₁₁H₁₀N₂O₄ 234.1, observed LCMS m/z 235.1 (M+H).

### Part D:

The mixture of pyrazole 638 (5.2 g, 30 mmol), DHP (11 mL, 120 mmol) and catalytic amount TFA (0.050 mL) was heated up to 60° C. and stirred for 6 hours. After cooling to room temperature, the excess amount of DHP was removed with concentration and the residue was purified with column gave the product 639 (5.5 g) as oil. HPLC-MS t_{R}=1.52 min (UV_{254 nm}); mass calculated for formula C₁₁H₁₆N₂O₃ 224.1, observed LCMS m/z 225.1 (M+H).

### Part E:

To the solution of ester 639 (5.5 g, 24.5 mmol) in THF (100 mL), LiAlH₄ (1N in THF, 55 mL) was added slowly at room temperature and the resulting mixture was stirred for two hours. To the mixture, water (1.65 mL) was added carefully and stirred for 10 min. Then, 15% NaOH (1.65 mL) was added and stirred for another 10 min followed by the addition of water (5 mL) and stirred for another 30 min. The mixture was filtered through celite and washed with EtOAc. After concentration, the crude product 640 was used in the next step directly without further purification. HPLC-MS t_{R}=1.18 min (UV_{254 nm}); mass calculated for formula C₉H₁₄N₂O₂ 182.1, observed LCMS m/z 183.1 (M+H).

### Part F:

The mixture of alcohol 640 (6.7 g, crude, -37 mmol), DBU (6.1 g, 40 mmol) and DPPA (11 g, 40 mmol) in THF (100 mL) was stirred at room temperature overnight. After concentration, the residue was diluted with EtOAc (300 mL) and washed with water, brine dried over Na₂SO₄. After concentration, the residue was purified with column (silica gel, hexane/EtOAc=20/80) gave the product 641 (6.2 g) as oil. HPLC-MS t_{R}=1.42 min (UV_{254 nm}); mass calculated for formula C₉H₁₃N₅O 207.1, observed LCMS m/z 208.2 (M+H).

### Part G:

The compound 641 (6.2 g, 29.9 mmol) was dissolved in the mixture of dioxane (100 mL) and resin supported PPh₃ (˜3 mmol/g, 15 g, 45 mmol) was added. The resulting mixture was stirred at room temperature for 1 hour. Then, the mixture of dioxane/H₂O (4:1, 100 mL) was added and the mixture was stirred overnight. The resin was removed by filtration and concentration gave the crude product 642 which was used in the next step without further purification. HPLC-MS t_{R}=0.23 min (UV₂₅₄ nm); mass calculated for formula C₉H₁₅N₃O 181.1, observed LCMS m/z 182.1 (M+H).

### Part H:

The crude compound 642 (˜29.9 mmol) was dissolved in dioxane (50 mL). HCl (con. 20 mL) was added and the mixture was stirred at room temperature for 2 hours. After concentration, the residue was diluted with H₂O, extracted with ethyl ether. The aqueous was concentrated and dried under vacuum. The crude product 643 was used in the next step without further purification.

### Part I:

Compound 644 was prepared using the peptide coupling conditions described in Example 1B. HPLC-MS t_{R}=1.32 min (UV_{254 nm}); mass calculated for formula C₁₅H₁₅N₅O₃ 313.1, observed LCMS m/z 314.2 (M+H).

### Part J:

Compound 645 was prepared using the hydrolysis conditions described in Example 8 part G. HPLC-MS t_{R}=0.65 min (UV_{254 nm}); mass calculated for formula C₁₃H₁₁N₅O₃ 285.1, observed LCMS m/z 286.1 (M+H).

### Part K:

Compound 646 was prepared using the peptide coupling conditions described in Example 1B Part I. HPLC-MS t_{R}=1.21 min (UV_{254 nm}); mass calculated for formula C₁₉H₂₄N₁₆O₃ 384.2, observed LCMS m/z 385.1 (M+H).

### Part L:

To the vial charged with compound 646 (0.039 g, 0.1 mmol), boronate (0.054 g, 0.2 mmol), CuOAc₂ (0.036 g, 0.2 mmol) and pyridine (0.016 g, 0.2 mmol), dioxane (2 mL) was added as solvent followed by the addition of 1 drop of water. The mixture was heated to 50° C. and stirred overnight without cap. After cooling down to room temperature, the mixture was purified with HPLC gave the product 647.

| | | | | Ret. |
|---|---|---|---|---|
| | | | MS m/z | Time |
| Compd # | Structure | EMW | (M⁺+ H) | (min) |
| 647 | 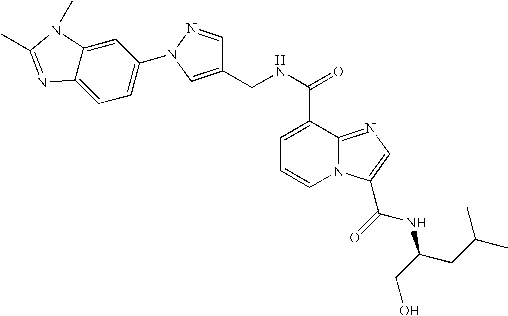 | 528.26 | 529.3 | 1.19 |

### Example 22

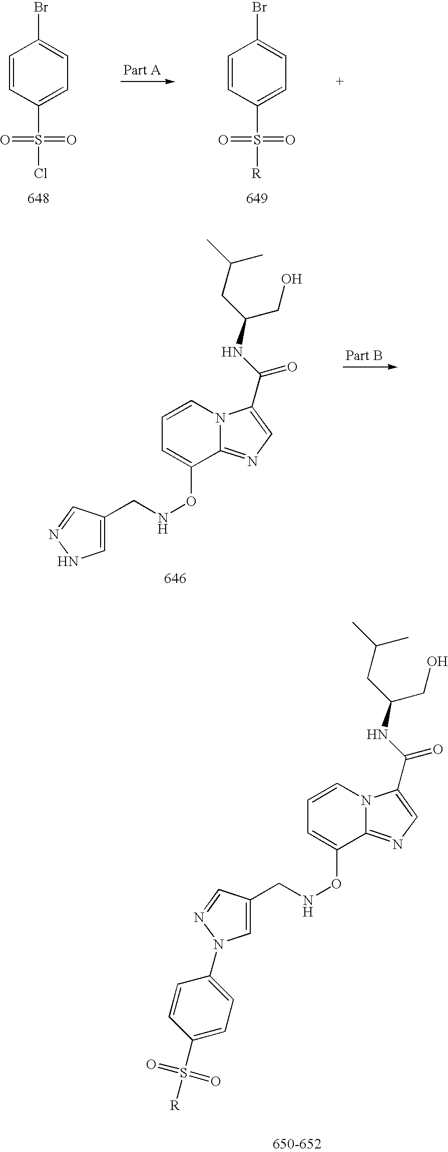

(wherein R is as identified in Table 24)

### Part A:

To a solution of compound 648 (1.00 g, 3.91 mmol) in dichloromethane (20 mL) was added diisopropylethylamine (0.75 mL, 4.30 mmol) at room temperature. The reaction mixture was cooled to 0° C. (ice-bath) and the corresponding amine (1.1 equivalents) added. The reaction mixture was allowed to warm to room temperature, stirred at ambient temperature for 16 hours, at which time LC-MS analysis indicated that the reaction was complete. The reaction mixture was concentrated under vacuum. Purification of by column chromatography ((SiO₂, 2% ethyl acetate/dichloromethane) afforded compound 649 as a white solid.

### Part B:

A mixture of compound 646 (0.139 mmol), cesium carbonate (0.091 g, 0.278 mmol), the bromide (1.1 equivalents) and anhydrous dimethylacetamide (1.5 mL) were added to the reaction vessel. The reaction vessel was flushed with Argon. Added copper (I) iodide (0.278 mmol) and 1,10-phenanthroline (0.051 g, 0.278 mmol). Flushed the reaction vessel again with argon and the mixture was stirred in a sealed tube for 20 hours at 140° C. LC-MS analysis of the reaction indicates that the reaction is complete. The mixture was then cooled to room temperature and filtered. The filtrate was concentrated. Purification by Prep-LC and conversion to a hydrochloride salt afforded to compound 650-652.

The compounds In Table 24 were synthesized using this procedure.

TABLE 24

| | | | | Ret. |
|---|---|---|---|---|
| | | | MS m/z | Time |
| Compd # | Structure | EMW | (M⁺+ H) | (min) |
| 650 | 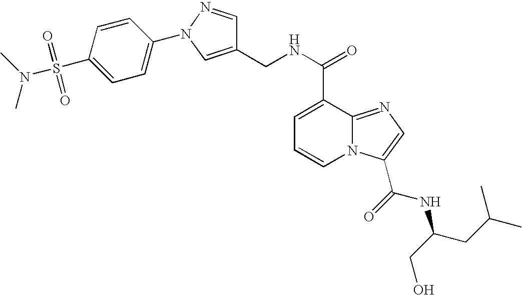 | 567.23 | | |
| 651 | 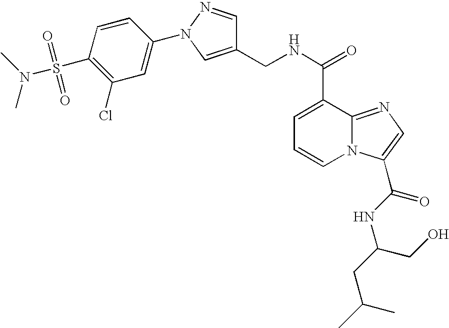 | 601.19 | 602.2 | 1.796 |
| 652 | 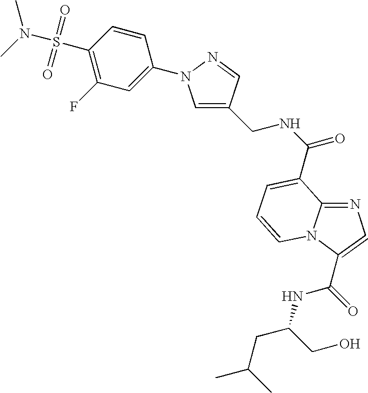 | 578.23 | 579.2 | 5.063 |

### Example 23

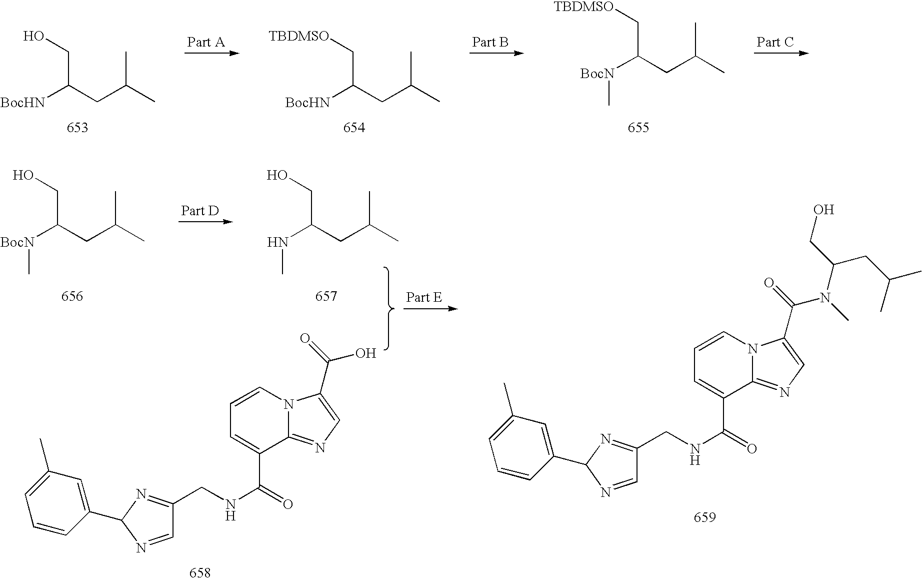

Compound 658 was synthesized using essentially the same procedure as described in Example 19.

### Part A:

The N-boc-L-lucinol compound 653 (2.2 g, 95%, 10 mmol) was dissolved in DCM (50 mL) and cooled to 10° C. The TBDMCl (1.5 g, 10 mmol), and imidazole (1.36 g, 20 mmol) were added. The mixture was allowed to warm to room temperature and stirred overnight. Then, the mixture was diluted with EtOAc (100 mL) and washed with water, brine and dried over Na₂SO₄. After concentration, the residue was purified with column (silica gel, hexane/EtOAc=95/5) gave the product 655 (3.25 g) as oil.

### Part B:

To the solution of compound 654 (3.25 g, 10 mmol) in THF (50 mL), NaH (0.600 g, 60% in oil, 15 mmol) was added carefully. The mixture was stirred at room temperature for 10 min, then MeI (20 mmol) was added. The resulting mixture was stirred overnight, then cooled to 0° C. with ice-water bath and H₂O was added carefully to quench the reaction. The aqueous was extracted with EtOAc and the organics was dried over Na₂SO₄. After concentration, the crude product 655 was used in the next step directly without further purification. HPLC

### Part C:

The crude compound 655 (3.19 g) was dissolved in THF (50 mL) and treated with Bu₄NF (12 mL, 1N in THF). The mixture was stirred at room temperature overnight and then concentrated. The residue was diluted with EtOAc (200 mL) and washed with water (50 mL×2), brine and dried over Na₂SO₄. After concentration, the crude product was purified with column (silica gel, hexane/EtOAc=50/50) gave product 656 (2.09 g) as oil.

### Part D:

The compound 656 (2.09 g, 9.0 mmol) was dissolved in dioxane (5 mL) and treated with HCl (6N, 10 mL). The mixture was stirred at room temperature for 1 hour, and then extracted with ethyl ether (40 mL). The aqueous was concentrated under vacuum and dried with lyphlization gave the product 657 (1.11 g) as white solid.

### Part E:

Compound 659 was prepared using the peptide coupling conditions described in Example 1B.

| | | | | Ret. |
|---|---|---|---|---|
| | | | MS m/z | Time |
| Compd # | Structure | EMW | (M⁺+ H) | (min) |
| 659 | 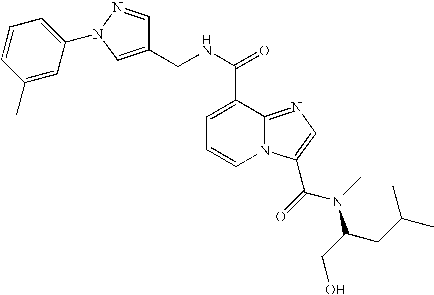 | 488.3 | 489.2 | 1.86 |

### Example 24

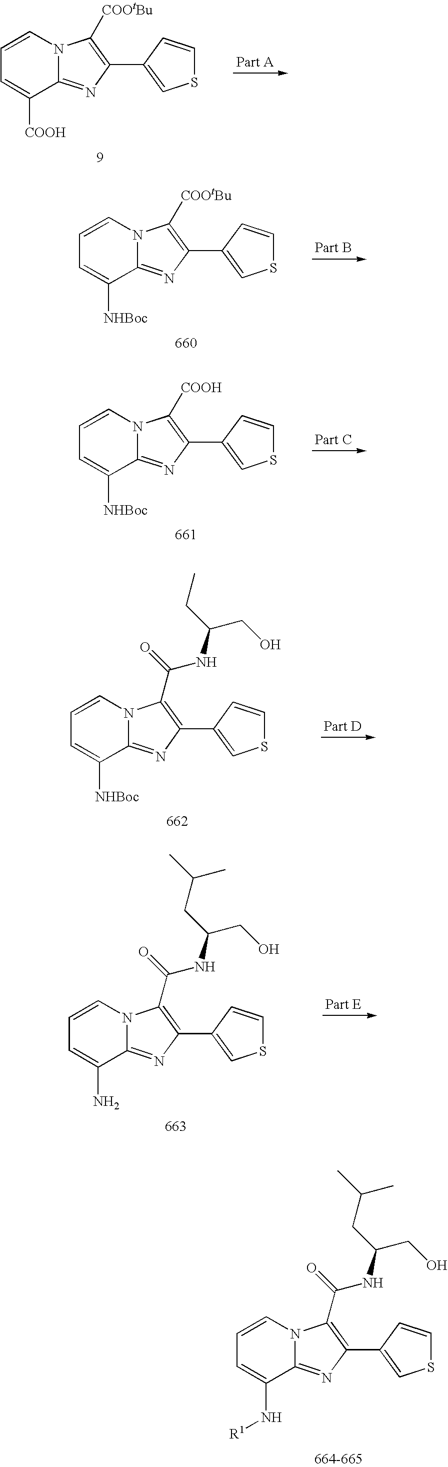

(wherein R¹ is identified in Table 25)

Compound 9 was prepared from procedures described in Example 1B.

### Part A:

A mixture of compound 9 (0.266 g, 0.77 mmol), diphenylphosphorylazide (0.334 mL, 1.54 mmol) and triethylamine (0.323 mL, 2.31 mmol) in t-butanol (10 mL) was heated at reflux for 16 hours. The reaction mixture was cooled to room temperature and monitored by LC-MS. The volatiles were removed in vacuo, and the crude purified by flash column chromatography to afford compound 660 as a white solid. HPLC-MS t_{R}=2.68 min (UV_{254 nm}); mass calculated for formula C₂₁H₂₅N₃O₄S 415.2, observed LCMS m/z 416.1 (M+H).

### Part B:

Compound 661 was prepared from compound 660 using procedures described in Example 1B, Part F.

### Part C:

Compound 662 was prepared from compound 661 using procedures described in Example 1B, Part G. HPLC-MS t_{R}=2.19 min (UV_{254 nm}); mass calculated for formula C₂₃H₃₀N₁₄O₄S 458.2, observed LCMS m/z 459.1 (M+H).

### Part D:

Compound 663 was prepared from compound 662 using procedures described in Example 1B, Part H. HPLC-MS t_{R}=1.27 min (UV_{254 nm}); mass calculated for formula C₁₈H₂₂N₁₄O₂S 358.1, observed LCMS m/z 359.1 (M+H).

### Part E:

Compounds 664-665 (Table 25) were prepared from compound 307 using procedures described in Example 1B, Part I.

TABLE 25

| | | | MS m/z | |
|---|---|---|---|---|
| Compd # | Structure | EMW | (M⁺+ H) | Ret. Time (min) |
| 664 | 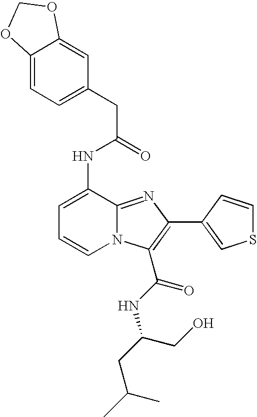 | 520.2 | 521.2 | 4.77 |
| 665 | 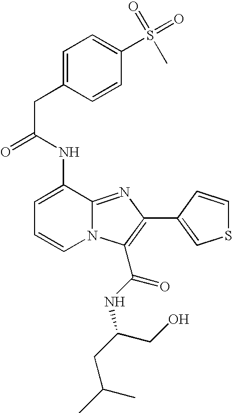 | 554.2 | 555.2 | 4.14 |

### Example 25A

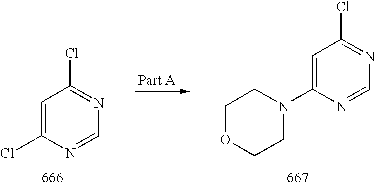

### Part A:

To the solution of compound 666 (0.300 g, 2.0 mmol) in dioxane (5 mL), DIEA (0.356 mL, 2.0 mmol) was added followed by the addition of morpholine (0.174 mL, 2.0 mmol). The mixture was stirred at room temperature over night and concentrated. The residue was purified with column (silica gel, DCM/EtOAc=50/50) to give the product 667 (0.320 g) as white solid. HPLC-MS t_{R}=1.12 min (UV_{254 nm}); mass calculated for formula C₈H₁₀ClN₃O 199.1, observed LCMS m/z 200.1 (M+H).

### Example 25B

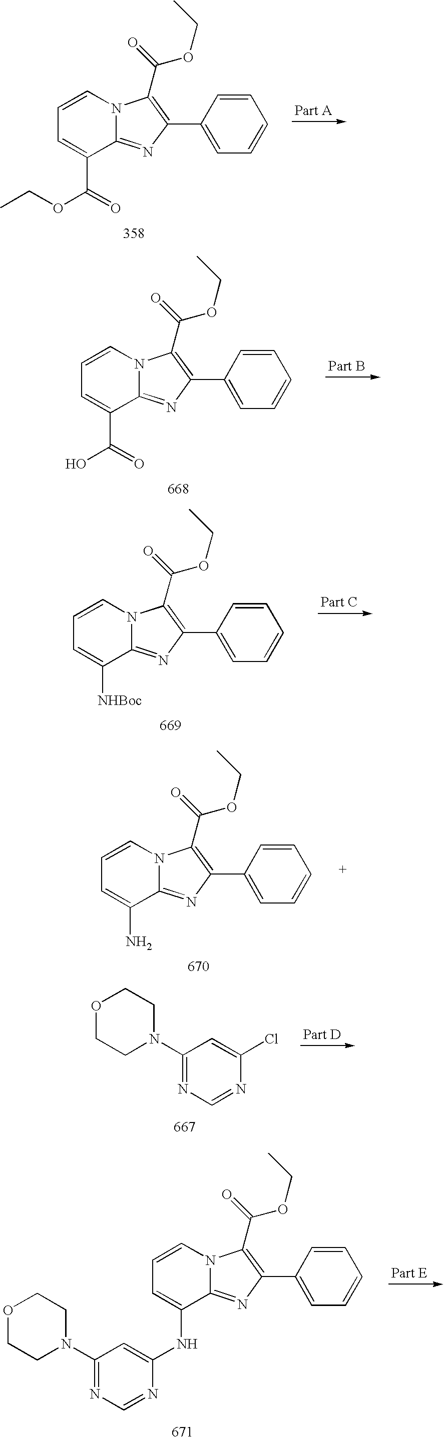

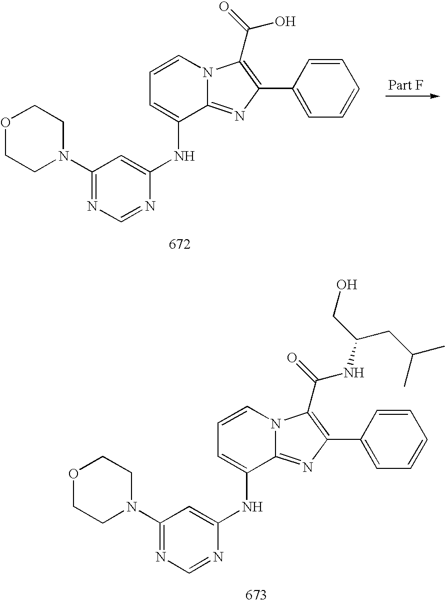

Compound 358 was synthesized in Example 8.

### Part A:

Compound 668 was prepared using the hydrolysis conditions described in Example 8 part E. HPLC-MS t_{R}=0.67 min (UV_{254 nm}); mass calculated for formula C₁₄H₁₁N₃O₂ 253.1, observed LCMS m/z 254.1 (M+H).

### Part B:

The monoacid 668 (0.212 g, 0.68 mmol) was dissolved in t-butyl alcohol (20 mL), TEA (0.096 mL, 0.68 mmol) and DPPA (0.187 g, 0.68 mmol) was added. The mixture was heated up to refluxed and stirred over night. After cooled to room temperature, the solvent was removed with concentration. The residue was purified with column (silica gel, Hexane/EtOAc=80/20) gave the product 669 (0.221 g) as oil. HPLC-MS t_{R}=2.73 min (UV_{254 nm}); mass calculated for formula C₂₁H₂₃N₃O₄ 381.2, observed LCMS m/z 382.1 (M+H).

### Part C:

Compound 670 was prepared using the same deprotecting conditions described in Example 8. HPLC-MS t_{R}=1.72 min (UV_{254 nm}); mass calculated for formula C₁₆H₁₆N₃O₂ 281.1, observed LCMS m/z 282.1 (M+H).

### Part D:

Under Argon, the vial was charged with 4-(6-chloropyrimidin-4-yl)-morpholine 667 (0.060 g, 0.3 mmol), compound 670 (0.168 mg, 0.6 mmol), Pd₂ dba₃ (0.016 g, 0.017 mmol), 1,3-Bis(2,6-di-i-propylphenyl)-4,5-dihydroimidazolium tetrafluoroborate (0.016 g, 0.35 mmol) and NaO^{t}Bu (0.096 g, 1.0 mmol). Dioxane (2 mL) was added as solvent and the vial was sealed under Argon flow. The mixture was heated up to 80° C. and stirred over night. After cooling to room temperature, the mixture was diluted with EtOAc (50 mL) and washed with NH₄Cl (sat. aq.), brine and dried over Na₂SO₄. After concentration, the residue was purified with column (silica gel, Hexane/EtOAc=60/40) gave the product 671 (0.069 g) as oil. HPLC-MS t_{R}=1.82 min (UV_{254 nm}); mass calculated for formula C₂₄H₂₄N₆O₃ 444.2, observed LCMS m/z 445.1 (M+H).

### Part E:

Compound 672 was prepared using the hydrolysis conditions described in Example 8 part G. HPLC-MS t_{R}=1.18 min (UV_{254 nm}); mass calculated for formula C₂₂H₂₀N₆O₃ 416.2, observed LCMS m/z 417.1 (M+H).

### Part F:

Compound 673 was prepared using the peptide coupling conditions described in Example 1B, Part I. HPLC-MS t_{R}=1.43 min (UV_{254 nm}); mass calculated for formula C₂₈H₃₂N₆O₃ 500.3, observed LCMS m/z 501.1 (M+H).

The compounds In Table 26 were synthesized using the same procedure described in Example 25B.

TABLE 26

| | | | | Ret. |
|---|---|---|---|---|
| | | | MS m/z | Time |
| Compd # | Structure | EMW | (M⁺+ H) | (min) |
| 672 | 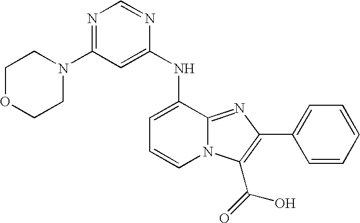 | 416.2 | 417.1 | 1.18 |
| 673 | 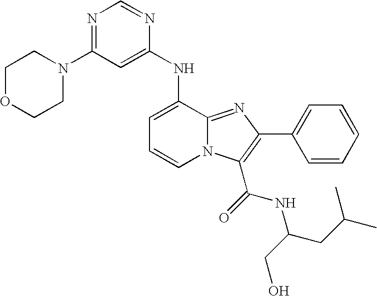 | | | |
| 674 | 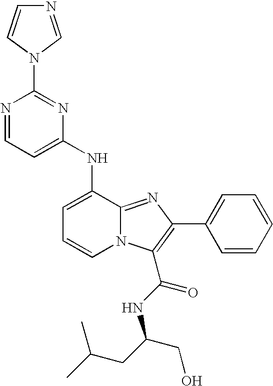 | 496.2 | 497.2 | 1.38 |
| 675 | 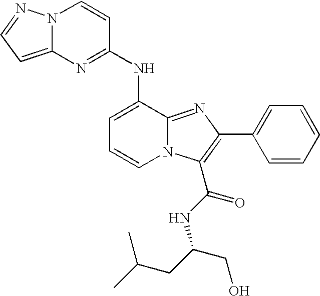 | 469.2 | 470.2 | 1.83 |
| 676 | 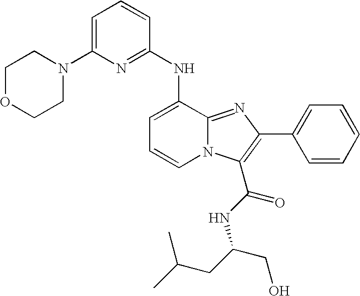 | 514.3 | 515.2 | 2.04 |
| 677 | 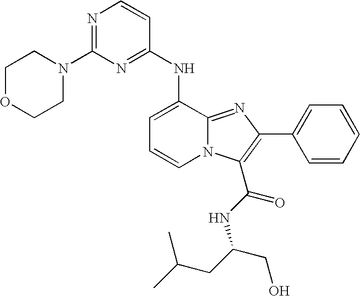 | 515.3 | 516.3 | 1.35 |
| 678 | 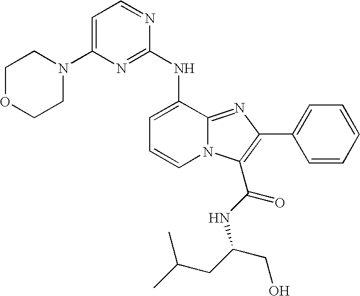 | 515.3 | 516.3 | 1.34 |
| 679 | 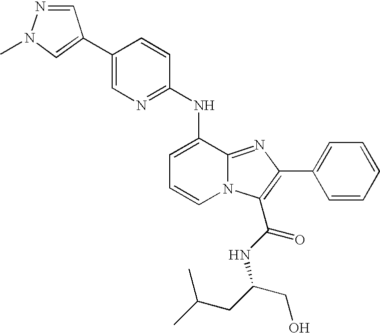 | 509.3 | 510.1 | 2.24 |
| 680 | 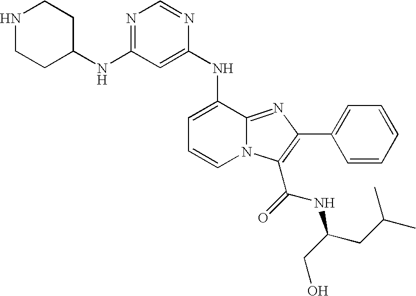 | 528.3 | 529.2 | 0.95 |

### Example 26

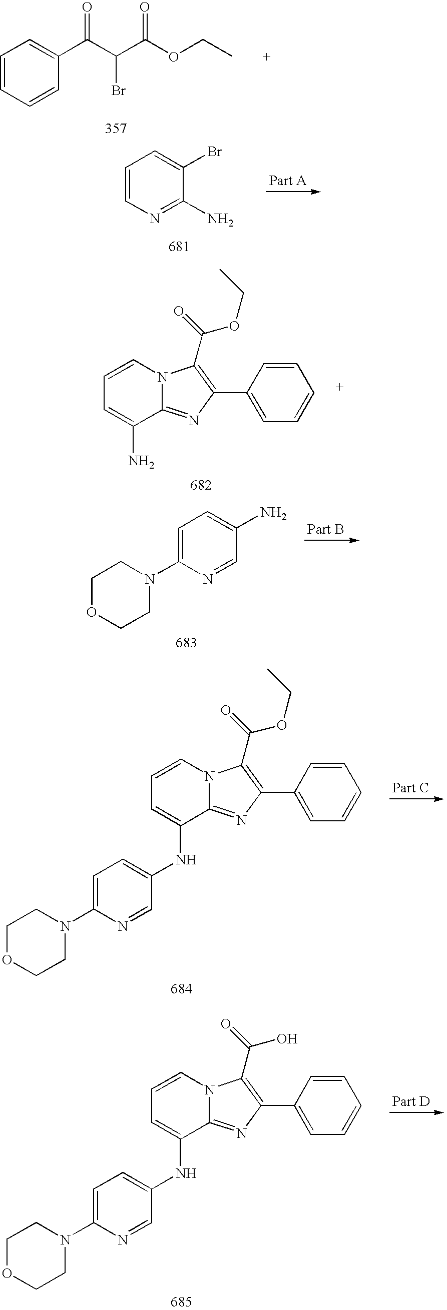

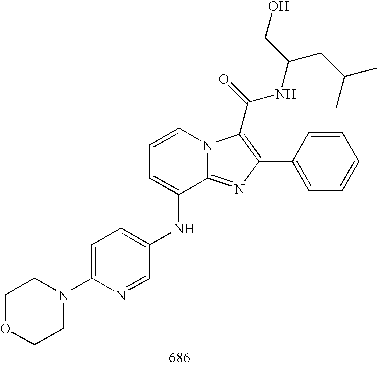

### Part A:

Compound 682 was prepared using the conditions described in Example 8 part C. HPLC-MS t_{R}=2.11 min (UV_{254 nm}); mass calculated for formula C₁₆H₁₃BrN₂O₂ 344.0, observed LCMS m/z 345.0 (M+H).

### Part B:

Compound 684 was prepared using the amination conditions described in Example 22 part D. HPLC-MS t_{R}=1.84 min (UV_{254 nm}); mass calculated for formula C₂₅H₂₅N₅O₃ 443.2, observed LCMS m/z 444.2 (M+H).

### Part C:

Compound 685 was prepared using the hydrolysis conditions described in Example 22 part G. HPLC-MS t_{R}=1.20 min (UV_{254 nm}); mass calculated for formula C₂₂H₁₉N₅O₃ 415.2, observed LCMS m/z 416.2 (M+H).

### Part C:

Compound 686 was prepared using the peptide coupling conditions described in Example 1B.

| | | | | Ret. |
|---|---|---|---|---|
| | | | MS m/z | Time |
| Compd # | Structure | EMW | (M⁺+ H) | (min) |
| 686 | 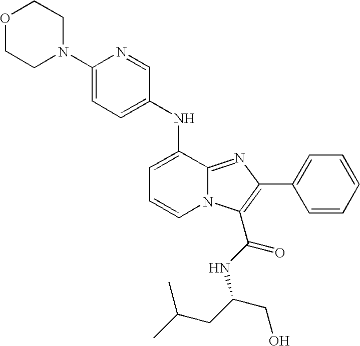 | 514.3 | 515.2 | 1.55 |

### Example 27

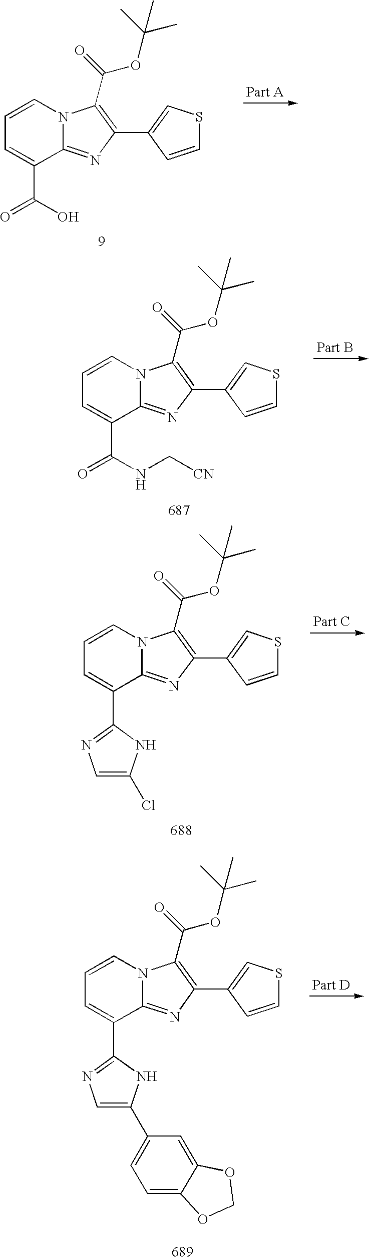

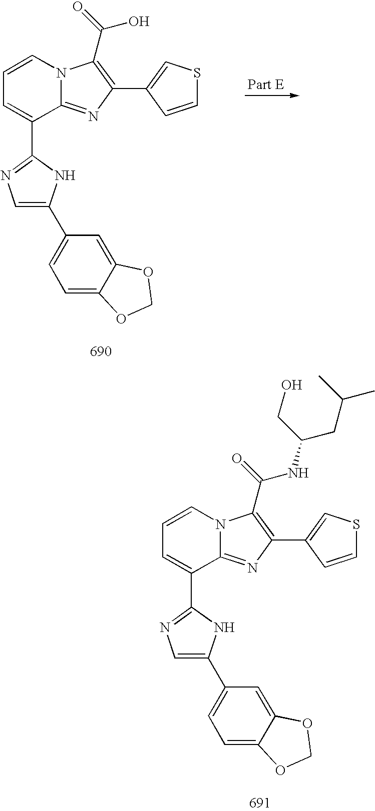

### Part A:

Compound 687 was prepared using the peptide coupling conditions described in Example 1B Part G. HPLC-MS t_{R}=2.89 min (UV_{254 nm}); mass calculated for formula C₁₉H₁₈N₄O₃S 382.1, observed LCMS m/z 383.0 (M+H).

### Part B:

The compound 687 (0.038 g, 0.1 mmol) was dissolved in CAN (5 mL), PPh₃ (0.066 g, 0.25 mmol) and CCl₄ (0.024 mL, 0.25 mmol) were added. The mixture was heated to 40° C. and stirred overnight. After concentration, the residue was took up with NaOH (0.5N, 4 mL) and stirred for another 10 min. The mixture was extracted with EtOAc (20 mL×3), and the organics was dried over Na₂SO₄. After concentration, the crude product was purified with column (silica gel, Hexane/EtOAc=70/30) gave the product 688 (0.031 g) as yellowish solid. HPLC-MS t_{R}=2.47 min (UV_{254 nm}); mass calculated for formula C₁₉H₁₇ClN₄O₂S 400.1, observed LCMS m/z 401.0 (M+H).

### Part C:

Under Ar, the chloroimidazole compound 688 (0.020 g, 0.05 mmol) in toluene (2.0 ml) was added to the flask which was charged with Pd₂ dba₃ (0.008 g, 0.01 mmol), 2-dicyclohexylphosphino-2′,4′,6′-tri-i-propyl-1,1′-biphenyl (0.019 g, 0.04 mmol), K₃PO₄ (0.212 g, 1.0 mmol), and boronic acid (0.017 g, 0.1 mmol). The mixture was thoroughly degassed by alternately connected the flask to vacuum and Argon. The resulting solution was heated up to 100° C. and stirred overnight and diluted by EtOAc after cooled to room temperature. The solid was removed by filter through Celite and washed with some EtOAc. Concentration to remove the solvent and the resulting residue was purified with column (silica gel, Hexane/EtOAc=50/50) gave the product 689 as oil. HPLC-MS t_{R}=2.23 min (UV_{254 nm}); mass calculated for formula C₂₆H₂₂N₄O₄S 486.1, observed LCMS m/z 487.0 (M+H).

### Part D:

Compound 689 (0.010 g, 0.02 mmol) was treated with HCl (con. 2 mL) and stirred at room temperature for 10 min. After concentration, the crude product 690 was used in the next step directly. HPLC-MS t_{R}=1.52 min (UV_{254 nm}); mass calculated for formula C₂₆H₂₂N₄O₄S 430.0, observed LCMS m/z 431.0 (M+H).

### Part E:

Compound 691 was prepared using the peptide coupling conditions described in Example 1B Part I.

The compounds in Table 27 were synthesized using the same procedure described in Example 27

TABLE 27

| Compd # | Structure | EMW | MS m/z (M⁺+ H) | Ret. Time (min) |
|---|---|---|---|---|
| 691 | 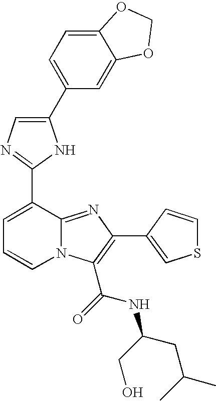 | 529.2 | 530.0 | 1.73 |
| 692 | 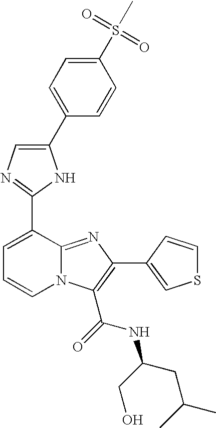 | 545.2 | 546.1 | 1.77 |

### Example-28

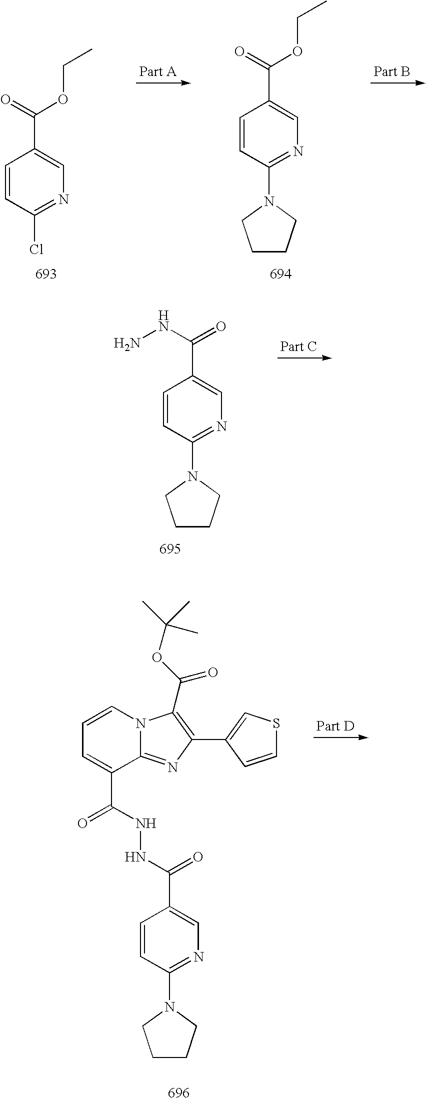

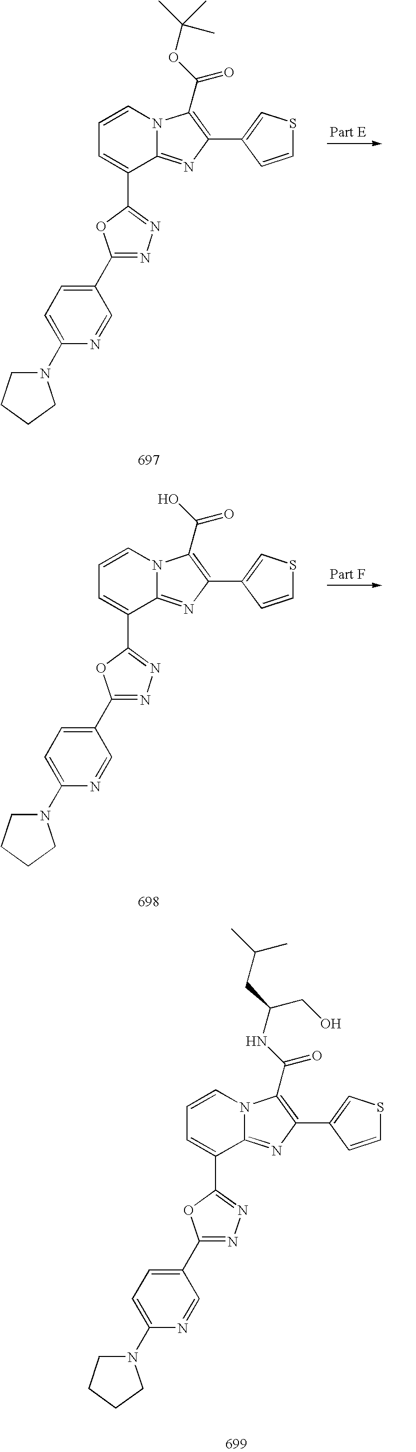

### Part A:

Ethyl-6-chloronicotinate 693 (5 mmol; 0.900 g) was dissolved in 5 mL of Pyrrolidine and refluxed for 14 hours. The Pyrrolidine was removed under vacuum and resulting gummy material diluted with Ethyl acetate and washed with water, brine and dried over anhydrous MgSO₄ and filtered and concentrated. Purification by silica column resulted in title compound (40%).

### Part B:

6-pyrrolidin-1-yl-nicotinic acid ethyl ester 694 obtained in the above step was dissolved in Ethanol (25 mL) and hydrazine hydrate (5 mL,) was added and the reaction mixture refluxed for 4 hours. Concentration of ethanol afforded the title compound, hydrazide 695 as crystalline compound (100%).

### Part C:

2-Thiphene-3-yl-imidazo[1,2-a]pyridine-3,8-dicarboxylic acid-3-t-butyl ester (0.5 mmol; 0.172 g) dissolved in dichloromethane (5 mL). To this (1-(3-dimethylaminopropyl)-3-ethylcarbodiimide (0.093 g; 1.2 eq; 0.6 mmol) was added. Followed by Diisopropyl ethyl amine (3 equivalents, 0.315 mL) was added and the solution stirred at room temperature for 15 minutes.

The activated acid was added with 0.55 mmol (0.115 g) solution of 6-pyrrolidin-1-yl-nicotinic acid hydrazide 695 (pre dissolved in to NMP; 0.5 mL). The solution was shaken at room temperature for 4 hours. LCMS analysis showed the completion of the reaction.

The reaction vessel added with water and extracted with EtOAc (60 mL). The EtOAc extracts were washed with brine, dried with anhydrous MgSO₄, filtered, and EOAc evaporated under vacuum. Purification by column chromatography (SiO₂,Hexane-ethyl acetate) afforded title compound 696.

### Part D:

8-[N′-(6-Pyrrolidin-1-yl-pyridine-3-carbonyl) hydrazinocarbonyl]-2-thiophen-30-imidazo[1,2-a]pyridine-3-carboxylic acid t-butyl ester 696 was dissolved in dichloromethane-carbon tetrachloride (1:1) and triphenyl phosphine on resin (3 mmol/g, 3 g) was added and the reaction was refluxed for 8 hours. The reaction cooled to room temperature, filtered off the resin. The filtrate was evaporated under vacuum. The resulting material was used in the next step with out purification.

### Part E:

8-[5-(6-pyrrolidin-1-yl-)[1,3,4-]oxadizol-2yl-]-2-thiophen-3-yl-imidazo[1,2-a]pyridine-3-carboxylic acid-tert-butyl ester 697 is dissolved in 4N HCl in dioxane and stirred for 2 hours. The dioxane/HCl was evaporated under vacuum to give title compound, free carboxylic acid. The crude product is dissolved in Acetonitrile-water and freeze dried, lyophilized to get a product in powder form, which used in the next step with out purification. Mass calculated formula: C23H18N6O3S; M.Wt=458.11; M+H=459.21]

### Part F:

8-[5-(6-pyrrolidin-1-yl-)[1,3,4-]oxadizol-2yl-]-2-thiophen-3yl-imidazo[1,2-a]pyridine-3-carboxylic acid 698 thus obtained was dissolved in NMP (2 mL), and HATU (1.2 eq), DIEA (3 equivalents) were added in sequence. L-Ieucinol (1.2 equivalents) was added and the reaction mixture stirred at room temperature for 3 hours. The reaction mixture was diluted with Ethyl acetate and water. The ethyl acetate layer washed with water, brine and dried over anhydrous magnesium sulfate. Filtered, and EtOAc removed under vacuum to get the title compound 699. This was purified by mass triggered Preparative HPLC to get 90% pure product.

| | | | MS m/z | Ret. |
|---|---|---|---|---|
| Compd # | Structure | EMW | (M⁺+ H) | Time (min) |
| 699 | 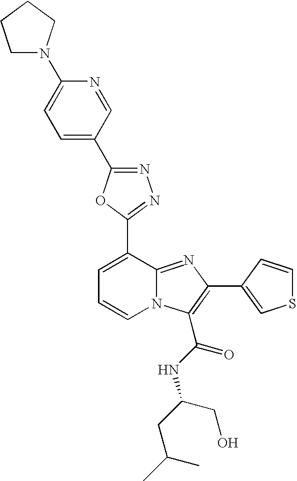 | 557.2 | 558.2 | 3.77 |

### Example 29

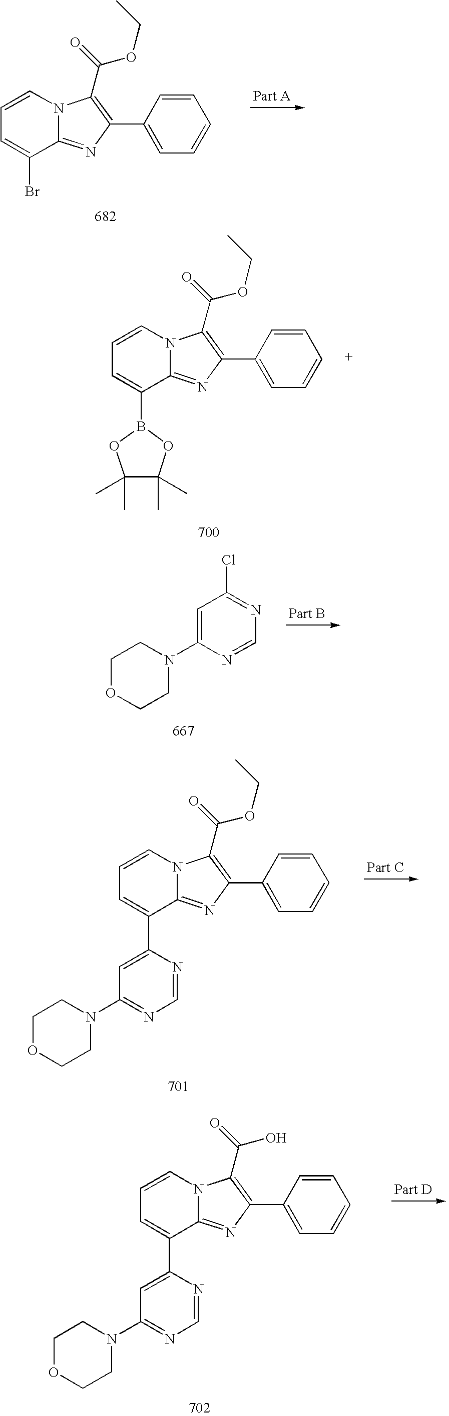

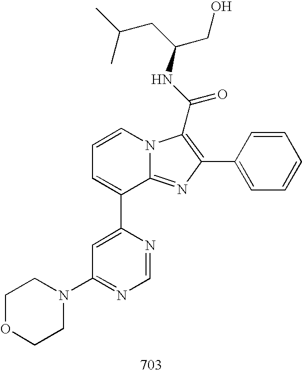

### Part A:

Compound 682 was prepared using the conditions described in Example 26. HPLC-MS t_{R}=2.11 min (UV_{254 nm}); mass calculated for formula C₁₆H₁₃BrN₂O₂ 344.0, observed LCMS m/z 345.0 (M+H).

### Part B:

To a 25 ml round bottom flask charged with bis(pinacolato)diboron (0.307 g, 1.2 mmol), (0.294 g, 3.0 mmol) of KOAc and (0.027 g, 0.03 mmol) of PdCl₂(dppf) was added a solution of compound 682 (0.375 g, 1.0 mmol) in DMSO (6 ml). The mixture was thoroughly degassed by alternately connected the flask to vacuum and Argon. This resulting mixture was then heated at 80° C. overnight, diluted by EtOAc (40 ml) and filtered through celite. After concentration, the residue was purified with column (silica gel, Hexane/EtOAc=60/40) to give the product 700 (0.301 g) as oil. HPLC-MS t_{R}=1.88 min (UV_{254 nm}); mass calculated for formula C₂₂H₂₅BN₂O₄ 392.2, observed LCMS m/z 393.1 (M+H).

### Part C:

Under Ar, the bornate compound 700 (0.050 g, 0.13 mmol) in dioxane (2.0 ml) was added to the flask which was charged with Pd(dppf)Cl₂ (0.008 g), K₃PO₄ (1.790 g, 0.4 mmol), and chloropyrimidine 667 (0.026 g, 0.13 mmol). The mixture was thoroughly degassed by alternately connected the flask to vacuum and Argon. The resulting solution was heated upto 80° C. and stirred overnight and diluted by EtOAc after cooled to room temperature. The solid was removed by filter through Celite and washed with some EtOAc. Concentration to remove the solvent and the resulting residue was purified with column (silica gel, Hexane/EtOAc=50/50) gave the product 701 as oil. HPLC-MS t_{R}=1.89 min (UV_{254 nm}); mass calculated for formula C₂₄H₂₃N₅O₃ 429.2, observed LCMS m/z 430.1 (M+H).

### Part D:

Compound 702 was prepared using the hydrolysis conditions described in Example 8 Part G. HPLC-MS t_{R}=1.14 min (UV_{254 nm}); mass calculated for formula C₂₂H₁₉N₁₅O₃ 401.1, observed LCMS m/z 402.1 (M+H).

### Part E:

The compound 703 (0.040 g, 0.1 mmol) was dissolved in DMF (2 mL), TIEA (0.018 mL, 0.1 mmol) and HATU (0.038 g, 0.1 mmol) were added at room temperature followed by the addition of L-lucinol (0.011 g, 0.1 mmol). The mixture was stirred over night and purified with HPLC.

| | | | MS m/z | Ret. |
|---|---|---|---|---|
| Compd # | Structure | EMW | (M⁺+ H) | Time (min) |
| 703 | 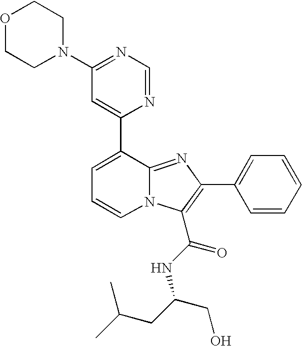 | 500.3 | 501.1 | 1.43 |

### Example 30A

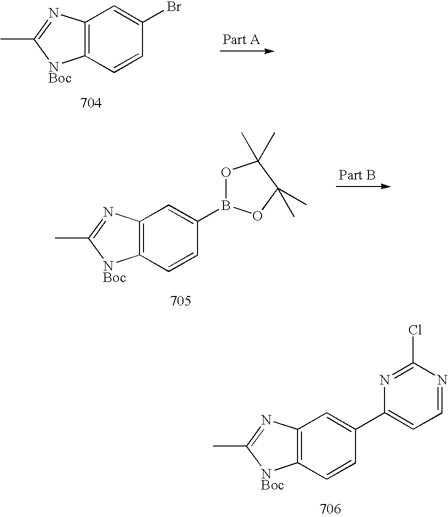

### Part A:

Compound 705 was prepared using the conditions described in Example 29 Part A. HPLC-MS t_{R}=2.33 min (UV_{254 nm}); mass calculated for formula C₁₉H₂₇BN₂O₄ 358.2, observed LCMS m/z 359.2 (M+H).

### Part B:

Compound 706 was prepared using the conditions described in Example 29 Part B. HPLC-MS t_{R}=2.07 min (UV_{254 nm}); mass calculated for formula C₁₇H₁₇ClN₄O₂ 344.1, observed LCMS m/z 345.1 (M+H).

### Example 30B

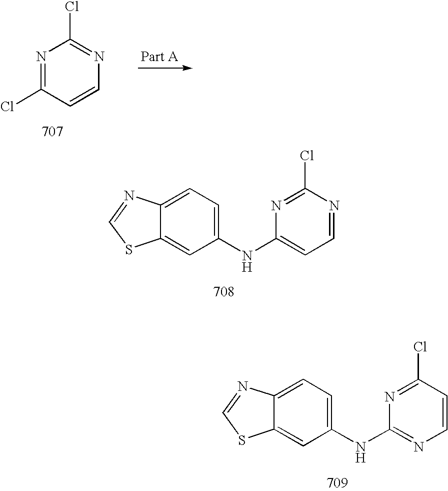

### Part A:

Under Argon, the vial was charged with 2,4-dichloropyrimidine 707 (0.149 g, 1.0 mmol), 6-aminobenzothiazole (0.150 g, 1.0 mmol), Pd₂ dba₃ (0.090 g, 0.1 mmol), 1,3-Bis(2,6-di-1-propylphenyl)-4,5-dihydroimidazolium tetrafluoroborate (0.095 g, 0.2 mmol) and NaO^{t}Bu (0.096 g, 1.0 mmol). Dioxane (2 mL) was added as solvent and the vial was sealed under Argon flow. The mixture was heated up to 80° C. and stirred over night. After cooling to room temperature, the mixture was diluted with EtOAc (50 mL) and washed with NH₄Cl (sat. aq.), brine and dried over Na₂SO₄. After concentration, the residue was purified with column (silica gel, Hexane/EtOAc=60/40) gave the product 708 and 709 as oil. 708: HPLC-MS t_{R}=1.35 min (UV_{254 nm}); mass calculated for formula C₁₁H₇ClN4S 262.0, observed LCMS m/z 263.0 (M+H). 709: HPLC-MS t_{R}=1.62 min (UV_{254 nm}); mass calculated for formula C₁₁H₇ClN₄S 262.0, observed LCMS m/z 263.0 (M+H).

### Example 30C

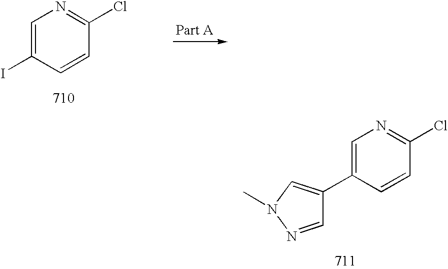

### Part A:

Compound 711 was prepared using the conditions described in Example 29 part D. HPLC-MS t_{R}=1.26 min (UV_{254 nm}); mass calculated for formula C₉H₈ClN₃ 193.0, observed LCMS m/z 194.0 (M+H).

### Example 30D

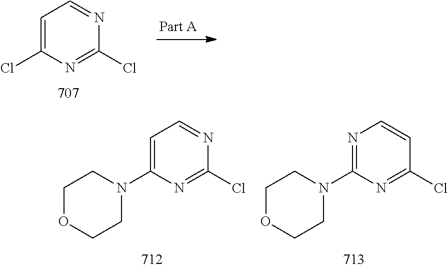

### Part A

Compound 712 and 713 were prepared using the same procedure and condition described in Example 29 part C.

### Example 30E

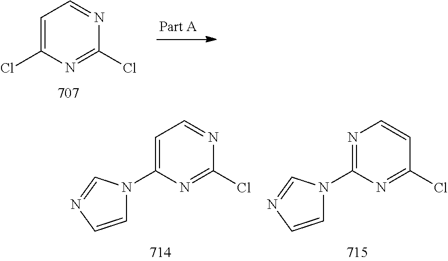

### Part A:

Compound 714 and 715 were synthesized from Compound 707 according to the procedures of Borowski et. al. (J. Med. Chem. 2000, 43, 1901 and the references therein.)

### Example 30F

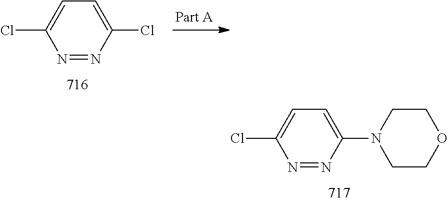

### Part A:

Compound 716 was prepared using the same procedure and condition described in Example 29 part C. HPLC-MS t_{R}=0.96 min (UV_{254 nm}); mass calculated for formula C₈H₁₀ClN₃O 199.1, observed LCMS m/z 200.1 (M+H).

### Example 30G

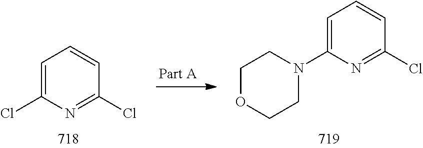

### Part A:

Compound 719 was prepared using the same procedure and condition described in Example 29 part C. HPLC-MS t_{R}=1.69 min (UV_{254 nm}); mass calculated for formula C9H11ClN2O 198.1, observed LCMS m/z 199.1 (M+H).

The compounds in Table 28 were prepared using the same procedure described in Example 29.

| | | | | Ret. |
|---|---|---|---|---|
| | | | MS m/z | Time |
| Compd # | Structure | EMW | (M⁺+ H) | (min) |
| 720 | 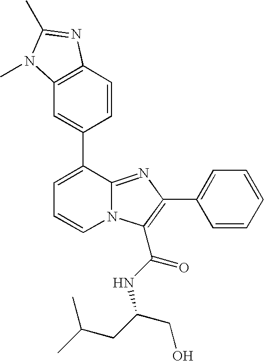 | 481.2 | 482.2 | 1.22 |
| 721 | 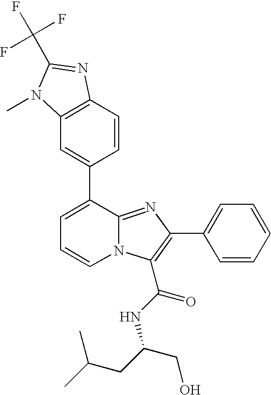 | 535.2 | 536.2 | 2.06 |
| 722 | 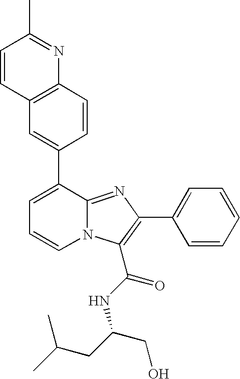 | 478.2 | 479.2 | 1.41 |
| 723 | 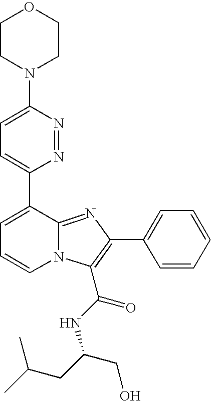 | 500.3 | 501.1 | 1.48 |
| 724 | 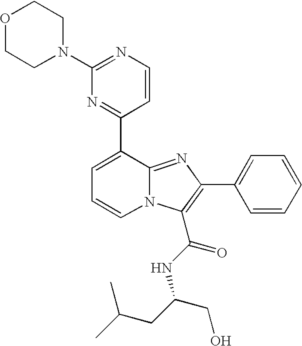 | 500.3 | 501.1 | 1.98 |
| 725 | 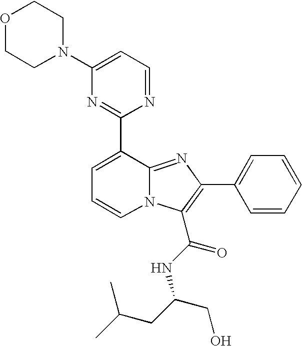 | 500.3 | 501.1 | 1.31 |
| 726 | 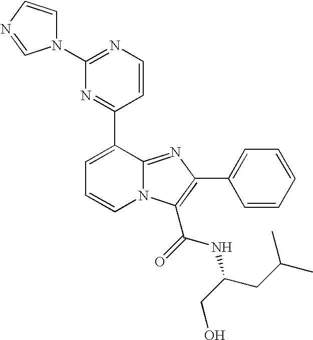 | 481.2 | 482.2 | 1.54 |
| 727 | 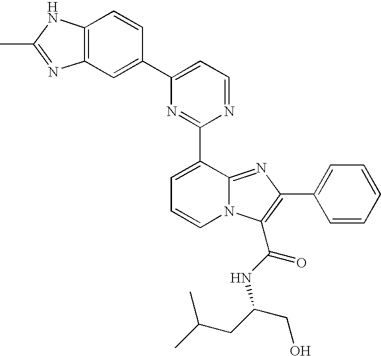 | 545.3 | 546.2 | 1.49 |

### Example 31

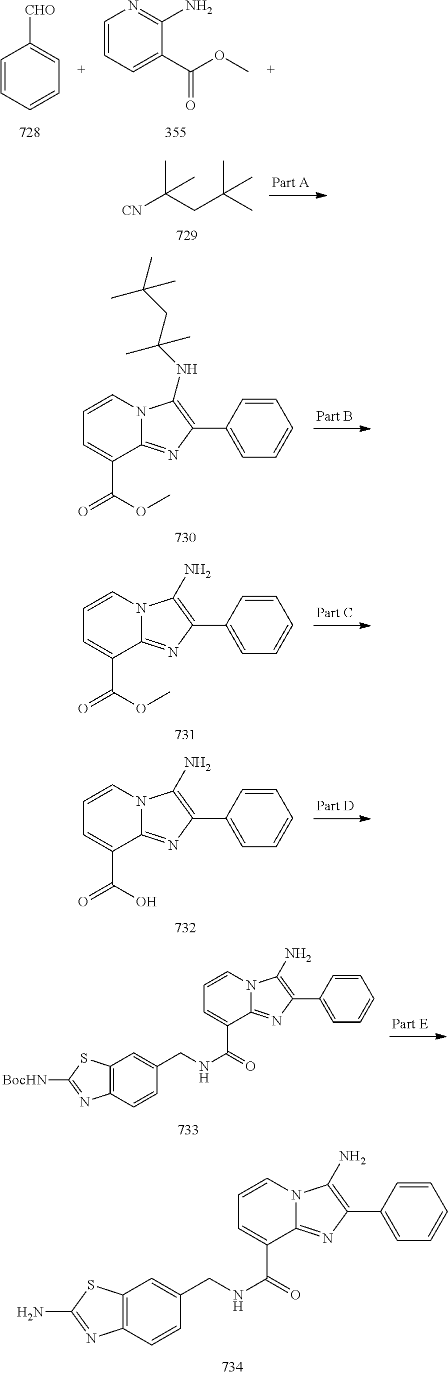

### Part A

The mixture of benzaldehyde 728 (1.06 g, 10 mmol), 2-aminopyridine 355 (1.52 g, 10 mmol), and 1,1,3,3-tetramethylbutyl isocyanide (1.94 mL, 90%, 10 mmol) in MeOH/DCM (1:3, 40 mL) was added Sc(OTf)₃ (0.492 g, 1.0 mmol). The reaction mixture was heated up to 70° C. and stirred for 3 days. After cooled down to room temperature, the solvent was removed by concentration and the residue was purified with column (silica gel, Hexane/EtOAc=70:30) gave the product 730 as yellow solid (2.1 g). HPLC-MS t_{R}=1.51 min (UV_{254 nm}), mass calculated for formula C₂₃H₂₉N₃O₂ 379.2, observed LCMS m/z 380.2 (M+H).

### Part B

The compound 730 (0.400 g) was dissolved in the mixture of DCM (5 mL) and TFA (5 mL) and stirred for 10 min. Then the solvent was removed with concentration and the resulting residue was treated aq. NaHCO₃ (40 mL). The aqueous was extracted with EtOAc (50 mL×3) and the organics was washed with brine and dried over Na₂SO₄. After concentration, the crude product 731 was used in the next step directly. HPLC-MS t_{R}=0.77 min (UV_{254 nm}); mass calculated for formula C₁₅H₁₃N₃O₂ 267.1, observed LCMS m/z 268.1 (M+H).

### Part C:

Compound 732 was prepared using the hydrolysis conditions described in Example 8. HPLC-MS t_{R}=0.67 min (UV_{254 nm}); mass calculated for formula C₁₄H₁₁N₃O₂ 253.1, observed LCMS m/z 254.1 (M+H).

### Part D:

Compound 733 was prepared using the peptide coupling conditions described in Example 8. HPLC-MS t_{R}=1.82 min (UV_{254 nm}); mass calculated for formula C₂₇H₂₆N₆O₃S 514.2, observed LCMS m/z 515.0 (M+H).

### Part E:

Compound 738 was prepared using the same deprotecting conditions described in Example 8.

| | | | | Ret. |
|---|---|---|---|---|
| | | | MS m/z | Time |
| Compd # | Structure | EMW | (M⁺+ H) | (min) |
| 734 | 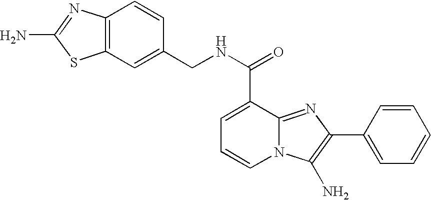 | 414.1 | 415.1 | 1.10 |

### Example 32

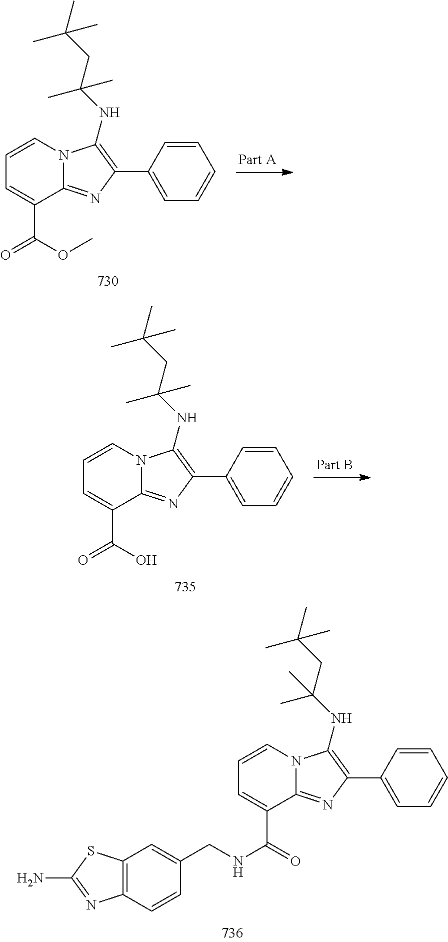

### Part A:

Compound 735 was prepared using the hydrolysis conditions described in Example 8. HPLC-MS t_{R}=1.64 min (UV_{254 nm}); mass calculated for formula C₂₂H₂₇N₃O₂ 365.2, observed LCMS m/z 366.3 (M+H).

### Part B:

Compound 736 was prepared using the peptide coupling conditions described in Example 8 with 2-amino-6-aminomethyl-benzothiazole

| | | | | Ret. |
|---|---|---|---|---|
| | | | MS m/z | Time |
| Compd # | Structure | EMW | (M⁺+ H) | (min) |
| 736 | 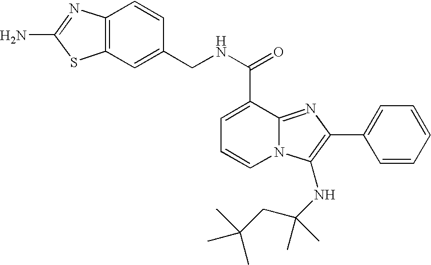 | 526.2 | 527.2 | 1.64 |

### Example 33

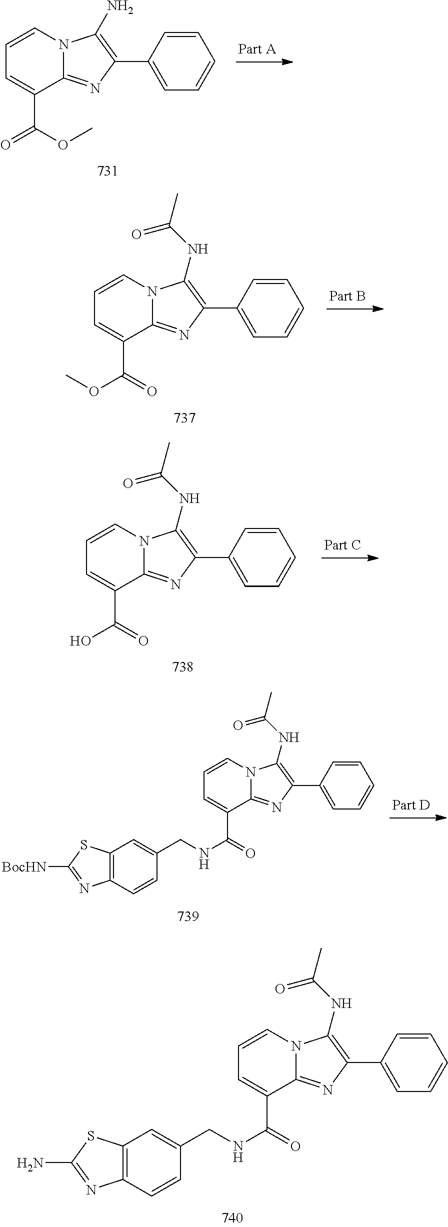

### Part A:

To the solution of 731 (0.027 g, 0.1 mmol) in DCM (5 mL), DIEA (0.100 mL, 0.6 mmol) was added followed by the addition of acetyl chloride (0.012 g, 0.15 mmol). The mixture was stirred at room temperature over night and diluted with EtOAc (50 mL). The organic was washed with water, brine and dried over Na₂SO₄. After concentration, the resulting residue was purified with column (silica gel, hexane/EtOAc=60/40) gave the product 737 as oil (0.023 g). HPLC-MS t_{R}=0.84 min (UV_{254 nm}); mass calculated for formula C₁₇H₁₅N₃O₃ 309.11, observed LCMS m/z 310.1 (M+H).

### Part B:

Compound 738 was prepared using the hydrolysis conditions described in Example 8. HPLC-MS t_{R}=0.69 min (UV_{254 nm}); mass calculated for formula C₁₆H₁₃N₃O₃ 295.01, observed LCMS m/z 296.0 (M+H).

### Part C:

Compound 739 was prepared using the peptide coupling conditions described in Example 8. HPLC-MS t_{R}=1.82 min (UV_{254 nm}); mass calculated for formula C₂₉H₂₈N₆O₄S 556.19, observed LCMS m/z 557.0 (M+H).

### Part D:

Compound 740 was prepared using the same deprotecting conditions described in Example 8. HPLC-MS t_{R}=1.12 min (UV_{254 nm}); mass calculated for formula C₂₄H₂₀N₆O₂S 456.1, observed LCMS m/z 457.0.1 (M+H).

The compounds in Table 29 were prepared using the same procedure in Example 33.

| | | | | Ret. |
|---|---|---|---|---|
| | | | MS m/z | Time |
| Compd # | Structure | EMW | (M⁺+ H) | (min) |
| 740 | 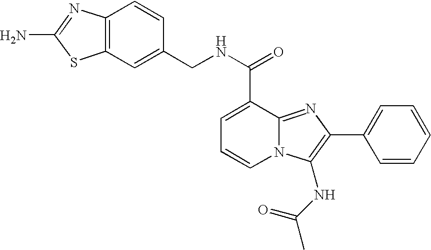 | 456.13 | 457.0 | 2.74 |
| 741 | 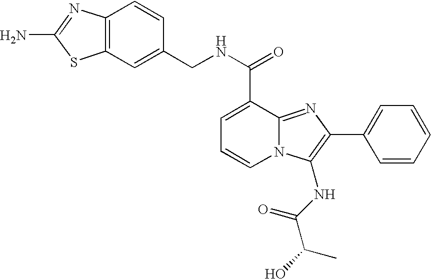 | 486.1 | 487.0 | 1.13 |
| 742 | 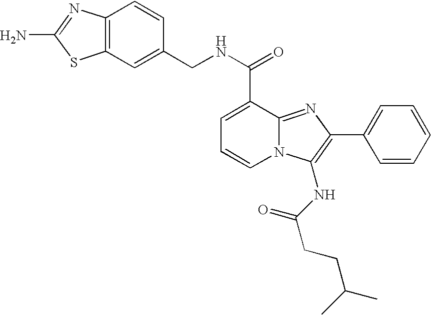 | 512.2 | 513.1 | 1.58 |
| 743 | 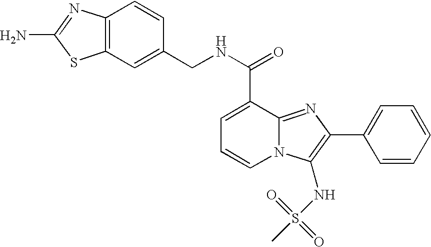 | 492.1 | 493 | 1.18 |

### Example 34

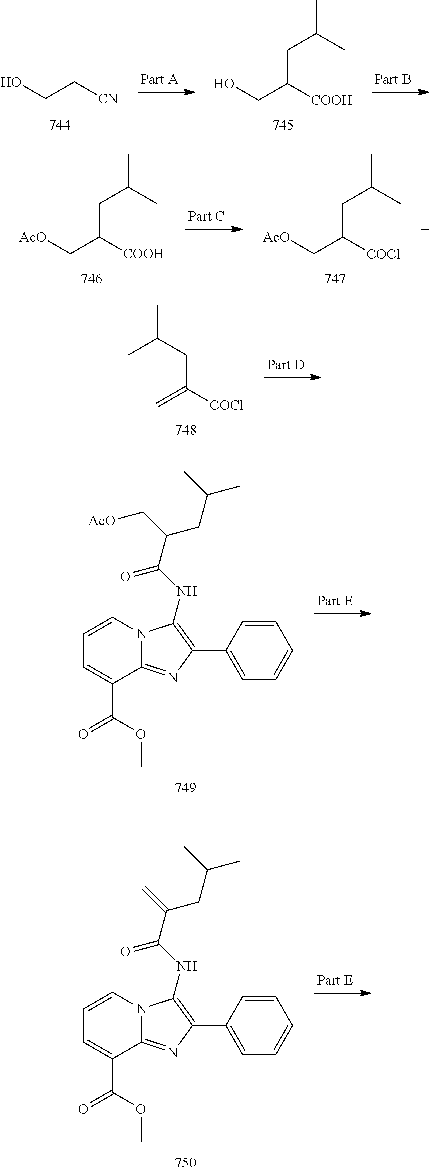

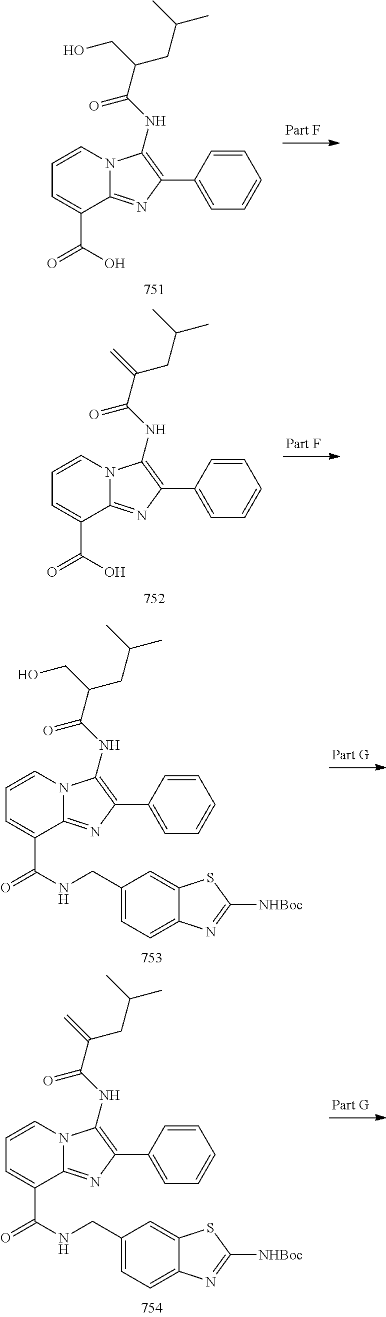

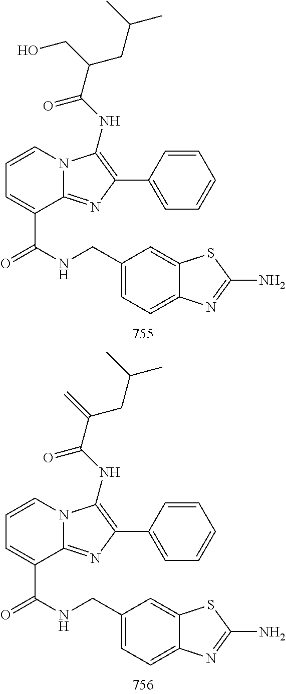

### Part A:

Compound 745 was synthesized from Compound 744 according to the procedures of Jung Dae Park et. al. (J. Med. Chem. 2002, 45, 911 and the references therein.).

### Part B:

To the mixture of compound 745 (0.146 g, 1.0 mmol) and DIEA (0.8 mL, 4.5 mmol) in DCM (10 mL), acetyl chloride (0.235 g, 3.0 mmol) was added. The resulting mixture was stirred at room temperature overnight and diluted with EtOAc (50 mL). The organic was washed with H₂O, NaHCO₃ (10% aq. 10 mL×3). The combined aqueous was treated with HCl (1N) to adjust pH to ˜5 and extracted with EtOAc. The organics was washed with brine and dried over Na₂SO₄. After concentration, the crude product 746 was used in the next step directly without further purification.

### Part C:

To the solution of compound 746 (0.125 g, 0.66 mmol) in dry DCM (3 mL), Oxalyl chloride (0.3 mL) was added dropwise at room temperature. The resulting mixture was stirred for 3 hours, and then the excess amount of oxalyl chloride and DCM was removed under vacuum. The mixture of 747, and 748 were used in the next step directly without purification.

### Part D:

Compound 749, and 750 were prepared using the same peptide coupling conditions described in Example 33, part A and separated by column. 749: HPLC-MS t_{R}=1.43 min (UV_{254 nm}), mass calculated for formula C24H27N3O5 437.2, observed LCMS m/z 438.1 (M+H). 750: HPLC-MS t_{R}=1.46 min (UV_{254 nm}); mass calculated for formula C₂₂H₂₃N₃O₃ 377.2, observed LCMS m/z 378.1 (M+H).

### Part E:

Compound 751 was prepared using the hydrolysis conditions described in Example 8. HPLC-MS t_{R}=1.20 min (UV_{254 nm}); mass calculated for formula C₂₁H₂₃N₃O₄ 381.2, observed LCMS m/z 382.1 (M+H).

Compound 752 was prepared using the hydrolysis conditions described in Example 8 HPLC-MS t_{R}=1.46 min (UV_{254 nm}); mass calculated for formula C₂₁H₂₁N₃O₃ 363.2, observed LCMS m/z 364.2 (M+H).

### Part F:

Compound 753 was prepared using the peptide coupling conditions described in Example 1. HPLC-MS t_{R}=2.03 min (UV_{254 nm}); mass calculated for formula C₃₄H₃₈N₆O₅S 642.3, observed LCMS m/z 643.2 (M+H).

Compound 754 was prepared using the peptide coupling conditions described in Example 8. HPLC-MS t_{R}=2.25 min (UV_{254 nm}); mass calculated for formula C₃₄H₃₆N₆O₄S 624.3, observed LCMS m/z 625.2 (M+H).

### Part G:

Compound 755 was prepared using the same deprotecting conditions described in Example 8. HPLC-MS t_{R}=1.40 min (UV_{254 nm}); mass calculated for formula C₂₉H₃₀N₆O₃S 542.2, observed LCMS m/z 543.1 (M+H).

Compound 756 was prepared using the same deprotecting conditions described in Example 8. HPLC-MS t_{R}=1.66 min (UV_{254 nm}); mass calculated for formula C₂₉H₂₈N₆O₂S 524.2, observed LCMS m/z 525.1 (M+H).

| | | | | Ret. |
|---|---|---|---|---|
| | | | MS m/z | Time |
| Compd # | Structure | EMW | (M⁺+ H) | (min) |
| 755 | 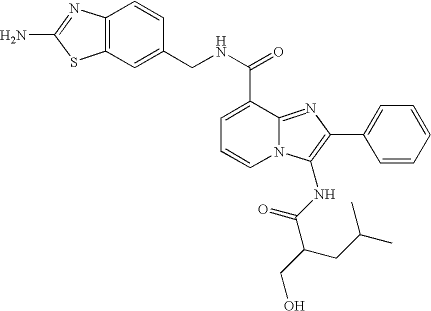 | 542.2 | 543.1 | 1.40 |
| 756 | 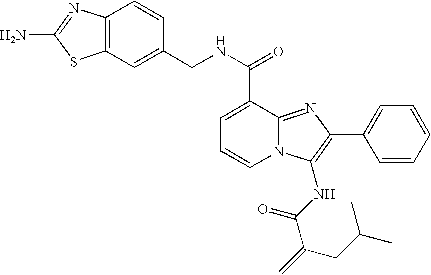 | 524.2 | 525.1 | 1.66 |

### Example 35

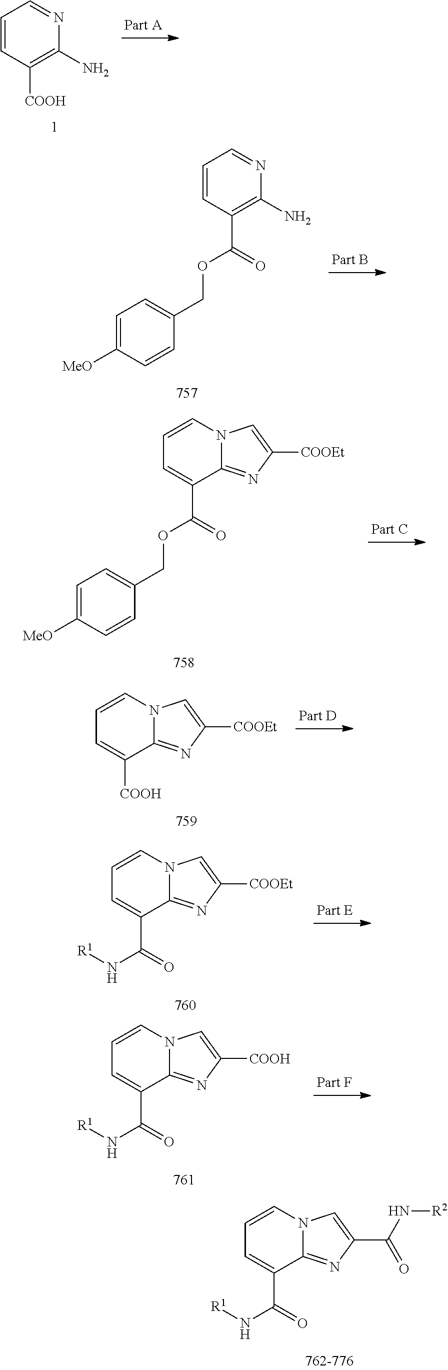

(wherein R¹ and R² are identified in Table 30)

### Part A:

To a solution of 2-aminonicotinic acid 1 (15.0 g, 109 mmol) in DCM (250 mL) was added thionyl chloride (22 g, 163 mmol). The resulting mixture was heated at reflux for 16 hours. The solution was cooled to room temperature then concentrated by reduced pressure. The resulting solid was re-dissolved in chloroform (150 mL), and then a pre-mixed solution of 4-methoxybenzyl alcohol (22.5 g, 163 mmol) and diisopropylethylamine (9.46 mL, 54.3 mmol) in chloroform (50 mL) was added to the acid chloride solution. The mixture was heated at reflux for 16 hours. The volatiles were removed in vacuo, ethyl acetate was added and the organic solution washed with saturated NaHCO₃(×1), brine (×1), dried over magnesium sulfate and concentrated. The isolated crude product was purified by flash column chromatography (SiO₂, dichloromethane/ethyl acetate-9:1) to afford compound 757 as a slightly yellow solid.

### Part B:

To a solution of compound 757 (2.7 g, 11 mmol) in DMF (5 mL) was added ethyl-bromopyruvate (4.1 g, 21 mmol) and cesium carbonate (6.8 g. 21 mmol). The reaction mixture was heated at 80° C. for 16 hours. The precipitates were removed by filtration, and the filtrate concentrated and then purified by flash column chromatography (SiO₂, ethyl acetate/hexanes—7:3) to afford compound 758 as a white solid.

### Part C:

To compound 758 (1.0 g) was added a mixture of trifluoroacetic acid (4.5 mL) and water (0.5 mL) and the reaction mixture was stirred at room temperature for 30 minutes. The solution was quenched with a mixture of acetonitrile (5 mL) and water (5 mL), and then concentrated to dryness to afford compound 759 as a white solid.

### Part D:

Compounds 760 were prepared using the coupling procedures described in Example 1B, Part G.

### Part E:

Compounds 761 were prepared using the saponification procedures described in Example 1B, Part D.

### Part F:

Compounds 762-776 (Table 30) were prepared using the coupling procedures described in Example 1B, Part E.

| | | | | Ret. |
|---|---|---|---|---|
| | | | MS m/z | Time |
| Compd # | Structure | EMW | (M⁺+ H) | (min) |
| 762 | 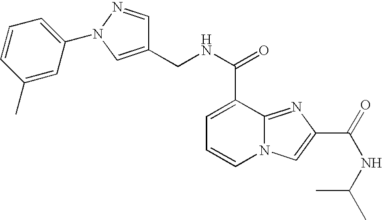 | 416.2 | 417.2 | 4.57 |
| 763 | 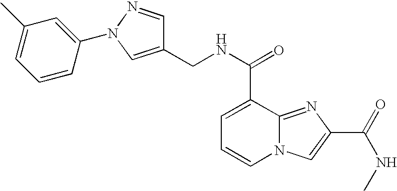 | 388.2 | 389.2 | 4.02 |
| 764 | 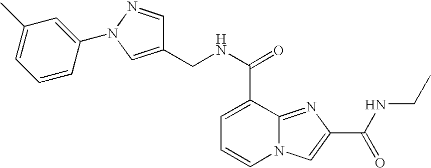 | 402.2 | 403.2 | 4.33 |
| 765 | 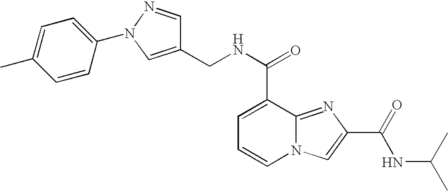 | 416.2 | 417.2 | 4.65 |
| 766 | 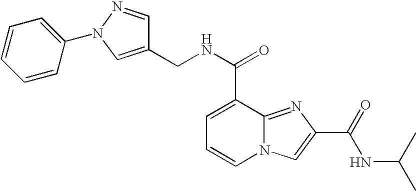 | 402.2 | 403.2 | 4.29 |
| 767 | 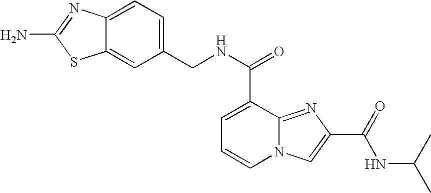 | 408.1 | 409.1 | 2.58 |
| 768 | 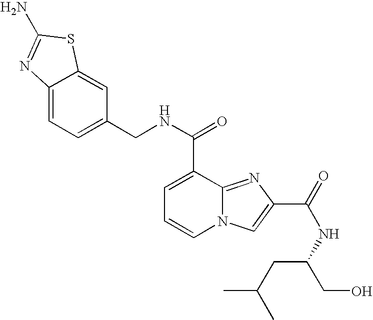 | 466.2 | 467.2 | 2.82 |
| 769 | 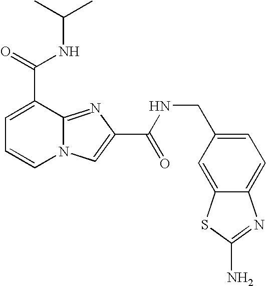 | 408.1 | 409.1 | 2.64 |
| 770 | 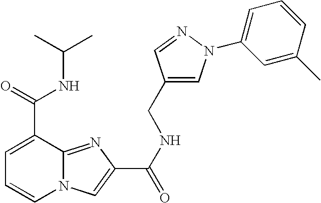 | 416.2 | 417.1 | 4.09 |
| 771 | 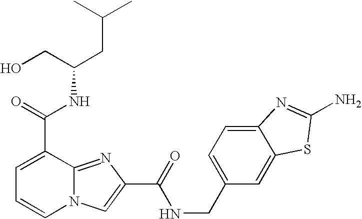 | 466.2 | 467.0 | 2.81 |
| 772 | 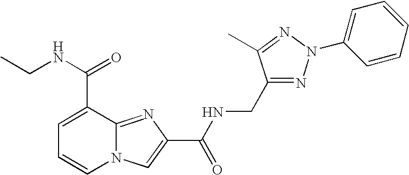 | 402.2 | 403.2 | 4.18 |
| 773 | 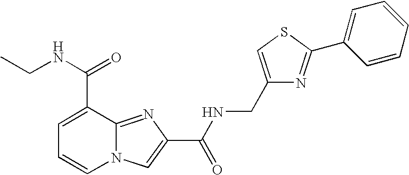 | 403.2 | 404.2 | 4.39 |
| 774 | 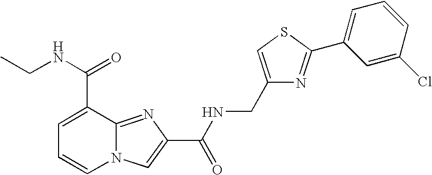 | 405.1 | 406.1 | 4.43 |
| 775 | 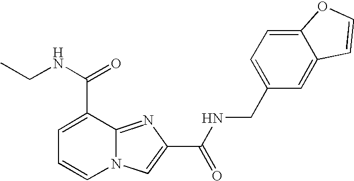 | 439.1 | 440.1 | 4.93 |
| 776 | 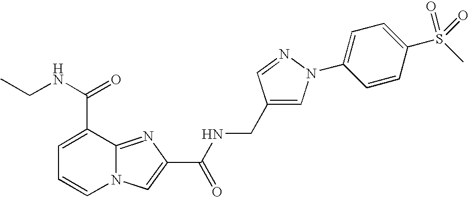 | 362.1 | 363.1 | 4.05 |

### Example 36

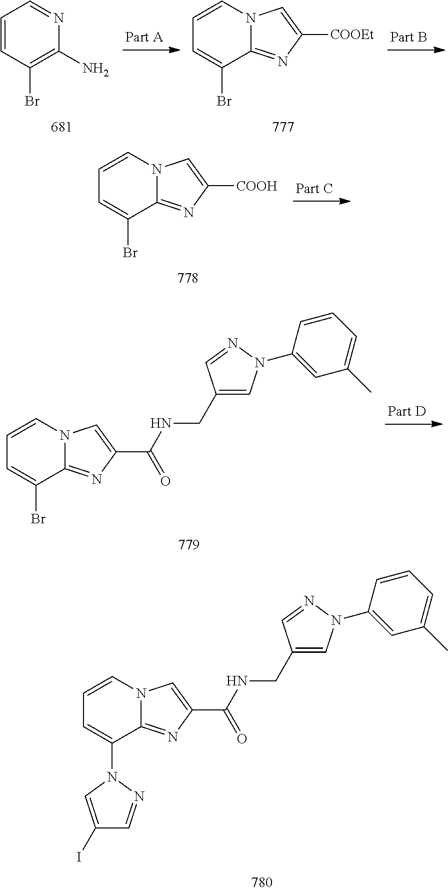

### Part A:

Compound 777 was prepared from the coupling of 2-amino-3-brmopyridine 681 and ethyl bromopyruvate using procedures described in Example 7A, Part A. HPLC-MS t_{R}=1.25 min (UV_{254 nm}); mass calculated for formula C₁₀H₉BrN₂O₂ 268.0, observed LCMS m/z 269.0 (M+H).

### Part B:

Compound 778 was prepared from compound 777 using the saponification procedures described in Example 1B, Part D. HPLC-MS t_{R}=0.51 min (UV_{254 nm}); mass calculated for formula C₈H₅BrN₂O₂ 240.0, observed LCMS m/z 241.0 (M+H).

### Part C:

Compound 779 was prepared from compound 778 using the coupling procedures described in Example 1B, Part G. HPLC-MS t_{R}=1.74 min (UV_{254 nm}); mass calculated for formula C₁₉H₁₆BrN₅O 409.1, observed LCMS m/z 410.0 (M+H).

### Part D:

4-Iodopyrazole (0.120 g, 0.61 mmol) was added to a solution of NMP (2 mL) containing sodium hydride (60%, 25 mg, 0.61 mmol) and then stirred at room temperature for 30 minutes. A solution of compound 779 (0.025 g, 0.061 mmol) in NMP (2 mL) was added and the reaction mixture heated at 110° C. for 120 hours. The reaction was monitored by LC-MS. Once the reaction was complete, the volatiles were removed in vacuo, and the isolated crude purified by Prep. LC to give 780.

| | | | | Ret. |
|---|---|---|---|---|
| | | | MS m/z | Time |
| Compd # | Structure | EMW | (M⁺+ H) | (min) |
| 780 | 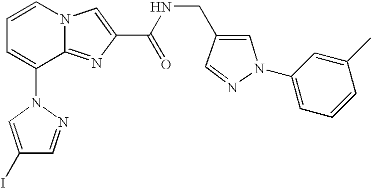 | 523.1 | 524.1 | 5.53 |

### Example 37

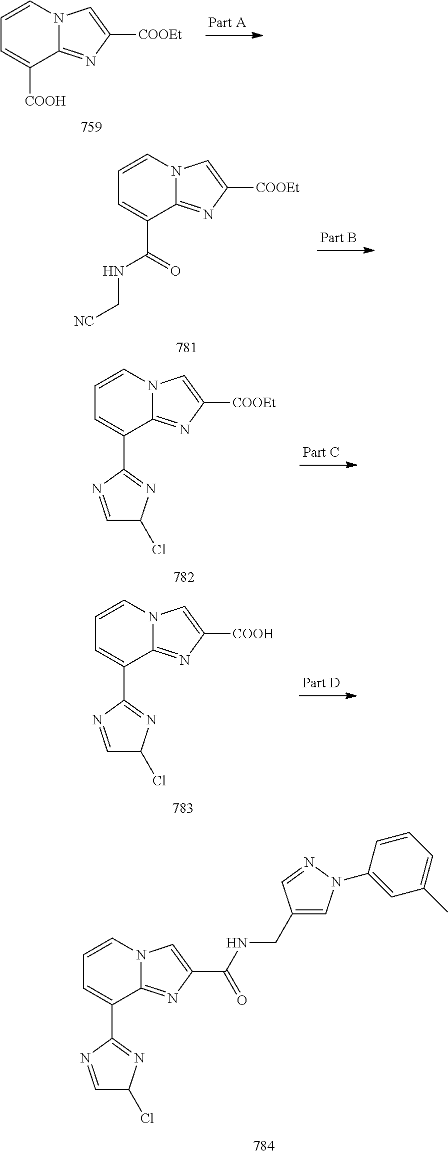

Compound 759 was prepared using procedures described in Example 35.

### Part A:

Compound 781 was prepared from the coupling of compound 759 and aminoacetonitrile using procedures described in Example 1B, Part G. HPLC-MS t_{R}=1.08 min (UV_{254 nm}); mass calculated for formula C₁₃H₁₂N₄O₃ 272.1, observed LCMS m/z 273.0 (M+H).

### Part B:

A mixture of compound 781 (0.022 g, 0.08 mmol), triphenylphosphine (0.053 g, 0.2 mmol), and carbon tetrachloride (0.020 mL, 0.2 mmol) in acetonitrile (5 mL) was heated at 45° C. for 16 hours. The reaction mixture was cooled to room temperature, concentrated, and dried to afford compound 782 which was taken forward directly to the next step. HPLC-MS t_{R}=1.53 min (UV_{254 nm}); mass calculated for formula C₁₃H₁₁ClN₄O₂ 290.1, observed LCMS m/z 291.1 (M+H).

### Part C:

Compound 783 was prepared from compound 781 using procedures described in Example 1B, Part D. HPLC-MS t_{R}=1.00 min (UV_{254 nm}); mass calculated for formula C₁₁H₇ClN₄O₂ 262.0, observed LCMS m/z 263.0 (M+H).

### Part D:

Compound 784 was prepared from compound 783 using the coupling procedures described in Example 1B, Part G.

The following ligand was synthesized using this procedure:

| | | | | Ret. |
|---|---|---|---|---|
| | | | MS m/z | Time |
| Compd # | Structure | EMW | (M⁺+ H) | (min) |
| 784 | 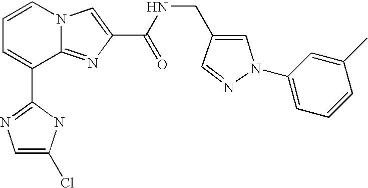 | 431.1 | 432.1 | 5.53 |

### Example 38

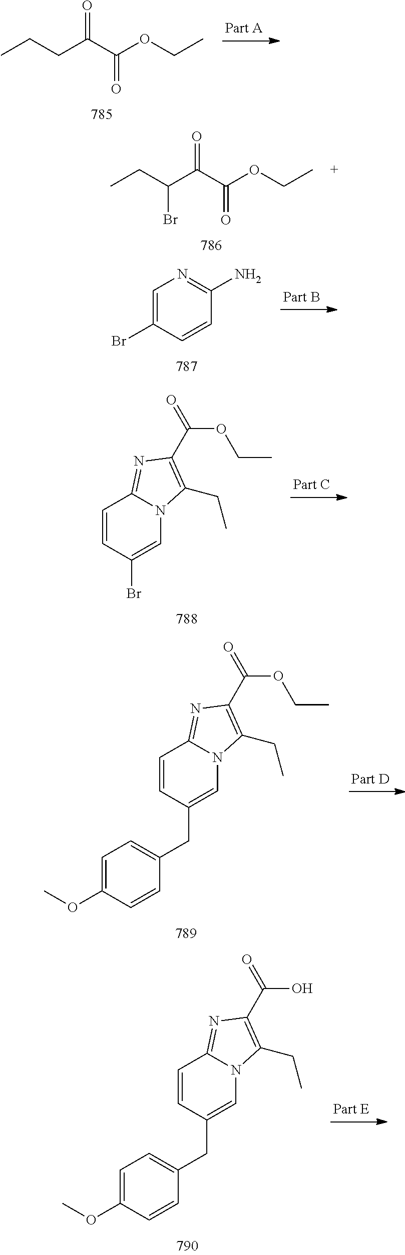

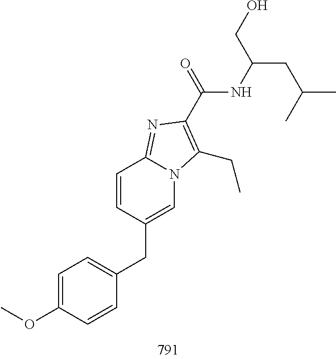

### Part A:

Compound 786 was prepared using the bromonation conditions described in Example 8 Part B.

### Part B:

Compound 787 was prepared using the cyclization conditions described in Example 8 Part C. HPLC-MS t_{R}=1.54 min (UV_{254 nm}); mass calculated for formula C₁₂H₁₃BrN₂O₂ 296.0, observed LCMS m/z 297.0 (M+H).

### Part C:

Under Ar, the bromine compound 788 (0.060 g, 0.2 mmol) in dioxane (2.0 ml) was added to the flask which was charged with Pd(dppf)Cl₂ (0.018 g, 0.02 mmol) followed by the addition of 4-methoxybenzylzinc chloride (0.089 g, 0.4 mmol). The mixture was thoroughly degassed by alternately connected the flask to vacuum and Argon. The resulting solution was heated upto 80° C. and stirred overnight and diluted by EtOAc after cooled to room temperature. The solid was removed by filter through Celite and washed with some EtOAc. Concentration to remove the solvent and the resulting residue was purified with column (silica gel, Hexane/EtOAc=40/60) gave the product 789 as oil. HPLC-MS t_{R}=1.48 min (UV_{254 nm}); mass calculated for formula C₂₀H₂₂N₂O₃ 338.2, observed LCMS m/z 339.1 (M+H).

### Part D:

Compound 790 was prepared using the hydrolysis conditions described in Example 8 Part G. HPLC-MS t_{R}=1.18 min (UV_{254 nm}); mass calculated for formula C₁₈H₁₈N₂O₃ 310.1, observed LCMS m/z 311.0 (M+H).

### Part E:

Compound 791 was prepared using the peptide coupling conditions described in Example 1B. HPLC-MS t_{R}=1.84 min (UV_{254 nm}); mass calculated for formula C₂₄H₃₁N₃O₃ 409.2, observed LCMS m/z 410.2 (M+H).

The compounds in Table 31 were synthesized using the same procedure:

| | | | | Ret. |
|---|---|---|---|---|
| | | | MS m/z | Time |
| Compd # | Structure | EMW | (M⁺+ H) | (min) |
| 791 | 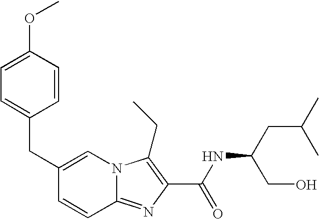 | 409.24 | 410.2 | |
| 792 | 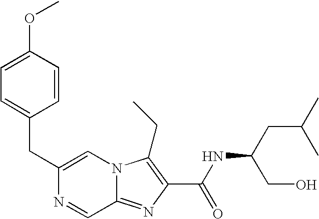 | 410.2 | 411.1 | 1.83 |
| 793 | 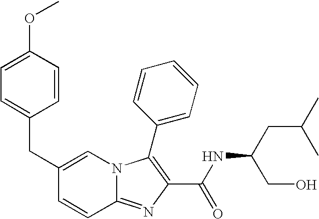 | 457.2 | 458.2 | 1.96 |
| 794 | 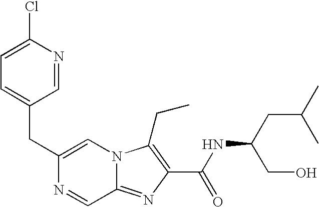 | 415.2 | 416.0 | 1.71 |

### Biological Assays:

### DELFIA (Dissociation Enhanced Lanthanide Fluorescence Immuno-Assay)

Before initiation of kinase reactions, compounds were pre-incubated with the enzyme for 10 minutes. Pre-incubation reactions contained 50 mM HEPES pH 7.3, 10 mM MgCl₂, 1 mM DTT, 75 mM NaCl, 1 mM EDTA, 1 mM EGTA, 0.01% CHAPS, 2 nM JNK1, 6 ug/mL biotinylated GST-ATF2, 0.1 mg/ml BSA, 5% DMSO and 0-100 μM compound in a total volume of 40 μL. After a 10 minute room temperature pre-incubation, 10 μL of 35 μM ATP was added to start the reaction (final concentration of ATP=7 uM). Reactions were incubated at room temperature for 30 minutes. A small aliquot (10 uL) was taken and quenched by adding into 190 uL of DELFIA Assay buffer containing 100 mM EDTA. The amount of phosphate transferred to biotinylated GST-ATF2 was measured using the Dissociation Enhanced Lanthanide Fluorescence Immuno-assay (DELFIA) from Perkin Elmer according to manufacturers protocol. Briefly, biotinylated GST-ATF2 was captured on streptavidin coated plates for 1 hour, washed twice, then incubated for 1 hour with a 1:1000 dilution of rabbit-anti-phospho-ATF2 antibody and a 1:3500 dilution of Europium-labeled anti-rabbit secondary antibody. Free antibody was removed with six washes, Europium was dissociated from the antibody, and Europium fluorescence was measured using an excitation wavelength of 340 nM and an emission wavelength of 615 nM. JNK2 and JNK3 kinase reactions were carried out similarly, with the exception that the final concentration of ATP was 4 uM and 2 uM, respectively.

### Cell Assay

### Jurkat IL2 Assay

One hundred microliters of cultured Jurkat cells (1,000,000/milliliter) in the following medium: RPM' 1640, 10% fetal bovine serum supplemented with glutamine, penicillin and streptomycin was added to a 96 well plate containing adherent anti-CD3 antibody (T-Cell Activation Plate, BD Biosciences #354725). An additional plate without attached antibody was also cultured with and without soluble anti-CD28 antibody and cells as additional anti-CD3 controls. Fifty microliters of medium containing serially diluted compound (0.4% DMSO) was added to compound wells, and 50 microliters of medium+0.4% DMSO was added to control wells in place of compound. Fifty microliters of medium containing anti-CD28 antibody, 1.6 micromolar, was next added to all wells except anti-CD28 controls. The cells, in a final volume of 200 microliters, were incubated in a cell culture cabinet (4% carbon dioxide) for 2 days at 37 C. After incubation 100 microliters of supernatant (cells are adherent) was removed from wells and IL2 production was quantified by ELISA, (Pierce Endogen Kit #EH2IL25). IL2 production was quantified on a Spectra Max Plus (Molecular Devices, Inc.) plate reader. Cell viability was determined by addition of 100 microliters of Promega CellTiter-Glo kit #G7571, followed by quantitation of fluorescence with a Victor² V 1420 fluorescence reader. IL2 inhibition and cell viability data were analyzed with GraphPad Prism software, (GraphPad Software, Inc.).

Compound Numbers: 13-16, 21-24, 27, 30, 33-40, 42-48, 51-74, 80, 84-94, 99, 101, 111, 112-131, 139-158, 162-172, 175, 177-181, 184, 186, 190, 191, 193-195, 200-235, 237-246, 271-307, 321-324, 326, 327, 354, 404-410, 444-453, 456, 457, 460-466, 468, 469, 471-489, 494-506, 542-545, 573, 574, 576, 578, 584, 588, 590, 593, 598-600, 605-611, 613-615, 619, 620, 622-629, 635, 647, 650-652, 664, 665, 672, 673-680, 686, 691, 692, 699, 703, 720-727, 734, 736, 740-743, 755, 756, 762-776, 780, 784, and 791-794 had a JNK1 IC₅₀ within the range of 6 to 100,000 nM.

Compound Numbers: 14, 16, 17, 22, 46, 47, 48, 56, 69, 93, 94, 111-115, 117, 118, 130, 131, 139, 140, 150, 154, -158, 204-206, 209, 213, 215-220, 224, 238, 242, 274, 277, 279, 280, 283, 285, 291, 292, 296, 298, 299, 300, 301, 305, 306, 307, 323, 324, 326, 327, 405, 445, 451, 452, 453, 456, 457, 460-466, 471, 472, 477, 478, 479, 480, 481, 483, 484, 485, 489, 490, 491, 502, 542, 543, 544, 545, 593, 598, 599, 605, 623-629, 647, 650, 651, 652, and 664 had a JNK1 IC₅₀ within the range of 6 to 100 nM.

Compound Numbers: 14, 16, 112, 114, 139, 156, 216, 218, 219, 277, 296, 300, 306, 307, 463, 478, 479, 483, 485, 491, 502, 598, 629, 647, 650, 651, and 652 had a JNK1 IC₅₀ within the range of 6 to 20 nM.

Compound Numbers: 14, 16, 112, 114, 139, 156, 216, 218, 219, 277, 296, 300, 306, 307, 463, 478, 479, 483, 485, 491, 502, 598, 629, 647, 650, 651, and 652 had a JNK1 IC₅₀ within the range of 6 to 20 nM.

Compound Numbers: 112, 478, 479, 502, 629, 651, and 652 had a JNK1 IC₅₀ within the range of 6 to 10 nM.

Compound Numbers: 14, 16, 17, 112, 114, 115, 130, 155, 216, 218, 219, 296, 299, 300, 301, 306, 307, 323, 327, 451, 456, 463, 478, 479, 483, 542, 544, 599 and 605 had a JNK2 IC₅₀ within the range of 4.0 to 46.0 nM.

Compound Numbers: 22, 42, 93, 111, 113, 205, 206, 215, and 452 had a JNK2 IC₅₀ within the range of 52.0 to 94.0 nM.

Compound Numbers: 15, 23, 48, 56, 62 and 291 had a JNK2 IC₅₀ within the range of 107.0 to 173.0 nM.

Compound Numbers: 13, 38, 178, 181, and 230 had a JNK2 IC₅₀ within the range of 201.0 to 666.0 nM.

Compound Numbers: 170, 350, and 351 had a JNK2 IC₅₀ within the range of 1070 to 11,500 nM.

Compound Numbers: 14, 16, 17, 22, 112, 114, 115, 130, 155, 215, 216, 218, 219, 296, 299, 300, 301, 306, 307, 323, 451, 456, 463, 478, 479, 483, 542, 544, 599, and 605 had a JNK3 IC₅₀ within the range of 9.0 to 50.0 nM.

Compound Numbers: 13, 38, 62, 93, 111, 113, 205, 206, 291, 327, and 452 had a JNK3 IC₅₀ within the range of 54.0 to 98.0 nM.

Compound Numbers: 15, 23, 42, 56, 170, and 181 had a JNK3 IC₅₀ within the range of 118.0 to 174.0 nM.

Compound Numbers: 48, 178, and 230 had a JNK3 IC₅₀ within the range of 209.0 to 479.0 nM.

Compound Number 350 had a JNK3 IC₅₀ of 16,100 nM, and compound Number 3561 had a JNK3 IC₅₀ of 10,000 nM.

JNK1 Data (in nM) for Compound Numbers 16, 112, 118, 478, 483, 544, 605, 647, 651, and 652 are given in the table below.

| | | JNK 1 |
|---|---|---|
| Compd # | Compound | IC₅₀ nM |
| 112 | 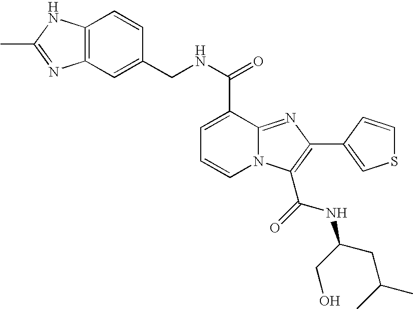 | 8 |
| 478 | 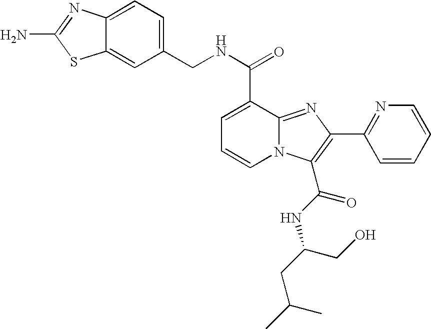 | 10 |
| 118 | 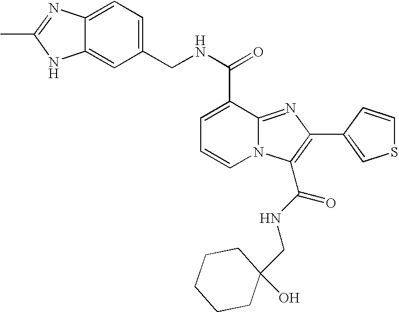 | 23 |
| 544 | 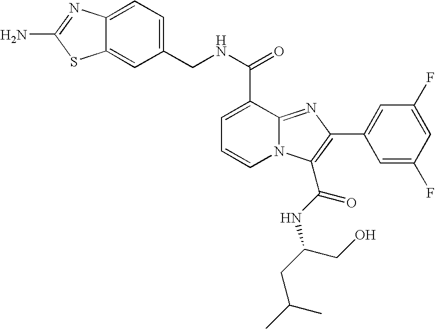 | 23 |
| 605 | 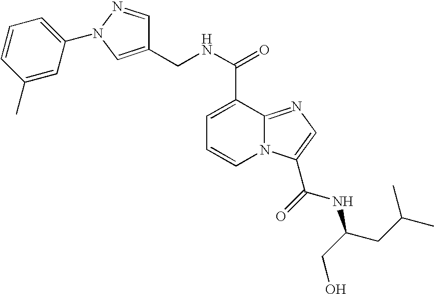 | 21 |
| 483 | 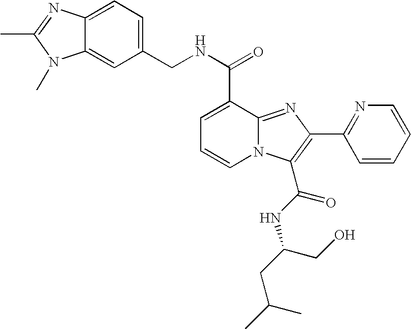 | 11 |
| 647 | 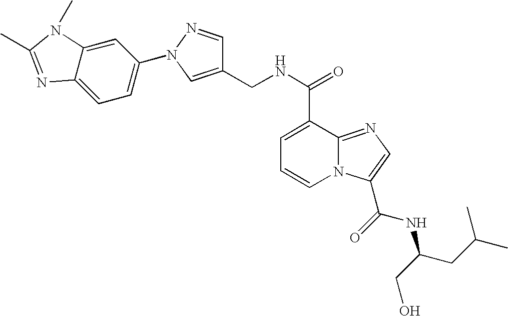 | 16 |
| 651 | 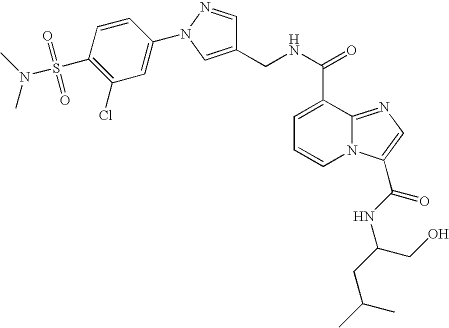 | 10 |
| 652 | 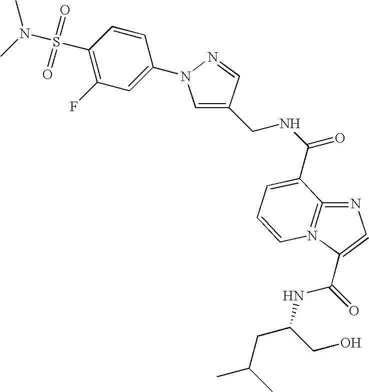 | 11 |
| 16 | 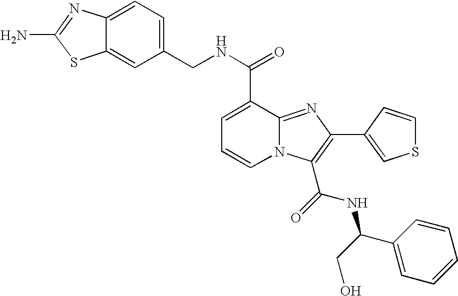 | 19 |

The compounds of this invention inhibit the activity of ERK1 and ERK2 Thus, this invention further provides a method of inhibiting ERK in mammals, especially humans, by the administration of an effective amount (e.g., a therapeutically effective amount) of one or more (e.g., one) compounds of this invention. The administration of the compounds of this invention to patients, to inhibit ERK1 and/or ERK2, is useful in the treatment of cancer.

In any of the methods of treating cancer described herein, unless stated otherwise, the methods can optionally include the administration of an effective amount of one or more (e.g., 1, 2 or 3, or 1 or 2, or 1) chemotherapeutic agents. The chemotherapeutic agents can be administered currently or sequentially with the compounds of this invention.

The methods of treating cancer described herein include methods wherein a combination of drugs (i.e., compounds, or pharmaceutically active ingredients, or pharmaceutical compositions) are used (i.e., the methods of treating cancer of this invention include combination therapies). Those skilled in the art will appreciate that the drugs are generally administered individually as a pharmaceutical composition. The use of a pharmaceutical composition comprising more than one drug is within the scope of this invention.

In any of the methods of treating cancer described herein, unless stated otherwise, the methods can optionally include the administration of an effective amount of radiation therapy. For radiation therapy, y-radiation is preferred.

Examples of cancers which may be treated by the methods of this invention include, but are not limited to: (A) lung cancer (e.g., lung adenocarcinoma and non small cell lung cancer), (B) pancreatic cancers (e.g., pancreatic carcinoma such as, for example, exocrine pancreatic carcinoma), (C) colon cancers (e.g., colorectal carcinomas, such as, for example, colon adenocarcinoma and colon adenoma), (D) myeloid leukemias (for example, acute myelogenous leukemia (AML), CML, and CMML), (E) thyroid cancer, (F) myelodysplastic syndrome (MDS), (G) bladder carcinoma, (H) epidermal carcinoma, (I) melanoma, (J) breast cancer, (K) prostate cancer, (L) head and neck cancers (e.g., squamous cell cancer of the head and neck), (M) ovarian cancer, (N) brain cancers (e.g., gliomas, such as glioma blastoma multiforme), (O) cancers of mesenchymal origin (e.g., fibrosarcomas and rhabdomyosarcomas), (P) sarcomas, (Q) tetracarcinomas, (R) nuroblastomas, (S) kidney carcinomas, (T) hepatomas, (U) non-Hodgkin's lymphoma, (V) multiple myeloma, and (W) anaplastic thyroid carcinoma.

Chemotherapeutic agents (antineoplastic agent) include but are not limited to: microtubule affecting agents, alkylating agents, antimetabolites, natural products and their derivatives, hormones and steroids (including synthetic analogs), and synthetics.

Examples of alkylating agents (including nitrogen mustards, ethylenimine derivatives, alkyl sulfonates, nitrosoureas and triazenes) include: Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Examples of antimetabolites (including folic acid antagonists, pyrimidine analogs, purine analogs and adenosine deaminase inhibitors) include: Methotrexate, 5-Fluorouracil, Floxuridine, Cytarabine, 6-Mercaptopurine, 6-Thioguanine, Fludarabine phosphate, Pentostatine, and Gemcitabine.

Examples of natural products and their derivatives (including vinca alkaloids, antitumor antibiotics, enzymes, lymphokines and epipodophyllotoxins) include: Vinblastine, Vincristine, Vindesine, Bleomycin, Dactinomycin, Daunorubicin, Doxorubicin, Epirubicin, Idarubicin, Paclitaxel (paclitaxel is a microtubule affecting agent and is commercially available as Taxol®), Paclitaxel derivatives (e.g. taxotere), Mithramycin, Deoxyco-formycin, Mitomycin-C, L-Asparaginase, Interferons (especially IFN-a), Etoposide, and Teniposide.

Examples of hormones and steroids (including synthetic analogs) include: 17a-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, and Zoladex. Examples of synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Examples of other chemotherapeutics include: Navelbene, CPT-11, Anastrazole, Letrazole, Capecitabinbe, Reloxafine, and Droloxafine.

A microtubule affecting agent (e.g., paclitaxel, a paclitaxel derivative or a paclitaxel-like compound), as used herein, is a compound that interferes with cellular mitosis, i.e., having an anti-mitotic effect, by affecting microtubule formation and/or action. Such agents can be, for instance, microtubule stabilizing agents or agents which disrupt microtubule formation.

Microtubule affecting agents, useful in the methods of this invention, are well known to those skilled in the art and include, but are not limited to: Allocolchicine (NSC 406042), Halichondrin B (NSC 609395), Colchicine (NSC 757), Colchicine derivatives (e.g., NSC 33410), Dolastatin 10 (NSC 376128), Maytansine (NSC 153858), Rhizoxin (NSC 332598), Paclitaxel (Taxol®, NSC 125973), Paclitaxel derivatives (e.g., Taxotere, NSC 608832), Thiocolchicine (NSC 361792), Trityl Cysteine (NSC 83265), Vinblastine Sulfate (NSC 49842), Vincristine Sulfate (NSC 67574), Epothilone A, Epothilone, Discodermolide (see Service, (1996) Science, 274:2009), Estramustine, Nocodazole, MAP4, and the like. Examples of such agents are described in, for example, Bulinski (1997) J. Cell Sci. 110:3055-3064, Panda (1997) Proc. Natl. Acad. Sci. USA 94:10560-10564, Muhlradt (1997) Cancer Res. 57:3344-3346, Nicolaou (1997) Nature 387:268-272, Vasquez (1997) Mol. Biol. Cell. 8:973-985, and Panda (1996) J. Biol. Chem. 271:29807-29812.

Chemotherapeutic agents with paclitaxel-like activity include, but are not limited to, paclitaxel and paclitaxel derivatives (paclitaxel-like compounds) and analogues. Paclitaxel and its derivatives (e.g. Taxol and Taxotere) are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

Compounds with possible tubulin polymerization activity can be screened in vitro. For example, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol., 89: 737-758. U.S. Pat. No. 5,569,720 also provides in vitro and in vivo assays for compounds with paclitaxel-like activity.

Thus, in the methods of this invention wherein at least one chemotherapeutic agent is used, examples of said chemotherapeutic agents include those selected from the group consisting of: microtubule affecting agents, alkylating agents, antimetabolites, natural products and their derivatives, hormones and steroids (including synthetic analogs), and synthetics.

In the methods of this invention wherein at least one chemotherapeutic agent is used, examples of said chemotherapeutic agents also include: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

In the methods of this invention wherein at least one chemotherapeutic agent is used, examples of such chemotherapeutic agents include:

(1) taxanes such as paclitaxel (TAXOL®) and/or docetaxel (Taxotere®);

(2) platinum coordinator compounds, such as, for example, carboplatin, cisplatin and oxaliplatin (e.g. Eloxatin);

(3) EGF inhibitors that are antibodies, such as: HER2 antibodies (such as, for example trastuzumab (Herceptie), Genentech, Inc.), Cetuximab (Erbitux, IMC-C225, ImClone Systems), EMD 72000 (Merck KGaA), anti-EFGR monoclonal antibody ABX (Abgenix), TheraClM-h-R3 (Center of Molecular Immunology), monoclonal antibody 425 (Merck KGaA), monoclonal antibody ICR-62 (ICR, Sutton, England); Herzyme (Elan Pharmaceutical Technologies and Ribozyme Pharmaceuticals), PKI 166 (Novartis), EKB 569 (Wyeth-Ayerst), GW 572016 (GlaxoSmithKline), Cl 1033 (Pfizer Global Research and Development), trastuzmab-maytansinoid conjugate (Genentech, Inc.), mitumomab (Imclone Systems and Merck KGaA) and Melvax II (Imclone Systems and Merck KgaA);

(4) EGF inhibitors that are small molecules, such as, Tarceva (TM) (OSI-774, OSI Pharmaceuticals, Inc.), and Iressa (ZD 1839, Astra Zeneca);

(5) VEGF inhibitors that are antibodies such as: bevacizumab (Genentech, Inc.), and IMC-1C11 (ImClone Systems), DC 101 (a KDR VEGF Receptor 2 from ImClone Systems);

(6) VEGF kinase inhibitors that are small molecules such as SU 5416 (from Sugen, Inc), SU 6688 (from Sugen, Inc.), Bay 43-9006 (a dual VEGF and bRAF inhibitor from Bayer Pharmaceuticals and Onyx Pharmaceuticals);

(7) estrogen receptor antagonists or selective estrogen receptor modulators (SERMs), such as tamoxifen, idoxifene, raloxifene, trans-2,3-dihydroraloxifene, levormeloxifene, droloxifene, MDL 103,323, and acolbifene (Schering Corp.);

(8) anti-tumor nucleoside derivatives such as 5-fluorouracil, gemcitabine, capecitabine, cytarabine (Ara-C), fludarabine (F-Ara-A), decitabine, and chlorodeoxyadenosine (Cda, 2-Cda);

(9) epothilones such as BMS-247550 (Bristol-Myers Squibb), and EP0906 (Novartis Pharmaceuticals);

(10) topoisomerase inhibitors such as topotecan (Glaxo SmithKline), and Camptosar (Pharmacia);

(11) vinca alkaloids, such as, navelbine (Anvar and Fabre, France), vincristine and vinblastine;

(12) antibodies that are inhibitors of αVβ3 integrins, such as, LM-609 (see, Clinical Cancer Research, Vol. 6, page 3056-3061, August 2000, the disclosure of which is incorporated herein by reference thereto);

(13) folate antagonists, such as Methotrexate (MTX), and Premetrexed (Alimta);

(14) ribonucleotide reductase inhibitors, such as Hydroxyurea (HU);

(15) anthracyclines, such as Daunorubicin, Doxorubicin (Adriamycin), and Idarubicin;

(16) biologics, such as interferon (e.g., Intron-A and Roferon), pegylated interferon (e.g., Peg-Intron and Pegasys), and Rituximab (Rituxan, antibody used for the treatment of non-Hodgkin's lymphoma);

(17) thalidomide (or related imid);

(18) Bcr/abl kinase inhibitors, such as, for example Gleevec (STI-571), AMN-17, ONO12380, SU11248 (Sunitinib) and BMS-354825

(19) MEK1 and/or MEK2 inhibitors, such as PD0325901 and Arry-142886 (AZD6244);

(20) IGF-1 and IGF-2 inhibitors that are small molecules, such as, for example, NVP-AEW541;

(21) small molecule inhibitors of RAF and BRAF kinases, such as, for example, BAY 43-9006 (Sorafenib);

(22) small molecule inhibitors of cell cycle dependent kinases such as CDK1, CDK2, CDK4 and CDK6, such as, for example, CYC202, BMS387032, and Flavopiridol;

(23) alkylating agents, such as, for example, Temodar® brand of temozolomide;

(24) farnesyl protein transferase inhibitors, such as, for example:
(a) Sarasar® brand of lonifarnib (i.e., 4-[2-[4-(3,10-dibromo-8-chloro-6,11-dihydro-5H-benzo[5,6]cyclohepta[1,2-b]byridin-11-yl)-1-piperidinyl)-2-oxoethyl]-1-piperidinecarboxamide, see for example, U.S. Pat. No. 5,874,442 issued Feb. 23, 1999, and U.S. Pat. No. 6,632,455 issued Oct. 14, 2003 the disclosures of each being incorporated herein by reference thereto),
(b) Zarnestra® brand of tipifarnib (i.e., (R)-6-amino[(4-chlorophenyl)(1-methyl-1H-imidazol-5-yl)methyl]-4-(3-chlorophenyl)-1-methyl-2(1H)-quinolinone, see for example, WO 97/16443 published May 9, 1997 and U.S. Pat. No. 5,968,952 issued Oct. 19, 1999, the disclosures of each being incorporated herein by reference thereto), and
(c) Bristol-Myers Squibb 214662:

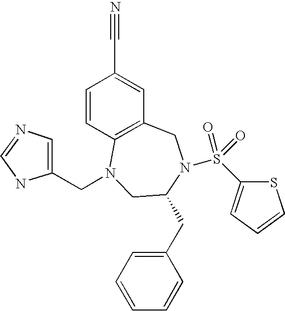

(see WO97/30992 published Aug. 28, 1997, U.S. Pat. No. 6,011,029 issued Jan. 4, 2000, and U.S. Pat. No. 6,455,523, the disclosures of each being incorporated herein by reference thereto).

The Bcr/abl kinase inhibitors, EGF receptor inhibitors, and HER-2 antibodies (EGF receptor inhibitors that are antibodies) described above are also known as signal transduction inhibitors. Therefore, chemotherapeutic agents, as used herein, include signal transduction inhibitors.

Typical signal transduction inhibitors, that are chemotherapeutic agents, include but are not limited to: (i) Bcr/abl kinase inhibitors such as, for example, STI 571 (Gleevec), (ii) Epidermal growth factor (EGF) receptor inhibitor such as, for example, Kinase inhibitors (lressa, OSI-774) and antibodies (Imclone: C225 [Goldstein et al. (1995), Clin Cancer Res. 1:1311-1318], and Abgenix: ABX-EGF) and (iii) HER-2/neu receptor inhibitors such as, for example, Herceptin® (trastuzumab).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA), the Physician's Desk Reference, 56^{th} Edition, 2002 (published by Medical Economics company, Inc. Montvale, N.J. 07645-1742), and the Physician's Desk Reference, 57^{th} Edition, 2003 (published by Thompson PDR, Montvale, N.J. 07645-1742); the disclosures of which is incorporated herein by reference thereto.

For example, the compound of formula 1.0 (e.g., a pharmaceutical composition comprising the compound of formula 1.0); can be administered orally (e.g., as a capsule), and the chemotherapeutic agents can be administered intravenously, usually as an IV solution. The use of a pharmaceutical composition comprising more than one drug is within the scope of this invention.

The compound of formula 1.0 and the chemotherapeutic agents are administered in therapeutically effective dosages to obtain clinically acceptable results, e.g., reduction or elimination of symptoms or of the tumor. Thus, the compound of formula 1.0 and chemotherapeutic agents can be administered concurrently or consecutively in a treatment protocol. The administration of the chemotherapeutic agents can be made according to treatment protocols already known in the art.

In general when more than one chemotherapeutic agent is used in the methods of this invention, the chemotherapeutic agents are administered on the same day either concurrently or consecutively in their standard dosage form. For example, the chemotherapeutic agents are usually administered intravenously, preferably by an IV drip using IV solutions well known in the art (e.g., isotonic saline (0.9% NaCl) or dextrose solution (e.g., 5% dextrose)).

When two or more chemotherapeutic agents are used, the chemotherapeutic agents are generally administered on the same day; however, those skilled in the art will appreciate that the chemotherapeutic agents can be administered on different days and in different weeks. The skilled clinician can administer the chemotherapeutic agents according to their recommended dosage schedule from the manufacturer of the agent and can adjust the schedule according to the needs of the patient, e.g., based on the patient's response to the treatment. For example, when gemcitabine is used in combination with a platinum coordinator compound, such as, for example, cisplatin, to treat lung cancer, both the gemcitabine and the cisplatin are given on the same day on day one of the treatment cycle, and then gemcitabine is given alone on day 8 and given alone again on day 15

The compounds of this invention and chemotherapeutic agents can be administered in a treatment protocol that usually lasts one to seven weeks, and is repeated typically from 6 to 12 times. Generally the treatment protocol can last one to four weeks. Treatment protocols of one to three weeks can also be used. A treatment protocol of one to two weeks can also be used. During this treatment protocol or cycle the compounds of this invention can be administered daily while the chemotherapeutic agents can be administered one or more times a week. Generally, a compound of this invention can be administered daily (i.e., once per day), and in one embodiment twice per day, and the chemotherapeutic agent is administered once a week or once every three weeks. For example, the taxanes (e.g., Paclitaxel (e.g., Taxol®) or Docetaxel (e.g., Taxotere®)) can be administered once a week or once every three weeks.

However, those skilled in the art will appreciate that treatment protocols can be varied according to the needs of the patient. Thus, the combination of compounds (drugs) used in the methods of this invention can be administered in variations of the protocols described above. For example, the compounds of this invention can be administered discontinuously rather than continuously during the treatment cycle. Thus, for example, during the treatment cycle the compounds of this invention can be administered daily for a week and then discontinued for a week, with this administration repeating during the treatment cycle. Or the compounds of this invention can be administered daily for two weeks and discontinued for a week, with this administration repeating during the treatment cycle. Thus, the compounds of this invention can be administered daily for one or more weeks during the cycle and discontinued for one or more weeks during the cycle, with this pattern of administration repeating during the treatment cycle. This discontinuous treatment can also be based upon numbers of days rather than a full week. For example, daily dosing for 1 to 6 days, no dosing for 1 to 6 days with this pattern repeating during the treatment protocol. The number of days (or weeks) wherein the compounds of this invention are not dosed do not have to equal the number of days (or weeks) wherein the compounds of this invention are dosed. Usually, if a discontinuous dosing protocol is used, the number of days or weeks that the compounds of this invention are dosed is at least equal or greater than the number of days or weeks that the compounds of this invention are not dosed.

The chemotherapeutic agent could be given by bolus or continuous infusion. The chemotherapeutic agent could be given daily to once every week, or once every two weeks, or once every three weeks, or once every four weeks during the treatment cycle. If administered daily during a treatment cycle, this daily dosing can be discontinuous over the number of weeks of the treatment cycle. For example, dosed for a week (or a number of days), no dosing for a week (or a number of days, with the pattern repeating during the treatment cycle.

The compounds of this invention can be administered orally, preferably as a solid dosage form, and in one embodiment as a capsule, and while the total therapeutically effective daily dose can be administered in one to four, or one to two divided doses per day, generally, the therapeutically effective dose is given once or twice a day, and in one embodiment twice a day. The compounds of this invention can be administered in an amount of about 50 to about 400 mg once per day, and can be administered in an amount of about 50 to about 300 mg once per day. The compounds of this invention are generally administered in an amount of about 50 to about 350 mg twice a day, usually 50 mg to about 200 mg twice a day, and in one embodiment about 75 mg to about 125 mg administered twice a day, and in another embodiment about 100 mg administered twice a day.

If the patient is responding, or is stable, after completion of the therapy cycle, the therapy cycle can be repeated according to the judgment of the skilled clinician. Upon completion of the therapy cycles, the patient can be continued on the compounds of this invention at the same dose that was administered in the treatment protocol, or, if the dose was less than 200 mg twice a day, the dose can be raised to 200 mg twice a day. This maintenance dose can be continued until the patient progresses or can no longer tolerate the dose (in which case the dose can be reduced and the patient can be continued on the reduced dose).

The chemotherapeutic agents, used with the compounds of this invention, are administered in their normally prescribed dosages during the treatment cycle (i.e., the chemotherapeutic agents are administered according to the standard of practice for the administration of these drugs). For example: (a) about 30 to about 300 mg/m² for the taxanes; (b) about 30 to about 100 mg/m² for Cisplatin; (c) AUC of about 2 to about 8 for Carboplatin; (d) about 2 to about 4 mg/m² for EGF inhibitors that are antibodies; (e) about 50 to about 500 mg/m² for EGF inhibitors that are small molecules; (f) about 1 to about 10 mg/m² for VEGF kinase inhibitors that are antibodies; (g) about 50 to about 2400 mg/m² for VEGF inhibitors that are small molecules; (h) about 1 to about 20 mg for SERMs; (i) about 500 to about 1250 mg/m² for the anti-tumor nucleosides 5-Fluorouracil, Gemcitabine and Capecitabine; (j) for the anti-tumor nucleoside Cytarabine (Ara-C) 100-200 mg/m²/day for 7 to 10 days every 3 to 4 weeks, and high doses for refractory leukemia and lymphoma, i.e., 1 to 3 gm/m² for one hour every 12 hours for 4-8 doses every 3 to four weeks; (k) for the anti-tumor nucleoside Fludarabine (F-ara-A) 10-25 mg/m²/day every 3 to 4 weeks; (l) for the anti-tumor nucleoside Decitabine 30 to 75 mg/m² for three days every 6 weeks for a maximum of 8 cycles; (m) for the anti-tumor nucleoside Chlorodeoxyadenosine (CdA, 2-CdA) 0.05-0.1 mg/kg/day as continuous infusion for up to 7 days every 3 to 4 weeks; (n) about 1 to about 100 mg/m² for epothilones; (o) about 1 to about 350 mg/m² for topoisomerase inhibitors; (p) about 1 to about 50 mg/m² for vinca alkaloids; (q) for the folate antagonist Methotrexate (MTX) 20-60 mg/m² by oral, IV or IM every 3 to 4 weeks, the intermediate dose regimen is 80-250 mg/m² IV over 60 minutes every 3 to 4 weeks, and the high dose regimen is 250-1000 mg/m² IV given with leucovorin every 3 to 4 weeks; (r) for the folate antagonist Premetrexed (Alimta) 300-600 mg/m² (10 minutes IV infusion day 1) every 3 weeks; (s) for the ribonucleotide reductase inhibitor Hydroxyurea (HU) 20-50 mg/kg/day (as needed to bring blood cell counts down); (t) the platinum coordinator compound Oxaliplatin (Eloxatin) 50-100 mg/m² every 3 to 4 weeks (preferably used for solid tumors such as non-small cell lung cancer, colorectal cancer and ovarian cancer); (u) for the anthracycline daunorubicin 10-50 mg/m²/day IV for 3-5 days every 3 to 4 weeks; (v) for the anthracycline Doxorubicin (Adriamycin) 50-100 mg/m² IV continuous infusion over 1-4 days every 3 to 4 weeks, or 10-40 mg/m² IV weekly; (w) for the anthracycline Idarubicin 10-30 mg/m² daily for 1-3 days as a slow IV infusion over 10-20 minutes every 3 to 4 weeks; (x) for the biologic interferon (Intron-A, Roferon) 5 to 20 million IU three times per week; (y) for the biologic pegylated interferon (Peg-intron, Pegasys) 3 to 4 micrograms/kg/day chronic sub cutaneous (until relapse or loss of activity); (z) for the biologic Rituximab (Rituxan) (antibody used for non-Hodgkin's lymphoma) 200-400 mg/m² IV weekly over 4-8 weeks for 6 months; (aa) for the alkylating agent temozolomide 75 mg/m² to 250 mg/m², for example, 150 mg/m², or for example, 200 mg/m², such as 200 mg/m² for 5 days; and (bb) for the MEK1 and/or MEK2 inhibitor PD0325901, 15 mg to 30 mg, for example, 15 mg daily for 21 days every 4 weeks.

Gleevec can be used orally in an amount of about 200 to about 800 mg/day.

Thalidomide (and related imids) can be used orally in amounts of about 200 to about 800 mg/day, and can be contiuously dosed or used until releapse or toxicity. See for example Mitsiades et al., “Apoptotic signaling induced by immunomodulatory thalidomide analogs in human multiple myeloma cells; therapeutic implications”, Blood, 99(12):4525-30, Jun. 15, 2002, the disclosure of which is incorporated herein by reference thereto.

The FPT inhibitor Sarasar® (brand of lonifarnib) can be administered orally (e.g., capsule) in amounts of about 50 to about 200 mg given twice a day, or in amounts of about 75 to about 125 mg given twice a day, or in amounts of about 100 to about 200 mg given twice a day, or in an amount of about 100 mg given twice a day.

Paclitaxel (e.g., Taxol®), for example, can be administered once per week in an amount of about 50 to about 100 mg/m² and in another example about 60 to about 80 mg/m². In another example Paclitaxel (e.g., Taxol®) can be administered once every three weeks in an amount of about 150 to about 250 mg/m² and in another example about 175 to about 225 mg/m².

In another example, Docetaxel (e.g., Taxotere®) can be administered once per week in an amount of about 10 to about 45 mg/m². In another example Docetaxel (e.g., Taxotere®) can be administered once every three weeks in an amount of about 50 to about 100 mg/m².

In another example Cisplatin can be administered once per week in an amount of about 20 to about 40 mg/m². In another example Cisplatin can be administered once every three weeks in an amount of about 60 to about 100 mg/m².

In another example Carboplatin can be administered once per week in an amount to provide an AUC of about 2 to about 3. In another example Carboplatin can be administered once every three weeks in an amount to provide an AUC of about 5 to about 8.

In another embodiment this invention is directed to a method of treating cancer in a patient in need of such treatment, said method comprising administering to said patient an effective amount of at least one (1, 2 or 3, or 1 or 2, or 1, and usually 1) compound of formula 1.0.

Another embodiment of this invention is directed to a method of treating cancer in a patient in need of such treatment, said method comprising administering to said patient an effective amount of at least one (1, 2 or 3, or 1 or 2, or 1, and usually 1) compound of formula 1.0 and an effective amount of a chemotherapeutic agent.

Another embodiment of this invention is directed to a method of treating cancer in a patient in need of such treatment, said method comprising administering to said patient an effective amount of at least one (1, 2 or 3, or 1 or 2, or 1, and usually 1) compound of formula 1.0 and an effective amount of a chemotherapeutic agent, wherein the chemotherapeutic agent is selected from the group consisting of: paclitaxel, docetaxel, carboplatin, cisplatin, gemcitabine, tamoxifen, Herceptin, Cetuximab, Tarceva, Iressa, bevacizumab, navelbine, IMC-1C11, SU5416 and SU6688.

Another embodiment of this invention is directed to a method of treating cancer in a patient in need of such treatment, said method comprising administering to said patient an effective amount of at least one (1, 2 or 3, or 1 or 2, or 1, and usually 1) compound of formula 1.0 and an effective amount of a chemotherapeutic agent, wherein the chemotherapeutic agent is selected from the group consisting of: paclitaxel, docetaxel, carboplatin, cisplatin, navelbine, gemcitabine, and Herceptin.

Another embodiment of this invention is directed to a method of treating cancer in a patient in need of such treatment, said method comprising administering to said patient an effective amount of at least one (1, 2 or 3, or 1 or 2, or 1, and usually 1) compound of formula 1.0 and an effective amount of a chemotherapeutic agent, wherein the chemotherapeutic agent is selected from the group consisting of: Cyclophasphamide, 5-Fluorouracil, Temozolomide, Vincristine, Cisplatin, Carboplatin, and Gemcitabine.

Another embodiment of this invention is directed to a method of treating cancer in a patient in need of such treatment, said method comprising administering to said patient an effective amount of at least one (1, 2 or 3, or 1 or 2, or 1, and usually 1) compound of formula 1.0 and an effective amount of a chemotherapeutic agent, wherein the chemotherapeutic agent is selected from the group consisting of: Gemcitabine, Cisplatin and Carboplatin.

This invention also provides a method of treating cancer in a patient in need of such treatment, said treatment comprising administering to said patient a therapeutically effective amount at least one (e.g., 1, 2 or 3, or 1 or 2, or 1, and usually 1) compound of formula 1.0, and therapeutically effective amounts of at least one (e.g., 1, 2 or 3, or 1 or 2, or 2, or 1) chemotherapeutic agent selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

This invention also provides a method of treating cancer in a patient in need of such treatment, said treatment comprising administering to said patient a therapeutically effective amount at least one (e.g., 1, 2 or 3, or 1 or 2, or 1, and usually 1) compound of formula 1.0, and therapeutically effective amounts of at least two (e.g., 2 or 3, or 2, and usually 2) different antineoplastic agents selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

This invention also provides a method of treating cancer in a patient in need of such treatment, said method comprising administering to said patient therapeutically effective amounts at least one (e.g., 1, 2 or 3, or 1 or 2, or 1, and usually 1) compound of formula 1.0, and an antineoplastic agent selected from the group consisting of: (1) EGF inhibitors that are antibodies, (2) EGF inhibitors that are small molecules, (3) VEGF inhibitors that are antibodies, and (4) VEGF inhibitors that are small molecules. Radiation therapy can also be used in conjunction with this above combination therapy, i.e., the above method using a combination of compounds of the invention and antineoplastic agent can also comprise the administration of a therapeutically effect amount of radiation.

This invention also provides a method of treating leukemias (e.g., acute myeloid leukemia (AML), and chronic myeloid leukemia (CML)) in a patient in need of such treatment, said method comprising administering to said patient therapeutically effective amounts at least one (e.g., 1, 2 or 3, or 1 or 2, or 1, and usually 1) compound of formula 1.0, and: (1) Gleevec and interferon to treat CML; (2) Gleevec and pegylated interferon to treat CML; (3) Gleevec to treat CML; (4) an anti-tumor nucleoside derivative (e.g., Ara-C) to treat AML; or (5) an anti-tumor nucleoside derivative (e.g., Ara-C) in combination with an anthracycline to treat AML.

This invention also provides a method of treating non-Hodgkin's lymphoma in a patient in need of such treatment, said method comprising administering therapeutically effective amounts at least one (e.g., 1, 2 or 3, or 1 or 2, or 1, and usually 1) compound of formula 1.0 and: (1) a biologic (e.g., Rituxan); (2) a biologic (e.g., Rituxan) and an anti-tumor nucleoside derivative (e.g., Fludarabine); or (3) Genasense (antisense to BCL-2).

This invention also provides a method of treating multiple myeloma in a patient in need of such treatment, said method comprising administering to said patient therapeutically effective amounts of at least one (e.g., 1, 2 or 3, or 1 or 2, or 1, and usually 1) compound of formula 1.0 and: (1) a proteosome inhibitor (e.g., PS-341 from Millenium); or (2) Thalidomide (or related imid).

This invention also provides a method of treating cancer in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, (3) EGF inhibitors that are antibodies, (4) EGF inhibitors that are small molecules, (5) VEGF inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, and (12) antibodies that are inhibitors of αVβ3 integrins.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, (3) EGF inhibitors that are antibodies, (4) EGF inhibitors that are small molecules, (5) VEGF inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, and (12) antibodies that are inhibitors of αVβ3 integrins.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, (3) anti-tumor nucleoside derivatives, (4) topoisomerase inhibitors, and (5) vinca alkaloids.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0, (b) carboplatin, and (c) paclitaxel.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0, (b) cisplatin, and (c) gemcitabine.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0, (b) carboplatin, and (c) gemcitabine.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0, (b) Carboplatin, and (c) Docetaxel.

This invention also provides a method of treating cancer in a patient in need of such treatment, said method comprising administering therapeutically effective amounts of: (a) at least one (e.g., 1, 2 or 3, or 1 or 2, or 1, and usually 1) compound of formula 1.0, and (b) an antineoplastic agent selected from the group consisting of: (1) EGF inhibitors that are antibodies, (2) EGF inhibitors that are small molecules, (3). VEGF inhibitors that are antibodies, (4) VEGF kinase inhibitors that are small molecules.

This invention also provides a method of treating squamous cell cancer of the head and neck, in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, and (2) platinum coordinator compounds.

This invention also provides a method of treating squamous cell cancer of the head and neck, in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, and (3) anti-tumor nucleoside derivatives (e.g., 5-Fluorouracil).

This invention also provides a method of treating CML in a patient in need of such treatment, said method comprising administering therapeutically effective amounts of: (a) at least one (e.g., 1, 2 or 3, or 1 or 2, or 1, and usually 1) compound of formula 1.0, (b) Gleevec, and (c) interferon (e.g., Intron-A).

This invention also provides a method of treating CML in a patient in need of such treatment comprising administering therapeutically effective amounts of: (a) at least one (e.g., 1, 2 or 3, or 1 or 2, or 1, and usually 1) compound of formula 1.0, (b) Gleevec; and (c) pegylated interferon (e.g., Peg-Intron, and Pegasys).

This invention also provides a method of treating CML in a patient in need of such treatment comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 161) and (b) Gleevec.

This invention also provides a method of treating CMML in a patient in need of such treatment, said method comprising administering to said patient therapeutically effective amounts of at least one (e.g., 1, 2 or 3, or 1 or 2, or 1, and usually 1) compound of formula 1.0.

This invention also provides a method of treating AML in a patient in need of such treatment, said method comprising administering to said patient therapeutically effective amounts of: (a) at least one (e.g., 1, 2 or 3, or 1 or 2, or 1, and usually 1) compound of formula 1.0, and (b) an anti-tumor nucleoside derivative (e.g., Cytarabine (i.e., Ara-C)).

This invention also provides a method of treating AML in a patient in need of such treatment, said method comprising administering to said patient therapeutically effective amounts of: (a) at least one (e.g., 1, 2 or 3, or 1 or 2, or 1, and usually 1) compound of formula 1.0, (b) an anti-tumor nucleoside derivative (e.g., Cytarabine (i.e., Ara-C)), and (c) an anthracycline.

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0, and (b) Rituximab (Rituxan).

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0, (b) Rituximab (Rituxan), and (c) an anti-tumor nucleoside derivative (e.g., Fludarabine (i.e., F-ara-A).

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0, and (b) Genasense (antisense to BCL-2).

This invention also provides a method of treating multiple myeloma in a patient in need of such treatment, said method comprising administering therapeutically effective amounts of: (a) at least one (e.g., 1, 2 or 3, or 1 or 2, or 1, and usually 1) compound of formula 1.0, and (b) a proteosome inhibitor (e.g., PS-341 (Millenium)).

This invention also provides a method of treating multiple myeloma in a patient in need of such treatment, said method comprising administering to said patient therapeutically effective amounts of: (a) at least one (e.g., 1, 2 or 3, or 1 or 2, or 1, and usually 1) compound of formula 1.0, and (b) Thalidomide or related imid.

This invention also provides a method of treating multiple myeloma in a patient in need of such treatment, said method comprising administering therapeutically effective amounts of: (a) at least one (e.g., 1, 2 or 3, or 1 or 2, or 1, and usually 1) compound of formula 1.0, and (b) Thalidomide.

This invention is also directed to the methods of treating cancer described herein, particularly those described above, wherein in addition to the administration of the compound of formula 1.0 and antineoplastic agents, radiation therapy is also administered prior to, during, or after the treatment cycle.

This invention also provides a method for treating cancer (e.g., lung cancer, prostate cancer and myeloid leukemias) in a patient in need of such treatment, said method comprising administering to said patient (1) an effective amount of at least one (e.g., 1, 2 or 3, or 1 or 2, or 1, and usually 1) compound of formula 1.0, in combination with (2) at least one (e.g., 1, 2 or 3, or 1 or 2, or 2, or 1) antineoplastic agent, microtubule affecting agent and/or radiation therapy.

This invention also provides a method of treating cancer in a patient in need of such treatment, said method comprising administering to said patient an effective amount of at least one (e.g., 1, 2 or 3, or 1 or 2, or 1, and usually 1) compound of formula 1.0 in combination with an effective amount of at least one (e.g., 1, 2 or 3, or 1 or 2, or 1, and usually 1) signal transduction inhibitor.

Thus, in one example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once per week in an amount of about 50 to about 100 mg/m², and in another example about 60 to about 80 mg/m², and (3) Carboplatin is administered once per week in an amount to provide an AUC of about 2 to about 3.

In another example (e.g., treating non small cell lung cancer): (1) the compound of formula 1.0 is administered in an amount of about 50 mg to about 200 mg twice a day, and in another example about 75 mg to about 125 mg administered twice a day, and yet in another example about 100 mg administered twice a day, (2) Paclitaxel (e.g., Taxol® is administered once per week in an amount of about 50 to about 100 mg/m², and in another example about 60 to about 80 mg/m², and (3) Cisplatin is administered once per week in an amount of about 20 to about 40 mg/m².

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere®) is administered once per week in an amount of about 10 to about 45 mg/m², and (3) Carboplatin is administered once per week in an amount to provide an AUC of about 2 to about 3.

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere®) is administered once per week in an amount of about 10 to about 45 mg/m², and (3) Cisplatin is administered once per week in an amount of about 20 to about 40 mg/m².

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once every three weeks in an amount of about 150 to about 250 mg/m², and in another example about 175 to about 225 mg/m², and in yet another example 175 mg/m², and (3) Carboplatin is administered once every three weeks in an amount to provide an AUC of about 5 to about 8, and in another example 6.

In another example of treating non small cell lung cancer: (1) the compound of formula 1.0 is administered in an amount of 100 mg administered twice a day, (2) Paclitaxel (e.g., Taxol® is administered once every three weeks in an amount of 175 mg/m², and (3) Carboplatin is administered once every three weeks in an amount to provide an AUC of 6.

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once every three weeks in an amount of about 150 to about 250 mg/m², and in another example about 175 to about 225 mg/m², and (3) Cisplatin is administered once every three weeks in an amount of about 60 to about 100 mg/m².

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere® is administered once every three weeks in an amount of about 50 to about 100 mg/m², and (3) Carboplatin is administered once every three weeks in an amount to provide an AUC of about 5 to about 8.

In another example (e.g., treating non small cell lung cancer): (1) the compound of formula 1.0 is administered in an amount of about 50 mg to about 200 mg twice a day, in another example about 75 mg to about 125 mg administered twice a day, and in yet another example about 100 mg administered twice a day, (2) Docetaxel (e.g., Taxotere® is administered once every three weeks in an amount of about 50 to about 100 mg/m², and (3) Cisplatin is administered once every three weeks in an amount of about 60 to about 100 mg/m².

In another example for treating non small cell lung cancer using the compounds of formula 1.0, Docetaxel and Carboplatin: (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere® is administered once every three weeks in an amount of about 75 mg/m², and (3) Carboplatin is administered once every three weeks in an amount to provide an AUC of about 6.

In another example of the treatments of non-small cell lung cancer described above the Docetaxel (e.g., Taxotere® and Cisplatin, the Docetaxel (e.g., Taxotere® and Carboplatin, the Paclitaxel (e.g., Taxol® and Carboplatin, or the Paclitaxel (e.g., Taxol® and Cisplatin are administered on the same day.

In another example (e.g., CML): (1) the compound of formula 1.0 is administered in an amount of about 100 mg to about 200 mg administered twice a day, (2) Gleevec is administered in an amount of about 400 to about 800 mg/day orally, and (3) interferon (Intron-A) is administered in an amount of about 5 to about 20 million IU three times per week.

In another example (e.g., CML): (1) the compound of formula 1.0 is administered in an amount of about 100 mg to about 200 mg administered twice a day, (2) Gleevec is administered in an amount of about 400 to about 800 mg/day orally, and (3) pegylated interferon (Peg-Intron or Pegasys) is administered in an amount of about 3 to about 6 micrograms/kg/day.

In another example (e.g., non-Hodgkin's lymphoma): (1) the compound of formula 1.0 is administered in an amount of about 50 mg to about 200 mg twice a day, and in another example about 75 mg to about 125 mg administered twice a day, and in yet another example about 100 mg administered twice a day, and (2) Genasense (antisense to BCL-2) is administered as a continuous IV infusion at a dose of about 2 to about 5 mg/kg/day (e.g., 3 mg/kg/day) for 5 to 7 days every 3 to 4 weeks.

In another example (e.g., multiple myeloma): (1) the compound of formula 1.0 is administered in an amount of about 50 mg to about 200 mg twice a day, and in another example about 75 mg to about 125 mg administered twice a day, and in yet another example about 100 mg administered twice a day, and (2) the proteosome inhibitor (e.g., PS-341-Millenium) is administered in an amount of about 1.5 mg/m^{2.} twice weekly for two consecutive weeks with a one week rest period.

In another example (e.g., multiple myeloma): (1) the compound of formula 1.0 is administered in an amount of about 50 mg to about 200 mg twice a day, and in another example about 75 mg to about 125 mg administered twice a day, and in yet another example about 100 mg administered twice a day, and (2) the Thalidomide (or related imid) is administered orally in an amount of about 200 to about 800 mg/day, with dosing being continuous until relapse or toxicity.

In one embodiment of the methods of treating cancer of this invention, the chemotherapeutic agents are selected from the group consisting of: paclitaxel, docetaxel, carboplatin, cisplatin, gemcitabine, tamoxifen, Herceptin, Cetuximab, Tarceva, Iressa, bevacizumab, navelbine, IMC-1C11, SU5416 and SU6688.

In another embodiment of the methods of treating cancer of this invention, the chemotherapeutic agents are selected from the group consisting of: paclitaxel, docetaxel, carboplatin, cisplatin, navelbine, gemcitabine, and Herceptin.

Thus, one embodiment of this invention is directed to a method of treating cancer comprising administering to a patient in need of such treatment therapeutically effective amounts of the compound of formula 1.0, a taxane, and a platinum coordination compound.

Another embodiment of this invention is directed to a method of treating cancer comprising administering to a patient in need of such treatment therapeutically effective amounts of the compound of formula 1.0, a taxane, and a platinum coordination compound, wherein said compound of formula 1.0 is administered every day, said taxane is administered once per week per cycle, and said platinum coordinator compound is administered once per week per cycle. In another embodiment the treatment is for one to four weeks per cycle.

Another embodiment of this invention is directed to a method of treating cancer comprising administering to a patient in need of such treatment therapeutically effective amounts of the compound of formula 1.0, a taxane, and a platinum coordination compound, wherein said compound of formula 1.0 is administered every day, said taxane is administered once every three weeks per cycle, and said platinum coordinator compound is administered once every three weeks per cycle. In another embodiment the treatment is for one to three weeks per cycle.

Another embodiment of this invention is directed to a method of treating cancer comprising administering to a patient in need of such treatment therapeutically effective amounts of the compound of formula 1.0, paclitaxel, and carboplatin. In another embodiment, said compound of formula 1.0 is administered every day, said paclitaxel is administered once per week per cycle, and said carboplatin is administered once per week per cycle. In another embodiment the treatment is for one to four weeks per cycle.

Another embodiment of this invention is directed to a method of treating cancer comprising administering to a patient in need of such treatment therapeutically effective amounts of the compound of formula 1.0, paclitaxel, and carboplatin. In another embodiment, said compound of formula 1.0 is administered every day, said paclitaxel is administered once every three weeks per cycle, and said carboplatin is administered once every three weeks per cycle. In another embodiment the treatment is for one to three weeks per cycle.

Another embodiment of this invention is directed to a method for treating non small cell lung cancer in a patient in need of such treatment comprising administering daily a therapeutically effective amount of the compound of formula 1.0, administering a therapeutically effective amount of carboplatin once a week per cycle, and administering a therapeutically effective amount of paclitaxel once a week per cycle, wherein the treatment is given for one to four weeks per cycle. In another embodiment said compound of formula 1.0 is administered twice per day. In another embodiment said carboplatin and said paclitaxel are administered on the same day, and in another embodiment said carboplatin and said paclitaxel are administered consecutively, and in another embodiment said carboplatin is administered after said paclitaxel.

Another embodiment of this invention is directed to a method for treating non small cell lung cancer in a patient in need of such treatment comprising administering daily a therapeutically effective amount of a compound of formula 1.0, administering a therapeutically effective amount of carboplatin once every three weeks per cycle, and administering a therapeutically effective amount of paclitaxel once every three weeks per cycle, wherein the treatment is given for one to three weeks. In another embodiment compound of formula 1.0 is administered twice per day. In another embodiment said carboplatin and said paclitaxel are administered on the same day, and in another embodiment said carboplatin and said paclitaxel are administered consecutively, and in another embodiment said carboplatin is administered after said paclitaxel.

Another embodiment of this invention is directed to a method for treating non small cell lung cancer in a patient in need of such treatment comprising administering about 50 to about 200 mg of a compound of formula 1.0 twice a day, administering carboplatin once per week per cycle in an amount to provide an AUC of about 2 to about 8 (and in another embodiment about 2 to about 3), and administering once per week per cycle about 60 to about 300 mg/m² (and in another embodiment about 50 to 100 mg/m², and in yet another embodiment about 60 to about 80 mg/m²) of paclitaxel, wherein the treatment is given for one to four weeks per cycle. In another embodiment said compound of formula 1.0 is administered in amount of about 75 to about 125 mg twice a day, and in another embodiment about 100 mg twice a day. In another embodiment said carboplatin and said paclitaxel are administered on the same day, and in another embodiment said carboplatin and said paclitaxel are administered consecutively, and in another embodiment said carboplatin is administered after said paclitaxel.

In another embodiment, this invention is directed to a method for treating non small cell lung cancer in a patient in need of such treatment comprising administering about 50 to about 200 mg of a compound of formula 1.0 twice a day, administering carboplatin once every three weeks per cycle in an amount to provide an AUC of about 2 to about 8 (in another embodiment about 5 to about 8, and in another embodiment 6), and administering once every three weeks per cycle about 150 to about 250 mg/m² (and in another embodiment about 175 to about 225 mg/m², and in another embodiment 175 mg/m²) of paclitaxel, wherein the treatment is given for one to three weeks. In another embodiment said compound of formula 1.0 is administered in an amount of about 75 to about 125 mg twice a day, and in another embodiment about 100 mg twice a day. In another embodiment said carboplatin and said paclitaxel are administered on the same day, and in another embodiment said carboplatin and said paclitaxel are administered consecutively, and in another embodiment said carboplatin is administered after said paclitaxel.

Other embodiments of this invention are directed to methods of treating cancer as described in the above embodiments (i.e., the embodiments directed to treating cancer and to treating non small cell lung cancer with a taxane and platinum coordinator compound) except that in place of paclitaxel and carboplatin the taxanes and platinum coordinator compounds used together in the methods are: (1) docetaxel (Taxotere®) and cisplatin; (2) paclitaxel and cisplatin; and (3) docetaxel and carboplatin. In another embodiment of the methods of this invention cisplatin is used in amounts of about 30 to about 100 mg/m². In the another embodiment of the methods of this invention docetaxel is used in amounts of about 30 to about 100 mg/m².

In another embodiment this invention is directed to a method of treating cancer comprising administering to a patient in need of such treatment therapeutically effective amounts of a compound of formula 1.0, a taxane, and an EGF inhibitor that is an antibody. In another embodiment the taxane used is paclitaxel, and the EGF inhibitor is a HER2 antibody (in one embodiment Herceptin) or Cetuximab, and in another embodiment Herceptin is used. The length of treatment, and the amounts and administration of said compound of formula 1.0 and the taxane are as described in the embodiments above. The EGF inhibitor that is an antibody is administered once a week per cycle, and in another embodiment is administered on the same day as the taxane, and in another embodiment is administered consecutively with the taxane. For example, Herceptin is administered in a loading dose of about 3 to about 5 mg/m² (in another embodiment about 4 mg/m²), and then is administered in a maintenance dose of about 2 mg/m² once per week per cycle for the remainder of the treatment cycle (usually the cycle is 1 to 4 weeks). In one embodiment the cancer treated is breast cancer.

In another embodiment this invention is directed to a method of treating cancer comprising administering to a patient in need of such treatment therapeutically effective amounts of: (1) a compound of formula 1.0, (2) a taxane, and (3) an antineoplastic agent selected from the group consisting of: (a) an EGF inhibitor that is a small molecule, (b) a VEGF inhibitor that is an antibody, and (c) a VEGF kinase inhibitor that is a small molecule. In another embodiment, the taxane paclitaxel or docetaxel is used. In another embodiment the antineoplastic agent is selected from the group consisting of: tarceva, Iressa, bevacizumab, SU5416, SU6688 and BAY 43-9006. The length of treatment, and the amounts and administration of said compound of formula 1.0 and the taxane are as described in the embodiments above. The VEGF kinase inhibitor that is an antibody is usually given once per week per cycle. The EGF and VEGF inhibitors that are small molecules are usually given daily per cycle. In another embodiment, the VEGF inhibitor that is an antibody is given on the same day as the taxane, and in another embodiment is administered concurrently with the taxane. In another embodiment, when the EGF inhibitor that is a small molecule or the VEGF inhibitor that is a small molecule is administered on the same day as the taxane, the administration is concurrently with the taxane. The EGF or VEGF kinase inhibitor is generally administered in an amount of about 10 to about 500 mg/m².

In another embodiment this invention is directed to a method of treating cancer comprising administering to a patient in need of such treatment therapeutically effective amounts of a compound of formula 1.0, an anti-tumor nucleoside derivative, and a platinum coordination compound.

Another embodiment of this invention is directed to a method of treating cancer comprising administering to a patient in need of such treatment therapeutically effective amounts of a compound of formula 1.0, an anti-tumor nucleoside derivative, and a platinum coordination compound, wherein said compound of formula 1.0 is administered every day, said anti-tumor nucleoside derivative is administered once per week per cycle, and said platinum coordinator compound is administered once per week per cycle. Although the treatment can be for one to four weeks per cycle, in one embodiment the treatment is for one to seven weeks per cycle.

Another embodiment of this invention is directed to a method of treating cancer comprising administering to a patient in need of such treatment therapeutically effective amounts of a compound of formula 1.0, an anti-tumor nucleoside derivative, and a platinum coordination compound, wherein said compound of formula 1.0 is administered every day, said an anti-tumor nucleoside derivative is administered once per week per cycle, and said platinum coordinator compound is administered once every three weeks per cycle. Although the treatment can be for one to four weeks per cycle, in one embodiment the treatment is for one to seven weeks per cycle.

Another embodiment of this invention is directed to a method of treating cancer comprising administering to a patient in need of such treatment therapeutically effective amounts of a compound of formula 1.0, gemcitabine, and cisplatin. In another embodiment, said compound of formula 1.0 is administered every day, said gemcitabine is administered once per week per cycle, and said cisplatin is administered once per week per cycle. In one embodiment the treatment is for one to seven weeks per cycle.

Another embodiment of this invention is directed to a method of treating cancer comprising administering to a patient in need of such treatment therapeutically effective amounts of a compound of formula 1.0, gemcitabine, and cisplatin. In another embodiment, said compound of formula 1.0 is administered every day, said gemcitabine is administered once per week per cycle, and said cisplatin is administered once every three weeks per cycle. In another embodiment the treatment is for one to seven weeks.

Another embodiment of this invention is directed to a method of treating cancer comprising administering to a patient in need of such treatment therapeutically effective amounts of a compound of formula 1.0, gemcitabine, and carboplatin. In another embodiment said compound of formula 1.0 is administered every day, said gemcitabine is administered once per week per cycle, and said carboplatin is administered once per week per cycle. In another embodiment the treatment is for one to seven weeks per cycle.

Another embodiment of this invention is directed to a method of treating cancer comprising administering to a patient in need of such treatment therapeutically effective amounts of a compound of formula 1.0, gemcitabine, and carboplatin. In another embodiment said compound of formula 1.0 is administered every day, said gemcitabine is administered once per week per cycle, and said carboplatin is administered once every three weeks per cycle. In another embodiment the treatment is for one to seven weeks per cycle.

In the above embodiments using gemcitabine, the compound of formula 1.0 and the platinum coordinator compound are administered as described above for the embodiments using taxanes. Gemcitabine is administered in an amount of about 500 to about 1250 mg/m². In one embodiment the gemcitabine is administered on the same day as the platinum coordinator compound, and in another embodiment consecutively with the platinum coordinator compound, and in another embodiment the gemcitabine is administered after the platinum coordinator compound.

Another embodiment of this invention is directed to a method of treating cancer in a patient in need of such treatment comprising administering to said patient a compound of formula 1.0 and an antineoplastic agent selected from: (1) EGF inhibitors that are antibodies, (2) EGF inhibitors that are small molecules, (3) VEGF inhibitors that are antibodies, and (4) VEGF kinase inhibitors that are small molecules all as described above. The treatment is for one to seven weeks per cycle, and generally for one to four weeks per cycle. The compound of formula 1.0 is administered in the same manner as described above for the other embodiments of this invention. The small molecule antineoplastic agents are usually administered daily, and the antibody antineoplastic agents are usually administered once per week per cycle. In one embodiment the antineoplastic agents are selected from the group consisting of: Herceptin, Cetuximab, Tarceva, Iressa, bevacizumab, IMC-1C11, SU5416, SU6688 and BAY 43-9006.

In the embodiments of this invention wherein a platinum coordinator compound is used as well as at least one other antineoplastic agent, and these drugs are administered consecutively, the platinum coordinator compound is generally administered after the other antineoplastic agents have been administered.

Other embodiments of this invention include the administration of a therapeutically effective amount of radiation to the patient in addition to the administration of a compound of formula 1.0 and antineoplastic agents in the embodiments described above. Radiation is administered according to techniques and protocols well know to those skilled in the art.

Another embodiment of this invention is directed to a pharmaceutical composition comprising at least two different chemotherapeutic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising a compound of formula 1.0 and at least two different antineoplastic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising a compound of formula 1.0 and at least one antineoplastic agent and a pharmaceutically acceptable carrier for intravenous administration. Preferably the pharmaceutically acceptable carrier is an isotonic saline solution (0.9% NaCl) or a dextrose solution (e.g., 5% dextrose).

Other embodiments of this invention are directed to the use of a combination of at least one (e.g., one) compound of formula 1.0 and drugs for the treatment of breast cancer, i.e., this invention is directed to a combination therapy for the treatment of breast cancer. Those skilled in the art will appreciate that the compounds of formula 1.0 and drugs are generally administered as individual pharmaceutical compositions. The use of a pharmaceutical composition comprising more than one drug is within the scope of this invention.

Thus, another embodiment of this invention is directed to a method of treating (or preventing) breast cancer (i.e., postmenopausal and premenopausal breast cancer, e.g., hormone-dependent breast cancer) in a patient in need of such treatment comprising administering to said patient a therapeutically effective amount of at least one (e.g., one) compound of formula 1.0 and a therapeutically effective amount of at least one antihormonal agent selected from the group consisting of: (a) aromatase inhibitors, (b) antiestrogens, and (c) LHRH analogues; and said treatment optionally including the administration of at least one chemotherapeutic agent.

The compound of formula 1.0 is preferably administered orally, and in one embodiment is administered in capsule form.

Examples of aromatase inhibitors include but are not limited to: Anastrozole (e.g., Arimidex), Letrozole (e.g., Femara), Exemestane (Aromasin), Fadrozole and Formestane (e.g., Lentaron).

Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: Goserelin (e.g., Zoladex) and Leuprolide (e.g., Leuprolide Acetate, such as Lupron or Lupron Depot).

Examples of chemotherapeutic agents include but are not limited to: Trastuzumab (e.g., Herceptin), Gefitinib (e.g., Iressa), Erlotinib (e.g., Erlotinib HCl, such as Tarceva), Bevacizumab (e.g., Avastin), Cetuximab (e.g., Erbitux), and Bortezomib (e.g., Velcade).

Preferably, when more than one antihormonal agent is used, each agent is selected from a different category of agent. For example, one agent is an aromatase inhibitor (e.g., Anastrozole, Letrozole, or Exemestane) and one agent is an antiestrogen (e.g., Tamoxifen or Fulvestrant).

Another embodiment of this invention is directed to a method of treating or preventing breast cancer in a patient in need of such treatment wherein said treatment comprises administering a therapeutically effective amount of at least one (e.g., one) compound of formula 1.0 and at least one antihormonal agent selected from the group consisting of: (a) aromatase inhibitors, (b) antiestrogens, and (c) LHRH analogues; and administering an effective amount of at least one chemotherapeutic agent.

Another embodiment of this invention is directed to a method of treating or preventing breast cancer in a patient in need of such treatment wherein said treatment comprises administering a therapeutically effective amount of at least one (e.g., one) compound of formula 1.0 and at least one antihormonal agent selected from the group consisting of: (a) aromatase inhibitors, (b) antiestrogens, and (c) LHRH analogues.

Another embodiment of this invention is directed to a method of treating or preventing breast cancer in a patient in need of such treatment wherein said treatment comprises administering a therapeutically effective amount of at least one (e.g., one) compound of formula 1.0 and at least one antihormonal agent selected from the group consisting of: (a) aromatase inhibitors, and (b) antiestrogens.

Another embodiment of this invention is directed to a method of treating or preventing breast cancer in a patient in need of such treatment wherein said treatment comprises administering a therapeutically effective amount of at least one (e.g., one) compound of formula 1.0, at least one antihormonal agent selected from the group consisting of: (a) aromatase inhibitors and (b) antiestrogens; and at least one chemotherapeutic agent.

Another embodiment of this invention is directed to a method of treating or preventing breast cancer in a patient in need of such treatment wherein said treatment comprises administering a therapeutically effective amount of at least one (e.g., one) compound of formula 1.0 and at least one aromatase inhibitor.

Another embodiment of this invention is directed to a method of treating or preventing breast cancer in a patient in need of such treatment wherein said treatment comprises administering a therapeutically effective amount of at least one (e.g., one) compound of formula 1.0, at least one aromatase inhibitor, and at least one chemotherapeutic agent.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene, and (c) LHRH analogues that are selected from the group consisting of: Goserelin and Leuprolide; and administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene, and (c) LHRH analogues that are selected from the group consisting of: Goserelin and Leuprolide.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, and (b) antiestrogens that are selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene; and administering an effective amount of at least one chemotherapeutic agents are selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one aromatase inhibitor selected from the group consisting of Anastrozole, Letrozole, Exemestane, Fadrozole and Formestane.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one aromatase inhibitor that is selected from the group consisting of Anastrozole, Letrozole, Exemestane, Fadrozole and Formestane; and (3) administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one aromatase inhibitor; and (3) at least one LHRH analogue.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one antiestrogen; and (3) at least one LHRH analogue.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one aromatase inhibitor that is selected from the group consisting of Anastrozole, Letrozole, Exemestane, Fadrozole and Formestane; and (3) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one antiestrogen that is selected from the group consisting of: Tamoxifen, Fulvestrant, Raloxifene, and Acolbifene; and (3) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of at least one (e.g., one) compound of formula 1.0 and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0 and Letrazole.

Another embodiment of this invention is directed to a method of treating or preventing breast cancer in a patient in need of such treatment wherein said treatment comprises administering a therapeutically effective amount of at least one (e.g., one) compound of formula 1.0 and Exemestane.

Another embodiment of this invention is directed to a method of treating or preventing breast cancer in a patient in need of such treatment wherein said treatment comprises administering a therapeutically effective amount of at least one (e.g., one) compound of formula 1.0 and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0 and Formestane.

Another embodiment of this invention is directed to a method of treating or preventing breast cancer in a patient in need of such treatment wherein said treatment comprises administering a therapeutically effective amount of at least one (e.g., one) compound of formula 1.0 and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0 and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 and Goserelin.

Another embodiment of this invention is directed to a method of treating or preventing breast cancer in a patient in need of such treatment wherein said treatment comprises administering a therapeutically effective amount of at least one (e.g., one) compound of formula 1.0 and Leuprolide.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Raloxifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Goserelin, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Leuprolein, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Goserelin, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Goserelin, and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide, and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Letrozole.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Exemestane.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Formestane.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Letrozole.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Exemestane.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Formestane.

Another embodiment of this invention is directed to the treatment or prevention of breast cancer in a patient in need of such treatment, said treatment comprising the administration of a therapeutically effective amount of at least one (e.g., one) compound of formula 1.0 and Anastrozole.

Another embodiment of this invention is directed to the treatment or prevention of breast cancer in a patient in need of such treatment, said treatment comprising the administration of a therapeutically effective amount of at least one (e.g., one) compound of formula 1.0 and Letrozole.

Another embodiment of this invention is directed to the treatment or prevention of breast cancer in a patient in need of such treatment, said treatment comprising the administration of a therapeutically effective amount of at least one (e.g., one) compound of formula 1.0 and Exemestane.

Another embodiment of this invention is directed to the treatment or prevention of breast cancer in a patient in need of such treatment, said treatment comprising the administration of a therapeutically effective amount of at least one (e.g., one) compound of formula 1.0 and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0 and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0, Anastrozole, and Fulvestrant.

Another embodiment of this invention is directed to the treatment or prevention of breast cancer in a patient in need of such treatment, said treatment comprising the administration of a therapeutically effective amount of at least one compound of formula I (e.g., one), Letrozole, and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0, Exemestane, and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0, Anastrozole, and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0, Letrozole, and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0, Exemestane, and Tamoxifen.

Other embodiments of this invention are directed to any of the above described embodiments for the treatment of Breast Cancer wherein the chemotherapeutic agent is Trastuzumab.

Other embodiments of this invention are directed to any of the above described embodiments for the treatment or prevention of Breast Cancer wherein the method is directed to the treatment of breast cancer.

The compound of formula 1.0, antihormonal agents and chemotherapeutic agents can be administered concurrently or sequentially.

The antihormonal agents and optional chemotherapeutic agents are administered according to their protocols, dosage amounts, and dosage forms that are well know to those skilled in the art (e.g., the Physician's Desk Reference or published literature). For example, for Tamoxifen, Fulvestrant, Raloxifene, Anastrozole, Letrozole, Exemestane, Leuprolide and Goserelin, see the Physician's Desk Reference, 57^{th} Edition, 2003, published by Thomas PDR at Montvale, N.J. 07645-1742, the disclosure of which is incorporated herein by reference thereto.

In general, in the embodiments directed to the methods of treating Breast Cancer: (1) the compound of formula 1.0 can be administered daily (e.g., once per day, and in one embodiment twice a day), (2) the aromatase inhibitors can be administered in accordance with the known protocol for the aromatase inhibitor used (e.g., once per day), (3) the antiestrogens can be administered in accordance with the known protocol for the antiestrogen used (e.g., from once a day to once a month), (4) the LHRH analogue can be administered in accordance with the known protocol for the LHRH analogue used (e.g., once a month to once every three months), and (5) the chemotherapeutic agent can be administered in accordance with the known protocol for the chemotherapeutic agent used (e.g., from once a day to once a week).

Radiation therapy, if administered in the above treatments for breast cancer, is generally administered according to known protocols before administration of the compound of formula 1.0, antihormonal agents and optional chemotherapeutic agents.

Treatment according to the methods of treating breast cancer is continuous (i.e., a continuous dosing schedule is followed). The treatment is continued until there is a complete response, or until the skilled clinician determines that the patient is not benefiting from the treatment (for example, when there is disease progression).

The continuous treatment protocol for breast cancer can be changed to a discontinuous treatment schedule if, in the judgment of the skilled clinician, the patient would benefit from a discontinuous treatment schedule with one or more of the administered drugs. For example, the compound of formula 1.0 can be given using a discontinous treatment schedule while the remaining drugs used in the treatment are given as described herein. An example of a discontinuous treatment protocol for the compound of formula 1.0 is a repeating cycle of three weeks with the compound of formula 1.0 followed by one week without the compound of formula 1.0.

After a complete response is achieved with the breast cancer treatment, maintenance therapy with the compound of formula 1.0 can be continued using the dosing described in the methods of this invention. Maintenance therapy can also include administration of the antihormonal agents using the dosing described in the methods of this invention. Maintenance therapy can just be with the antihormonal agents. For example, after a complete response is achieved, an aromatase inhibitor (e.g., Anastrozole, Letrozole or Exemestane) can be continued for up to five years. Or, for example, an antiestrogen, e.g., Tamoxifen, may be used for up to five years after a complete response is achieved. Or, for example, an antiestrogen (e.g., Tamoxifen) can be used for up to five years after a complete response is achieved followed by the use of an aromatase inhibitor (e.g., Anastrozole, Letrozole or Exemestane) for up to five years.

In the embodiments directed to the treatment of breast cancer described above, the compound of formula 1.0 is administered continuously in a total daily dose of about 100 mg to about 600 mg. Usually this amount is administered in divided doses, and in one embodiment this amount is administered twice a day. In one embodiment the compound of formula 1.0 is dosed twice a day in an amount of about 50 mg to about 300 mg per dose. In another embodiment the compound of formula 1.0 is dosed twice a day in an amount of about 100 mg to about 200 mg per dose. Examples include the compound of formula 1.0 being dosed twice a day at 100 mg per dose. Examples also include the compound of formula 1.0 being dosed twice a day at 200 mg per dose.

Anastrozole is administered p.o. and is dosed once a day in amounts of about 0.5 to about 10 mg per dose, and in one embodiment in an amount of about 1.0 mg per dose.

Letrozole is administered p.o. and is dosed once a day in amounts of about 1.0 to about 10 mg per dose, and in one embodiment in an amount of about 2.5 mg per dose.

Exemestane is administered p.o. and is dosed once a day in amounts of about 10 to about 50 mg per dose, and in one embodiment in an amount of about 25 mg per dose.

Fadrozole is administered p.o. and is dosed twice a day in amounts of about 0.5 to about 10 mg per dose, and in one embodiment in an amount of about 2.0 mg per dose.

Formestane is administered i.m. and is dosed once every two weeks in amounts of about 100 to about 500 mg per dose, and in one embodiment in an amount of about 250 mg per dose.

Tamoxifen is administered p.o. and is dosed once a day in amounts of about 10 to about 100 mg per dose, and in one embodiment in an amount of about 20 mg per dose.

Fulvestrant is administered i.m. and is dosed once a month in amounts of about 100 to about 1000 mg per dose, and in one embodiment in an amount of about 250 mg per dose.

Raloxifene is administered p.o. and is dosed once a day in amounts of about 10 to about 120 mg per dose, and in one embodiment in an amount of about 60 mg per dose.

Acolbifene is administered p.o. and is dosed once a day in amounts of about 5 to about 20 mg per dose, and in one embodiment in an amount of about 20 mg per dose.

Goserelin is administered s.c. and is dosed once a month, or once every three months, in amounts of about 2 to about 20 mg per dose, and in one embodiment in an amount of about 3.6 mg per dose when administered once a month, and in another embodiment in an amount of about 10.8 mg per dose when administered once every three months.

Leuprolide is administered s.c. and is dosed once a month, or once every three months, in amounts of about 2 to about 20 mg per dose, and in one embodiment in an amount of about 3.75 mg per dose when administered once a month, and in another embodiment in an amount of about 11.25 mg per dose when administered once every three months.

Trastuzumab is administered by i.v. and is dosed once a week in amounts of about 2 to about 20 mpk per dose, and in one embodiment in an amount of about 2 mpk per dose. Trastuzumab is generally initially administered in a loading dose that is generally twice the dose of the weekly dose. Thus, for example, a 4 mpk loading dose is administered and then dosing is 2 mpk per dose per week.

Gefitinib is administered p.o. and is dosed once a day in amounts of about 100 to about 1000 mg per dose, and in one embodiment in an amount of about 250 mg per dose.

Erlotinib is administered p.o. and is dosed once a day in amounts of about 100 to about 500 mg per dose, and in one embodiment in an amount of about 150 mg per dose.

Bevacizumab is administered i.v. and is dosed once every two weeks in amounts of about 2.5 to about 15 mg per kilogram of body weight per dose, and in one embodiment in an amount of about 10 mg per kilogram per dose.

Cetuximab is administered i.v. and is dosed once a week in amounts of about 200 to about 500 mg per meter squared dose, and in one embodiment in an amount of about 250 mg per meter squared per dose.

Bortezomib is administered i.v. and is dosed twice a week for 2 weeks followed by a 10 day rest period (21 day treatment cycle) for a maximum of 8 treatment cycles in amounts of about 1.0 to about 2.5 mg per meter squared per dose, and in one embodiment in an amount of about 1.3 mg per meter squared per dose.

Thus in one embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Anastrozole p.o. in an amount of about 0.5 to about 10 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Anastrozole in an amount of about 1.0 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Letrozole p.o. in an amount of about 1.0 to about 10 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Letrozole p.o. in an amount of about 2.5 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Exemestane p.o. in an amount of about 10 to about 50 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Exemestane in an amount of about 25 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, and (2) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, and (2) Tamoxifen p.o. in an amount of about 20 mg per dose wherein each dose is given once a day.

In other embodiments of the invention breast cancer is treated in a patient in need of such treatment wherein said treatment comprises the administration of the compound of formula 1.0, one of the aromatase inhibitors (e.g., Anastrozole, Letrozole, or Exemestane, and in one embodiment Anastrozole), and one of the antiestrogens (e.g., Fulvestrant or Tamoxifen), wherein the compound of formula 1.0, aromatase inhibitor and antiestrogen are administered in the dosages described above.

Thus, for example in another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Anastrozole p.o. in an amount of about 0.5 to about 10 mg per dose wherein each dose is given once a day, and (3) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 p.o in an amount of about 100 to 200 mg per dose, wherein each dose is administered twice a day, (2) Anastrozole p.o. in an amount of about 1.0 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Letrozole p.o in an amount of about 1.0 to about 10 mg per dose wherein each dose is given once a day, and (3) Fulvestrant in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Letrozole p.o. in an amount of about 2.5 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Exemestane p.o. in an amount of about 10 to about 50 mg per dose wherein each dose is given once a day, and (3) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Exemestane p.o. in an amount of about 25 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Anastrozole p.o. in an amount of about 0.5 to about 10 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Anastrozole p.o. in an amount of about 1.0 mg per dose wherein each dose is given once a day, and (3) Tamoxifen p.o. in an amount of about 20 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Letrozole p.o. in an amount of about 1.0 to about 10 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Letrozole p.o. in an amount of about 2.5 mg per dose wherein each dose is given once a day, and (3) Tamoxifen p.o. in an amount of about 20 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Exemestane p.o. in an amount of about 10 to about 50 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Exemestane p.o. in an amount of about 25 mg per dose wherein each dose is given once a day, and (3) Tamoxifen p.o. in an amount of about 20 mg per dose wherein each dose is given once a day.

Those skilled in the art will appreciate that when other combinations of antihormonal agents are used, the individual antihormonal agent is used in the amounts specified above for that individual antihormonal agent.

Other embodiments of the treatment of Breast Cancer are directed to the methods of treating Breast Cancer described above wherein the compound of formula 1.0 is dosed twice a day in an amount of about 100 mg per dose.

Other embodiments of the treatment of Breast Cancer are directed to the methods of treating Breast Cancer described above wherein the compound of formula 1.0 is dosed twice a day in an amount of about 200 mg per dose.

Other embodiments of the treatment of Breast Cancer are directed to the methods of treating Breast Cancer described above wherein a chemotherapeutic agent is administered in addition to the compound of formula 1.0 and antihormonal agent (or antihormonal agents). In these embodiments the dosage ranges of the compound of formula 1.0 and antihormonal agents are as those described above in the combination therapies, or those described above for the individual compound of formula I and antihormonal agents, and the dosages of the chemotherapeutic agents are those described above for the individual chemotherapeutic agent. The dosages for the chemotherapeutic agents are well known in the art.

Other embodiments of this invention are directed to pharmaceutical compositions comprising the compound of formula 1.0 and at least one antihormonal agent and a pharmaceutically acceptable carrier.

Other embodiments of this invention are directed to pharmaceutical compositions comprising the compound of formula 1.0, at least one antihormonal agent, at least one chemotherapeutic agent, and a pharmaceutically acceptable carrier.

Other embodiments of this invention are directed to pharmaceutical compositions comprising the compound of formula 1.0, at least one chemotherapeutic agent, and a pharmaceutically acceptable carrier.

Those skilled in the art will appreciate that the compounds (drugs) used in the methods of this invention are available to the skilled clinician in pharmaceutical compositions (dosage forms) from the manufacturer and are used in those compositions. So, the recitation of the compound or class of compounds in the above described methods can be replaced with a recitation of a pharmaceutical composition comprising the particular compound or class of compounds. For example, the embodiment directed to a method of treating cancer comprising administering to a patient in need of such treatment therapeutically effective amounts of the compound of formula 1.0, a taxane, and a platinum coordination compound, includes within its scope a method of treating cancer comprising administering to a patient in need of such treatment therapeutically effective amounts of a pharmaceutical composition comprising the compound of formula 1.0, a pharmaceutical composition comprising a taxane, and a pharmaceutical composition comprising a platinum coordination compound.

Those skilled in the art will recognize that the actual dosages and protocols for administration employed in the methods of this invention may be varied according to the judgment of the skilled clinician. The actual dosage employed may be varied depending upon the requirements of the patient and the severity of the condition being treated. Determination of the proper dosage for a particular situation is within the skill of the art. A determination to vary the dosages and protocols for administration may be made after the skilled clinician takes into account such factors as the patient's age, condition and size, as well as the severity of the disease (e.g., cancer) being treated and the response of the patient to the treatment.

The amount and frequency of administration of the compound of formula 1.0 and the chemotherapeutic agents (in the methods wherein cancer is treated) will be regulated according to the judgment of the attending clinician (physician) considering such factors as age, condition and size of the patient as well as severity of the disease (e.g., cancer) being treated.

The chemotherapeutic agent can be administered according to therapeutic protocols well known in the art. It will be apparent to those skilled in the art that the administration of the chemotherapeutic agent can be varied depending on the cancer being treated and the known effects of the chemotherapeutic agent on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the cancer to the administered therapeutic agents.

The initial administration can be made according to established protocols known in the art, and then, based upon the observed effects, the dosage, modes of administration and times of administration can be modified by the skilled clinician.

The particular choice of chemotherapeutic agent will depend upon the diagnosis of the attending physicians and their judgement of the condition of the patient and the appropriate treatment protocol.

The determination of the order of administration, and the number of repetitions of administration of the chemotherapeutic agent during a treatment protocol, is well within the knowledge of the skilled physician after evaluation of the cancer being treated and the condition of the patient.

Thus, in accordance with experience and knowledge, the practicing physician can modify each protocol for the administration of an chemotherapeutic agent according to the individual patient's needs, as the treatment proceeds. All such modifications are within the scope of the present invention.

The particular choice of antihormonal agents, optional chemotherapeutic agents and optional radiation will depend upon the diagnosis of the attending physicians and their judgment of the condition of the patient and the appropriate treatment protocol.

The determination of the order of administration, and the number of repetitions of administration of the antihormonal agents, optional chemotherapeutic agents and optional radiation during a treatment protocol, is well within the knowledge of the skilled physician after evaluation of the breast cancer being treated and the condition of the patient.

Thus, in accordance with experience and knowledge, the practicing physician can modify each protocol for the administration of antihormonal agents, optional chemotherapeutic agents and optional radiation according to the individual patient's needs, as the treatment proceeds. All such modifications are within the scope of the present invention.

The attending clinician, in judging whether treatment is effective at the dosage administered, will consider the general well-being of the patient as well as more definite signs such as relief of the disease (e.g. for cancer, the relief of cancer-related symptoms (e.g., pain, cough (for lung cancer), and shortness of breath (for lung cancer), inhibition of tumor growth, actual shrinkage of the tumor, or inhibition of metastasis). Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington: The Science and Practice of Pharmacy, 20^{th} Edition, (2000), Lippincott Williams & Wilkins, Baltimore, Md.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparations subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg according to the particular application.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in two to four divided doses.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of the formula: 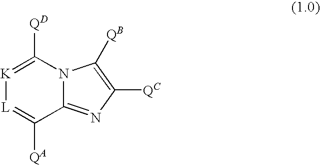
or the pharmaceutically acceptable salts, esters, and solvates thereof, wherein:
K is selected from the group consisting of: CH, N, —C(alkyl)-, —C(aryl)-, —C(halo)-, and —C(R^{C})— wherein R^{C} is selected from the group consisting of: 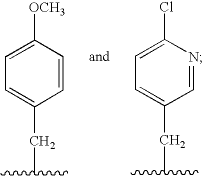
L is CH or N;
Q^{A} is selected from the group consisting of:
(A) —C(O)NR¹R²;
(B) —N(R¹⁴)₂;
(C) unsubstituted heteroaryl;
(D) substituted heteroaryl, and wherein said substituted heteroaryl is substituted with one or more substituents selected from the group consisting of: (1) halo, (2) heteroaryl, benzo fused heteroaryl, (3) heterocycloalkyl, (4) benzodioxolyl, (5) aryl, (6) substituted aryl wherein the substituent is —S(O)₂alkyl, (7) alkyl, (8) —CF₃; 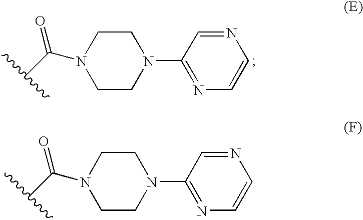
substituted with one or more substituents selected from the group consisting of:
(1) -(alkylene)₁₋₆-heterocycloalkyl,
(2) aryl,
(3) substituted aryl,
(4) —C(O)R¹¹,
(5) —C(O)-aryl (e.g. —C(O)phenyl), and
(6) -(alkylene)₁₋₆-N(R¹²)₂, and
wherein said substituted aryl moiety (3) (e.g., substituted phenyl) is substituted with one or more substitutents independently selected from the group consisting of: halo (e.g., Cl and F), and —CN; 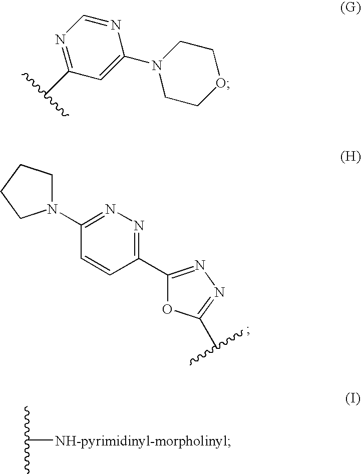
(J) H;
(K) —C(O)-heterocycloalkyl-heteroaryl;
(L) —C(O)-piperazinyl-(alkylene)₁₋₆-substituted aryl wherein the substituents are independently selected from halo;
(M) —C(O)-heterocycloalkyl-(alkylene)₁₋₆-heterocycloalkyl;
(N) —C(O)-piperazinyl-(alkylene)₁₋₆-heteroaryl;
(O) alkyl (e.g., C₁₋₆alkyl);
(P) —C(O)-heterocycloalkyl wherein said heterocycloalkyl is substituted with -(alkylene)₁₋₆-N(R¹²)₂ wherein each R¹² is independently selected;
(O) —C(O)-heterocycloalkyl-(alkylene)₁₋₆-(alkyl substituted heterocycloalkyl);
(R) -(alkylene)₁₋₆-benzo[1,3]dioxolyl;
(S) -(alkylene)₁₋₆-N(R¹)(R²) wherein R¹ and R² are as defined above,
(T) —NH-heteroaryl-heteroaryl
(U) —NH-(fused heteroarylheteroaryl);
(V) —NH-(substituted heteroaryl);
(W) —NH-heteroaryl-NH-heterocycloalkyl;
(X) biaryl;
(Y) biheteroaryl;
(Z) substituted biaryl; and
(AA) substituted biheteroaryl;
Q^{B} is selected from the group consisting of:
(A) —C(O)NR¹⁵R¹⁶;
(B) —C(O)—R²¹;
(C) H;
(D) —N(R¹²)₂, wherein each R¹² is independently selected;
(E) —CH₂OH;
(F) —CH₂OCH₃;
(G) —CH₂SCH₃,
(H) —CH₂N(R^{B}) wherein each R^{B} is independently selected from the group consisting of: H, alkyl, cycloalkyl, heterocycloalkyl, heteroaryl, and aryl;
(I) —N(R¹²)₂ wherein each R¹² is independently selected;
(J) —NH—C(O)-alkyl;
(K) —NH—C(O)-(hydroxyl substituted alkyl);
(L) —NH—S(O)₂-alkyl;
(M) —NH—C(O)—C(═CH₂)CH₂(CH₃)₂;
(N) —NH—C(O)—C(O)—CH₂(CH₃)₂;
(O) alkyl; and
(P) aryl;
Q^{C} is selected from the group consisting of:
(A) heteroaryl;
(B) heterocycloalkyl;
(C) H;
(D) alkyl;
(E) —C(O)N(R¹²)₂;
(F) cycloalkyl;
(G) halo;
(H) —CN,
(I) —CF₃;
(J) —CH₂CF₃;
(K) —SR^{A} wherein R^{A} is selected from the group consisting of: alkyl, cycloalkyl, heterocycloalkyl, heteroaryl, and aryl;
(L) —N(R^{B})₂ wherein each R^{B} is independently selected from the group consisting of: H, alkyl, cycloalkyl, heterocycloalkyl, heteroaryl, and aryl;
(M) —OR^{A} wherein R^{A} is as defined above;
(N) —C(O)R^{A} wherein R^{A} is as defined above;
(O) aryl;
(P) arylalkyl-;
(O) heteroarylalkyl-;
(R) substituted aryl and wherein there are 1 to 3 substituents on said substituted aryl;
(S) substituted heteroaryl;
(T) substituted heteroarylalkyl;
(U) substituted aralkyl; 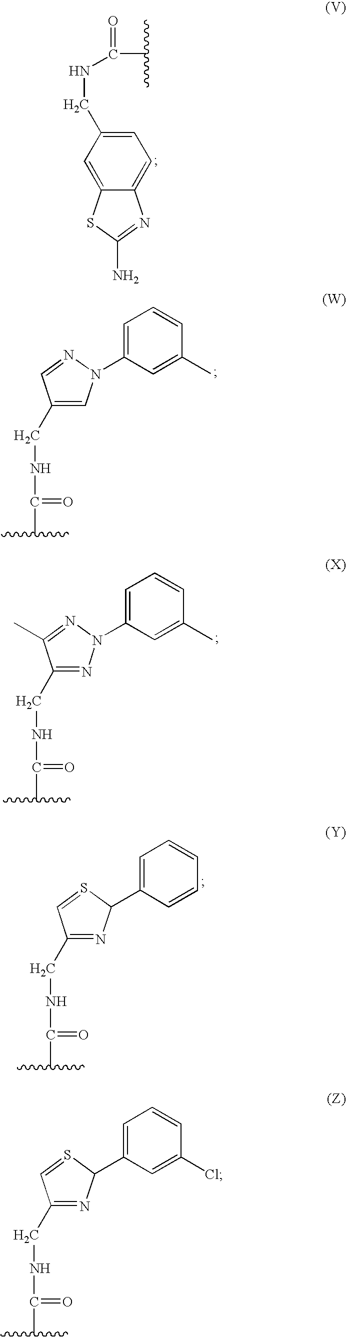 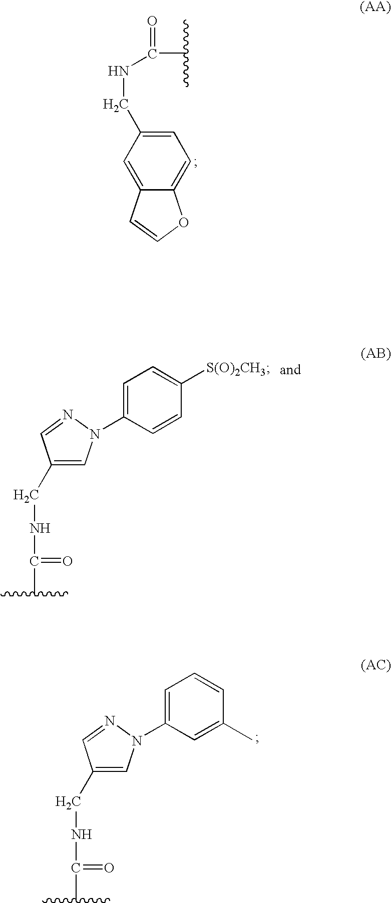
Q^{D} is selected from the group consisting of: H and alkyl;
R¹ and R² are each independently selected from the group consisting of:
(1) H;
(2) unsubstituted -(alkylene)₁₋₆-benzoheteroaryl;
(3) substituted -(alkylene)₁₋₆-benzoheteroaryl, and wherein:
(a) either the alkylene or benzoheteroaryl moieties are substituted, or both the alkylene and benzoheteroaryl moieties are substituted,
(b) when the alkylene moiety is substituted the substitutents are independently selected from the group consisting of: alkyl, cycloalkyl, —C(O)OH, —C(O)Oalkyl, and wherein the substituted alkylene moieties comprise R or S stereochemical centers, and
(c) when the benzoheteroaryl moiety is substituted the substituents are independently selected from the group consisting of: (1) —NH₂, (2) —NH(alkyl), (3) —NHC(O)(alkyl), (4) alkyl, (5) —S(alkyl), and (6) heteroaryl;
(4) unsubstituted -(alkylene)₁₋₆-heteroaryl;
(5) substituted -(alkylene)₁₋₆-heteroaryl substituted with one or more substitutents independently selected from the group consisting of: halo, —C(O)N(R⁶)₂, and —NHS(O)₂R⁷, wherein each R⁶ is independently selected from the group consisting of H and alkyl, and wherein R⁷ is alkyl;
(6) unsubstituted -benzoheteroaryl;
(7) substituted -benzoheteroaryl, and wherein said substituted benzoheteroaryl is substituted with one or more substitutents independently selected from the group consisting of: heteroaryl, heterocycloalkyl, and —S(alkyl);
(8) heteroaryl;
(9) substituted heteroaryl substituted with one or more substitutents independently selected from the group consisting of: heteroaryl, heterocycloalkyl, and —S(alkyl);
(10) aryl;
(11) substituted aryl substituted with one or more substitutents independently selected from the group consisting of: heteroaryl, heterocycloalkyl, and —S(alkyl); 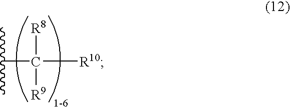
(13) unsubstituted -(alkylene)₁₋₆-heterocycloalkyl;
(14) substituted -(alkylene)₁₋₆-heterocycloalkyl, and wherein said substituted moiety (14) is substituted with one or more substituents selected from the group consisting of —SO₂R¹³, and wherein R¹³ is selected from the group consisting of:
(a) alkyl,
(b) aryl,
(c) substituted aryl,
(d) heteroaryl,
(e) substituted heteroaryl,
(f) -(alkylene)₁₋₆heterocylcoalkyl,
(g) -(alkylene)₁₋₆-heteroaryl,
(h) —C(O)R¹¹,
(i) —C(O)aryl,
(j) -(alkylene)₁₋₆N(R¹²)₂, and
(k) wherein said substituted groups (c) and (e) of said moiety (14) are independently substituted with one or more substitutents independently selected from the group consisting of: (i) halo, (ii) —OH, (iii) —OR¹¹, (iv) —CF₃, (v) —S(O)₂R¹¹, and (vi) —S(O)₂N(R¹²)₂;
(15) -(alkylene)₁₋₆-bicyclic bridged cycloalkyl;
(16) -(alkylene)₁₋₆-bicyclic bridged heterocycloalkyl;
(17) -(alkylene)₁₋₆-bicyclic bridged spirocycloalkyl;
(18) -(alkylene)₁₋₆-bicyclic bridged spiroheterocycloalkyl;
(19) -(alkylene)₁₋₆-(substituted heteroaryl) wherein the substituents on said heteroaryl are independently selected from the group consisting of: —C(O)N(R¹²)₂ wherein each R¹² is independently selected, —NHS(O)₂-alkyl;
(20) -cycloalkyl-benzodioxolyl;
(21) -cycloalkyl-(substituted aryl) wherein the substituents are independently selected from the group consisting of methylene dioxy and —S(O)₂CH₃;
(22) alkyl;
(23) cycloalkyl;
(24) alkyl;
(25) hydroxyl substituted alkyl;
R⁸ and R⁹ are each independently selected from the group consisting of: H, alkyl, cycloalkyl, C(O)OH, —C(O)OR¹¹, substituted alkyl and substituted cycloalkyl;
R¹⁰ is selected from the group consisting of:
(a) aryl (e.g., phenyl),
(b) substituted aryl,
(c) heteroaryl,
(d) substituted heteroaryl,
(e) benzoheteroaryl,
(f) heterocycloalkyl,
(g) substituted heterocycloalkyl,
(h) -piperidinyl-S(O)₂-(alkyl substituted heteroaryl),
(i) -piperidinyl-S(O)₂-aryl-heteroaryl),
(j) -piperidinyl-C(O)-pyridyl,
(k) -piperidinyl-C(O)-alkyl,
(l) -piperidinyl-(substituted aryl) wherein said substituents are independently selected from the groups consisting of: halo and CN,
(m) -piperidinyl-pyridyl,
(n) benzodioxolyl,
(o) -heteroaryl-NH-cycloalkylalkyl, and
(p) -heteroaryl-NH-cycloalkyl;
wherein said substituted R⁸, R⁹ and R¹⁰ groups are substituted with one or more substitutents independently selected from the group consisting of:
(a) halo,
(b) —OH,
(c) —OR¹¹,
(d) —CF₃,
(e) heterocycloalkyl,
(f) substituted heterocycloalkyl,
(g) heteroaryl,
(h) substituted heteroaryl,
(i) aryl,
(j) substituted aryl,
(k) —C(O)OR¹¹,
(l) —N(R¹²)₂,
(m) alkyl,
(n) cycloalkyl,
(o) —SO₂R¹¹,
(p) —N(alkyl)-cycloalkyl,
(q) —C(O)OH,
(r) benzoheteroaryl, and
(s) substituted benzoheteroaryl,
and wherein said substituted groups (f), (h), and (j) are independently substituted with one or more substitutents independently selected from the group consisting of:
(i) halo,
(ii) —OH,
(iii) —OR¹¹,
(iv) —CF₃,
(v) —S(O)₂R¹¹,
(vi) —S(O)₂N(R¹²)₂,
(vii)=0,
(viii) substituted benzoheteroaryl substituted with 1 to 3 groups independently selected from the group consisting of: C₁ to C₆ alkyl, cycloalkyl, —NH₂, —NH(C₁ to C₆ alkyl), and —N(C₁ to C₆ alkyl)₂ wherein each alkyl is independently selected,
(ix) alkyl,
(x) CN,
(xi) cycloalkyl,
(xii) —C(O)-morpholinyl,
(xiii) amino,
(xiv) alkylamino,
(xv) and dialkylamino;
R¹¹ is alkyl;
each R¹² is independently selected from the group consisting of H, alkyl, and hydroxyl substituted alkyl;
each R¹⁴ is independently selected from the group consisting of: H, —C(O)—(CH₂)₁₋₂-aryl, substituted aryl, and benzodioxyl, and wherein said substituted aryl is substituted with one or more substituents independently selected from the group consisting of: halo, —OH, —OR¹¹ (wherein R¹¹ is as previously described), —CN, —CF₃, alkyl, —NH₂ and —NO₂;
R¹⁵ and R¹⁶ are each independently selected from the group consisting of:
(1) hydroxyl substituted alkyl,
(2) alkyl,
(3) —SO₂R¹¹,
(4) unsubstituted -(alkylene)₁₋₆-R¹⁷ wherein R¹⁷ is selected from the group consisting of: (a) heterocycloalkyl, (b) heteroaryl, and (c) cycloalkyl, 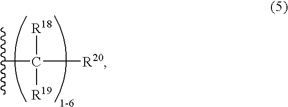
(6) —C(O)-alkyl,
(7) substituted alkyl wherein said substituents are selected from the group consisting of —OR¹¹,
(8) saturated bicyclic rings,
(9) hydroxyl substituted -(alkylene)₁₋₆-cycloalkyl,
(10) H,
(11) heterocycloalkyl substituted with heterocycloalkyl,
(12) cycloalkyl, and
(13) cycloalkyl substituted with 1 to 2 —OH groups,
(14) -(alkylene)₁₋₆-aryl,
(15) -(alkylene)₁₋₆-aryl substituted with 1 to 2 substituents independently selected from the group consisting —OH and alkylamino,
(16) -(alkylene)₁₋₆-heteroaryl substituted with 1 to 2 substituents independently selected from the group consisting —OH and alkylamino;
(17) heterocycloalkyl,
(18) substituted heterocycloalkyl,
(19) -(alkylene)₁₋₆-heterocycloalkyl wherein said alkylene moiety is substituted with hydroxyl,
(20) -(alkylene)₁₋₆-C(O)OH,
(21) fused hydroxyl substituted benzocycloalkyl
(22) fused hydroxyl substituted arylheteroaryl,
(23) hydroxyl-(alkylene)₁₋₆-cycloalkyl,
(24) hydroxyl-(alkylene)₁₋₆-bridged cycloalkyl,
(25) hydroxyl-(alkylene)₁₋₆-spirocycloalkyl,
(26) hydroxyl-(alkylene)₁₋₆-bridged heterocycloalkyl,
(27) hydroxyl-(alkylene)₁₋₆-spiroheterocycloalkyl, and
(28) heterocycloalkyl;
each R¹⁸ and each R¹⁹ is independently selected from the group consisting of: H, alkyl, and hydroxyalkyl-;
R²⁰ is selected from the group consisting of:
(a) aryl,
(b) substituted aryl,
(c) heteroaryl,
(d) benzo fused heteroaryl,
(e) -(alkylene)₁₋₆-heteroaryl,
(f) -(alkylene)₁₋₆aryl,
(g) -(alkylene)₁₋₆aryl substituted with —OH,
(h) benzoheteroaryl-(alkylene)₁₋₆-,
(i) cycloalkylalkyl,
(j) cycloalkyl (e.g., hexyl),
(k) heterocycloalkyl,
(l) -(alkylene)₁₋₆aryl substituted with halo,
(m) -(alkylene)₁₋₆-S-alkyl,
(n)-(alkylene)₁₋₆-O-alkyl,
(o)-(alkylene)₁₋₆-N-alkyl,
(p) -(alkylene)₁₋₆-cycloalkyl,
and wherein said substituted aryl is substituted with one or more substituents independently selected from the group consisting of: halo, —OH, —OR¹¹, —CN, —CF₃, alkyl, —NH₂ and —NO₂;
R²¹ is selected from the group consisting of:
(1) heterocycloalkyl,
(2) benzo fused cycloalkyl,
(3) cycloalkyl,
(4) multicyclic cycloalkyl ring, and
(5) substituted heterocycloalkyl substituted with one or more substituents independently selected from the group consisting of: (a) hydroxyl substituted alkyl, (b) —OH, (c)-(alkylene)₁₋₆C(O)O-(alkyl)₁₋₆, (d) aryl, and (e) substituted aryl wherein said substituted aryl is substituted with one or more substitutents independently selected from the group consisting of: halo, and
(6) heterocycloalkyl substituted with 1 to 3 substituents selected from the group consisting of: amino, alkylamino, dialkylamino, and —C(O)alkyl,
(7) heterocycloalkyl,
(8) hydroxy substituted heterocycloalkyl), and
(9) —OH.

2. The compound of claim 1 wherein K is CH.

3. The compound of claim 1 wherein L is CH.

4. The compound of claim 1 wherein K is CH and L is CH.

5. The compound of claim 1 wherein one of R¹ and R² is H, and the other is selected from the group consisting of:

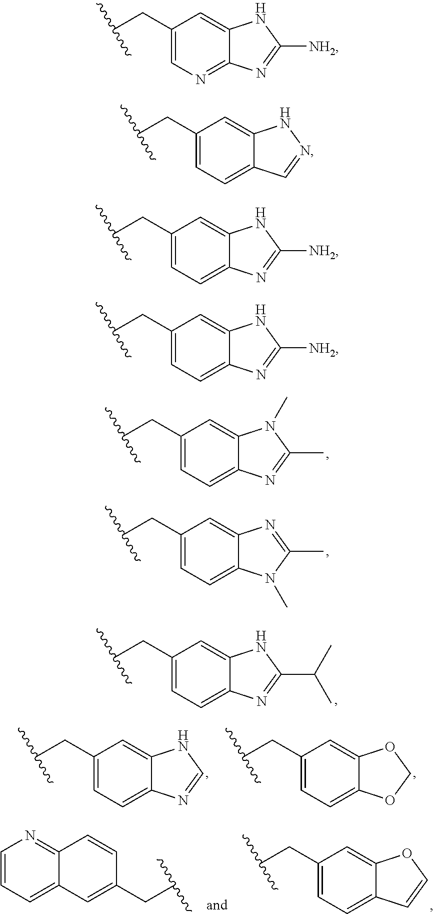

6. The compound of claim 1 wherein Q^{A} is selected from the group consisting of:

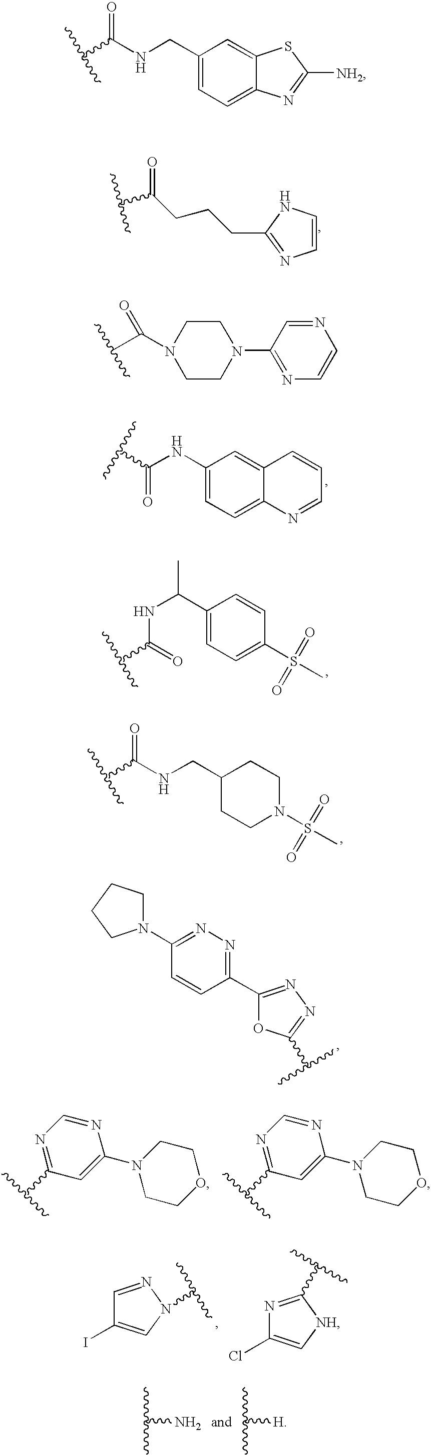

7. The compound of claim 1 wherein Q^{A} is:

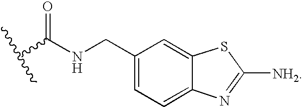

8. The compound of claim 1 wherein Q^{A} is:

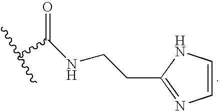

9. The compound of claim 1 wherein Q^{A} is:

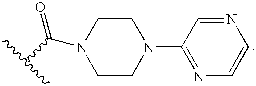

10. The compound of claim 1 wherein Q^{A} is:

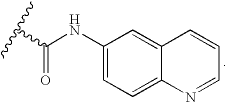

11. The compound of claim 1 wherein Q^{A} is:

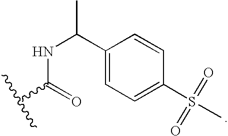

12. The compound of claim 1 wherein Q^{A} is:

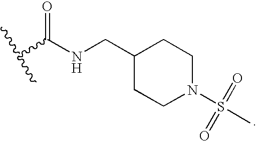

13. The compound of claim 1 wherein Q^{A} is:

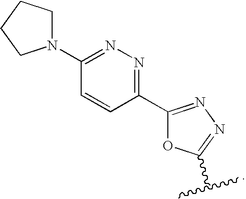

14. The compound of claim 1 wherein Q^{A} is:

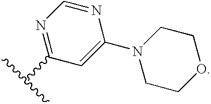

15. The compound of claim 1 wherein Q^{A} is:

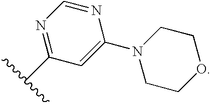

16. The compound of claim 1 wherein Q^{A} is:

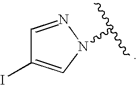

17. The compound of claim 1 wherein Q^{A} is:

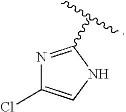

18. The compound of claim 1 wherein Q^{A} is —NH₂.

19. The compound of claim 1 wherein Q^{A} is H.

20. The compound of claim 1 wherein Q^{B} is selected from the group consisting of:

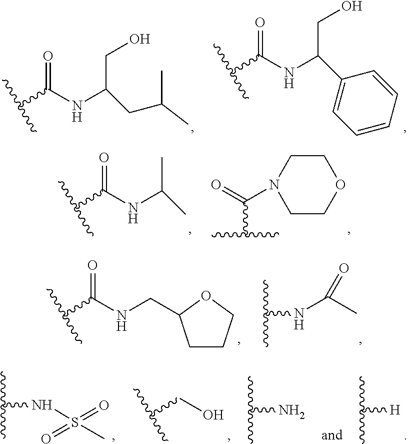

21. The compound of claim 1 wherein Q^{B} is:

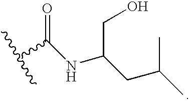

22. The compound of claim 1 wherein Q^{B} is:

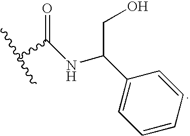

23. The compound of claim 1 wherein Q^{B} is:

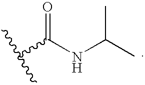

24. The compound of claim 1 wherein Q^{B} is

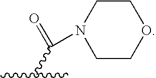

25. The compound of claim 1 wherein Q^{B} is:

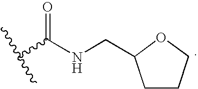

26. The compound of claim 1 wherein Q^{B} is:

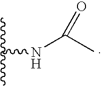

27. The compound of claim 1 wherein Q^{B} is:

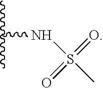

28. The compound of claim 1 wherein Q^{B} is:

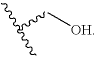

29. The compound of claim 1 wherein Q^{B} is —NH₂.

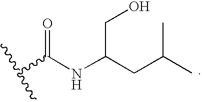

30. The compound of claim 1 wherein Q^{B} is H.

31. The compound of claim 1 wherein Q^{B} is selected from the group consisting of:

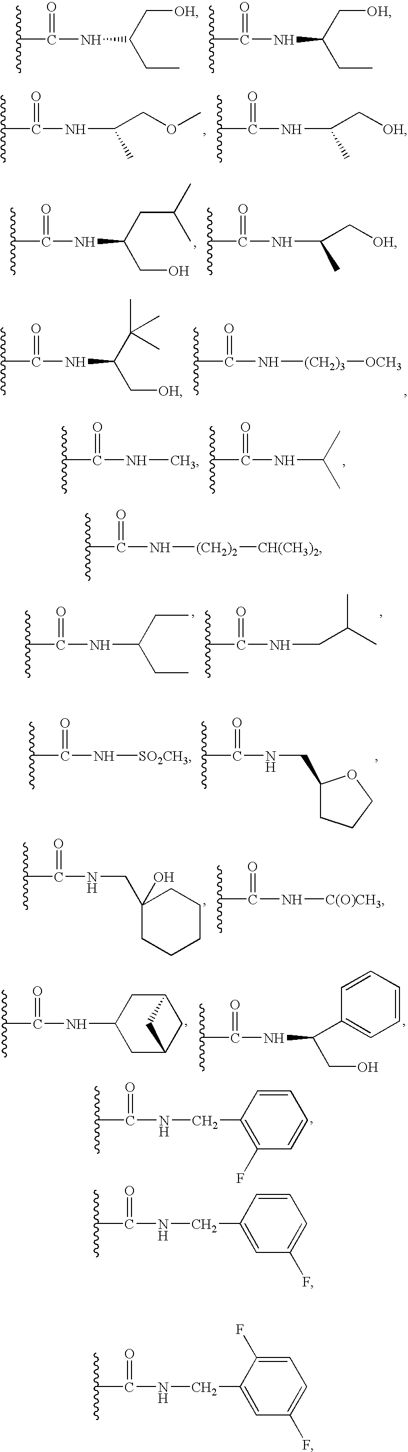

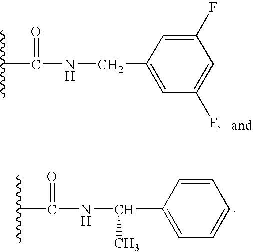

32. The compound of claim 1 wherein Q^{C} is selected from the group consisting of:

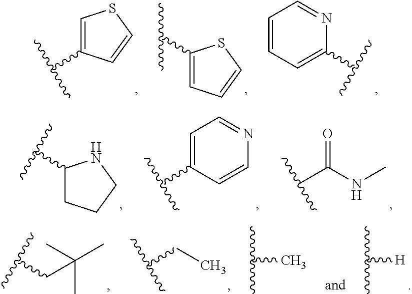

33. The compound of claim 1 wherein Q^{C} is:

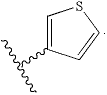

34. The compound of claim 1 wherein Q^{C} is:

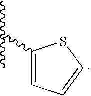

35. The compound of claim 1 wherein Q^{C} is:

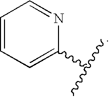

36. The compound of claim 1 wherein Q^{C} is:

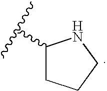

37. The compound of claim 1 wherein Q^{C} is:

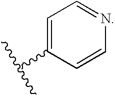

38. The compound of claim 1 wherein Q^{C} is:

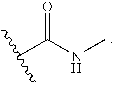

39. The compound of claim 1 wherein Q^{C} is:

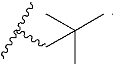

40. The compound of claim 1 wherein Q^{C} is:

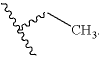

41. The compound of claim 1 wherein Q^{C} is —CH₃.

42. The compound of claim 1 wherein Q^{C} is H.

43. The compound of claim 1 wherein said compound is selected from the group consisting of Compound Numbers: 13-94, 97-101, 111-125, 130, 131, 139, 140, 150, 154-158, 162, 167, 170-246, 271-289, 291-303, 305-307, 321-324, 326-328, 350-354, 404-410, 444-506, 542-546, 573-576, 578, 584, 588, 590, 593, 597, 598-600, 605-629, 635, 647, 650-652, 659, 664-665, 673-680, 686, 691, 692, 699, 703, 720-727, 734, 736, 740-743, 755, 756, 762-776, 780, 784, and 791-794.

44. The compound of claim 1 wherein said compound is selected from the group consisting of: Compound Numbers: 14, 16, 17, 22, 46, 47, 48, 56, 69, 93, 94, 111-115, 117, 118, 130, 131, 139, 140, 150, 154, -158, 204-206, 209, 213, 215-220, 224, 238, 242, 274, 277, 279, 280, 283, 285, 291, 292, 296, 298, 299, 300, 301, 305, 306, 307, 323, 324, 326, 327, 405, 445, 451, 452, 453, 456, 457, 460-466, 471, 472, 477, 478, 479, 480, 481, 483, 484, 485, 489, 490, 491, 502, 542, 543, 544, 545, 593, 598, 599, 605, 623-629, 647, 650, 651, 652, and 664.

45. The compound of claim 1 wherein said compound is selected from the group consisting of Compound Numbers: 14, 16, 112, 114, 139, 156, 216, 218, 219, 277, 296, 300, 306, 307, 463, 478, 479, 483, 485, 491, 502, 598, 629, 647, 650, 651, and 652.

46. The compound of claim 1 wherein said compound is selected from the group consistent of: Compound Numbers: 112, 478, 479, 502, 629, 651, and 652.

47. A pharmaceutical composition comprising an effective amount of a compound of claim 1 and a pharmaceutically acceptable carrier.

48. A method of treating a JNK1 mediated disease or condition in a patient in need of such treatment comprising administering to said patient an effective amount of at least one compound of claim 1.

49. A method of treating a ERK mediated disease or condition in a patient in need of such treatment comprising administering to said patient an effective amount of at least one compound of claim 1.

50. A method of treating cancer in a patient in need of such treatment comprising administering to said patient an effective amount of at least one compound of claim 1.

51. A method of treating a disease or condition in a patient in need of such treatment comprising administering to said patient an effective amount of at least one pound of claim 1, and wherein said disease or condition is selected from the group consisting of: inflammation, rheumatoid arthritis, asthma, multiple sclerosis, inflammatory bowel disease, psorisis, diabetes, autoimmune disorders, metabolic diseases, neurological diseases, pain and cardiovascular diseases.
